(19) United States
(12) Patent Application Publication
(10) Pub. No.: US 2024/0360153 A1
(43) Pub. Date: Oct. 31, 2024

(54) SELECTIVE RAPAMYCIN ANALOGS AND USES THEREOF

(71) Applicant: Regeneron Pharmaceuticals, Inc., Tarrytown, NY (US)

(72) Inventors: David Glass, Cortlandt Manor, NY (US); Joseph P. Vacca, Telford, PA (US); Tea Shavlakadze, Hawthorne, NY (US); Chester A. Metcalf, III, Ardsley, NY (US); William Olson, Yorktown Heights, NY (US)

(73) Assignee: Regeneron Pharmaceuticals, Inc., Tarrytown, NY (US)

(21) Appl. No.: 18/628,171

(22) Filed: Apr. 5, 2024

Related U.S. Application Data
(60) Provisional application No. 63/494,839, filed on Apr. 7, 2023.
(60) Provisional application No. 63/515,184, filed on Jul. 24, 2023.
(60) Provisional application No. 63/624,875, filed on Jan. 25, 2024.

(51) Int. Cl. C07D 498/18; A61K 31/439; A61K 31/444; A61K 31/4709; A61K 31/497; A61K 39/395

(52) U.S. Cl. CPC C07D 498/18; A61K 31/439; A61K 31/444; A61K 31/4709; A61K 31/497; A61K 39/3955

(57) ABSTRACT

Compounds and pharmaceutical compositions for use in the treatment of a disorder or disease mediated by the mTOR pathway through more selective binding to FKBP12 of the group of FK506 binding proteins (FKBPs) are disclosed. With an unusual pharmacokinetic profile and an enhanced pharmacodynamic selectivity among target FKBPs, those compounds are useful for the treatment of age- or aging-related diseases, diabetes, cancers, as well as inflammation-associated disorders.

### CROSS REFERENCE TO RELATED APPLICATIONS

This application claims priority to U.S. Provisional Application No. 63/494,839, filed Apr. 7, 2023; U.S. Provisional Application No. 63/515,184, filed Jul. 24, 2023; and U.S. Provisional Application No. 63/624,875, filed Jan. 25, 2024, all of which are hereby incorporated by reference in their entireties.

### TECHNICAL FIELD

Provided herein are FKBP12-selective rapamycin analogs, pharmaceutical compositions comprising the analogs, and methods of treating diseases, disorders, and conditions comprising administering the analogs or pharmaceutical compositions thereof.

### BACKGROUND OF THE INVENTION

Rapamycin is a known macrolide antibiotic produced by Streptomyces hygoscopius, see e.g., McAlpine, J. B., et al., J. Antibiotics (1991) 44:688; Schreiber, S. L.; et al., J. Am. Chem. Soc. (1991) 113:7433; and U.S. Pat. No. 3,929,992. The following numbering convention for rapamycin and its derivatives used herein is shown below:

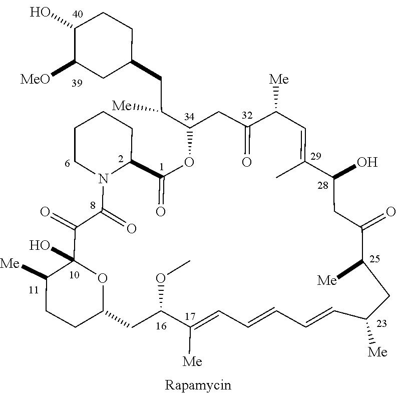

Rapamycin is a potent immunosuppressant used to prevent rejection of organ transplants and to treat certain types of cancers. It has also been shown to be useful in preventing or treating systemic lupus erythematosus, insulin-dependent diabetes mellitus, skin disorders such as psoriasis, smooth muscle cell proliferation and intimal thickening following vascular injury, adult T-cell leukemia/lymphoma, malignant carcinomas, cardiac inflammatory disease, anemia, and increased neurite outgrowth. Additionally, rapamycin analogs (so called “rapalogs”) have been shown to be efficacious against liver fibrosis. See, e.g., Liver Int. (2014) 34 (10): 1513-21.

In eukaryotic cells, rapamycin and its analogs (rapalogs) inhibit TOR (Target of Rapamycin) signaling. In mammalian cells, mTOR (mammalian target of rapamycin) exists in two distinct multiprotein complexes, described as the mTORC1 complex and the mTORC2 complex, both of which sense the availability of nutrients and energy, and integrate inputs from growth factors and stress signaling. mTORC1 integrates signals from growth factors and nutrients and controls cell growth and metabolism. Laplante M. et al. Cell. (2012) 149 (2): 274-93. mTORC1 is a key regulator of protein translation and autophagy.

In animal models, rapalogs extend lifespan and delay the onset of age-related diseases. Aging, like other biological processes, is regulated by signaling pathways such as the TOR pathway (named “TOR” in this case, to include the yeast and C elegans systems) and, in mammals, the mTORC1 pathway. Modulation of TOR and mTORC1 signaling prolongs lifespan and delays the onset of age-related diseases in a wide array of organisms, from flies to mammals. For instance, inhibition of the TOR pathway by genetic mutation extended lifespan in yeast, C. elegans, and drosophila, and inhibition of the mTORC1 pathway extended lifespan in mice (Kaeberlein et al., Science (2005) 310:1193-1196; Kapahi et al., Curr Biol (2004) 14:885-890; Selman et al., Science (2009) 326:140-144; Vellai et al., Nature (2003) 426:620). In addition, the mTORC1 inhibitor rapamycin extended the lifespan of mice even when administered late in life (Harrison et al., Nature (2009) 460 (7253): 392-395). These data raise the possibility that drugs that target the mammalian TOR (mTOR) pathway will have therapeutic effects in aging and age-related diseases in humans (M. Leslie, Science, 2013, 342). For example, J. Mannick et al. describe in Sci Transl Med. (2014) 6 (268): 268ra 179 that mTOR inhibition improves the immune function in the elderly.

Mitochondrial myopathy (MM) is the most common manifestation of adult-onset mitochondrial disease and shows a multifaceted tissue-specific stress response: (1) transcriptional response, including metabolic cytokines FGF21 and GDF15; (2) remodeling of one-carbon metabolism; and, (3) the mitochondrial unfolded protein response (Khan et al., Cell Metabolism 26, 419-428, Aug. 1, 2017). mTORC1 inhibition by rapamycin downregulated all components of ISRmt (the integrated mitochondrial stress response), improved all MM hallmarks, and reversed the progression of even late-stage MM, without inducing mitochondrial biogenesis. Thus, rapamycin and rapalogs are considered to be of potential value in providing the unmet needs of many clinical fronts.

Epilepsy which is due to mutations of the Tsc1/Tsc2 complex can be treated by rapamycin or rapalogs. (Zeng et al, Ann. Neurology, 2008 April; 63 (4) 444-453). It would be advantageous to have a rapalog which could inhibit epilepsy without perturbing other tissues where mTORC1 is expressed-thus a more selective mTORC1 inhibitor would be desireable in that setting.

mTORC1 is a key regulator of protein translation and autophagy. The mTORC1 complex is sensitive to allosteric mTOR inhibitors, such as rapamycin and rapalogs. The mode of action of rapamycin and previously-produced rapalogs involves the formation of an intracellular complex with FK506 binding proteins, which could include FKBP12, FKBP12.6, FKBP13, FKBP25, FKBP51 or FKBP52 (these six FKBPs will be referenced herein as “FKBP” or “FKBPs”), followed by the binding of the FKBP-rapalog complex to the FR^{B} (FK506-rapamycin binding) domain of mTOR (Marz A. M. et al. Mol Cell Biol. (2013) 33 (7): 1357-1367). Such interaction of the FKBP-rapalog complex with mTORC1 results in allosteric inhibition of the complex. Rapamycin and rapalogs, such as RAD001 (everolimus), have gained clinical relevance by inhibiting the activity of mTORC1, which is associated with both benign and malignant proliferation disorders (Royce M. E. et al. Breast Cancer (Auckl). (2015) 9:73-79; Pleniceanu O. et al. Kidney Int Rep. (2018) 3 (1): 155-159). The various FKBPs are expressed to different degrees in various cell types and organs found in the body.

It has also been reported that rapamycin treatment selectively targets CD40-mediated B cell proliferation and differentiation (Atsuko Sakata et al., Immunology Letters. Volume 68, Issues 2-3, 1 Jun. 1999, Pages 301-309). CD40 is a cell surface receptor that is part of the tumor necrosis factor (TNF) receptor superfamily. CD40 is expressed on antigen-presenting cells such as B cells, macrophages, and dendritic cells, as well as some non-immune cells and tumors (Dakal et al, Immunobiology 2020, 225:151899). Activation of resting B cells requires an initial triggering of the B cell antigen receptor (BCR) and secondary stimuli through various cytokine receptors and B cell activation molecules including CD40.

The interaction of CD40 with its ligand CD40L provides a co-stimulatory signal that is essential to the survival of many cell types and is required for functions of immune response such as germinal center formation, antibody responses to T-dependent antigens, and “licensing” dendritic cells to mature and become potent to trigger T-cell activation and differentiation (see, e.g., Kawabe et al., Immunity 1994, 1:167-178; Elgueta et al., Immunol. Rev. 2009, 229:152-172).

CD40-CD40L signaling is implicated in autoimmune conditions that are largely driven by autoantibodies, such as systemic rheumatic diseases where autoantibodies play an important role in disease progression (such as multiple sclerosis, autoimmune nephritis, rheumatoid arthritis, Sjogren's syndrome, and systemic lupus erythematosus) as well as non-rheumatic conditions having an autoantibody component (such as myasthenia gravis, Grave's disease, and neuromyelitis optica)(see, Karnell et al., Adv Drug Delivery Rev. 2019, 141:92-103). Additionally, because CD40-CD40L signaling is essential for activation of antigen-presenting cells, blocking antigen presentation by dendritic cells or B cells may impact CD8+ T cell responses in some diseases, such as multiple sclerosis (see, e.g., Denic et al., Expert Opin Ther Targets 2013, 17:1053-1066). Altered CD40-CD40L signaling is also implicated in other diseases and conditions such as cardiovascular disease and transplantation (see, e.g., Dakal et al, Immunobiology 2020, 225:151899; Pamukcu et al., Ann. Med. 2011, 43:331; 340; Pinelli et al., Immunotherapy 2015, 7:399-410).

For example, immunosuppression with CD40 costimulatory blockade plus rapamycin provided long-term islet and kidney allograft survival (90, 94, > 120, > 120, and >120 days), with only one recipient developing evidence of allograft rejection. The CD40/rapamycin regimen was also tested in four kidney-alone transplant recipients. All four recipients achieved long-term renal allograft survival (100% at day 120), which was superior to renal allograft survival (62.9% at day 120) with triple immunosuppressive regimen (tacrolimus, mycophenolate mofetil, and steroids)(T. Oura 1, K. Hotta, et al., American Journal of Transplantation. Volume 17, Issue 3, March 2017, Pages 646-656).

Since different FKBPs are differentially expressed in different tissues, and since there are some settings where it is desireable to avoid mTORC1 inhibition in particular cell types, rapalogs that are selective to individual FKBPs would be needed to increase the ability to target specific tissues. For instance, a rapalog that is selective to FKBP12 could target tissues with high FKBP12 expression, but spare tissues that have low (or no) levels of FKBP12. FKBP-selective analogs would provide unique and clinically advantageous benefits for patients, as these may avoid adverse events caused by widespread inhibition of mTORC1.

What is also needed are rapalogs that can be administered in combination with an anti-CD40 antibody for the treatment of diseases where autoantibodies play an important role in disease progression, to induce an immune response, and/or to prevent organ transplant rejection.

What is also needed are rapalogs that can be administered in combination with immunotherapeutic agents which target T-cells to specific tumors, such that the rapalog does not inhibit or interfere the immunotherapeutic approach.

### SUMMARY OF THE INVENTION

In one aspect, provided herein is a compound of Formula I:

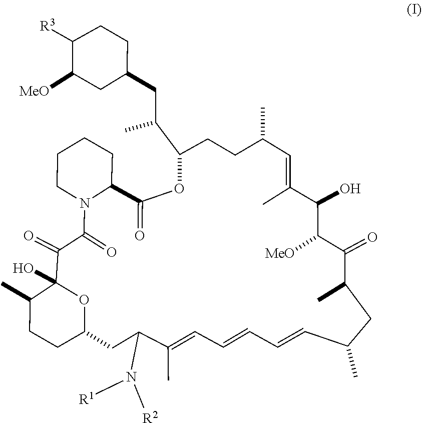

or a pharmaceutically acceptable salt thereof;
wherein R¹ is hydrogen, C₁-₆ alkyl, heterocyclyl, aryl, heteroaryl, —C₀₋₆alkylene-SO₂R⁴, or —C₀₋₆alkylene-SO₂R⁵; wherein the C₁-₆ alkyl, heterocyclyl, aryl, and heteroaryl are optionally substituted with one or two R^{1a} groups;

R² is heterocyclyl, aryl, heteroaryl, —C₀₋₆alkylene-SO₂R⁴, or —C₀₋₆alkylene-SO₂R⁵; wherein the heterocyclyl, aryl, and heteroaryl are optionally substituted with one or two R^{2a} groups;
or R¹ and R² together with the attached nitrogen form a N-linked heteroaryl or N-linked heterocyclyl optionally substituted with one or two R^{1a} groups;
R³ is selected from the group consisting of —OR^{a}, 3-6 membered heterocyclyl, and 3-6 membered heteroaryl;
R^{a} is selected from the group consisting of H, —P(O)(R^{b}) 2, —C(O)R^{c}, —C(O)OR^{c}, C₁₋₆ alkyl, and C₁₋₆ hydroxyalkyl;
each R^{b} is independently selected from the group consisting of H and C₁-₆ alkyl;
R^{c} is selected from the group consisting of H, C₁₋₆ alkyl, and C₁₋₆ hydroxyalkyl;
R⁴ is C₂₋₆alkyl, C₃₋₆cycloalkyl, C₁₋₆ hydroxyalkyl, heterocyclyl, aryl, or heteroaryl; wherein the heterocyclyl, aryl, and heteroaryl are optionally substituted with one or two R^{4a} groups; and
R⁵ is heterocyclyl, aryl, or heteroaryl; wherein the heterocyclyl, aryl, and heteroaryl are optionally substituted with one or two R^{5a} groups;
each of the one or two R^{1a}, R^{2a}, R^{4a}, and R^{5a} groups, when present, are independently selected from C₁₋₆ alkyl, C₁₋₆ hydroxyalkyl, hydroxy, halo, C₁-6haloalkyl, C₁₋₆ alkoxy, C₁-₆haloalkoxy, and cyano; or any two R^{1a}, R^{2a}, R^{4a}, and R^{5a} groups, when present on the same carbon, are taken together to form an oxo group;

wherein when R¹ is hydrogen, R² is not-C₀₋₆ alkylene-SO₂R⁴.

In one embodiment, the compound of Formula (I) is a compound of Formula (Ia):

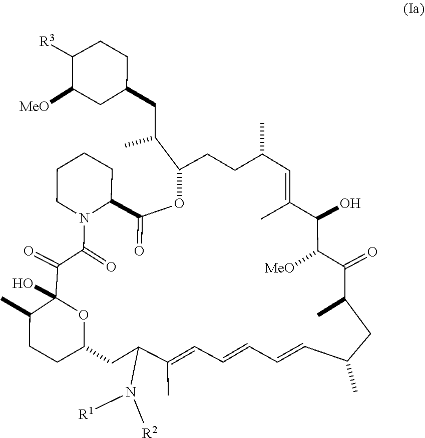

or a pharmaceutically acceptable salt thereof;
wherein R¹ is hydrogen or C₁-₆ alkyl;
R² is heterocyclyl, aryl, or heteroaryl optionally substituted with one or two R^{2a} groups;
R³ is selected from the group consisting of —OR^{a}, a 3-6 membered heterocyclyl, and 3-6 membered heteroaryl;
R^{a} is selected from the group consisting of H, —P(O)(R^{b})₂, —C(O)R^{c}, —C(O)OR^{c}, C₁₋₆ alkyl, and C₁₋₆ hydroxyalkyl;
each R^{b} is independently selected from the group consisting of H and C₁₋₆ alkyl;
R^{c} is selected from the group consisting of H, C₁-₆ alkyl, and C₁₋₆ hydroxyalkyl; and
each of the one or two R^{2a} groups, when present, are independently selected from C₁-₆alkyl, C₁₋₆ hydroxyalkyl, hydroxy, halo, C₁₋₆ haloalkyl, C₁₋₆ alkoxy, C₁₋₆ haloalkoxy, and cyano;
or any two R^{2a} groups, when present on the same carbon, are taken together to form an oxo group.

In one embodiment, the compound of Formula (I) is a compound of Formula (Ib):

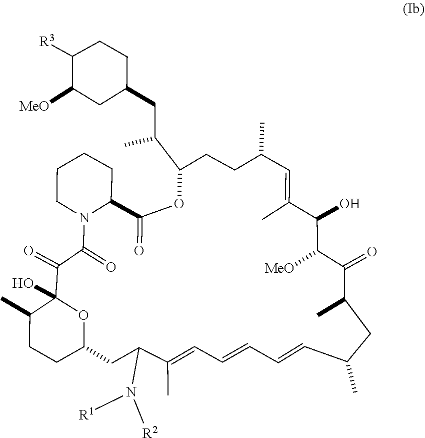

or a pharmaceutically acceptable salt thereof;
wherein R¹ and R² together with the attached nitrogen form a N-linked heteroaryl or N-linked heterocyclyl optionally substituted with one or two R^{1a} groups;
R³ is selected from the group consisting of —OR^{a}, a 3-6 membered heterocyclyl, and 3-6 membered heteroaryl;
R^{a} is selected from the group consisting of H, —P(O)(R^{b})₂, —C(O)R^{c}, —C(O)OR^{c}, C₁-₆alkyl, and C₁₋₆ hydroxyalkyl;
each R^{b} is independently selected from the group consisting of H and C₁-₆ alkyl;
R^{c} is selected from the group consisting of H, C₁-₆ alkyl, and C₁₋₆ hydroxyalkyl;
each of the one or two R^{1a} groups, when present, are independently selected from C₁-₆alkyl, C₁₋₆ hydroxyalkyl, hydroxy, halo, C₁₋₆ haloalkyl, C₁₋₆ alkoxy, C₁₋₆ haloalkoxy, and cyano;
or two R^{1a} groups, when present on the same carbon, are taken together to form an oxo group.

In one embodiment, the compound of Formula (I) is a compound of Formula (Ic):

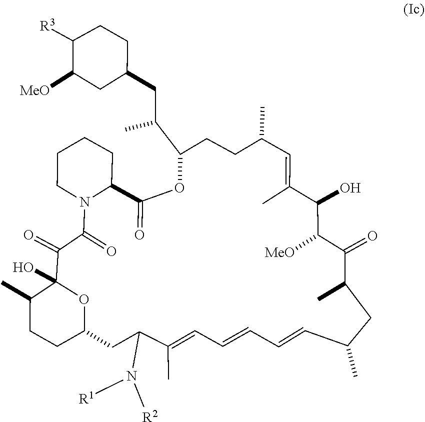

or a pharmaceutically acceptable salt thereof;
wherein R¹ is hydrogen or C₁-₆ alkyl;
R² is —C₀₋₆alkylene-SO₂R⁴ or —C₀₋₆alkylene-SO₂R⁵;
R³ is selected from the group consisting of —OR^{a}, a 3-6 membered heterocyclyl, and 3-6 membered heteroaryl;
R^{a} is selected from the group consisting of H, —P(O)(R^{b})₂, —C(O)R^{c}, —C(O)OR^{c}, C₁-₆ alkyl, and C₁₋₆ hydroxyalkyl;
each R^{b} is independently selected from the group consisting of H and C₁-₆ alkyl;
R^{c} is selected from the group consisting of H, C₁-₆ alkyl, and C₁₋₆ hydroxyalkyl;
R⁴ is C₂₋₆ alkyl, C₃₋₆cycloalkyl, C₁₋₆ hydroxyalkyl, heterocyclyl, aryl, or heteroaryl; wherein the heterocyclyl, aryl, and heteroaryl are optionally substituted with one or two R^{4a} groups;
R⁵ is heterocyclyl, aryl, or heteroaryl; wherein the heterocyclyl, aryl, and heteroaryl are optionally substituted with one or two R^{5a} groups;
each of the one or two R^{4a} and R^{5a} groups, when present, are independently selected from C₁₋₆ alkyl, C₁₋₆ hydroxyalkyl, hydroxy, halo, C₁-6haloalkyl, C₁₋₆ alkoxy, C₁₋₆ haloalkoxy, and cyano; or any two R^{4a} and R^{5a} groups, when present on the same carbon, are taken together to form an oxo group;
wherein when R¹ is hydrogen, R² is not-C₀₋₆ alkylene-SO₂R⁴.

The present disclosure provides at least the following embodiments:
a) A compound of Formula (P-I), Formula (P-Ia), Formula (P-Ib), Formula (P-Ic), Formula (P-2), Formula (I), Formula (Ia), Formula (Ib), Formula (Ic), or a pharmaceutically acceptable salt thereof;
b) A compound selected from Compound 3-30, Compound 43-65, or Compound 67-74 or a pharmaceutically acceptable salt thereof;
c) The compound of (a) or (b) wherein the compound binds to FKBP12 selectively over other FK506 binding proteins, in comparison to Everolimus;
d) A pharmaceutical composition comprising a compound of Formula (P-I), Formula (P-Ia), Formula (P-Ib), Formula (P-Ic), Formula (P-2), Formula (I), Formula (Ia), Formula (Ib), Formula (Ic), or a pharmaceutically acceptable salt thereof in a pharmaceutically acceptable excipient, diluent, or carrier;
e) A pharmaceutical composition comprising a compound of (b) or (c) in a pharmaceutically acceptable excipient, diluent, or carrier;
f) A pharmaceutical composition comprising a compound of (a)-(c) and at least one additional therapeutically active agent in a pharmaceutically acceptable excipient, diluent, or carrier;
g) A combination therapy comprising a therapeutically effective amount of a compound of (a)-(c) or a pharmaceutical composition of (d)-(f), and an anti-CD40 antibody;
h) A method of treating a disease or disorder mediated by the mTOR pathway in a subject in need thereof comprising administering a therapeutically effective amount of a compound of (a)-(c), a pharmaceutical composition of (d)-(f), or a combination therapy of (g);
i) The method of (h) wherein the target tissue, organ, or cells associated with the pathology of the disease or disorder has FKBP12 levels sufficient to inhibit mTORC1;
j) The method of (h) or (i) wherein the therapeutically effective amount of a compound of (a)-(c) or pharmaceutical composition of (d)-(f) has high enough affinity binding to FKBP12 sufficient to inhibit mTORC1;
k) The method of (h)-(j) wherein the wherein the disease or disorder is selected from sarcopenia, skin atrophy, cherry angiomas, seborrheic keratoses, brain atrophy, atherosclerosis, arteriosclerosis, pulmonary emphysema, osteoporosis, osteoarthritis, high blood pressure, erectile dysfunction, cataracts, macular degeneration, glaucoma, stroke, cerebrovascular disease (strokes), chronic kidney disease, diabetes-associated kidney disease, impaired hepatic function, liver fibrosis, autoimmune hepatitis, endometrial hyperplasia, metabolic dysfunction, renovascular disease, hearing loss, mobility disability, cognitive decline, tendon stiffness, heart dysfunction such as cardiac hypertrophy and/or systolic and/or diastolic dysfunction and/or hypertension, heart dysfunction which results in a decline in ejection fraction, immune senescence, Parkinson's disease, Alzheimer's disease, cancer, immune-senescence leading to cancer due to a decrease in immune-surveillance, infections due to an decline in immune-function, chronic obstructive pulmonary disease (COPD), obesity, loss of taste, loss of olfaction, arthritis, cancer in which the tumor has elevated levels of mTORC1 signaling and/or sufficient levels of FKBP12 so as to allow inhibition of mTORC1, and type II diabetes;
l) The method of (h)-(j) wherein the disease or disorder is an age-related disorder or disease selected from sarcopenia; skin atrophy; cherry angiomas; seborrheic keratoses; brain atrophy; atherosclerosis; arteriosclerosis; pulmonary emphysema; osteoporosis; osteoarthritis; high blood pressure; erectile dysfunction; cataracts; macular degeneration; glaucoma; stroke; cerebrovascular disease (strokes); chronic kidney disease; diabetes-associated kidney disease; impaired hepatic function; liver fibrosis; autoimmune hepatitis; endometrial hyperplasia; metabolic dysfunction; renovascular disease; hearing loss; mobility disability; cognitive decline; tendon stiffness; heart dysfunction, such as cardiac hypertrophy and/or systolic and/or diastolic dysfunction and/or hypertension; heart dysfunction that results in a decline in ejection fraction; immune senescence; Parkinson's disease; Alzheimer's disease; cancer; immune-senescence leading to cancer due to a decrease in immune-surveillance; infections due to an decline in immune-function; chronic obstructive pulmonary disease (COPD); obesity; loss of taste; loss of olfaction; arthritis; cancer in which the tumor has elevated levels of mTORC1 signaling and/or sufficient levels of FKBP12 so as to allow inhibition of mTORC1; and, type II diabetes;
m) The method of (h)-(j) wherein the disease or disorder is cancer;
n) The method of (m) wherein the cancer is selected from renal cancer, renal cell carcinoma, colorectal cancer, uterine sarcoma, endometrial uterine cancer, endometrial cancer, breast cancer, ovarian cancer, cervical cancer, gastric cancer, fibro-sarcoma, pancreatic cancer, liver cancer, melanoma, leukemia, multiple myeloma, nasopharyngeal cancer, prostate cancer, lung cancer, glioblastoma, bladder cancer, mesothelioma, head cancer, rhabdomyosarcoma, sarcoma, lymphoma, and neck cancer;
o) The method of (k) wherein the disease is Alzheimer's disease,
p) The method of (h) wherein the disease is Graft-versus-Host Disease (GvHD).
q) The method of (h) wherein the disease is facial angiofibroma associated with tuberous sclerosis complex.
r) The method of (h) wherein the disease is advanced unresectable or metastatic malignant perivascular epithelioid cell tumors;
s) A method of inducing immune tolerance and/or preventing transplant organ rejection in a subject in need thereof comprising administering a therapeutically effective amount of a compound of (a)-(c), a pharmaceutical composition of (d)-(f), or a combination therapy of (g);
t) The method of any one of claims (h)-(s) wherein the compound is a compound selected from Compounds 16-19, 26-28, 45, 50, 63, 68-72, and 74.
u) A compound of (a)-(c), a pharmaceutical composition of (d)-(f), or a combination therapy of (g) for use in the treatment of a disease or disorder;
v) A compound of (a)-(c), a pharmaceutical composition of (d)-(f), or a combination therapy of (g) for use in the treatment of a disease or disorder mediated by the mTOR pathway in a subject in need thereof;
w) The compound of (u)-(v) wherein the compound is selected from Compounds 16-19, 26-28, 45, 50, 63, 68-72, and 74;
x) Use of a therapeutically effective amount of a compound of (a)-(c), a pharmaceutical composition of (d)-(f), or a combination therapy of (g) in the manufacture of a medicament for the treatment of a disease or disorder;
y) Use of a therapeutically effective amount of a compound of (a)-(c), a pharmaceutical composition of (d)-(f), or a combination therapy of (g) in the manufacture of a medicament for the treatment of a disease or disorder mediated by the mTOR pathway in a subject in need thereof; and
z) The use of (x)-(y) wherein the compound is selected from Compounds 16-19, 26-28, 45, 50, 63, 68-72, and 74.

In some embodiments, the compounds and pharmaceutical compositions disclosed herein for use in the treatment of a disorder or disease mediated by the mTOR pathway are more selective in binding to FKBP12 than others of the group of FK506 binding proteins (FKBPs).

In some other embodiments, the compounds and pharmaceutical compositions disclosed herein for use in the treatment of a disorder or disease mediated by the mTOR pathway show an unusual and surprising pharmacokinetic profile and an enhanced pharmacodynamic selectivity among target FKBPs, those compounds and pharmaceutical composition matters are useful for the treatment of age- or aging-related diseases, diabetes-related complications, cancers, as well as inflammation-associated disorders.

Yet in some other embodiments, the compounds and pharmaceutical compositions pathway, wherein the compound inhibits S6K1 phosphorylation at least two-fold less efficiently than the rapalog RAD001 in FKBP12 KO cells in comparison to FKBP12 expressing cells.

In some other embodiments, the compounds and pharmaceutical compositions disclosed herein for use in the treatment of a disorder or disease mediated by the mTOR pathway, wherein the compound inhibits S6K1 phosphorylation at least ten-fold less efficiently than the rapalog RAD001 in FKBP12 KO cells in comparison to FKBP12 expressing cells.

In some other embodiments, the compounds and pharmaceutical compositions disclosed herein for use in the treatment of a disorder or disease mediated by the mTOR pathway, wherein the compound inhibits S6K1 phosphorylation at least 100-fold less efficiently than the rapalog RAD001 in FKBP12 KO cells in comparison to FKBP12 expressing cells.

In some other embodiments, the compounds and pharmaceutical compositions disclosed herein for use in the treatment of a disorder or disease mediated by the mTOR pathway, wherein the compound requires about 10-fold higher concentration, in comparison to the rapalog RAD001, in FKBP12 KO cells in comparison to FKBP12 expressing cells, to achieve 20% inhibition of S6K1 cell signaling.

In some other embodiments, the compounds and pharmaceutical compositions disclosed herein for use in the treatment of a disorder or disease mediated by the mTOR pathway, wherein the compound requires about 100-fold higher concentration, in comparison to the rapalog RAD001, in FKBP12 KO cells in comparison to FKBP12 expressing cells, to achieve 20% inhibition of S6K1 cell signaling.

In some other embodiments, the compounds and pharmaceutical compositions disclosed herein for use in the treatment of a disorder or disease mediated by the mTOR pathway, wherein the compound requires about 500-fold higher concentration, in comparison to the rapalog RAD001, in FKBP12 KO cells in comparison to FKBP12 expressing cells, to achieve 20% inhibition of S6K1 cell signaling.

In some other embodiments, the compounds and pharmaceutical compositions disclosed herein for use in the treatment of a disorder or disease mediated by the mTOR pathway, wherein the compound requires about 1000-fold higher concentration, in comparison to the rapalog RAD001, in FKBP12 KO cells in comparison to FKBP12 expressing cells, to achieve 20% inhibition of S6K1 cell signaling.

In some other embodiments, the compounds and pharmaceutical compositions pathway, wherein the compound requires about 10-fold higher concentration, in comparison to the rapalog RAD001, in FKBP12 KO cells in comparison to FKBP12 expressing cells, to achieve 30% inhibition of S6K1 cell signaling.

In some other embodiments, the compounds and pharmaceutical compositions disclosed herein for use in the treatment of a disorder or disease mediated by the mTOR pathway, wherein the compound requires about 100-fold higher concentration, in comparison to the rapalog RAD001, in FKBP12 KO cells in comparison to FKBP12 expressing cells, to achieve 30% inhibition of S6K1 cell signaling.

In some other embodiments, the compounds and pharmaceutical compositions disclosed herein for use in the treatment of a disorder or disease mediated by the mTOR pathway, wherein the compound requires about 500-fold higher concentration, in comparison to the rapalog RAD001, in FKBP12 KO cells in comparison to FKBP12 expressing cells, to achieve 30% inhibition of S6K1 cell signaling.

In some other embodiments, the compounds and pharmaceutical compositions disclosed herein for use in the treatment of a disorder or disease mediated by the mTOR pathway, wherein the compound requires about 1000-fold higher concentration, in comparison to the rapalog RAD001, in FKBP12 KO cells in comparison to FKBP12 expressing cells, to achieve 30% inhibition of S6K1 cell signaling.

Yet in some other embodiments, an “FKBP12-selective rapalog” is a rapalog which is approximately 10-fold to approximately 1000-fold less potent than RAD001 in a cell line that does not express FKBP12 (e.g., FKBP12 knock-out cells in comparison to FKBP12 expressing cells). “Potency,” as used in this context, can be expressed as the concentration of rapalog required to achieve a 20% inhibition of S6K1 (Thr389)phosphorylation in a cell-based assay such as that used in Example 3 and FIGS. 1-11 disclosed herein. For example, a rapalog is considered FKBP12-selective if the IC20 of the rapalog in an S6K1 (Thr389)phosphorylation inhibition assay using an FKBP12 knock-out cell line is at least 20× greater (e.g., 30×, 50×, 100×, 200×, 500×, 1000× greater) than the IC20 of RAD001 in the same assay with the same cell line, which normally expresses FKBP12.

In some other embodiments, an “FKBP12-selective rapalog” is a rapalog which is approximately 10-fold to approximately 1000-fold less potent than RAD001 in a cell line that does not express FKBP12 (e.g., FKBP12 knock-out cells in comparison to FKBP12 expressing cells). “Potency,” as used in this context, can be expressed as the concentration of rapalog required to achieve a 30% inhibition of S6K1 (Thr389)phosphorylation in a cell-based assay such as that used in Example 3 and FIGS. 1-11 disclosed herein. For example, a rapalog is considered FKBP12-selective if the IC30 of the rapalog in an S6K1 (Thr389)phosphorylation inhibition assay using an FKBP12 knock-out cell line is at least 20× greater (e.g., 30×, 50×, 100×, 200×, 500×, 1000× greater) than the IC30 of RAD001 in the same assay with the same cell line, which normally expresses FKBP12.

In an embodiment, the compound of Formula (I) or a pharmaceutically acceptable salt thereof has higher affinity binding to FKBP12, sufficient to inhibit mTORC1, e.g., as compared to rapamycin or RAD001.

In an embodiment, a compound of (I), (P-1), (P-2), (Ia)-(Ic), or (P-Ia)-(P-Ic), or a pharmaceutically acceptable salt thereof may complex with FKBP12 to bind and inhibit mTORC1 more potently as compared to rapamycin or RAD001.

In an embodiment, the higher affinity binding to FKBP12 results in greater efficacy, e.g., as compared to rapamycin or RAD001.

In an embodiment, efficacy of treatment is determined empirically, e.g., as compared to rapamycin or RAD001.

Yet in another aspect, the disclosure provides a method of treating a disease or disorder in a subject having, or previously determined to have, FKBP12 levels sufficient to inhibit mTORC1, the method comprising administering to the subject in need thereof a therapeutically effective amount of a compound of (I), (P-1), (P-2), (Ia)-(Ic), or (P-Ia)-(P-Ic), or a pharmaceutically acceptable salt thereof, or a pharmaceutical composition described herein, or a pharmaceutical combination described herein.

In another aspect, the disclosure provides a method for treating an age-related disease or disorder in a subject in need thereof, the method comprising administering to the subject a therapeutically effective amount of a compound of (I), (P-1), (P-2), (Ia)-(Ic), or (P-Ia)-(P-Ic), or a pharmaceutically acceptable salt thereof, or a pharmaceutical composition described herein, or a pharmaceutical combination described herein.

In an embodiment, the disease or disorder is selected from sarcopenia, skin atrophy, cherry angiomas, seborrheic keratoses, brain atrophy (also referred to as dementia), atherosclerosis, arteriosclerosis, pulmonary emphysema, osteoporosis, osteoarthritis, high blood pressure, erectile dysfunction, cataracts, macular degeneration, glaucoma, stroke, cerebrovascular disease (strokes), chronic kidney disease, diabetes-associated kidney disease, impaired hepatic function, liver fibrosis, autoimmune hepatitis, endometrial hyperplasia, metabolic dysfunction, renovascular disease, hearing loss, mobility disability (e.g., frailty), cognitive decline, tendon stiffness, heart dysfunction such as cardiac hypertrophy and/or systolic and/or diastolic dysfunction and/or hypertension, heart dysfunction which results in a decline in ejection fraction, immune senescence, Parkinson's disease, Alzheimer's disease, cancer, immune-senescence leading to cancer due to a decrease in immune-surveillance, infections due to an decline in immune-function, chronic obstructive pulmonary disease (COPD), obesity, loss of taste, loss of olfaction, arthritis, and type II diabetes (including complications stemming from diabetes, such as kidney failure, blindness and neuropathy).

In another aspect, the disclosure provides a method for treating a disease or disorder in a subject in need thereof, the method comprising administering to the subject a therapeutically effective amount of a compound of (I), (P-1), (P-2), (Ia)-(Ic), or (P-Ia)-(P-Ic), or a pharmaceutically acceptable salt thereof, or a pharmaceutical composition, or a pharmaceutical combination thereof, wherein the disorder or disease is selected from:
Acute or chronic organ or tissue transplant rejection;
Transplant vasculopathies;
Smooth muscle cell proliferation and migration leading to vessel intimal thickening, blood vessel obstruction, obstructive coronary atherosclerosis, restenosis;
Autoimmune diseases and inflammatory conditions;
Asthma;
Multi-drug resistance (MDR);
Fungal infections;
Inflammation;
Infection;
Age-related diseases;
Neurodegenerative diseases;
Proliferative disorders, e.g., cancer;
Seizures and seizure related disorders; and
Mitochondrial myopathy and mitochondrial stress.

In another aspect, the disclosure provides a method for treating cancer in a subject in need thereof, the method comprising administering to the subject a therapeutically effective amount of a compound of Formula (I) or a pharmaceutically acceptable salt thereof, or a pharmaceutical composition described herein, or a pharmaceutical combination described herein.

In an embodiment, the method further comprises a PD-1/PDL-1 inhibitor.

In an embodiment, the cancer is selected from renal cancer, renal cell carcinoma, colorectal cancer, uterine sarcoma, endometrial uterine cancer, endometrial cancer, breast cancer, ovarian cancer, cervical cancer, gastric cancer, fibro-sarcoma, pancreatic cancer, liver cancer, melanoma, leukemia, multiple myeloma, nasopharyngeal cancer, prostate cancer, lung cancer, glioblastoma, bladder cancer, mesothelioma, head cancer, rhabdomyosarcoma, sarcoma, lymphoma, and neck cancer.

In an embodiment, the disorder is a liver disorder that includes the process of fibrosis and/or inflammation, e.g., liver fibrosis that occurs in end-stage liver disease; liver cirrhosis; liver failure due to toxicity; non-alcohol-associated hepatic steatosis or NASH; and alcohol-associated steatosis.

In an embodiment, the disorder is a kidney disorder that includes the process of fibrosis or inflammation in the kidney, e.g., kidney fibrosis, which occurs as a result of acute kidney injury, leading to chronic kidney disease and diabetic nephropathy.

In an embodiment, the disorder is a heart dysfunction, e.g., myocardial infarction or cardiac hypertrophy. In an embodiment, the heart dysfunction is systolic and/or diastolic dysfunction. In an embodiment, the heart dysfunction is hypertension. In an embodiment, the heart dysfunction results in a decline in ejection fraction.

In an embodiment, the disorder is an immune-senescence leading to cancer due to a decrease in immune-surveillance.

In an embodiment, the disorder is cancer, including tumors which are treated by immunotherapy, and those which have been previously treated by either rapamycin, RAD001, or another rapalog. In an embodiment, the cancer includes tumors where the mTOR pathway is shown to be activated, including settings where there is a mutation in the Tsc1 gene, or where the tumor microenvironment is appropriately treated by a rapalog.

The details of one or more embodiments of the disclosure are set forth herein. Other features, objects, and advantages of the disclosure will be apparent from the Figures, the Detailed Description, the Examples, and the Claims.

### BRIEF DESCRIPTION OF DRAWINGS

FIG. 1A is a line graph showing inhibition of S6K1 (Thr389) in wild-type 293T cells. Cells were treated for 2 hours with the following compounds: RAD001 (everolimus; dotted line with circles) and Compound 44 (solid line with squares). All treatments were performed in duplicates. The Y axis represents percent inhibition relative to the S6K1 (Thr389) level in cells treated with media plus dimethyl sulfoxide (DMSO). The X axis represents concentrations for the two compounds.

FIG. 1B is a line graph showing inhibition of S6K1 (Thr389) in FKBP12 knock-out 293T cells. Cells were treated for 2 hours with the following compounds: RAD001 (everolimus; dotted line with circles) and Compound 44 (solid line with squares). All treatments were performed in duplicates. The Y axis represents percent inhibition relative to the S6K1 (Thr389) level in cells treated with media plus dimethyl sulfoxide (DMSO). The X axis represents concentrations for the two compounds.

FIG. 1C is a line graph showing inhibition of S6K1 (Thr389) in wild-type 293T cells. Cells were treated for 2 hours with the following compounds: RAD001 (everolimus; dotted line with circles) and Compound 45 (solid line with squares). All treatments were performed in duplicates. The Y axis represents percent inhibition relative to the S6K1 (Thr389) level in cells treated with media plus dimethyl sulfoxide (DMSO). The X axis represents concentrations for the two compounds.

FIG. 1D is a line graph showing inhibition of S6K1 (Thr389) in FKBP12 knock-out 293T cells. Cells were treated for 2 hours with the following compounds: RAD001 (everolimus; dotted line with circles) and Compound 45 (solid line with squares). All treatments were performed in duplicates. The Y axis represents percent inhibition relative to the S6K1 (Thr389) level in cells treated with media plus dimethyl sulfoxide (DMSO). The X axis represents concentrations for the two compounds.

FIG. 2A is a line graph showing inhibition of S6K1 (Thr389) in wild-type 293T cells. Cells were treated for 2 hours with the following compounds: RAD001 (everolimus; dotted line with circles) and Compound 18 (solid line with squares). All treatments were performed in duplicates. The Y axis represents percent inhibition relative to the S6K1 (Thr389) level in cells treated with media plus dimethyl sulfoxide (DMSO). The X axis represents concentrations for the two compounds.

FIG. 2B is a line graph showing inhibition of S6K1 (Thr389) in FKBP12 knock-out 293T cells. Cells were treated for 2 hours with the following compounds: RAD001 (everolimus; dotted line with circles) and Compound 18 (solid line with squares). All treatments were performed in duplicates. The Y axis represents percent inhibition relative to the S6K1 (Thr389) level in cells treated with media plus dimethyl sulfoxide (DMSO). The X axis represents concentrations for the two compounds.

FIG. 3A is a line graph showing inhibition of S6K1 (Thr389) in wild-type 293T cells. Cells were treated for 2 hours with the following compounds: RAD001 (everolimus; dotted line with circles) and Compound 16 (solid line with squares). All treatments were performed in duplicates. The Y axis represents percent inhibition relative to the S6K1 (Thr389) level in cells treated with media plus dimethyl sulfoxide (DMSO). The X axis represents concentrations for the two compounds.

FIG. 3B is a line graph showing inhibition of S6K1 (Thr389) in FKBP12 knock-out 293T cells. Cells were treated for 2 hours with the following compounds: RAD001 (everolimus; dotted line with circles) and Compound 16 (solid line with squares). All treatments were performed in duplicates. The Y axis represents percent inhibition relative to the S6K1 (Thr389) level in cells treated with media plus dimethyl sulfoxide (DMSO). The X axis represents concentrations for the two compounds.

FIG. 4A is a line graph showing inhibition of S6K1 (Thr389) in wild-type 293T cells. Cells were treated for 2 hours with the following compounds: RAD001 (everolimus; dotted line with circles) and Compounds 26 (solid line with squares)) and Compound 28 (solid line with crosses). All treatments were performed in duplicates. The Y axis represents percent inhibition relative to the S6K1 (Thr389) level in cells treated with media plus dimethyl sulfoxide (DMSO). The X axis represents concentrations for the two compounds.

FIG. 4B is a line graph showing inhibition of S6K1 (Thr389) in FKBP12 knock-out 293T cells. Cells were treated for 2 hours with the following compounds: RAD001 (everolimus; dotted line with circles) and Compounds 26 (solid line with squares) and Compound 28 (solid line with crosses). All treatments were performed in duplicates. The Y axis represents percent inhibition relative to the S6K1 (Thr389) level in cells treated with media plus dimethyl sulfoxide (DMSO). The X axis represents concentrations for the two compounds.

FIG. 5A is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 47 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents Log10 molar (M) concentrations for the compounds.

FIG. 5B is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 47 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents Log10 molar (M) concentrations for the compounds.

FIG. 5C is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 48 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents Log10 molar (M) concentrations for the compounds.

FIG. 5D is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 48 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents Log10 molar (M) concentrations for the compounds.

FIG. 5E is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 49 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 5F is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 49 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents Log10 molar (M) concentrations for the two compounds.

FIG. 6A is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 50 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 6B is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 50 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 6C is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 51 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 6D is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 51 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 6E is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 52 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 6F is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 52 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 7A is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 57 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 7B is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 57 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 7C is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 58 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 7D is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 58 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 7E is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 59 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 7F is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 59 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 8A is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 63 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 8B is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 63 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 9A is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 69 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 9B is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 69 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 10A is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 72 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 10B is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 72 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 11A is a line graph showing inhibition of S6K1 (Thr389)phosphorylation in wild-type (WT) 293T cells with increasing concentrations of compound treatment. Cells were treated for 2 hours with the following compounds: RAD001 (everolimus; dotted line with circles); Compound 73 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 11B is a line graph showing inhibition of S6K1 (Thr389)phosphorylation in FKBP12 knock-out (KO) 293T cells with increasing concentrations of compound treatment. Cells were treated for 2 hours with the following compounds: RAD001 (everolimus; dotted line with circles); Compound 73 (solid line with squares). All treatments were performed in duplicates. The Y axis shows S6K1 (Thr389)phosphorylation values calculated as a percent (%) of the value in control cells treated with media plus dimethyl sulfoxide (DMSO) only. The S6K1 (Thr389)phosphorylation value in control DMSO treated cells is set to 100%. The X axis represents concentrations for the two compounds.

FIG. 12A is a graph showing mouse plasma pharmacokinetic profile of comparator compound RAD001 following IV (2 mg/kg) and PO (10 mg/kg) administration. Y axis, set to Log 10 scale, shows compound concentration (ng/ml) in plasma. X axis shows time points (hours) for plasma collection following compound administration.

FIG. 12B is a graph showing mouse plasma pharmacokinetic profile of Compound 26 following IV (2 mg/kg) and PO (20 mg/kg) administration. Y axis, set to Log 10 scale shows compound concentration (ng/ml) in plasma. X axis shows time points (hours) for plasma collection following compound administration.

FIG. 12C is a graph showing mouse plasma pharmacokinetic profile of Compound 45 following IV (2 mg/kg) and PO (20 mg/kg) administration. Y axis, set to Log 10 scale shows compound concentration, ng/ml in plasma. X axis shows time points (hours) for plasma collection following compound administration.

FIG. 12D is a graph showing mouse plasma pharmacokinetic profile of Compound 69 following IV (2 mg/kg) and PO (10 mg/kg) administration. Y axis, set to Log 10 scale, shows compound concentration, ng/ml in plasma. X axis shows time points (hours) for plasma collection following compound administration.

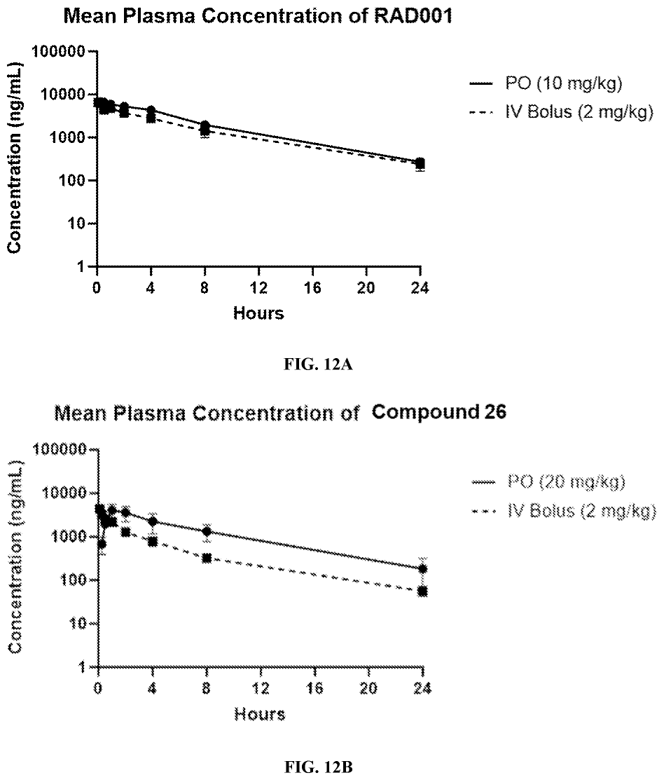

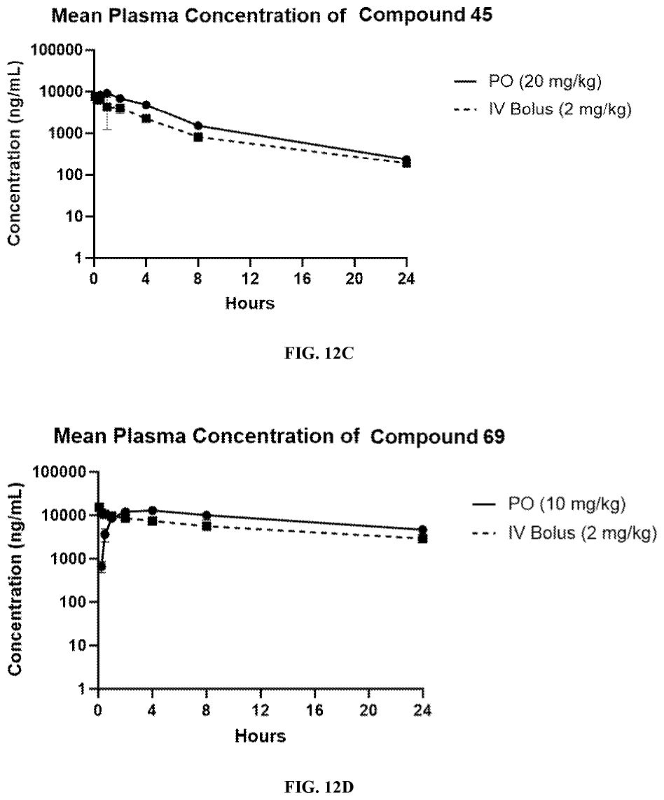

The data depicted in FIG. 12A-FIG. 12D of pharmacokinetic studies of selected compounds in mice are further summarized in Table 4 shown in the Detailed Description below.

### DETAILED DESCRIPTION OF THE INVENTION

### Definitions

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term provided herein, these Definitions prevail unless stated otherwise.

As used herein, “alkyl” refers to a monovalent and saturated hydrocarbon radical moiety. Alkyl is optionally substituted and can be linear, branched, or cyclic, (i.e., cycloalkyl). Alkyl includes, but is not limited to, those radicals having one to twenty carbon atoms, for example, C₁₋₂₀ alkyl; one to twelve carbon atoms, for example, C₁₋₁₂ alkyl; one to eight carbon atoms, for example, C₁₋₈ alkyl; one to six carbon atoms, for example, C₁-₆ alkyl; and one to three carbon atoms for example, C₁₋₃ alkyl. Examples of alkyl moieties include, but are not limited to, methyl, ethyl, n-propyl, i-propyl, n-butyl, s-butyl, t-butyl, i-butyl, a pentyl moiety, a hexyl moiety, cyclopropyl, cyclobutyl, cyclopentyl, and cyclohexyl. A pentyl moiety includes, but is not limited to, n-pentyl and i-pentyl. A hexyl moiety includes, but is not limited to, n-hexyl.

As used herein, “haloalkyl” refers to alkyl, as defined herein, wherein the alkyl includes at least one substituent selected from a halogen, e.g., F, Cl, Br, or I.

As used herein, “hydroxyalkyl” refers to alkyl, as defined herein, wherein the alkyl includes at least one hydroxy group.

As used herein, “alkylene” refers to a divalent alkyl group. Unless specified otherwise, alkylene includes, but is not limited to, one to twenty carbon atoms. The alkylene group is optionally substituted as described herein for alkyl. In some embodiments, alkylene is unsubstituted. Examples of alkylene moieties include —CH₂—, —CH₂CH₂—, —CH₂CH₂CH₂—, —CH₂CH₂CH₂CH₂—, and the like.

Designation of an amino acid or an amino acid residue without specifying stereochemistry is intended to encompass the L-form of the amino acid or amino acid residue, the D-form of the amino acid or amino acid residue, or a racemic mixture thereof.

As used herein, “alkoxy” refers to a monovalent and saturated hydrocarbon radical moiety wherein the hydrocarbon includes a single bond to an oxygen atom and wherein the radical is localized on the oxygen atom, for example, CH₃CH₂—O— for ethoxy. Alkoxy substituents bond to the compound which they substitute through this oxygen atom of the alkoxy substituent. Alkoxy is optionally substituted and can be linear, branched, or cyclic, for example, cycloalkoxy. Alkoxy includes, but is not limited to, those radicals having one to twenty carbon atoms, for example, C₁₋₂₀ alkoxy; one to twelve carbon atoms, for example, C₁-₁₂ alkoxy; one to eight carbon atoms, for example, C₁₋₈ alkoxy; one to six carbon atoms, for example, C₁-₆ alkoxy; and one to three carbon atoms, for example, C₁₋₃ alkoxy. Examples of alkoxy moieties include, but are not limited to, methoxy, ethoxy, n-propoxy, i-propoxy, n-butoxy, s-butoxy, t-butoxy, i-butoxy, a pentoxy moiety, a hexoxy moiety, cyclopropoxy, cyclobutoxy, cyclopentoxy, and cyclohexoxy.

As used herein, “haloalkoxy” refers to alkoxy, as defined herein, wherein the hydrocarbon is substituted with at least one halogen, e.g., F, Cl, Br, or I . . .

As used herein, “aryl” refers to a monovalent C₅-C₁₅ carbocyclic ring system which comprises at least one aromatic ring wherein the aryl ring system is mono, di, or tricyclic. The aryl may be attached to the main structure through any of its rings, i.e. any aromatic or nonaromatic ring. In some or any embodiments, the aryl group may be a bridged (where chemically feasible) or non-bridged, spirocyclic (where chemically feasible) or not spirocyclic, and/or fused or not fused multicyclic group. In some or any embodiments, aryl is phenyl, naphthyl, bicyclo[4.2.0]octa-1,3,5-trienyl, indanyl, fluorenyl, 6,7,8,9-tetrahydro-5H-benzo[7]annulenyl,

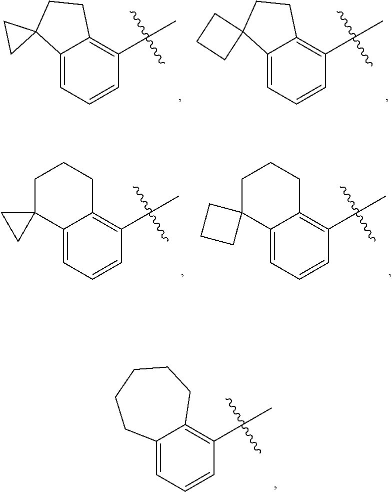 [SMILES: *c1cccc2c1CCC21CC1 ; *c1cccc2c1CCC21CCC1 ; *c1cccc2c1CCCC21CC1 ; *c1cccc2c1CCCC21CCC1 ; *c1cccc2c1CCCCC2]

or tetrahydronaphthyl. When aryl is substituted, it can be substituted on any ring, i.e. on any aromatic or nonaromatic ring comprised by aryl. In some or any embodiments, aryl is phenyl, naphthyl, tetrahydronaphthyl, fluorenyl, 6,7,8,9-tetrahydro-5H-benzo[7]annulenyl, or indanyl

As used herein, “heteroaryl” refers to a monocyclic aromatic ring system or multicyclic aromatic ring system wherein one or more (in some or any embodiments, 1, 2, 3, or 4) of the ring atoms is a heteroatom independently selected from O, S(O)₀₋₂, NH, and N, and the remaining ring atoms are carbon atoms, and where the ring may be optionally substituted as described herein. The heteroaryl group is bonded to the rest of the molecule through any atom in the ring system, valency rules permitting. In certain embodiments, each ring of a heteroaryl group can contain one or two O atoms, one or two S atoms, and/or one to four N atoms, or a combination thereof, provided that the total number of heteroatoms in each ring is four or less and each ring contains at least one carbon atom. In certain embodiments, the heteroaryl has from 5 to 20, from 5 to 15, or from 5 to 10 ring atoms. When heteroaryl is substituted, it can be substituted on any ring. In some embodiments, heteroaryl includes, but is not limited to furanyl, pyrrolyl, pyrazolyl, imidazolyl, triazolyl, tetrazolyl, thienyl, pyridinyl, pyrimidinyl, pyridazinyl, pyrazinyl, indolyl, quinolinyl, isoquinolinyl, oxazolyl, isoxazolyl, thienopyridinyl, thienopyrimidinyl, azaindolinyl and the like. In some embodiments, a heteroaryl is

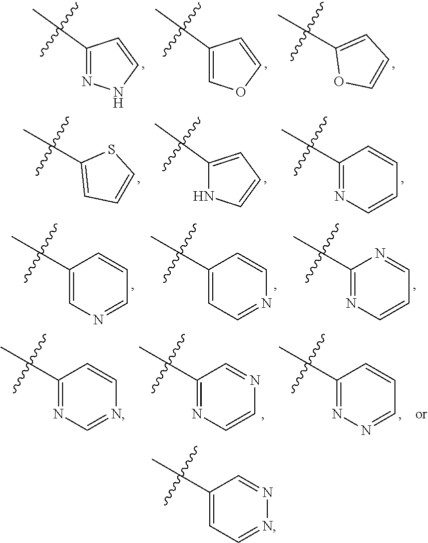

wherein [symbol] indicates the point of attachment of the heteroaryl to the rest of the molecule. In some embodiments, the heteroaryl when substituted one or more hydroxy can be named or drawn as either the keto or enol tautomer. For example, 2,4 (1H,3H)-dioxo-pyrimidinyl, 2,4-dihydroxy-pyrimidinyl; 2 (1H)-oxo-4-hydroxypyrimidinyl, 4-hydroxy-2 (3H)-oxo-pyrimidinyl, and 2-hydroxy-4 (3H)-oxo-pyrimidinyl are within the scope of heteroaryl when substituted with two hydroxy. In some embodiments, the “heteroaryl” is N-linked.

In certain embodiments, monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl and triazolyl. In certain embodiments, bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. In certain embodiments, tricyclic heteroaryl groups include, but are not limited to, acridinyl, benzindolyl, carbazolyl, dibenzofuranyl, perimidinyl, phenanthrolinyl, phenanthridinyl, and phenazinyl. In some or any embodiments, heteroaryl is indolyl, furanyl, pyridinyl, pyrimidinyl, imidazolyl, or pyrazolyl; each of which is optionally substituted with 1, 2, 3, or 4 groups as defined throughout the specification, including in some embodiments with group(s) independently selected from C₁-₆alkyl, hydroxy, halo, halo-C₁₋₆ alkyl, C₁₋₆ alkoxy, cyano, or phenyl.

As used herein, “heterocycloalkyl” or “heterocyclyl” refers to a monovalent monocyclic non-aromatic ring system and/or a multicyclic ring system that contains at least one non-aromatic ring; wherein one or more (in some or any embodiments, 1, 2, 3, or 4) of the non-aromatic monocyclic ring atoms is a heteroatom independently selected from O, S(O)₀₋₂, and N, and the remaining ring atoms are carbon atoms; and wherein one or more (in some or any embodiments, 1, 2, 3, or 4) of any of the ring atoms in the multicyclic ring system is a heteroatom(s) independently selected from O, S(O)₀₋₂, and N, and the remaining ring atoms are carbon. The term “heterocyclic” does not include fully aromatic ring(s), i.e. does not include imidazole, pyrimidine, pyridine, and the like. In some or any embodiments, the heterocyclic ring comprises one or two heteroatom(s) which are independently selected from nitrogen and oxygen. In some or any embodiments, the heterocyclic ring comprises one or two heteroatom(s) which are oxygen. In some or any embodiments, the heterocyclic ring comprises one or two heteroatom(s) which are nitrogen (where the nitrogen is substituted as described in any aspect or embodiment described herein). In some or any embodiments, heterocyclic is multicyclic and comprises one heteroatom in a non-aromatic ring, or comprises one heteroatom in an aromatic ring, or comprises two heteroatoms in an aromatic ring, or comprises two heteroatoms where one is in an aromatic ring and the other is in a non-aromatic ring. In some or any embodiments, the heterocyclic group has from 3 to 20, 3 to 15, 3 to 10, 3 to 8, 4 to 7, or 5 to 6 ring atoms. In some or any embodiments, the heterocyclic is a monocyclic, bicyclic, tricyclic, or tetracyclic ring system. In some or any embodiments, the heterocyclic group may be a bridged or non-bridged, spirocyclic or not spirocyclic, and/or fused or not fused multicyclic group. One or more of the nitrogen and sulfur atoms may be optionally oxidized, one or more of the nitrogen atoms may be optionally quaternized, one or more of the carbon atoms may be optionally replaced with

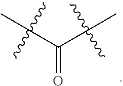 [SMILES: *C(*)=O]

Some rings may be partially or fully saturated, or aromatic provided that heterocyclic is not fully aromatic. The monocyclic and multicyclic heterocyclic rings may be attached to the main structure at any heteroatom or carbon atom which results in a stable compound. The multicyclic heterocyclic may be attached to the main structure through any of its rings, including any aromatic or nonaromatic ring, regardless of whether the ring contains a heteroatom. In some or any embodiments, heterocyclic is “heterocycloalkyl” which is 1) a saturated or partially unsaturated (but not aromatic) monovalent monocyclic group which contains at least one ring heteroatom, as described herein, or 2) a saturated or partially unsaturated (but not aromatic) monovalent bi- or tri-cyclic group in which at least one ring contains at least one heteroatom as described herein. When heterocyclic and heterocycloalkyl are substituted, they can be substituted on any ring, i.e. on any aromatic or nonaromatic ring comprised by heterocyclic and heterocycloalkyl. In some or any embodiments, such heterocyclic includes, but are not limited to, azepinyl, benzodioxanyl, benzodioxolyl, 3,4-dihydro-2H-benzo[b][1,4]oxazinyl, 3,4-dihydro-2H-benzo[b][1,4]dioxepinyl, 1,3-dihydroisobenzofuranyl, benzofuranonyl, benzopyranonyl, benzopyranyl, dihydrobenzofuranyl,

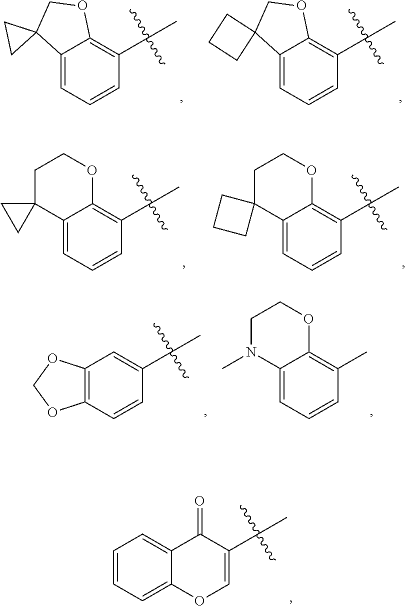

benzotetrahydrothienyl, 2,2-dioxo-1,3-dihydrobenzo[c]thienyl, benzothiopyranyl, benzoxazinyl, ß-carbolinyl, chromanyl, chromonyl, cinnolinyl, coumarinyl, decahydroquinolinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, 2,4-dioxo-imidazolidinyl, imidazolinyl, indolinyl, 2-oxo-indolinyl, isobenzotetrahydrofuranyl, isobenzotetrahydrothienyl, isochromanyl, isocoumarinyl, isoindolinyl, 1-oxo-isoindolinyl, 1,3-dioxo-isoindolinyl, isothiazolidinyl, isoxazolidinyl, 3-oxo-isoxazolidinyl, morpholinyl, 3,5-dioxo-morpholinyl, octahydroindolyl, octahydroisoindolyl, 1-oxo-octahydroisoindolyl, 1,3-dioxo-hexahydroisoindolyl, oxazolidinonyl, oxazolidinyl, oxiranyl, piperazinyl, 2,6-dioxo-piperazinyl, piperidinyl, 2,6-dioxo-piperidinyl, 4-piperidonyl, pyrazolidinyl, pyrazolinyl, pyrrolidinyl, pyrrolinyl, 2-oxopyrrolidinyl, 2,5-dioxopyrrolidinyl, quinuclidinyl, tetrahydrofuryl, tetrahydroisoquinolinyl, tetrahydropyranyl, tetrahydrothienyl, thiamorpholinyl, thiomorpholinyl, 3,5-dioxo-thiomorpholinyl, thiazolidinyl, 2,4-dioxo-thiazolidinyl, tetrahydroquinolinyl, phenothiazinyl, phenoxazinyl, xanthenyl, and 1,3,5-trithianyl. In some or any embodiments, heterocyclic is benzo-1,4-dioxanyl, benzodioxolyl, indolinyl, 2-oxo-indolinyl, pyrrolidinyl, piperidinyl, 2,3-dihydrobenzofuranyl, or decahydroquinolinyl; each of which is optionally substituted with 1, 2, 3, or 4 groups as defined throughout the specification, including in some or any embodiments with group(s) independently selected from halo, alkyl, and phenyl. In some embodiments, heterocycloalkyl is pyrrolidinyl.

As used herein, “cyano” refers to —CN.

The term “oxo” as used herein and unless otherwise specified, refers to a keto group (C═O). An oxo group that is a substituent of a nonaromatic carbon results in a conversion of a —CH₂— to —C═O. An oxo group that is a substituent of an aromatic carbon results in a conversion of —CH— to —C═O. When a substituent is oxo, then two hydrogens on the atom are replaced. When an oxo group substitutes aromatic moieties, the corresponding partially unsaturated ring replaces the aromatic ring. For example, a pyridyl group substituted by an oxo group is a pyridone. The person of ordinary skill in the art will appreciate that in some embodiments that such a group, e.g. pyridone and 2,4 (1H,3H)-dioxo-pyrimidinyl, can exist in its tautomeric form, e.g. hydroxypyridine and 2,4-dihydroxypyrimidinyl, respectively.

As used herein, “optionally substituted” when used to describe a radical moiety, for example, optionally substituted alkyl, means that such moiety is optionally bonded to one or more substituents. Examples of such substituents include, but are not limited to, halo, cyano, nitro, amino, hydroxyl, optionally substituted haloalkyl, aminoalkyl, hydroxyalkyl, azido, epoxy, optionally substituted heteroaryl, optionally substituted heterocycloalkyl,

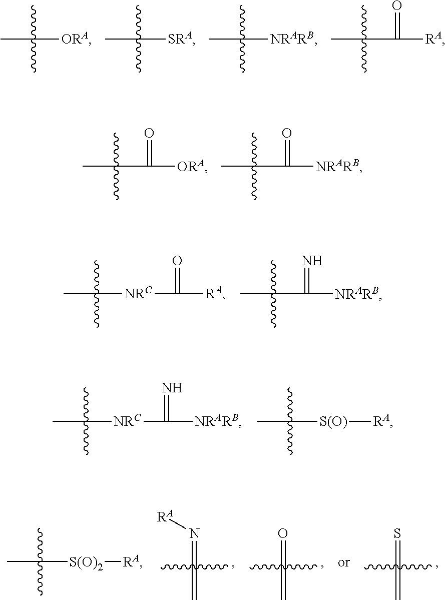

wherein R^{A}, R^{B}, and R^{C} are, independently at each occurrence, hydrogen, alkyl, alkenyl, alkynyl, aryl, alkylaryl, arylalkyl, heteroalkyl, heteroaryl, or heterocycloalkyl, or R^{A} and R^{B} together with the atoms to which they are bonded, form a saturated or unsaturated carbocyclic ring, wherein the ring is optionally substituted, and wherein one or more ring atoms is optionally replaced with a heteroatom. In certain embodiments, when a radical moiety is optionally substituted with an optionally substituted heteroaryl, optionally substituted heterocycloalkyl, or optionally substituted saturated or unsaturated carbocyclic ring, the substituents on the optionally substituted heteroaryl, optionally substituted heterocycloalkyl, or optionally substituted saturated or unsaturated carbocyclic ring, if they are substituted, are not substituted with substituents which are further optionally substituted with additional substituents. In some embodiments, when a group described herein is optionally substituted, the substituent bonded to the group is unsubstituted unless otherwise specified.

As used herein, the term “epimer” is one of a pair of diastereomers that have the opposite configuration at only one stereogenic center, or chiral center, out of at least two. For example, a compound of Formula (I), (Ia), (Ib), or (Ic) with (R)-stereochemistry at the C-16 position is epimer of the corresponding compound with(S)-stereochemistry.

As used herein, “diastereomeric excess (de)” refers to a dimensionless mole ratio describing the purity of chiral substances that contain more than one stereogenic center. For example, a diastereomeric excess of zero would indicate an equimolar mixture of diastereoisomers. By way of further example, diastereomeric excess of ninety-nine would indicate a nearly stereopure diastereomeric compound (i.e., large excess of one diastereomer over the other). Diastereomeric excess may be calculated via a similar method to ee. As would be appreciated by a person of skill, de is usually reported as percent de (% de). % de may be calculated in a similar manner to % cc.

As used herein, “therapeutically effective amount” refers to an amount (e.g., of a compound) that is sufficient to provide a therapeutic benefit to a patient in the treatment or management of a disease or disorder, or to delay or minimize one or more symptoms associated with the disease or disorder.

Certain groups, moieties, substituents, and atoms are depicted with a wiggly line that intersects a bond or bonds to indicate the atom through which the groups, moieties, substituents, atoms are bonded. For example, a phenyl group that is substituted with a propyl group depicted as:

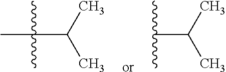

has the following structure:

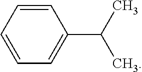

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains. For example, the group,

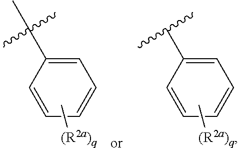

wherein subscript q is an integer from zero to two and in which the positions of substituent R^{2a} are described generically, for example, not directly attached to any vertex of the bond line structure, for example, a specific ring carbon atom, includes the following, non-limiting examples of groups in which the substituent R^{2a} is bonded to a specific ring carbon atom:

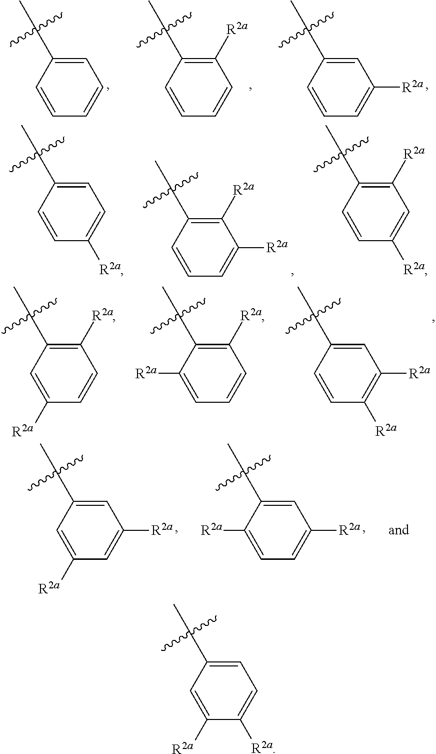

### FKB12 Selective Rapamycin Analogs

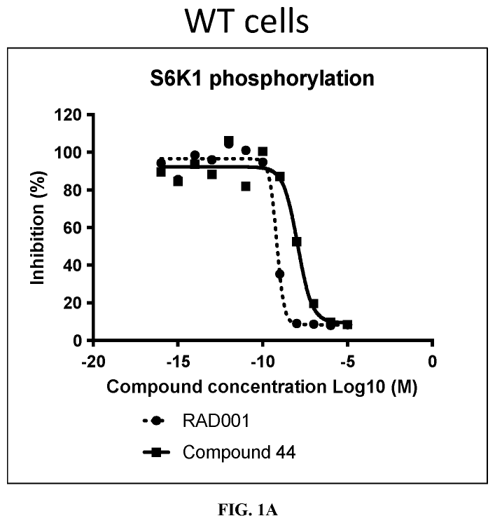

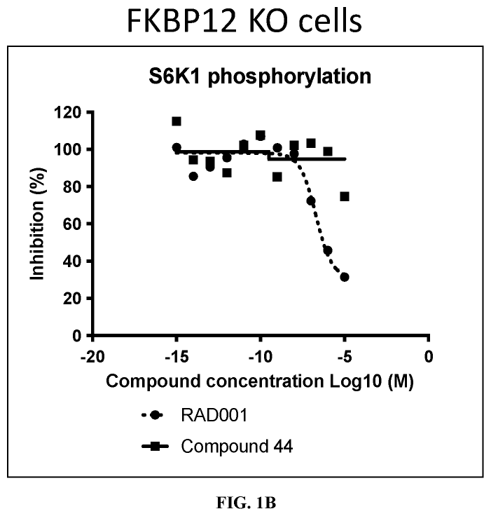

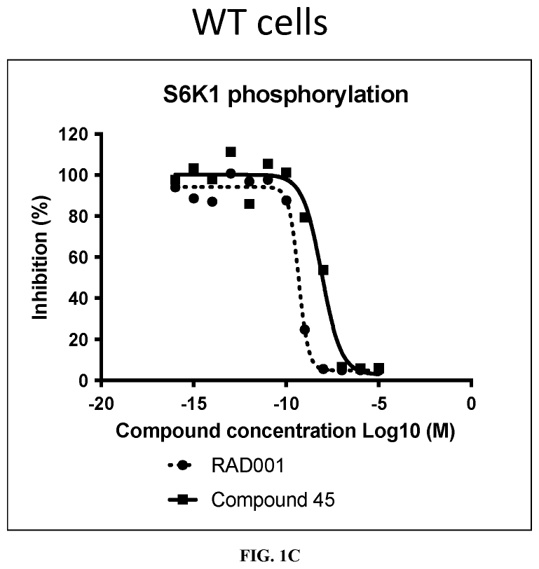

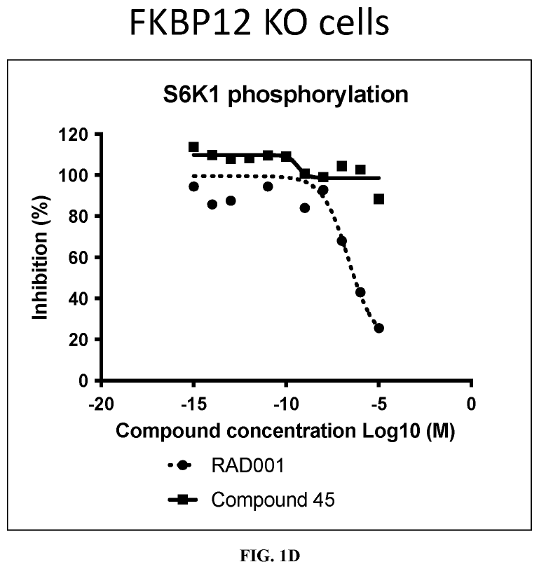

As used herein, “FKBP12 selective” refers to the requirement for FKBP12 for inhibition of mTORC1 phosphorylation of S6K1, as shown in FIG. 1C; compound 45 is able to inhibit S6K1 phosphorylation in the presence of FKBP12; however, when FKBP12 is knocked out, there is much less inhibition of S6K1 phosphorylation with compound 45 relative to RAD001, a rapalog that can act through other FKBPs.

Alternatively, an “FKBP12-selective rapalog” is a rapalog, while it is potent in an assay using a wild-type of cells, it is approximately 10-fold to approximately 1000-fold less potent than RAD001 in a cell line that does not express FKBP12 (e.g., FKBP12 knock-out cells). “Potency,” as used in this context, can be expressed as the concentration of rapalog required to achieve a 20% inhibition of S6K1 (Thr389)phosphorylation in a cell-based assay such as that used in Example 3 and FIGS. 1-11. For example, a rapalog is considered FKBP12-selective if the rapalog inhibits about 20% of S6K1 (Thr389)phosphorylation inhibition in assay using an FKBP12 knock-out cell line with at least 20× (e.g. 20×, 30×, 50×, 100×, 200×, 500×, 1000×, etc.) lower potency compared to similar level of inhibition achieved by RAD001 in the same assay with the same cell line, which normally expresses FKBP12.

Alternatively, an “FKBP12-selective rapalog” is a rapalog, while it is potent in an assay using a wild-type of cells, it is approximately 10-fold to approximately 1000-fold less potent than RAD001 in a cell line that does not express FKBP12 (e.g., FKBP12 knock-out cells). “Potency,” as used in this context, can be expressed as the concentration of rapalog required to achieve a 30% inhibition of S6K1 (Thr389)phosphorylation in a cell-based assay such as that used in Example 3 and FIGS. 1-11. For example, a rapalog is considered FKBP12-selective if the rapalog inhibits about 30% of S6K1 (Thr389)phosphorylation inhibition in assay using an FKBP12 knock-out cell line with at least 20× (e.g. 20×, 30×, 50×, 100×, 200×, 500×, 1000×, etc.) lower potency compared to similar level of inhibition achieved by RAD001 in the same assay with the same cell line, which normally expresses FKBP12.

In one embodiment, the compound of Formula (I) is a compound of Formula (P-I):

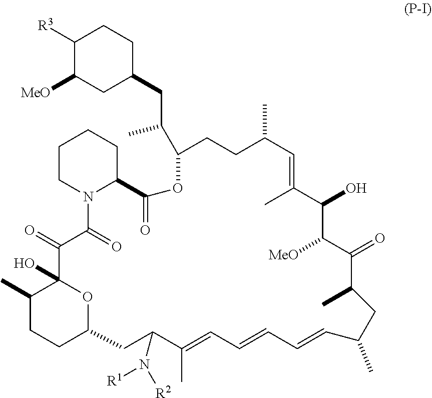

or a pharmaceutically acceptable salt thereof;
wherein R¹ is hydrogen, C₁-₆ alkyl, heterocyclyl, aryl, heteroaryl, —C₀₋₆alkylene-SO₂R⁴, or —C₀₋₆alkylene-SO₂R⁵; wherein the heterocyclyl, aryl, heteroaryl are optionally substituted with one or two R^{1a} groups;
R² is heterocyclyl, aryl, heteroaryl, —C₀₋₆ alkylene-SO₂R⁴, or —C₀₋₆ alkylene-SO₂R⁵; wherein the heterocyclyl, aryl, and heteroaryl are optionally substituted with one or two R^{2a} groups;
or R¹ and R² together with the attached nitrogen form a N-linked heteroaryl;
R³ is selected from the group consisting of —OR^{a}, 3-6 membered heterocyclyl, and 3-6 membered heteroaryl;
R^{a} is selected from the group consisting of H, —P(O)(R^{b})₂, —C(O)R^{c}, —C(O)OR^{c}, C₁-₆ alkyl, and C₁₋₆ hydroxyalkyl;
each R^{b} is independently selected from the group consisting of H and C₁-₆ alkyl;
R^{c} is selected from the group consisting of H, C₁-₆ alkyl, and C₁₋₆ hydroxyalkyl;
R⁴ is C₂₋₆ alkyl, C₃₋₆cycloalkyl, C₁₋₆ hydroxyalkyl, heterocyclyl, aryl, or heteroaryl; wherein the heterocyclyl, aryl, and heteroaryl are optionally substituted with one or two R^{4a} groups; and
R⁵ is heterocyclyl, aryl, or heteroaryl; wherein the heterocyclyl, aryl, and heteroaryl are optionally substituted with one or two R^{5a} groups;
each of the one or two R^{1a}, R^{2a}, R^{4a}, and R^{5a} groups, when present, are independently selected from C₁₋₆ alkyl, C₁₋₆ hydroxyalkyl, hydroxy, halo, C₁-6haloalkyl, and C₁₋₆ alkoxy; or any two R^{1a}, R^{2a}, R^{4a}, and R^{5a} groups, when present on the same carbon, are taken together to form an oxo group;
wherein when R¹ is hydrogen, R² is not-C₀₋₆ alkylene-SO₂R⁴.

In one embodiment, the compound of Formula (Ia) is a compound of Formula (P-Ia):

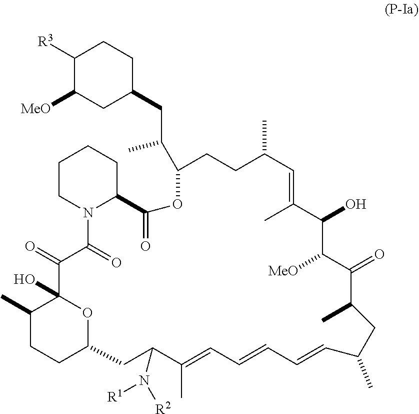

or a pharmaceutically acceptable salt thereof;
wherein R¹ is hydrogen or C₁-₆ alkyl;
R² is heterocyclyl, aryl, or heteroaryl optionally substituted with one or two R^{2a} groups;
R³ is selected from the group consisting of —OR^{a}, a 3-6 membered heterocyclyl, and 3-6 membered heteroaryl;
R^{a} is selected from the group consisting of H, —P(O)(R^{b})₂, —C(O)R^{c}, —C(O)OR^{c}, C₁-₆alkyl, and C₁₋₆ hydroxyalkyl;
each R^{b} is independently selected from the group consisting of H and C₁₋₆ alkyl;
R^{c} is selected from the group consisting of H, C₁-₆ alkyl, and C₁₋₆ hydroxyalkyl; and
each of the one or two R^{2a} groups, when present, are independently selected from C₁-₆alkyl, C₁₋₆ hydroxyalkyl, hydroxy, halo, C₁₋₆ haloalkyl, and C₁₋₆ alkoxy; or any two R^{2a} groups, when present on the same carbon, are taken together to form an oxo group.

In one embodiment, the compound of Formula (Ib) is a compound of Formula (P-Ib):

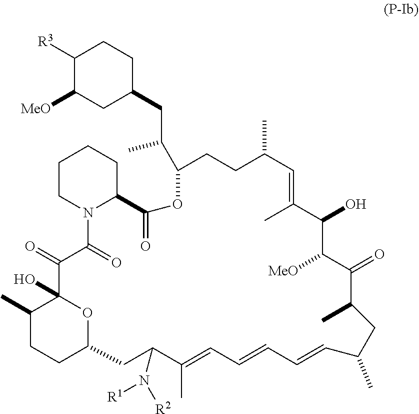

or a pharmaceutically acceptable salt thereof;
wherein R¹ and R² together with the attached nitrogen form a N-linked heteroaryl optionally substituted with one or two R^{1a} groups;
R³ is selected from the group consisting of —OR^{a}, a 3-6 membered heterocyclyl, and 3-6 membered heteroaryl;
R^{a} is selected from the group consisting of H, —P(O)(R^{b})₂, —C(O)R^{c}, —C(O)OR^{c}, C₁-₆alkyl, and C₁₋₆ hydroxyalkyl;
each R^{b} is independently selected from the group consisting of H and C₁-₆ alkyl;
R^{c} is selected from the group consisting of H, C₁-₆ alkyl, and C₁₋₆ hydroxyalkyl;
each of the one or two R^{1a} groups, when present, are independently selected from C₁-₆alkyl, C₁₋₆ hydroxyalkyl, hydroxy, halo, C₁₋₆ haloalkyl, and C₁₋₆ alkoxy; or two R^{1a} groups, when present on the same carbon, are taken together to form an oxo group.

In one embodiment, the compound of Formula (Ic) is a compound of Formula (P-Ic):

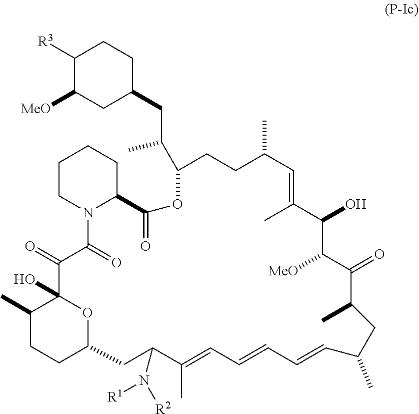

or a pharmaceutically acceptable salt thereof;
wherein R¹ is hydrogen or C₁-₆ alkyl;
R² is —C₀₋₆alkylene-SO₂R⁴ or —C₀₋₆alkylene-SO₂R⁵;
R³ is selected from the group consisting of —OR^{a}, a 3-6 membered heterocyclyl, and 3-6 membered heteroaryl;
R^{a} is selected from the group consisting of H, —P(O)(R^{b})₂, —C(O)Re, —C(O)OR^{c}, C₁-₆ alkyl, and C₁₋₆ hydroxyalkyl;
each R^{b} is independently selected from the group consisting of H and C₁-₆ alkyl;
R^{c} is selected from the group consisting of H, C₁-₆ alkyl, and C₁₋₆ hydroxyalkyl;
R⁴ is C₂₋₆ alkyl, C₃₋₆cycloalkyl, C₁₋₆ hydroxyalkyl, heterocyclyl, aryl, or heteroaryl; wherein the heterocyclyl, aryl, and heteroaryl are optionally substituted with one or two R^{4a} groups;
R⁵ is heterocyclyl, aryl, or heteroaryl; wherein the heterocyclyl, aryl, and heteroaryl are optionally substituted with one or two R^{5a} groups;
each of the one or two R^{4a} and R^{5a} groups, when present, are independently selected from C₁₋₆ alkyl, C₁₋₆ hydroxyalkyl, hydroxy, halo, C₁-6haloalkyl, and C₁₋₆ alkoxy; or any two R^{4a} and R^{5a} groups, when present on the same carbon, are taken together to form an oxo group;
wherein when R¹ is hydrogen, R² is not-C₀₋₆ alkylene-SO₂R⁴.

In one embodiment, the compound of Formula (I) is a compound of Formula (P-2):

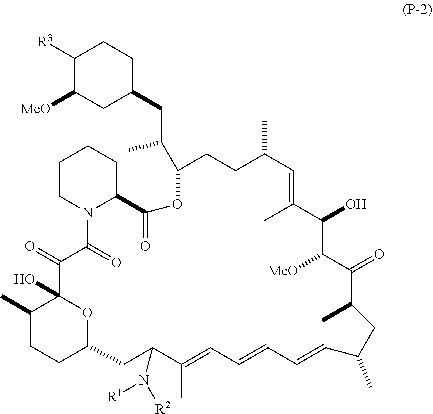

or a pharmaceutically acceptable salt thereof;
wherein R¹ is hydrogen, C₁-₆ alkyl, heterocyclyl, aryl, heteroaryl, —C₀₋₆alkylene-SO₂R⁴, or —C₀₋₆alkylene-SO₂R⁵; wherein the heterocyclyl, aryl, and heteroaryl are optionally substituted with one or two R^{1a} groups;
R² is heterocyclyl, aryl, heteroaryl, —C₀₋₆alkylene-SO₂R⁴, or —C₀₋₆alkylene-SO₂R⁵; wherein the heterocyclyl, aryl, and heteroaryl are optionally substituted with one or two R^{2a} groups;
or R¹ and R² together with the attached nitrogen form a N-linked heteroaryl or N-linked heterocyclyl optionally substituted with one or two R^{1a} groups;
R³ is selected from the group consisting of —OR^{a}, 3-6 membered heterocyclyl, and 3-6 membered heteroaryl;
R^{a} is selected from the group consisting of H, —P(O)(R^{b})₂, —C(O)R^{c}, —C(O)OR^{c}, C₁-₆alkyl, and C₁₋₆ hydroxyalkyl;
each R^{b} is independently selected from the group consisting of H and C₁-₆ alkyl;
R^{c} is selected from the group consisting of H, C₁₋₆ alkyl, and C₁₋₆ hydroxyalkyl;
R⁴ is C₂₋₆alkyl, C₃₋₆cycloalkyl, C₁₋₆ hydroxyalkyl, heterocyclyl, aryl, or heteroaryl; wherein the heterocyclyl, aryl, and heteroaryl are optionally substituted with one or two R^{4a} groups; and
R⁵ is heterocyclyl, aryl, or heteroaryl; wherein the heterocyclyl, aryl, and heteroaryl are optionally substituted with one or two R^{5a} groups;
each of the one or two R^{1a}, R^{2a}, R^{4a}, and R^{5a} groups, when present, are independently selected from C₁₋₆alkyl, C₁₋₆ hydroxyalkyl, hydroxy, halo, C₁₋₆ haloalkyl, C₁₋₆ alkoxy, C₁₋₆haloalkoxy, and cyano; or any two R^{1a}, R^{2a}, R^{4a}, and R^{5a} groups, when present on the same carbon, are taken together to form an oxo group;
wherein when R¹ is hydrogen, R² is not-C₀₋₆ alkylene-SO₂R⁴.

In one embodiment of Formula (I), (Ia)-(Ic), (P-1), (P-2), or (P-Ia)-(P-Ic), R³ is —OR^{a}. In one embodiment of Formula (I), (Ia)-(Ic), (P-1), (P-2), or (P-Ia)-(P-Ic), R³ is —OH. In one embodiment of Formula (I), (Ia)-(Ic), (P-1), (P-2), or (P-Ia)-(P-Ic), R³ is —OP(O)(R^{b})₂. In one embodiment of Formula ((I), (Ia)-(Ic), (P-1), (P-2), or (P-Ia)-(P-Ic), R³ is —OP(O)H₂. In one embodiment of Formula (I), (Ia)-(Ic), (P-1), (P-2), or (P-Ia)-(P-Ic), R³ is —OP(O)(C₁₋₆ alkyl)₂. In one embodiment of Formula (I), (Ia)-(Ic), (P-1), (P-2), or (P-Ia)-(P-Ic), R³ is —OP(O)(Me)₂. In one embodiment of Formula (I), (Ia)-(Ic), (P-1), (P-2), or (P-Ia)-(P-Ic), R³ is —OP(O)(C₁-₆alkyl)(H). In one embodiment of Formula (I), (Ia)-(Ic), (P-1), (P-2), or (P-Ia)-(P-Ic), R³ is —OP(O)(Mc)(H). In one embodiment of Formula (I), (Ia)-(Ic), (P-1), (P-2), or (P-Ia)-(P-Ic), R³ is —C(O)R°. In one embodiment of Formula (I), (Ia)-(Ic), (P-1), (P-2), or (P-Ia)-(P-Ic), R³ is —C(O)H. In one embodiment of Formula (I), (Ia)-(Ic), (P-1), (P-2), or (P-Ia)-(P-Ic), R³ is —C(O) C₁₋₆ alkyl. In one embodiment of Formula (I), (Ia)-(Ic), (P-1), (P-2), or (P-Ia)-(P-Ic), R³ is —C(O) C₁₋₆hydroxyalkyl. In one embodiment of Formula (I), (Ia)-(Ic), (P-1), (P-2), or (P-Ia)-(P-Ic), R³ is —C(O)OR^{c}. In one embodiment of Formula (I), (Ia)-(Ic), (P-1), (P-2), or (P-Ia)-(P-Ic), R³ is —COOH. In one embodiment of Formula (I), (Ia)-(Ic), (P-1), (P-2), or (P-Ia)-(P-Ic), R³ is —C(O)OC₁₋₆alkyl. In one embodiment of Formula (I), (Ia)-(Ic), (P-1), (P-2), or (P-Ia)-(P-Ic), R³ is —C(O)OC₁₋₆hydroxyalkyl. In one embodiment of Formula (I), (Ia)-(Ic), (P-1), (P-2), or (P-Ia)-(P-Ic), R³ is —OC₁₋₆alkyl. In one embodiment of Formula (I), (Ia)-(Ic), (P-1), (P-2), or (P-Ia)-(P-Ic), R³ is —OC₁₋₆hydroxyalkyl.

In one embodiment of Formula (I), (Ia)-(Ic), (P-1), (P-2), or (P-Ia)-(P-Ic), R³ is 3-6-membered heterocyclyl. In one embodiment of Formula (I), (Ia)-(Ic), (P-1), (P-2), or (P-Ia)-(P-Ic), R³ is 3-6-membered heterocyclyl containing one, two, or three atoms selected from nitrogen, oxygen, and sulfur. In one embodiment of Formula (I), (Ia)-(Ic), (P-1), (P-2), or (P-Ia)-(P-Ic), R³ is 3-6-membered heterocyclyl containing one nitrogen atom. In one embodiment of Formula (I), (Ia)-(Ic), (P-1), (P-2), or (P-Ia)-(P-Ic), R³ is 3-6-membered heterocyclyl containing two or three nitrogen atoms.

In one embodiment of Formula (I), (Ia)-(Ic), (P-1), (P-2), or (P-Ia)-(P-Ic), R³ is 3-6-membered heteroaryl. In one embodiment of Formula (I), (Ia)-(Ic), (P-1), (P-2), or (P-Ia)-(P-Ic), R³ is 3-6-membered heteroaryl containing one, two, three, or four atoms selected from nitrogen, oxygen, and sulfur atoms. In one embodiment of Formula (I), (Ia)-(Ic), (P-1), (P-2), or (P-Ia)-(P-Ic), R³ is 3-6-membered heteroaryl containing one nitrogen atom. In one embodiment of Formula (I), (Ia)-(Ic), (P-1), (P-2), or (P-Ia)-(P-Ic), R³ is 3-6-membered heteroaryl containing two or three nitrogen atoms. In one embodiment of Formula (I), (Ia)-(Ic), (P-1), (P-2), or (P-Ia)-(P-Ic), R³ is 3-6-membered heteroaryl containing four nitrogen atoms. In one embodiment of Formula (I), (Ia)-(Ic), (P-1), (P-2), or (P-Ia)-(P-Ic), R³ is a tetrazole or a triazole. In one embodiment of Formula (I), (Ia)-(Ic), (P-1), (P-2), or (P-Ia)-(P-Ic), R³ is

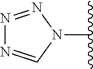

wherein [symbol] is the point of attachment to the rest of the compound.

In one embodiment of Formula (I), (Ia)-(Ic), (P-1), (P-2), or (P-Ia)-(P-Ic), including any of the foregoing, R¹ is hydrogen. In one embodiment of Formula (I), (Ia)-(Ic), (P-1), (P-2), or (P-Ia)-(P-Ic), including any of the foregoing, R¹ is C₁-6alkyl. In one embodiment of Formula (I), (P-I), or (P-2), including any of the foregoing, R¹ is heterocyclyl, aryl, or heteroaryl. In one embodiment of Formula (I), (P-I), or (P-2), including any of the foregoing, R¹ is heterocyclyl, aryl, or heteroaryl. In one embodiment of Formula (I), (P-I), or (P-2), including any of the foregoing, R¹ is —C₀₋₆alkylene-SO₂R⁴ or —C₀₋₆alkylene-SO₂R⁵.

In one embodiment of Formula (I), (Ia)-(Ic), (P-1), (P-2), or (P-Ia)-(P-Ic), including any of the foregoing, R¹ is C₁-6alkyl wherein the C₁₋₆ alkyl is unsubstituted. In one embodiment of Formula (I), (Ia)-(Ic), (P-1), (P-2), or (P-Ia)-(P-Ic), including any of the foregoing, R¹ is C₁-₆alkyl wherein the C₁₋₆ alkyl is substituted with one or two R^{2a} groups. In one embodiment of Formula (I), (Ia)-(Ic), (P-1), (P-2), or (P-Ia)-(P-Ic), including any of the foregoing, R¹ is C₁-6alkyl wherein the C₁₋₆ alkyl is substituted with C₁₋₆ hydroxyalkyl. In one embodiment of Formula (I), (Ia)-(Ic), (P-1), (P-2), or (P-Ia)-(P-Ic), including any of the foregoing, R¹ is

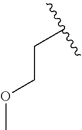

wherein [symbol] is the point of attachment to the rest of the compound. In one embodiment of Formula (I), (Ia)-(Ic), (P-1), (P-2), or (P-Ia)-(P-Ic), including any of the foregoing, R¹ is C₁-₆alkyl wherein the C₁₋₆ alkyl is substituted with C₁₋₆ alkoxy. In one embodiment of Formula (I), (Ia)-(Ic), (P-1), (P-2), or (P-Ia)-(P-Ic), including any of the foregoing, R¹ is

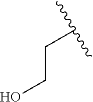

wherein [symbol] is the point of attachment to the rest of the compound.

In one embodiment of Formula (I), (P-I), (Ia), (P-Ia), or (P-2), including any of the foregoing, R² is heterocyclyl wherein the heterocyclyl is unsubstituted. In one embodiment of Formula (I), (P-I), (Ia), (P-Ia), or (P-2), including any of the foregoing, R² is heterocyclyl wherein the heterocyclyl is substituted with one or two R^{2a} groups. In one embodiment of Formula (I), (P-I), (Ia), (P-Ia), or (P-2), including any of the foregoing, R² is a 5-12 membered monocyclic or bicyclic heterocyclyl containing one, two, or three atoms selected from nitrogen, oxygen, and sulfur wherein when a nitrogen is present, the nitrogen is optionally substituted with C₁₋₆ alkyl or C₃₋₆cycloalkyl and wherein the heterocyclyl is optionally substituted with one or two R^{2a} groups.

In one embodiment of Formula (I), (P-I), (Ia), (P-Ia), or (P-2), including any of the foregoing, R² is aryl wherein the aryl is unsubstituted. In one embodiment of Formula (I), (P-I), (Ia), (P-Ia), or (P-2), including any of the foregoing, R² is a 5-12 membered monocyclic or bicyclic aryl wherein the aryl is optionally substituted with one or two R^{2a} groups. In one embodiment of Formula (I), (P-I), (Ia), (P-Ia), or (P-2), including any of the foregoing, R² is unsubstituted phenyl. In one embodiment of Formula (I), (P-I), (Ia), (P-Ia), or (P-2), including any of the foregoing, R² is phenyl substituted with one or two R^{2a} groups. In one embodiment of Formula (I), (P-I), (Ia), (P-Ia), or (P-2), including any of the foregoing, R² is phenyl substituted with one R^{2a} group selected from halo, C₁-6alkoxy, C₁₋₆ alkyl, and C₁₋₆ haloalkyl.

In one embodiment of Formula (I), (P-I), (Ia), (P-Ia), or (P-2), including any of the foregoing, R² is heteroaryl wherein the heteroaryl is unsubstituted. In one embodiment of Formula (I), (P-I), (Ia), (P-Ia), or (P-2), including any of the foregoing, R² is heteroaryl wherein the heteroaryl is optionally substituted with one or two R^{2a} groups. In one embodiment of Formula (I), (P-I), (Ia), (P-Ia), or (P-2), including any of the foregoing, R² is a 5-12 membered monocylic or bicyclic heteroaryl containing one, two, three, or four atoms selected from nitrogen, oxygen, and sulfur wherein when a nitrogen is present, the nitrogen is optionally substituted with C₁₋₆ alkyl or C₃₋₆cycloalkyl and wherein the heteroaryl is optionally substituted with one or two R^{2a} groups.

In one embodiment of Formula (I), (P-I), (Ia), (P-Ia), or (P-2), including any of the foregoing, R² is a 5-12 membered monocylic heteroaryl containing one nitrogen atom optionally substituted with C₁₋₆ alkyl or C₃₋₆cycloalkyl and wherein the heteroaryl is optionally substituted with two R^{2a} groups on the same carbon that are joined together to form an oxo group.

In one embodiment of Formula (I), (P-I), (Ia), (P-Ia), or (P-2), including any of the foregoing, R² is selected from

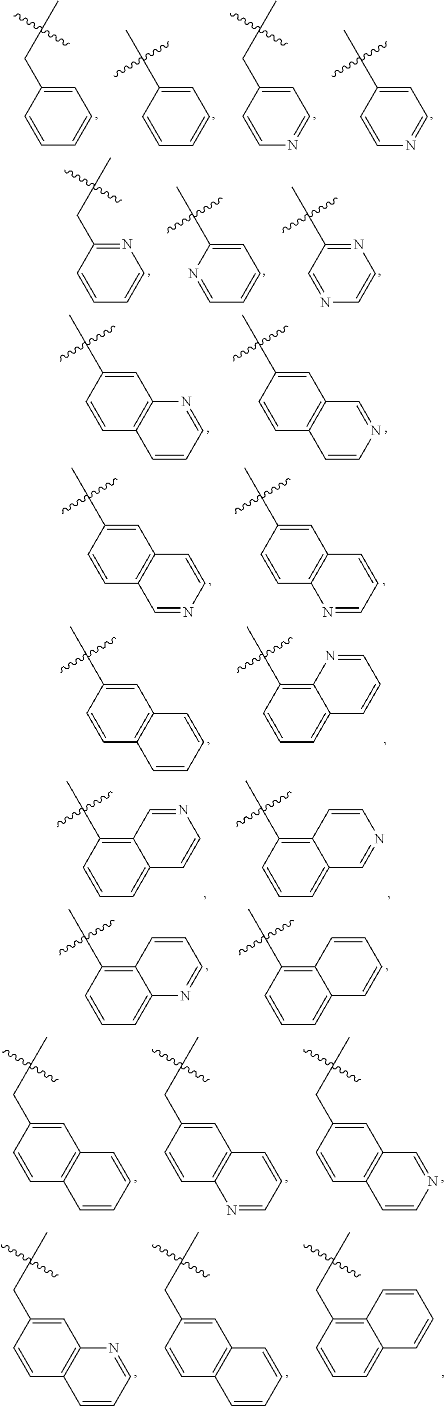

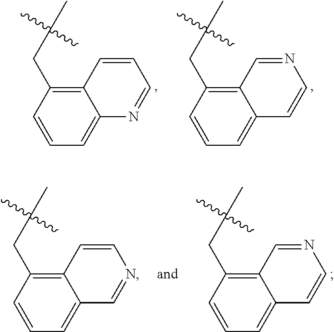

wherein [symbol] is the point of attachment to the rest of the compound.

In one embodiment of Formula (I), (P-I), (Ia), (P-Ia), or (P-2), including any of the foregoing, R² is selected from

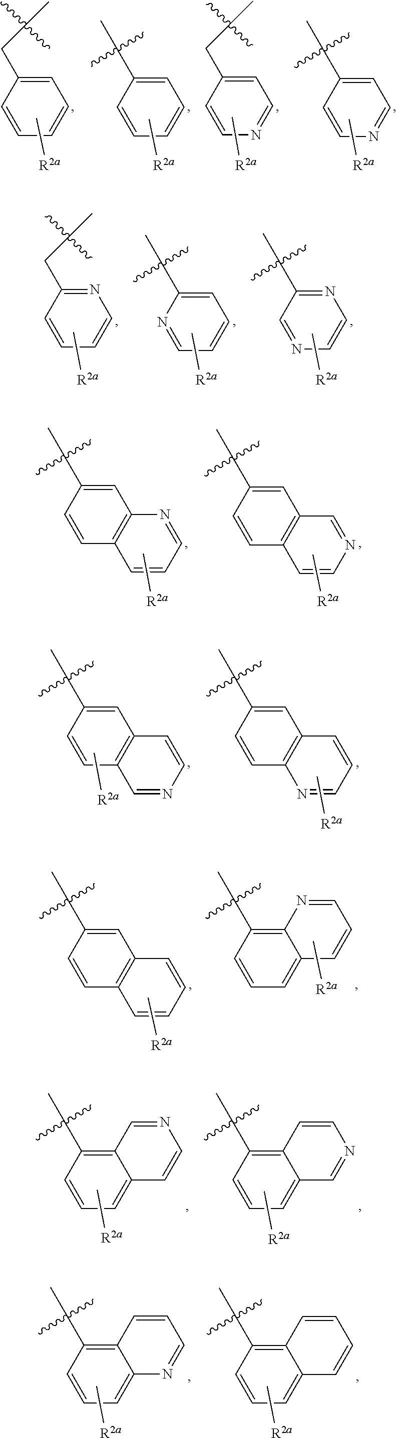

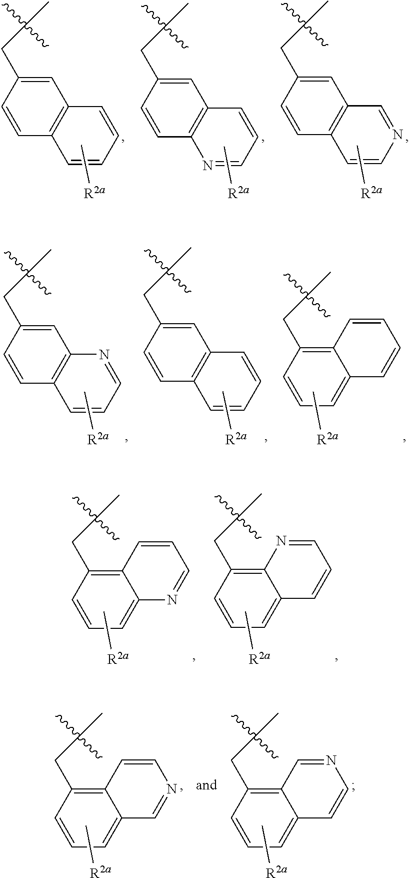

wherein [symbol] is the point of attachment to the rest of the compound; and R^{2a} is as defined herein.

In one embodiment of Formula (I), (P-I), (Ia), (P-Ia), or (P-2), including any of the foregoing, R^{2a} is selected from C₁₋₆ alkyl, C₁₋₆ haloalkyl, halogen, C₁₋₆ alkoxy, cyano, and C₁-₆haloalkoxy. In one embodiment of Formula (I), (P-I), (Ia), (P-Ia), or (P-2), including any of the foregoing, R^{2a} is C₁₋₆ hydroxyalkyl or C₁₋₆ alkoxy.

In one embodiment of Formula (I), (P-I), (Ia), (P-Ia), or (P-2), including any of the foregoing, R² is selected from

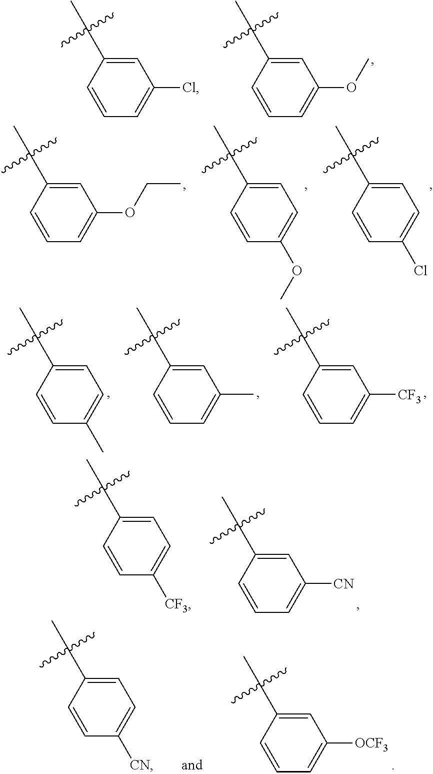

In one embodiment of Formula (I), (P-I), (Ia), (P-Ia), or (P-2), including any of the foregoing, R² is selected from

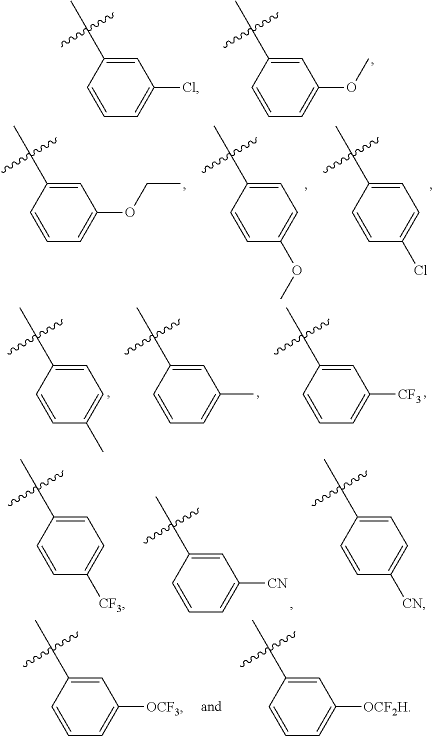

In one embodiment of Formula (I), (P-I), (Ia), (P-Ia), or (P-2), including any of the foregoing, R² is selected from

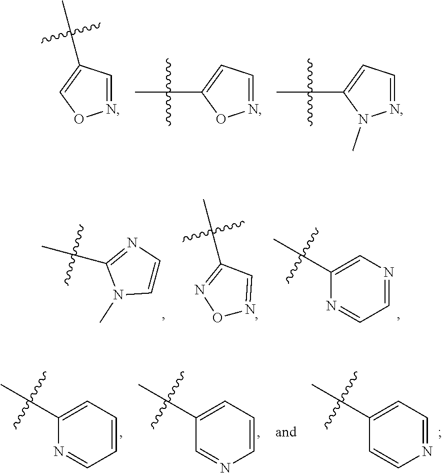

wherein [symbol] is the point of attachment to the rest of the compound.

In one embodiment of Formula (I), (P-I), (Ia), (P-Ia), or (P-2), including any of the foregoing, R² is selected from

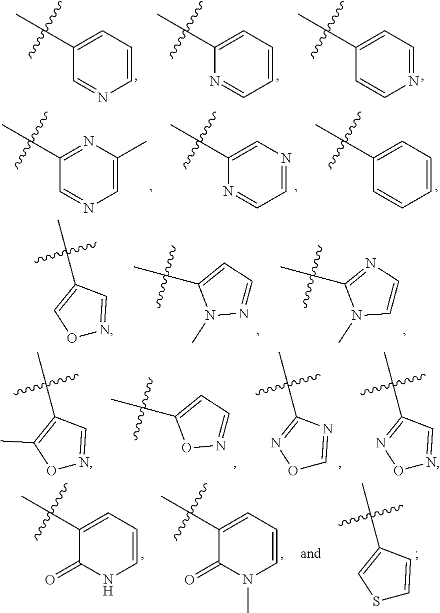

wherein [symbol] is the point of attachment to the rest of the compound.

In one embodiment of Formula (I), (P-I), (Ia), (P-Ia), or (P-2), including any of the foregoing, R¹ is hydrogen and R² is selected from

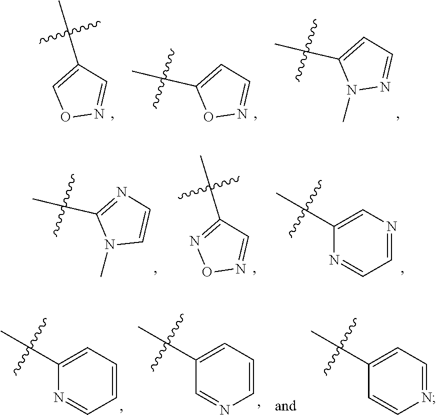

wherein [symbol] is the point of attachment to the rest of the compound.

In one embodiment of Formula (I), (P-I), (Ia), (P-Ia), or (P-2), including any of the foregoing, R¹ is methyl and R² is selected from

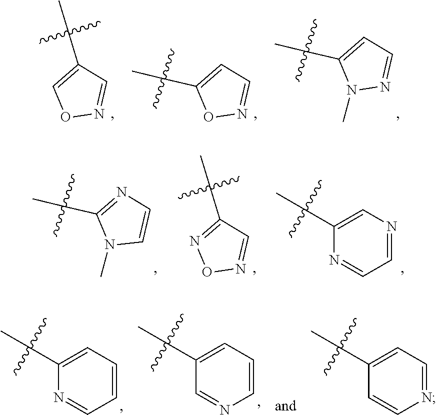

wherein [symbol] is the point of attachment to the rest of the compound.

In one embodiment of Formula (I), (P-I), (Ia), (P-Ia), or (P-2), including any of the foregoing, R¹ is hydrogen and R² is selected from

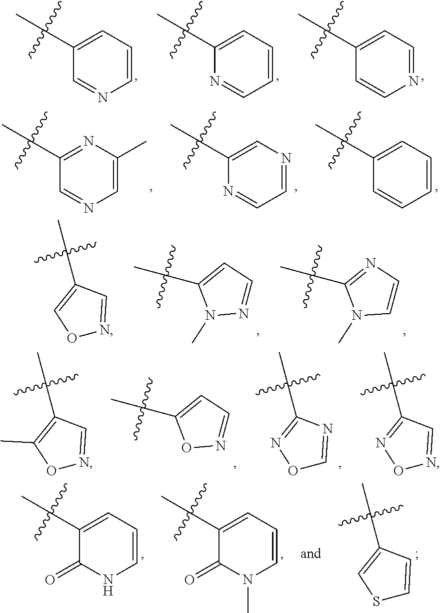

wherein [symbol] is the point of attachment to the rest of the compound.

In one embodiment of Formula (I), (P-I), (Ia), (P-Ia), or (P-2), including any of the foregoing, R¹ is methyl and R² is selected from

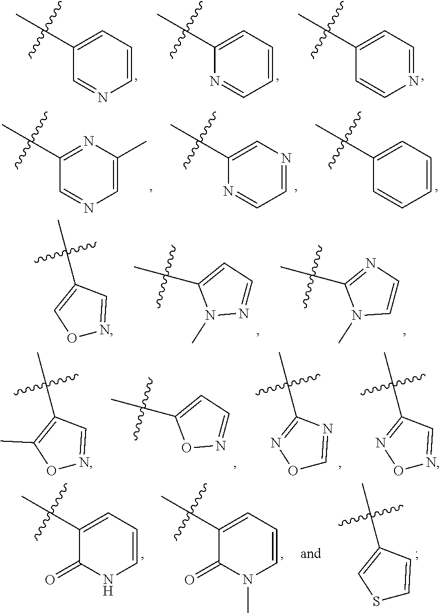

wherein [symbol] is the point of attachment to the rest of the compound.

In one embodiment of Formula (I), (P-I), (Ia), (P-Ia), or (P-2), including any of the foregoing, R¹ is hydrogen and R² is selected from

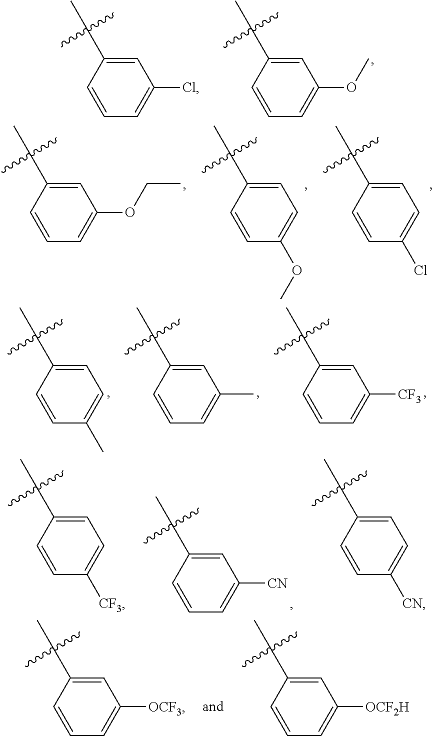

wherein [symbol] is the point of attachment to the rest of the compound.

In one embodiment of Formula (I), (P-I), (Ia), (P-Ia), or (P-2), including any of the foregoing, R¹ is C₁-₆ alkyl wherein the C₁-₆ alkyl is unsubstituted or substituted with C₁₋₆hydroxyalkyl or C₁₋₆ alkoxy and R² is

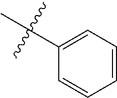

wherein [symbol] is the point of attachment to the rest of the compound.

In one embodiment of Formula (I), (P-I), (Ia), (P-Ia), or (P-2), including any of the foregoing, R¹ is hydrogen; R² is selected from

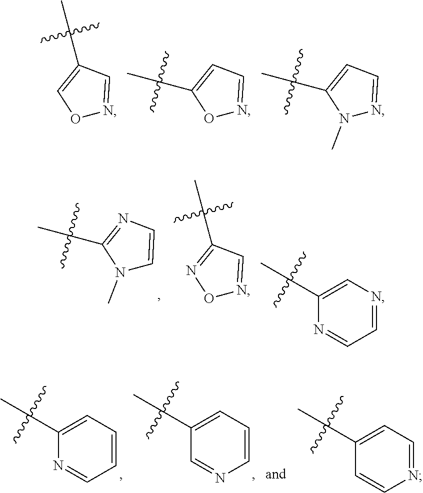

and, R³ is OH; wherein [symbol] is the point of attachment to the rest of the compound.

In one embodiment of Formula (I), (P-I), (Ia), (P-Ia), or (P-2), including any of the foregoing, R¹ is methyl; R² is selected from

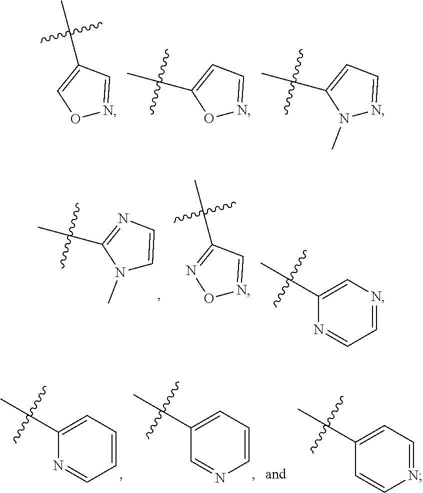

and, R³ is OH; wherein [symbol] is the point of attachment to the rest of the compound.

In one embodiment of Formula (I), (P-I), (Ia), (P-Ia), or (P-2), including any of the foregoing, R¹ is hydrogen; R² is selected from

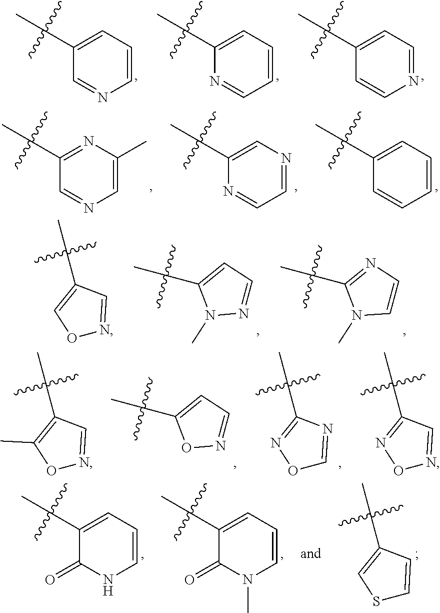

and, R³ is OH; wherein [symbol] is the point of attachment to the rest of the compound.

In one embodiment of Formula (I), (P-I), (Ia), (P-Ia), or (P-2), including any of the foregoing, R¹ is methyl; R² is selected from

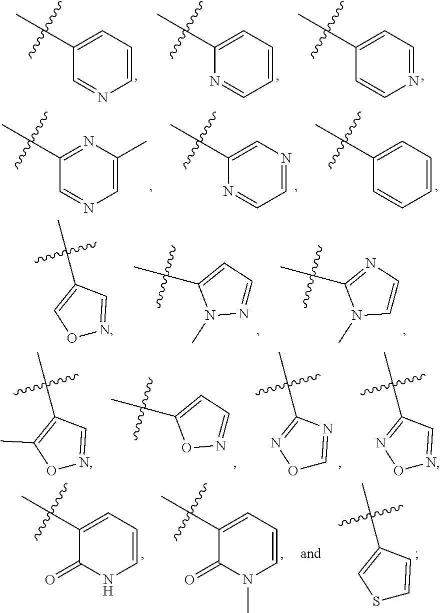

and, R³ is OH; wherein [symbol] is the point of attachment to the rest of the compound.

In one embodiment of Formula (I), (P-I), (Ia), (P-Ia), or (P-2), including any of the foregoing, R¹ is hydrogen; R² is selected from

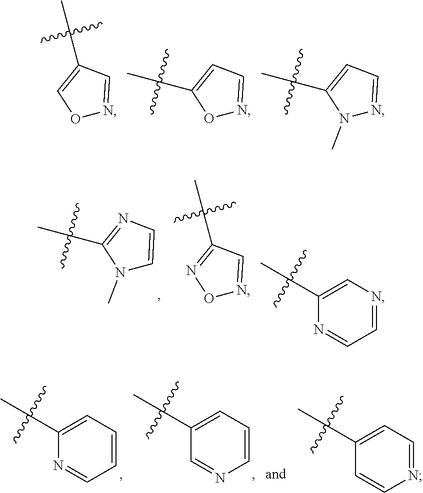

and, R³ is —OH, —OP(O)(R^{b})₂, or a 3-6-membered heteroaryl containing one, two, three, or four atoms selected from nitrogen, oxygen, and sulfur atoms; wherein [symbol] is the point of attachment to the rest of the compound.

In one embodiment of Formula (I), (P-I), (Ia), (P-Ia), or (P-2), including any of the foregoing, R¹ is hydrogen; R² is selected from

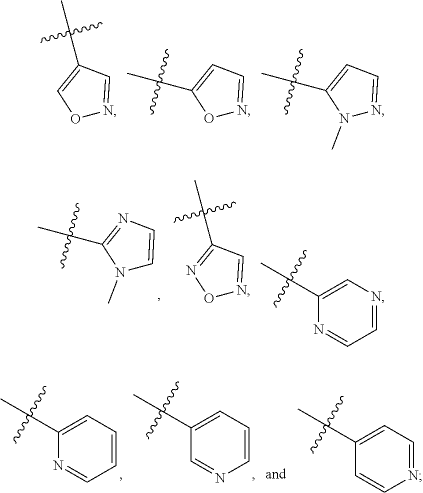

and, R³ is —OH, —OP(O)(Me)₂, or

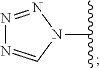

wherein [symbol] is the point of attachment to the rest of the compound.

In one embodiment of Formula (I), (P-I), (Ia), (P-Ia), or (P-2), including any of the foregoing, R¹ is methyl; R² is selected from

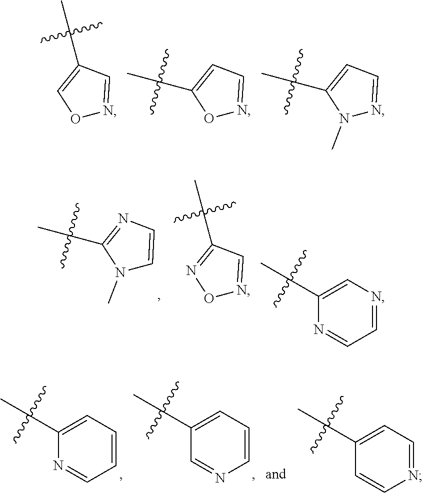

and, R³ is —OH, —OP(O)(R^{b})₂, or a 3-6-membered heteroaryl containing one, two, three, or four atoms selected from nitrogen, oxygen, and sulfur atoms; wherein [symbol] is the point of attachment to the rest of the compound.

In one embodiment of Formula (I), (P-I), (Ia), (P-Ia), or (P-2), including any of the foregoing, R¹ is methyl; R² is selected from

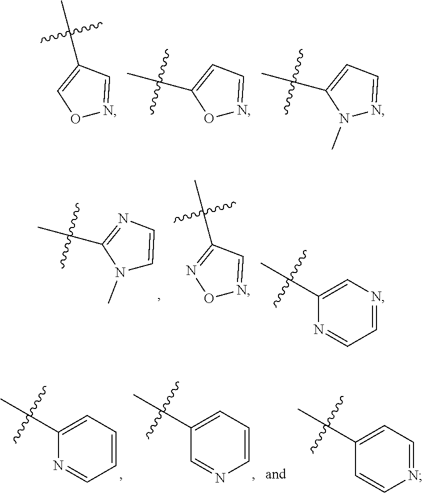

and, R³ is —OH, —OP(O)(Me)₂, or

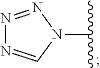

wherein [symbol] is the point of attachment to the rest of the compound.

In one embodiment of Formula (I), (P-I), (Ia), (P-Ia), or (P-2), including any of the foregoing, R¹ is hydrogen; R² is selected from

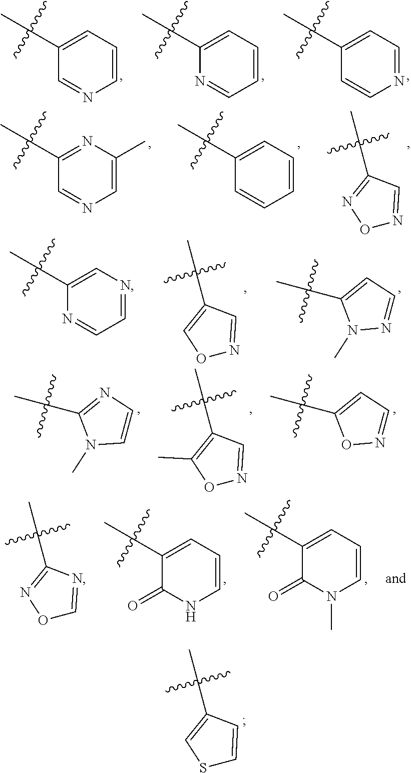

and, R³ is —OH, —OP(O)(R^{b})₂, or a 3-6-membered heteroaryl containing one, two, three, or four atoms selected from nitrogen, oxygen, and sulfur atoms; wherein [symbol] is the point of attachment to the rest of the compound.

In one embodiment of Formula (I), (P-I), (Ia), (P-Ia), or (P-2), including any of the foregoing, R¹ is hydrogen; R² is selected from

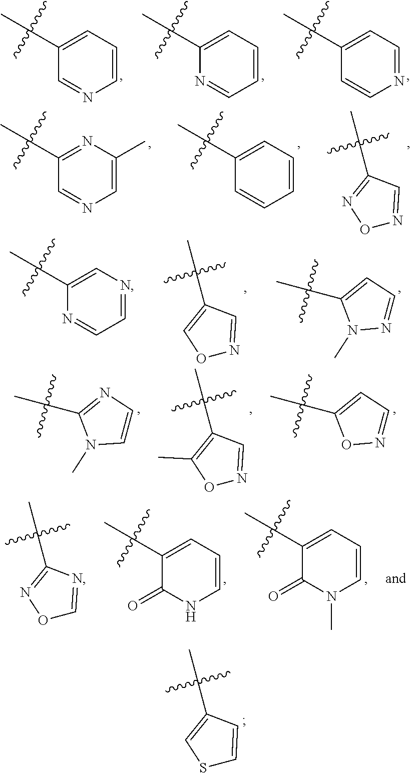

and, R³ is —OH, —OP(O)(Me)₂, or

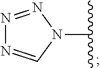

wherein [symbol] is the point of attachment to the rest of the compound.

In one embodiment of Formula (I), (P-I), (Ia), (P-Ia), or (P-2), including any of the foregoing, R¹ is methyl; R² is selected from

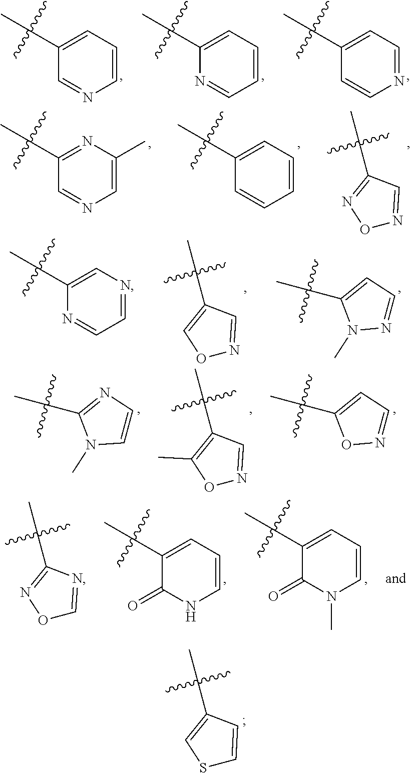

and, R³ is —OH, —OP(O)(R^{b})₂, or a 3-6-membered heteroaryl containing one, two, three, or four atoms selected from nitrogen, oxygen, and sulfur atoms; wherein [symbol] is the point of attachment to the rest of the compound.

In one embodiment of Formula (I), (P-I), (Ia), (P-Ia), or (P-2), including any of the foregoing, R¹ is methyl; R² is selected from

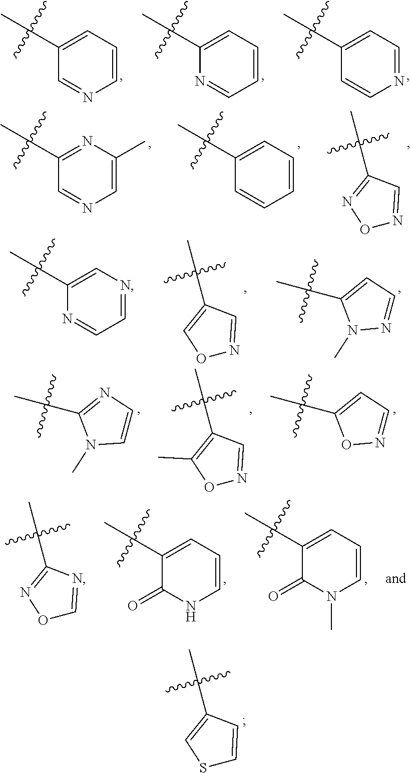

and, R³ is —OH, —OP(O)(Me)₂, or

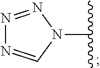

wherein [symbol] is the point of attachment to the rest of the compound.

In one embodiment of Formula (I), (P-I), (Ia), (P-Ia), or (P-2), including any of the foregoing, R¹ is hydrogen; R² is selected from

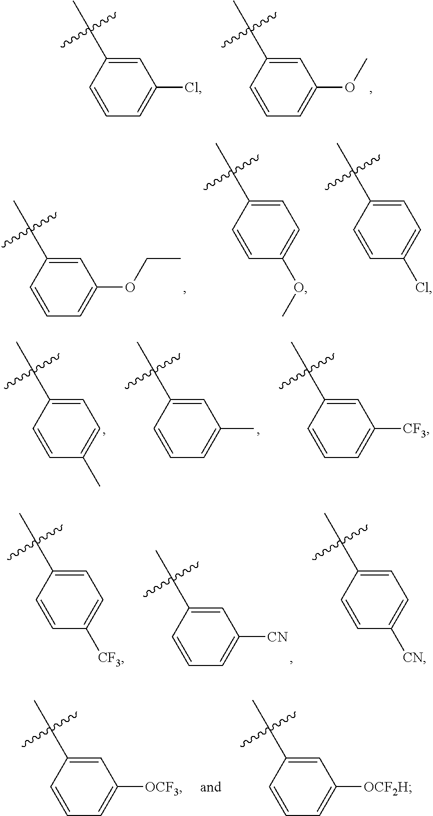

and, R³ is —OH, wherein [symbol] is the point of attachment to the rest of the compound.

In one embodiment of Formula (I), (P-I), (Ia), (P-Ia), or (P-2), including any of the foregoing, R¹ is C₁₋₆ alkyl wherein the C₁-₆ alkyl is unsubstituted or substituted with C₁₋₆hydroxyalkyl or C₁₋₆ alkoxy; R² is

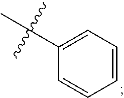 [SMILES: *c1ccccc1]

and, R³ is —OH, wherein [symbol] is the point of attachment to the rest of the compound.

In one embodiment of Formula (I), (P-I), (Ib), (P-Ib), or (P-2), including any of the foregoing, R¹ and R² are joined together to form is a N-linked 5-12 membered monocyclic or bicyclic heteroaryl containing one, two, three, or four atoms selected from nitrogen, oxygen, and sulfur wherein the heteroaryl is unsubstituted. In one embodiment of Formula (I), (P-I), (Ib), (P-Ib), or (P-2), including any of the foregoing, R¹ and R² are joined together to form is a N-linked 5-12 membered monocyclic or bicyclic heteroaryl containing one, two, three, or four atoms selected from nitrogen, oxygen, and sulfur, including the nitrogen to which R¹ and R² are attached, wherein the heteroaryl is optionally substituted with one or two R^{1a} groups. In one embodiment of Formula (I), (P-I), (Ib), (P-Ib), or (P-2), including any of the foregoing, R¹ and R² are joined together to form is a N-linked 5-12 membered monocyclic or bicyclic heteroaryl containing one, two, three, or four atoms selected from nitrogen, oxygen, and sulfur, including the nitrogen to which R¹ and R² are attached, wherein the heteroaryl is optionally substituted with one R^{1a} groups. In one embodiment of Formula (I), (P-I), (Ib), (P-Ib), or (P-2), including any of the foregoing, R¹ and R² are joined together to form is a N-linked 5-12 membered monocyclic or bicyclic heteroaryl containing one, two, three, or four nitrogen atoms, including the nitrogen to which R¹ and R² are attached, wherein the heteroaryl is optionally substituted with one or two R^{1a} groups.

In one embodiment of Formula (I), (P-I), (Ib), (P-Ib), or (P-2), including any of the foregoing, R¹ and R² are joined together to form is a N-linked 5-12 membered monocyclic or bicyclic heterocyclyl containing one, two, three, or four atoms selected from nitrogen, oxygen, and sulfur wherein the heterocyclyl is unsubstituted. In one embodiment of Formula (I), (P-I), (Ib), (P-Ib), or (P-2), including any of the foregoing, R¹ and R² are joined together to form a N-linked 5-12 membered monocyclic or bicyclic heterocyclyl containing one, two, three, or four atoms selected from nitrogen, oxygen, and sulfur, including the nitrogen to which R¹ and R² are attached, wherein the heterocyclyl is optionally substituted with one or two R^{1a} groups. In one embodiment of Formula (I), (P-I), (Ib), (P-Ib), or (P-2), including any of the foregoing, R¹ and R² are joined together to form is a N-linked 5-12 membered monocyclic or bicyclic heterocyclyl containing one, two, three, or four atoms selected from nitrogen, oxygen, and sulfur, including the nitrogen to which R¹ and R² are attached, wherein the heterocyclyl is optionally substituted with one R^{1a} groups. In one embodiment of Formula (I), (P-I), (Ib), (P-Ib), or (P-2), including any of the foregoing, R¹ and R² are joined together to form a N-linked 5-12 membered monocyclic or bicyclic heterocyclyl containing one, two, three, or four nitrogen atoms, including the nitrogen to which R¹ and R² are attached, wherein the heterocyclyl is optionally substituted with one or two R^{1a} groups. In one embodiment of Formula (I), (P-I), (Ib), (P-Ib), or (P-2), including any of the foregoing, R¹ and R² are joined together to form a N-linked 5-12 membered monocyclic or bicyclic heterocyclyl containing one nitrogen atom, which is the nitrogen to which R¹ and R² are attached, and S(O)₀₋₂, wherein the heterocyclyl is optionally substituted with one or two R^{1a} groups. In one embodiment of Formula (I), (P-I), (Ib), (P-Ib), or (P-2), including any of the foregoing, R¹ and R² are joined together to form a N-linked 5-12 membered monocyclic or bicyclic heterocyclyl containing one nitrogen atom, which is the nitrogen to which R¹ and R² are attached, and S(O)₂, wherein the heterocyclyl is optionally substituted with one or two R^{1a} groups.

In one embodiment of Formula (I), (P-I), (Ib), (P-Ib), or (P-2), including any of the foregoing, R¹ and R² are joined together to form

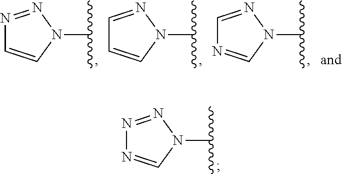

wherein [symbol] is the point of attachment to the rest of the compound.

In one embodiment of Formula (I), (P-I), (Ib), (P-Ib), or (P-2), including any of the foregoing, R¹ and R² are joined together to form

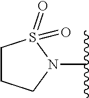

wherein [symbol] is the point of attachment to the rest of the compound.

In one embodiment of Formula (I), (P-I), (Ib), (P-Ib), or (P-2), including any of the foregoing, R¹ and R² are joined together to form

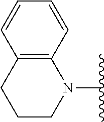

wherein [symbol] is the point of attachment to the rest of the compound.

In one embodiment of Formula (I), (P-I), (Ib), (P-Ib), or (P-2), including any of the foregoing, R¹ and R² are joined together to form

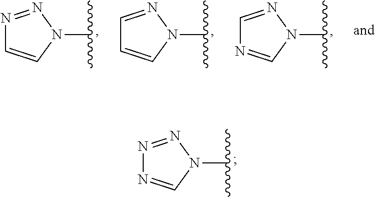

and R³ is OH; wherein [symbol] is the point of attachment to the rest of the compound.

In one embodiment of Formula (I), (P-I), (Ib), (P-Ib), or (P-2), including any of the foregoing, R¹ and R² are joined together to form

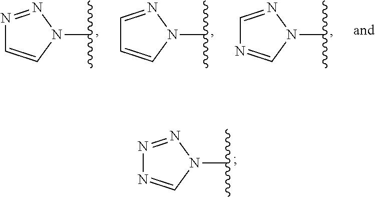

and, R³ is —OH, —OP(O)(R^{b})₂, or a 3-6-membered heteroaryl containing one, two, three, or four atoms selected from nitrogen, oxygen, and sulfur atoms; wherein [symbol] is the point of attachment to the rest of the compound.

In one embodiment of Formula (I), (P-I), (Ib), (P-Ib), or (P-2), including any of the foregoing, R¹ and R² are joined together to form

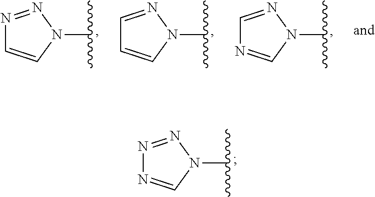

and, R³ is —OH, —OP(O)(Me)₂, or

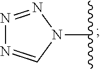

wherein [symbol] is the point of attachment to the rest of the compound.

In one embodiment of Formula (I), (P-I), (Ic), (P-Ic), or (P-2), including any of the foregoing, R² is —C₀₋₆alkylene-SO₂R⁴. In one embodiment of Formula (I), (P-I), (Ic), (P-Ic), or (P-2), R² is —C₀₋₆alkylene-SO₂R⁵.

In one embodiment of Formula (I), (P-I), (Ic), (P-Ic), or (P-2), including any of the foregoing, R² is —C₀₋₆alkylene-SO₂R⁴ and R⁴ is C₂₋₆alkyl. In one embodiment of Formula (I), (P-I), (Ic), (P-Ic), or (P-2), including any of the foregoing, R² is —C₀₋₆alkylene-SO₂R⁴ and R⁴ is C₃₋₆cycloalkyl. In one embodiment of Formula (I), (P-I), (Ic), (P-Ic), or (P-2), including any of the foregoing, R² is —C₀₋₆alkylene-SO₂R⁴ and R⁴ is C₁₋₆hydroxyalkyl. In one embodiment of Formula (I), (P-I), (Ic), (P-Ic), or (P-2), including any of the foregoing, R² is —C₀₋₆alkylene-SO₂R⁴ and R⁴ is heterocyclyl and the heterocyclyl is optionally substituted with one or two R^{4a} groups. In one embodiment of Formula (I), (P-I), (Ic), (P-Ic), or (P-2), including any of the foregoing, R² is —C₀₋₆alkylene-SO₂R⁴ and R⁴ is aryl and the aryl is optionally substituted with one or two R^{4a} groups. In one embodiment of Formula (I), (P-I), (Ic), (P-Ic), or (P-2), including any of the foregoing, R² is —C₀₋₆alkylene-SO₂R⁴ and R⁴ is heteroaryl and the heteroaryl is optionally substituted with one or two R^{4a} groups.

In one embodiment of Formula (I), (P-I), (Ic), (P-Ic), or (P-2), including any of the foregoing, R² is —C₀₋₆alkylene-SO₂R⁵ and R⁵ is heterocyclyl and the heterocyclyl is optionally substituted with one or two R^{5a} groups. In one embodiment of Formula (I), (P-I), (Ic), (P-Ic), or (P-2), including any of the foregoing, R² is —C₀₋₆alkylene-SO₂R⁵ and R⁵ is aryl and the aryl is optionally substituted with one or two R^{5a} groups. In one embodiment of Formula (I), (P-I), (Ic), (P-Ic), or (P-2), including any of the foregoing, R² is —C₀₋₆alkylene-SO₂R⁵ and R⁵ is heteroaryl and the heteroaryl is optionally substituted with one or two R^{5a} groups.

In one embodiment of Formula (I), (P-I), (Ic), (P-Ic), or (P-2), including any of the foregoing, R² is —C₀₋₆alkylene-SO₂R⁵ and R⁵ is a 5-12 membered monocylic heteroaryl containing two atoms selected from nitrogen and oxygen and wherein R⁵ is optionally substituted with one or two R^{5a} groups. In one embodiment of Formula (I), (P-I), (Ic), (P-Ic), or (P-2), including any of the foregoing, R⁵ is selected from

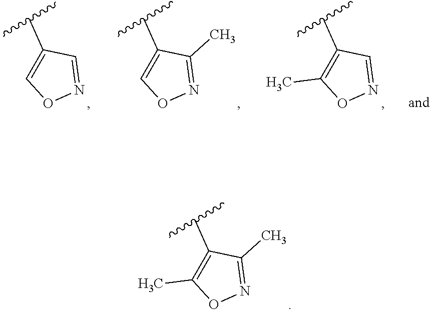

In one embodiment of Formula (I), (P-I), (Ic), (P-Ic), or (P-2), including any of the foregoing, R¹ is methyl and R² is —C₀₋₆alkylene-SO₂R⁴. In one embodiment of Formula (I), (P-I), (Ic), (P-Ic), or (P-2), including any of the foregoing, R¹ is hydrogen and R² is —C₀₋₆alkylene-SO₂R⁵. In one embodiment of Formula (I), (P-I), (Ic), (P-Ic), or (P-2), including any of the foregoing, R¹ is methyl and R² is —C₀₋₆alkylene-SO₂R⁵.

In one embodiment of Formula (I), (P-I), (Ic), (P-Ic), or (P-2), including any of the foregoing, R² is selected from

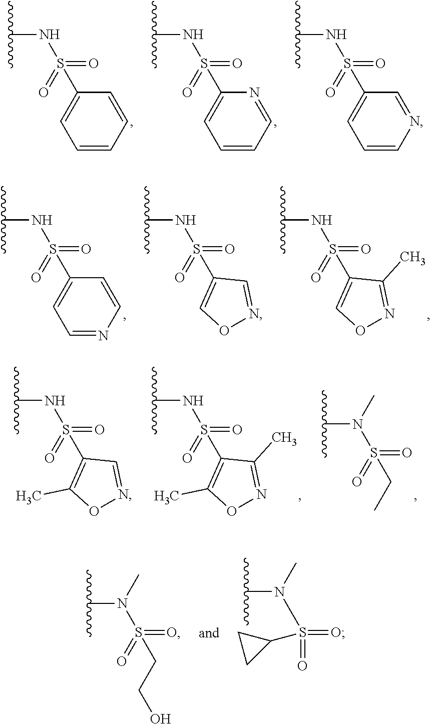

wherein [symbol] is the point of attachment to the rest of the compound.

In one embodiment of Formula (I), (P-I), (Ic), (P-Ic), or (P-2), including any of the foregoing, R¹ is hydrogen, R² is selected from

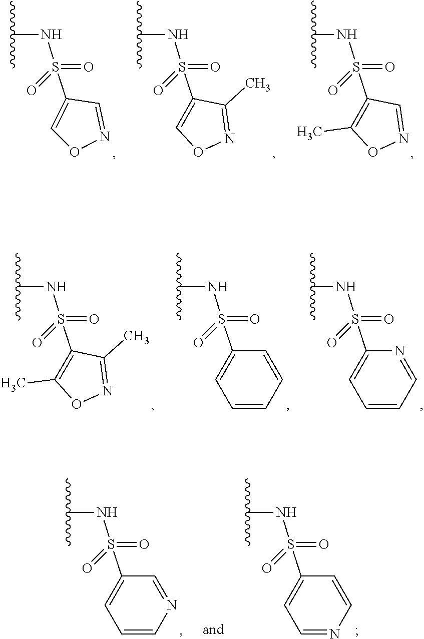

and R³ is —OH; wherein [symbol] is the point of attachment to the rest of the compound.

In one embodiment of Formula (I), (P-I), (Ic), (P-Ic), or (P-2), including any of the foregoing, R¹ is methyl, R² is selected from

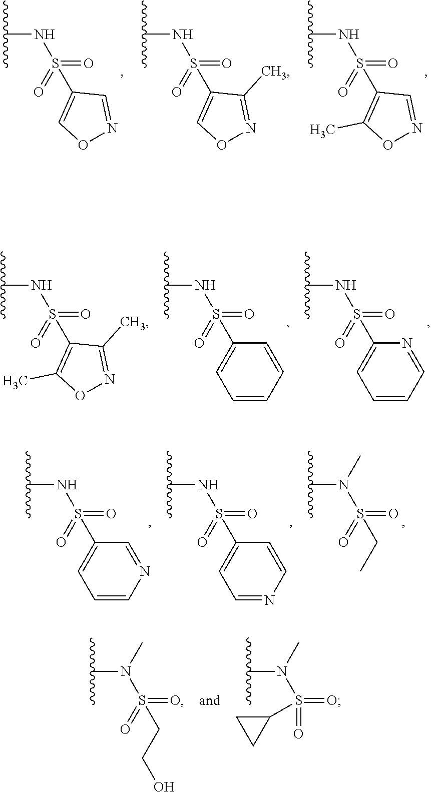

and R³ is —OH; wherein [symbol] is the point of attachment to the rest of the compound.

In one embodiment of Formula (I), (P-I), (Ic), (P-Ic), or (P-2), including any of the foregoing, R² is selected from

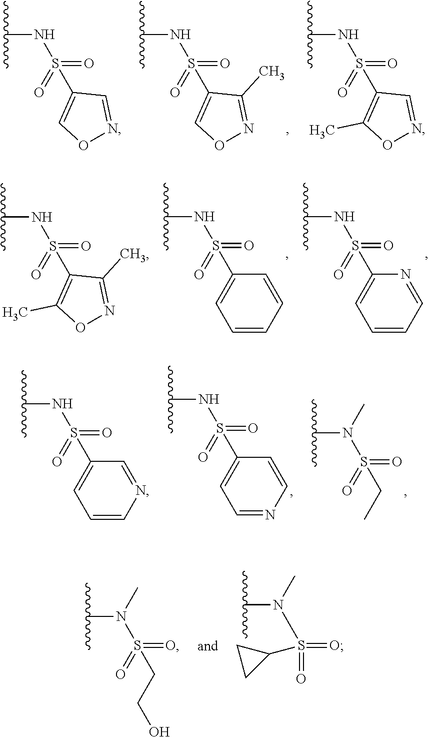

and R³ is —OH; wherein [symbol] is the point of attachment to the rest of the compound.

In one embodiment of Formula (I), (P-I), (Ic), (P-Ic), or (P-2), including any of the foregoing, R¹ is methyl; R² is —C₀₋₆alkylene-SO₂R⁴; and R³ is —OH, —OP(O)(R^{b})₂, or a 3-6-membered heteroaryl containing one, two, three, or four atoms selected from nitrogen, oxygen, and sulfur atoms. In one embodiment of Formula (I), (P-I), (Ic), (P-Ic), or (P-2), including any of the foregoing, R¹ is hydrogen; R² is —C₀₋₆alkylene-SO₂R⁵; and R³ is —OH, —OP(O)(R^{b})₂, or a 3-6-membered heteroaryl containing one, two, three, or four atoms selected from nitrogen, oxygen, and sulfur atoms. In one embodiment of Formula (I), (Ic), or (P-2) including any of the foregoing, R¹ is methyl; R² is —C₀₋₆alkylene-SO₂R⁵; and, R³ is —OH, —OP(O)(R^{b})₂, or a 3-6-membered heteroaryl containing one, two, three, or four atoms selected from nitrogen, oxygen, and sulfur atoms.

In one embodiment of Formula (I), (P-I), (Ic), (P-Ic), or (P-2), including any of the foregoing, R¹ is methyl; R² is —C₀₋₆alkylene-SO₂R⁴; and, R³ is —OH, —OP(O)(Me)₂, or

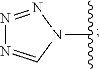

wherein [symbol] is the point of attachment to the rest of the compound. In one embodiment of Formula (I), (Ic), or (P-2), including any of the foregoing, R¹ is hydrogen; R² is —C₀₋₆alkylene-SO₂R⁵; and R³ is —OH, —OP(O)(Me)₂, or

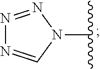

wherein [symbol] is the point of attachment to the rest of the compound. In one embodiment of Formula (I), (P-I), (Ic), (P-Ic), or (P-2), including any of the foregoing, R¹ is methyl; R² is —C₀₋₆alkylene-SO₂R⁵; and, R³ is —OH, —OP(O)(Me)₂, or

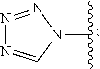

wherein [symbol] is the point of attachment to the rest of the compound.

In one embodiment of Formula (I), (P-I), (Ic), (P-Ic), or (P-2), including any of the foregoing, R¹ is hydrogen, R² is selected from

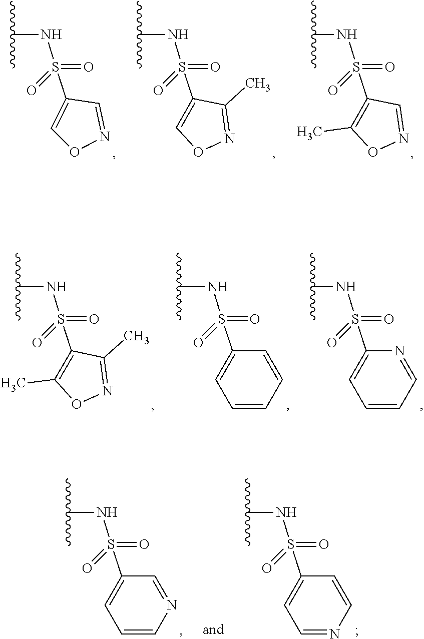

and R³ is —OH, —OP(O)(R^{b})₂, or a 3-6-membered heteroaryl containing one, two, three, or four atoms selected from nitrogen, oxygen, and sulfur atoms; wherein [symbol] is the point of attachment to the rest of 7 the compound.

In one embodiment of Formula (I), (P-I), (Ic), (P-Ic), or (P-2), including any of the foregoing, R¹ is methyl, R² is selected from

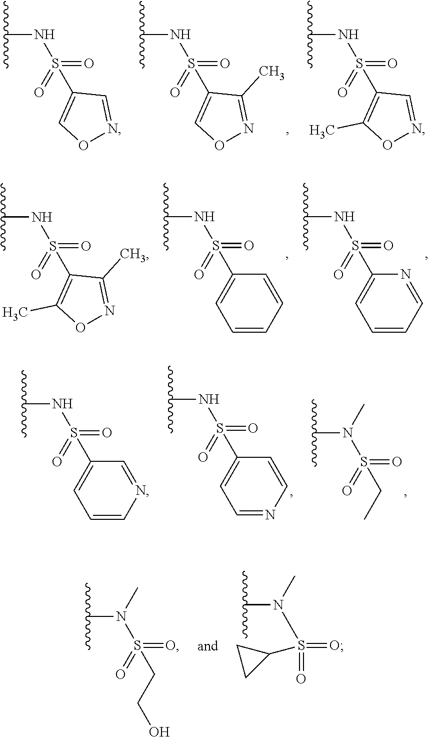

and R³ is —OH, —OP(O)(R^{b})₂, or a 3-6-membered heteroaryl containing one, two, three, or four atoms selected from nitrogen, oxygen, and sulfur atoms; wherein [symbol] is the point of attachment to the rest of the compound.

In one embodiment of Formula (I), (P-I), (Ic), (P-Ic), or (P-2), including any of the foregoing, R¹ is hydrogen, R² is selected from

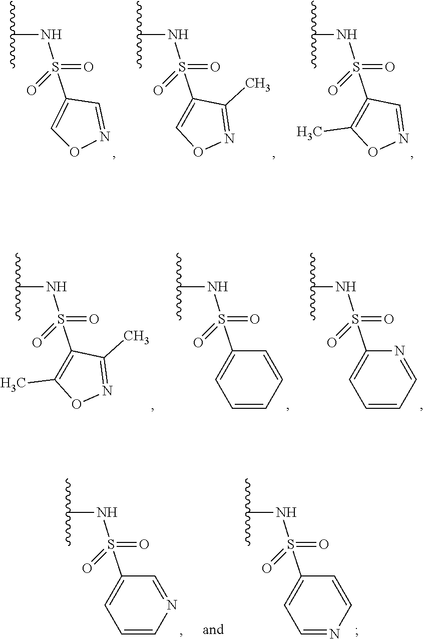

and R³ is —OH, —OP(O)(Me)₂, or

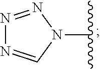

wherein [symbol] is the point of attachment to the rest of the compound.

In one embodiment of Formula (I), (P-I), (Ic), (P-Ic), or (P-2), including any of the foregoing, R¹ is methyl, R² is selected from

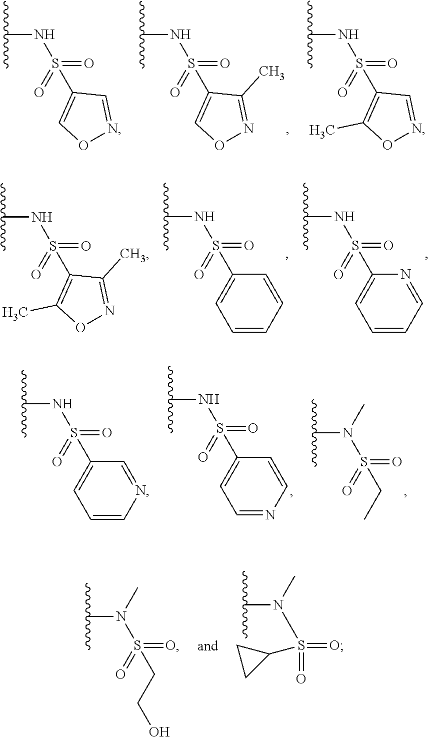

and R³ is —OH, —OP(O)(Me)₂, or

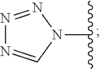

wherein [symbol] is the point of attachment to the rest of the compound.

In one embodiment of Formula (I), (P-I), (Ic), (P-Ic), or (P-2), including any of the foregoing, R⁴ is selected from

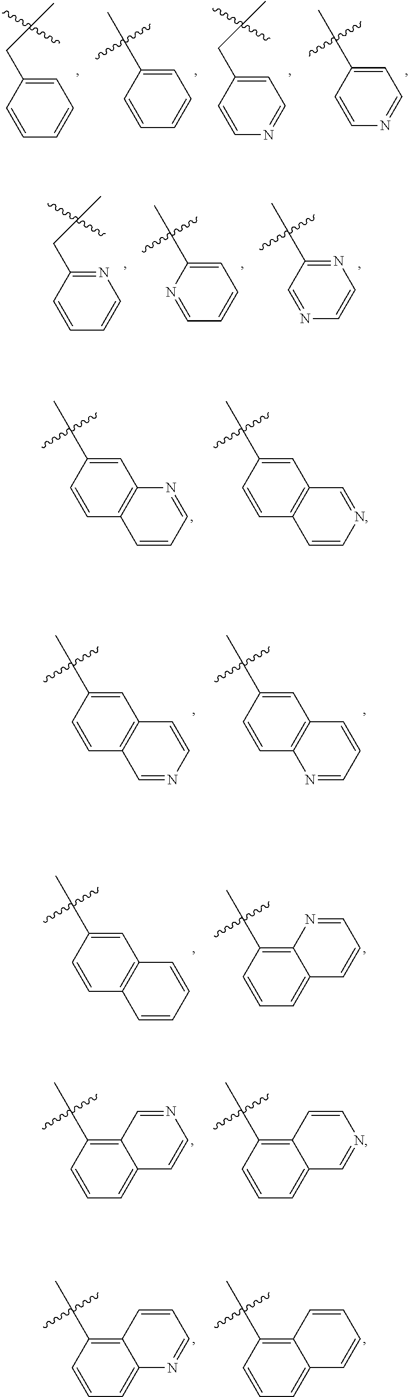

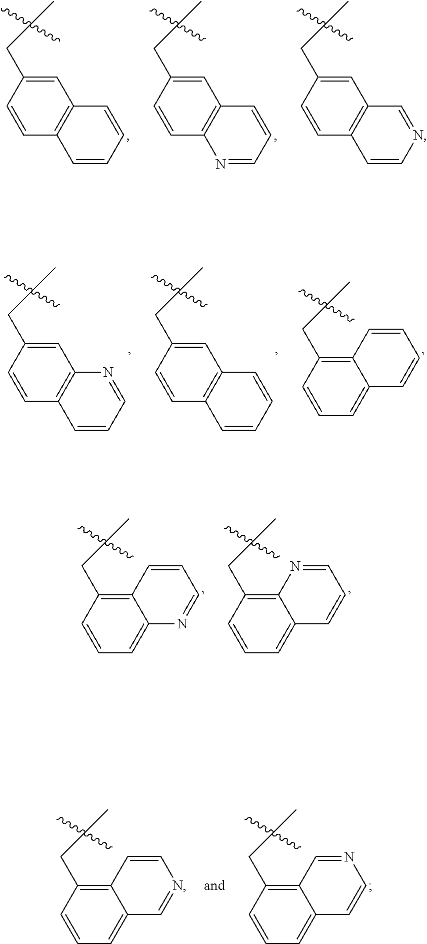

wherein [symbol] is the point of attachment to the rest of the compound.

In one embodiment of Formula (I). (P-I). (Ic). (P-Ic), or (P-2), including any of the foregoing. R⁴ is selected from

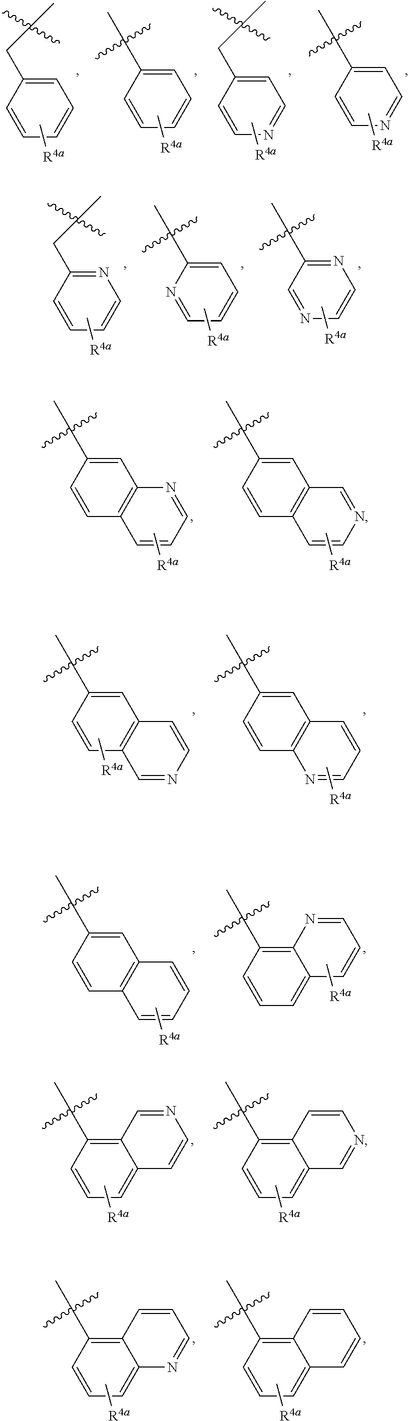

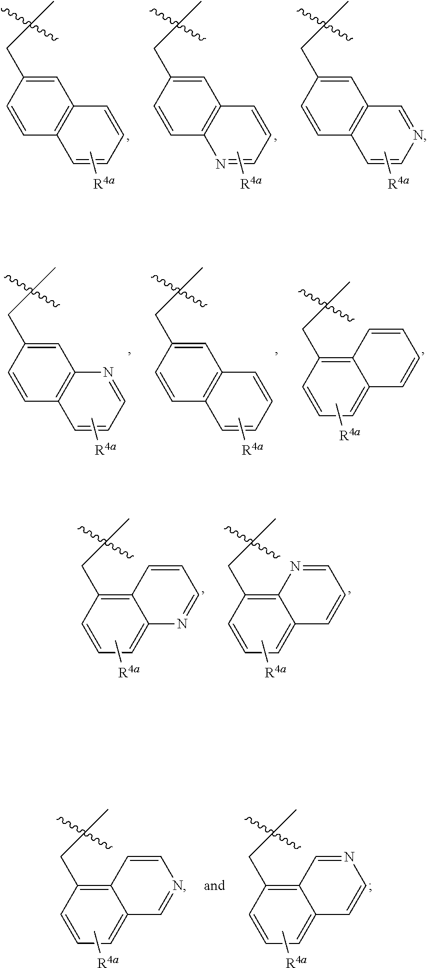

wherein [symbol] is the point of attachment to the rest of the compound; and R^{4a} is as defined herein.

In one embodiment of Formula (I), (P-I), (Ic), (P-Ic), or (P-2), including any of the foregoing, R⁴ is selected from

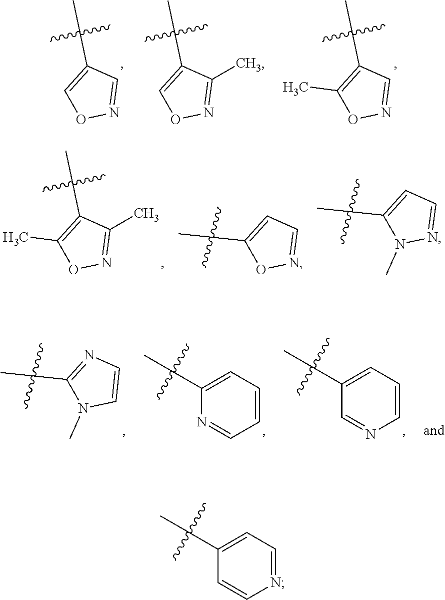

wherein [symbol] is the point of attachment to the rest of the compound.

In one embodiment of Formula (I), (P-I), (Ic), (P-Ic), or (P-2), including any of the foregoing, R⁵ is selected from

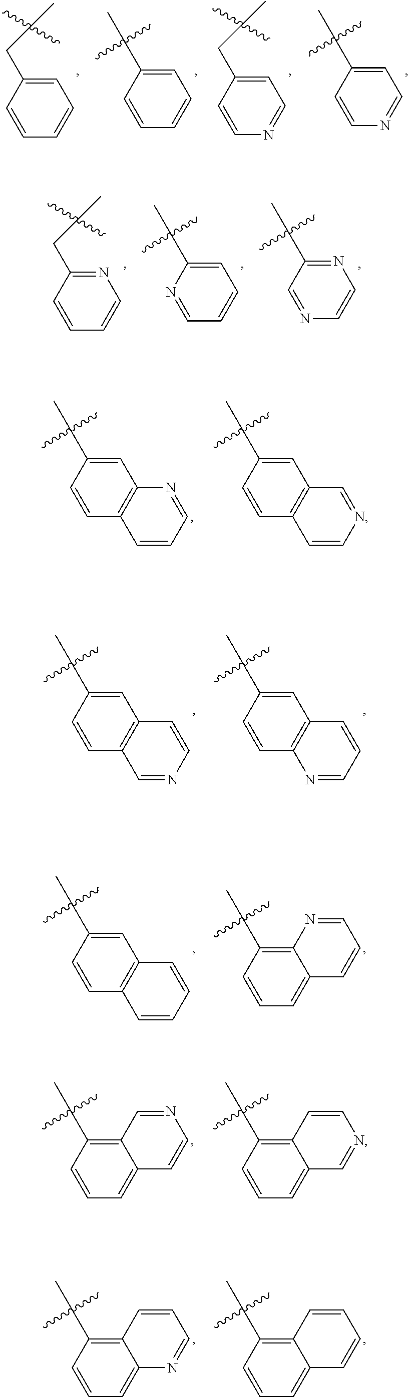

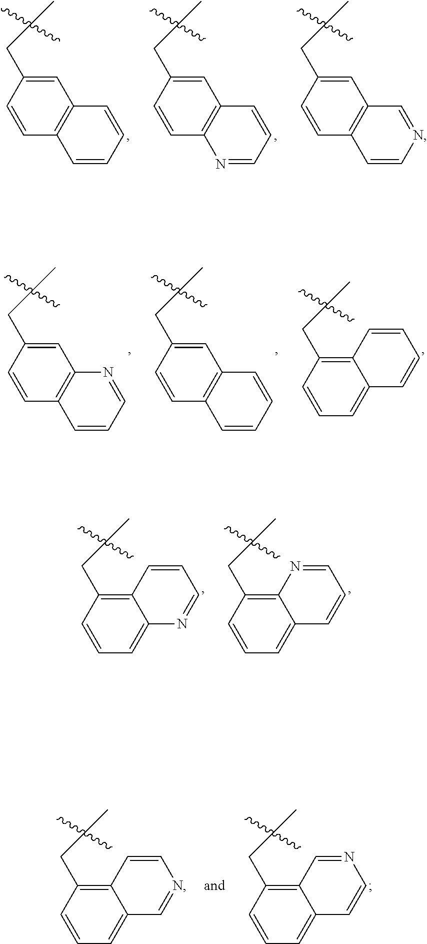

wherein [symbol] is the point of attachment to the rest of the compound.

In one embodiment of Formula (I), (P-I), (Ic), (P-Ic), or (P-2), including any of the foregoing, R⁵ is selected from

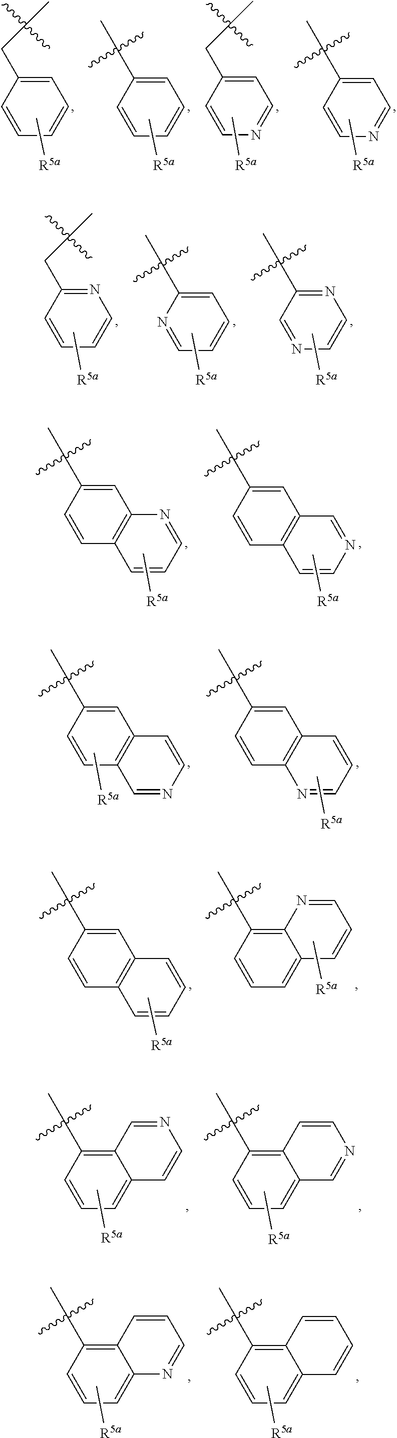

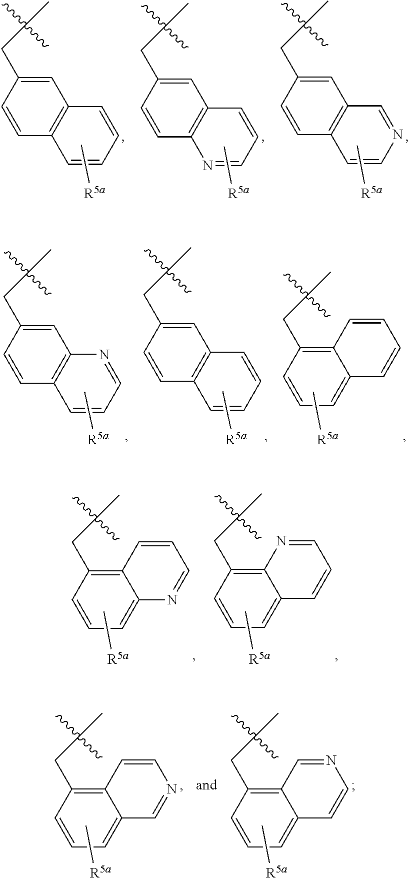

wherein [symbol] is the point of attachment to the rest of the compound; and R^{5a} is as defined herein.

In one embodiment of Formula (I), (P-I), (Ic), (P-Ic), or (P-2), including any of the foregoing, R⁴ is selected from

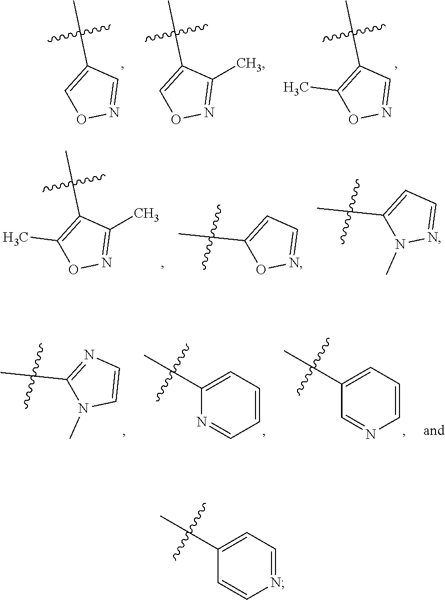

wherein [symbol] is the point of attachment to the rest of the compound.

In one embodiment, the compound of Formula (I) is of the formula:

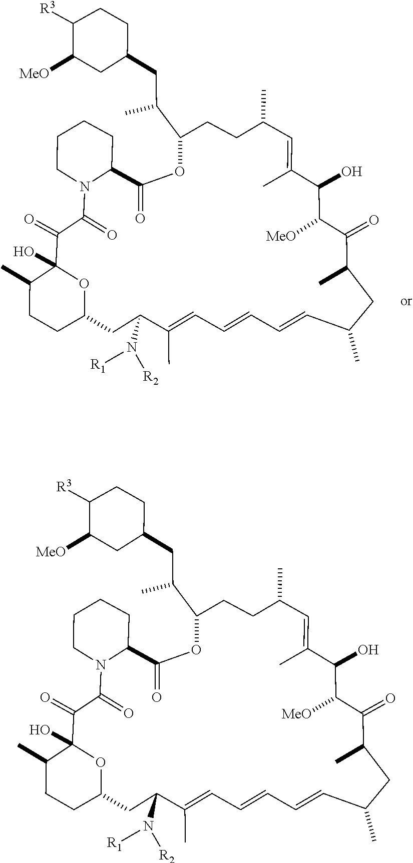

or a pharmaceutically acceptable salt thereof; wherein R¹, R², and R³ are as described in any of the embodiments for Formula (I) described herein.

Non-limiting examples of compounds of Formula (Ia) include:

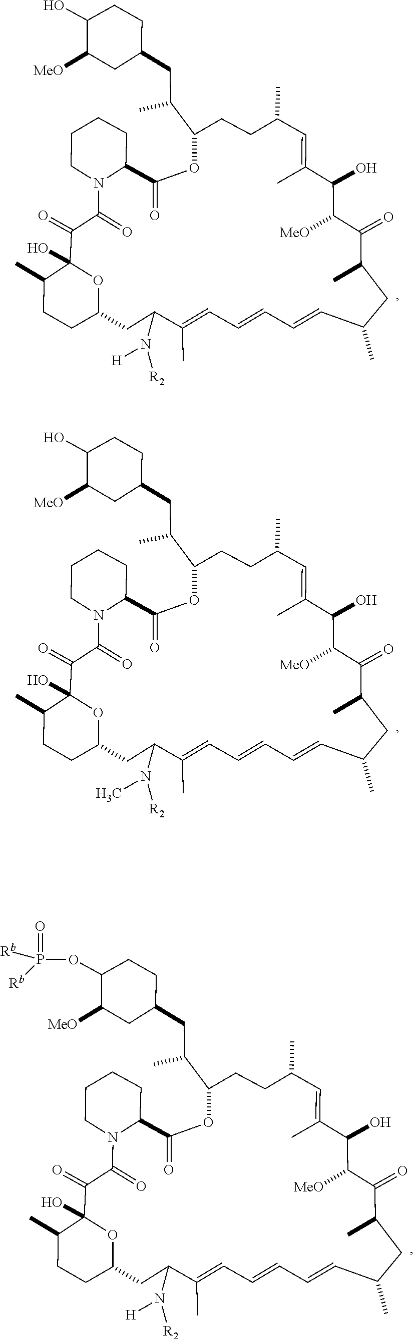

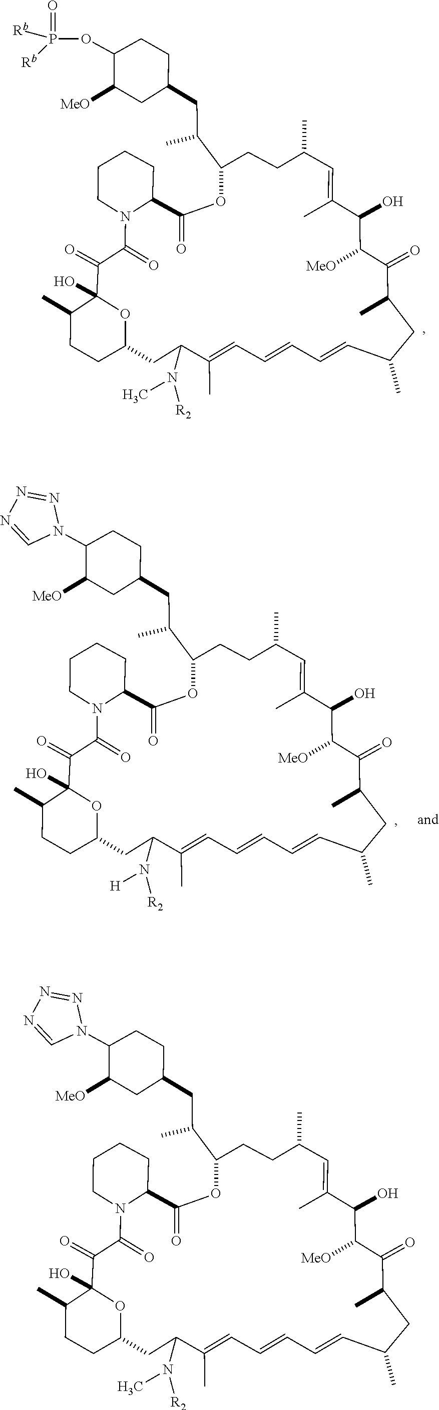

or a pharmaceutically acceptable salt thereof; wherein R² and R^{b} are as described in any of the embodiments for Formula (Ia) described herein.

Non-limiting examples of compounds of Formula (Ib) include:

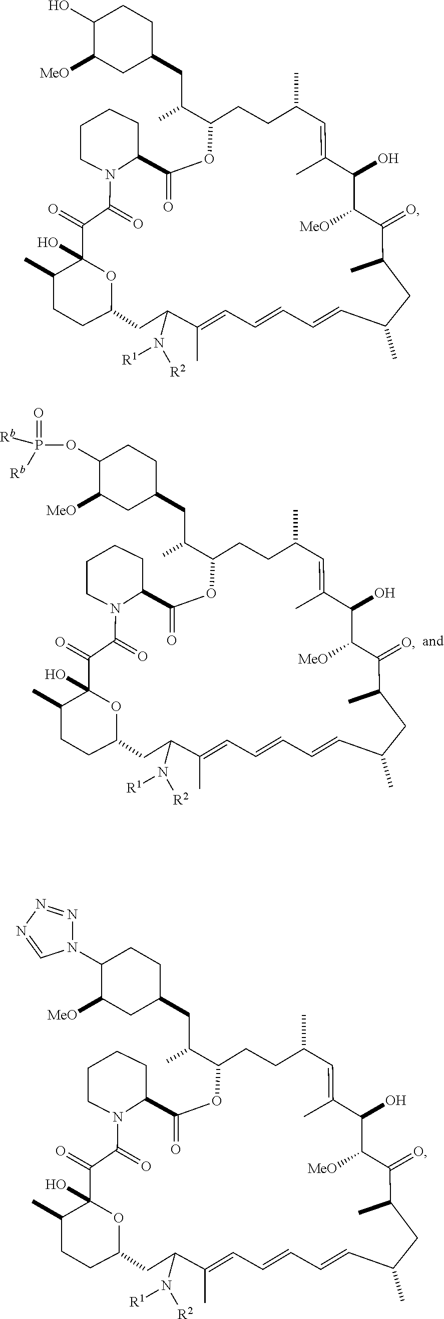

or a pharmaceutically acceptable salt thereof; wherein R′. R², and R^{b} are as described in any of the embodiments for Formula (Ib) described herein.

Non-limiting examples of compounds of Formula (Ic) include:

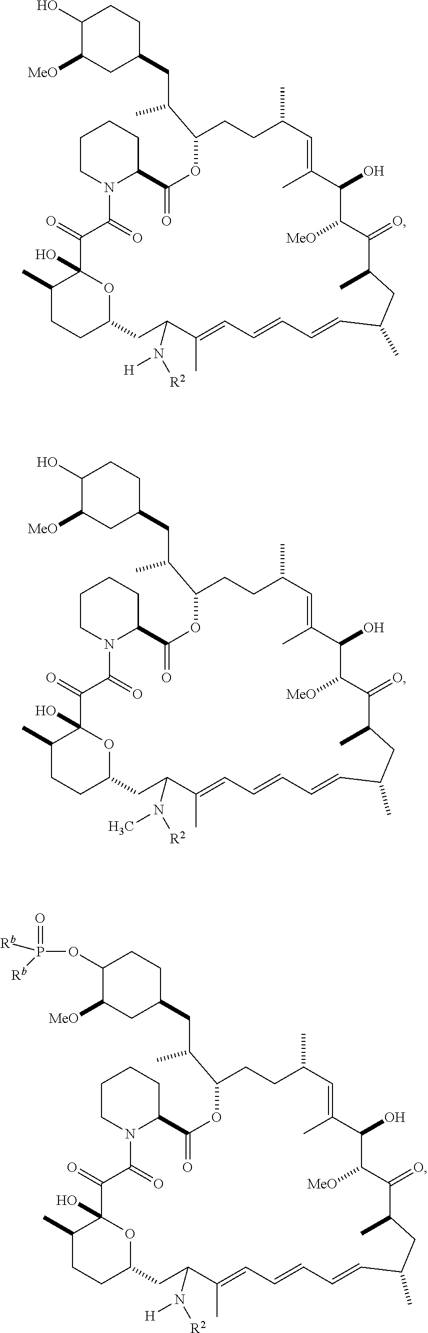

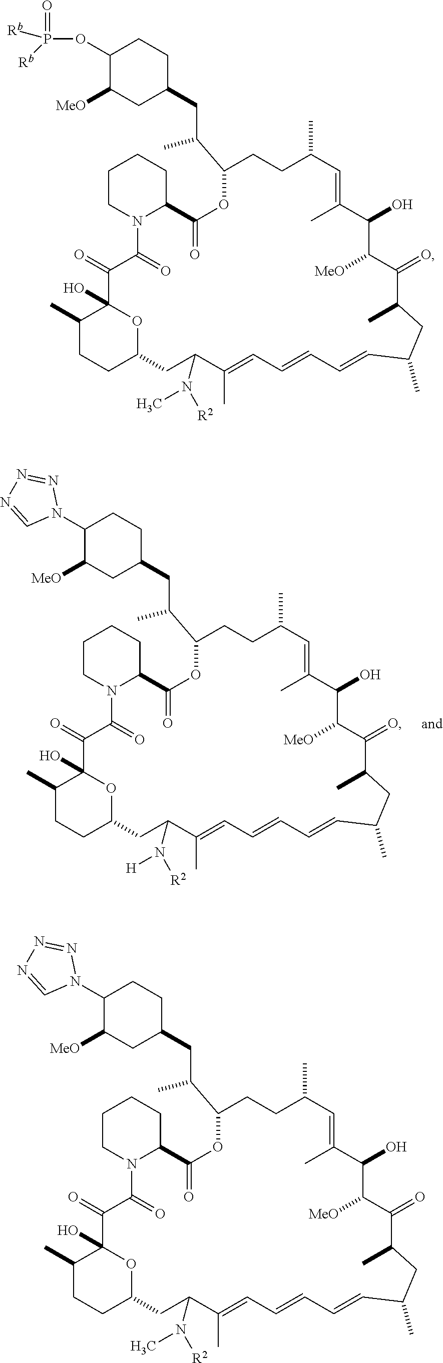

or a pharmaceutically acceptable salt thereof; wherein R² and R^{b} are as described in any of the embodiments for Formula (Ic) described herein.

In certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de of zero. In certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de greater than zero. For example, in certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de of about ten. In certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de of about twenty-five. In certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de of about fifty. In certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de of about seventy-five. In certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de of about eighty. In certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de of about eighty-five. In certain embodiments the compound of Formula (P-2), Formula (P-I), Formula (P-Ia), Formula (P-Ib), Formula (P-Ic), Formula (I), Formula (Ia), Formula (Ib), or Formula (Ic) has a de or % de of about ninety. In certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de of about ninety-five. In certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de of about ninety-seven. In certain embodiments, the compound of Formula (P-2), Formula (P-I), Formula (P-Ia), Formula (P-Ib), Formula (P-Ic), Formula (I), Formula (Ia), Formula (Ib), or Formula (Ic) has a de or % de of about ninety-eight. In certain embodiments, the compound of Formula (P-2), Formula (P-I), Formula (P-Ia), Formula (P-Ib), Formula (P-Ic), Formula (P-2), Formula (I), Formula (Ia), Formula (Ib), or Formula (Ic) has a de or % de of about ninety-ninety. In certain embodiments, the compounds described herein have a de or % de of one-hundred.

In one embodiment, the compound of Formula (P-2), Formula (P-I), Formula (P-Ia), Formula (P-Ib), Formula (P-Ic), Formula (I), Formula (Ia), Formula (Ib), or Formula (Ic) or a pharmaceutically acceptable salt thereof is at least about 50%, at least about 60%, at least about 70%, at least about 75%, at least about 80%, at least about 85%, at least about 90%, at least about 95%, at least about 97%, at least about 99%, or even 100% by weight the C-16 (R)-epimer based solely on the weight of the compound of Formula (P-2), Formula (P-I), Formula (P-Ia), Formula (P-Ib), Formula (P-Ic), Formula (I), Formula (Ia), Formula (Ib), or Formula (Ic)(i.e., excluding the weight of a pharmaceutically acceptable salt, if the compound exists as a pharmaceutically acceptable salt). In one embodiment, the compound of Formula (P-2), Formula (P-I), Formula (P-Ia), Formula (P-Ib), Formula (P-Ic), Formula (I), (Ia), (Ib), or (Ic) or a pharmaceutically acceptable salt thereof, is about 85%-95% the (R)—C-16 epimer. In one embodiment, the compound of Formula (P-2), Formula (P-I), Formula (P-Ia), Formula (P-Ib), Formula (P-Ic), Formula (I), (Ia), (Ib), or (Ic) or a pharmaceutically acceptable salt thereof, is about 90%-95% the (R)—C-16 epimer.

In one embodiment, the compound of Formula (P-2), Formula (P-I), Formula (P-Ia), Formula (P-Ib), Formula (P-Ic), Formula (I), Formula (Ia), Formula (Ib), or Formula (Ic) or a pharmaceutically acceptable salt thereof is 50% by weight the C-16 (S)-epimer and 50% by the weight C-16 (R)-epimer based solely on the weight of the compound of Formula (P-2), Formula (P-I), Formula (P-Ia), Formula (P-Ib), Formula (P-Ic), Formula (I), Formula (Ia), Formula (Ib), or Formula (Ic)(i.e., excluding the weight of a pharmaceutically acceptable salt, if the compound exists as a pharmaceutically acceptable salt).

In one embodiment, the compound of Formula (P-2), Formula (P-I), Formula (P-Ia), Formula (P-Ib), Formula (P-Ic), Formula (I), Formula (Ia), Formula (Ib), or Formula (Ic) or a pharmaceutically acceptable salt thereof is at least about 50%, at least about 60%, at least about 70%, at least about 75%, at least about 80%, at least about 85%, at least about 90%, at least about 95%, at least about 97%, at least about 99%, or even 100% by weight the C-16 (S)-epimer based solely on the weight of the compound of Formula (P-2), Formula (P-I), Formula (P-Ia), Formula (P-Ib), Formula (P-Ic), Formula (I), Formula (Ia), Formula (Ib), or Formula (Ic)(i.e., excluding the weight of a pharmaceutically acceptable salt, if the compound exists as a pharmaceutically acceptable salt). In one embodiment, the compound of Formula (P-2), Formula (P-I), Formula (P-Ia), Formula (P-Ib), Formula (P-Ic), Formula (I), (Ia), (Ib), or (Ic) or a pharmaceutically acceptable salt thereof, is about 85%-95% the(S)—C-16 epimer. In one embodiment, the compound of Formula (P-2), Formula (P-I), Formula (P-Ia), Formula (P-Ib), Formula (P-Ic), Formula (I), (Ia), (Ib), or (Ic) or a pharmaceutically acceptable salt thereof, is about 90%-95% the(S)—C-16 epimer.

In some embodiments, the compounds and pharmaceutical compositions disclosed herein for use in the treatment of a disorder or disease mediated by the mTOR pathway are more selective in binding to FKBP12 than others of the group of FK506 binding proteins (FKBPs).

In some other embodiments, the compounds and pharmaceutical compositions disclosed herein for use in the treatment of a disorder or disease mediated by the mTOR pathway show an unusual and surprising pharmacokinetic profile and an enhanced pharmacodynamic selectivity among target FKBPs, those compounds and pharmaceutical composition matters are useful for the treatment of age- or aging-related diseases, complications of diabetes, cancers, as well as inflammation-associated disorders.

Yet in some other embodiments, the compounds and pharmaceutical compositions pathway, wherein the compound inhibits S6K1 phosphorylation at least two-fold less efficiently than the rapalog RAD001 in FKBP12 KO cells in comparison to FKBP12 expressing cells.

In some other embodiments, the compounds and pharmaceutical compositions disclosed herein for use in the treatment of a disorder or disease mediated by the mTOR pathway, wherein the compound inhibits S6K1 phosphorylation at least 10-fold less efficiently than the rapalog RAD001 in FKBP12 KO cells in comparison to FKBP12 expressing cells.

In some other embodiments, the compounds and pharmaceutical compositions disclosed herein for use in the treatment of a disorder or disease mediated by the mTOR pathway, wherein the compound inhibits S6K1 phosphorylation at least 100-fold less efficiently than the rapalog RAD001 in FKBP12 KO cells in comparison to FKBP12 expressing cells.

In some other embodiments, the compounds and pharmaceutical compositions disclosed herein for use in the treatment of a disorder or disease mediated by the mTOR pathway, wherein the compound requires about 10-fold higher concentration, in comparison to the rapalog RAD001, in FKBP12 KO cells in comparison to FKBP12 expressing cells, to achieve 20% inhibition of S6K1 cell signaling.

In some other embodiments, the compounds and pharmaceutical compositions disclosed herein for use in the treatment of a disorder or disease mediated by the mTOR pathway, wherein the compound requires about 20-fold higher concentration, in comparison to the rapalog RAD001, in FKBP12 KO cells in comparison to FKBP12 expressing cells, to achieve 20% inhibition of S6K1 cell signaling.

In some other embodiments, the compounds and pharmaceutical compositions disclosed herein for use in the treatment of a disorder or disease mediated by the mTOR pathway, wherein the compound requires about 100-fold higher concentration, in comparison to the rapalog RAD001, in FKBP12 KO cells in comparison to FKBP12 expressing cells, to achieve 20% inhibition of S6K1 cell signaling.

In some other embodiments, the compounds and pharmaceutical compositions disclosed herein for use in the treatment of a disorder or disease mediated by the mTOR pathway, wherein the compound requires about 500-fold higher concentration, in comparison to the rapalog RAD001, in FKBP12 KO cells in comparison to FKBP12 expressing cells, to achieve 20% inhibition of S6K1 cell signaling.

In some other embodiments, the compounds and pharmaceutical compositions pathway, wherein the compound requires about 1000-fold higher concentration, in comparison to the rapalog RAD001, in FKBP12 KO cells in comparison to FKBP12 expressing cells, to achieve 20% inhibition of S6K1 cell signaling.

Yet in some other embodiments, an “FKBP12-selective rapalog” is a rapalog, while it is potent in an assay using a wild-type of cells, it is approximately 10-fold to approximately 1000-fold less potent than RAD001 in a cell line that does not express FKBP12 (e.g., FKBP12 knock-out cells). “Potency,” as used in this context, can be expressed as the concentration of rapalog required to achieve a 20% inhibition of S6K1 (Thr389)phosphorylation in a cell-based assay such as that used in Example 3 and FIGS. 1-11. For example, a rapalog is considered FKBP12-selective if the rapalog inhibits about 20% of S6K1 (Thr389)phosphorylation inhibition in assay using an FKBP12 knock-out cell line with at least 20× (e.g. 25×, 30×, 50×, 100×, 200×, 500×, 1000×, etc.) lower potency compared to similar level of inhibition achieved by RAD001 in the same assay with the same cell line, which normally expresses FKBP12.

In some other embodiments, the compounds and pharmaceutical compositions disclosed herein for use in the treatment of a disorder or disease mediated by the mTOR pathway, wherein the compound requires about 20-fold higher concentration, in comparison to the rapalog RAD001, in FKBP12 KO cells in comparison to FKBP12 expressing cells, to achieve 30% inhibition of S6K1 cell signaling.

In some other embodiments, the compounds and pharmaceutical compositions disclosed herein for use in the treatment of a disorder or disease mediated by the mTOR pathway, wherein the compound requires about 100-fold higher concentration, in comparison to the rapalog RAD001, in FKBP12 KO cells in comparison to FKBP12 expressing cells, to achieve 30% inhibition of S6K1 cell signaling.

In some other embodiments, the compounds and pharmaceutical compositions disclosed herein for use in the treatment of a disorder or disease mediated by the mTOR pathway, wherein the compound requires about 500-fold higher concentration, in comparison to the rapalog RAD001, in FKBP12 KO cells in comparison to FKBP12 expressing cells, to achieve 30% inhibition of S6K1 cell signaling.

In some other embodiments, the compounds and pharmaceutical compositions disclosed herein for use in the treatment of a disorder or disease mediated by the mTOR pathway, wherein the compound requires about 1000-fold higher concentration, in comparison to the rapalog RAD001, in FKBP12 KO cells in comparison to FKBP12 expressing cells, to achieve 30% inhibition of S6K1 cell signaling.

Yet in some other embodiments, an “FKBP12-selective rapalog” is a rapalog, while it is potent in an assay using a wild-type of cells, it is approximately 20-fold to approximately 1000-fold less potent than RAD001 in a cell line that does not express FKBP12 (e.g., FKBP12 knock-out cells). “Potency,” as used in this context, can be expressed as the concentration of rapalog required to achieve a 30% inhibition of S6K1 (Thr389)phosphorylation in a cell-based assay such as that used in Example 3 and FIGS. 1-11. For example, a rapalog is considered FKBP12-selective if the rapalog inhibits about 30% of S6K1 (Thr389)phosphorylation inhibition in assay using an FKBP12 knock-out cell line with at least 20× (e.g. 25×, 30×, 50×, 100×, 200×, 500×, 1000×, etc.) lower potency compared to similar level of inhibition achieved by RAD001 in the same assay with the same cell line, which normally expresses FKBP12. Non-limiting compounds of the present invention include:

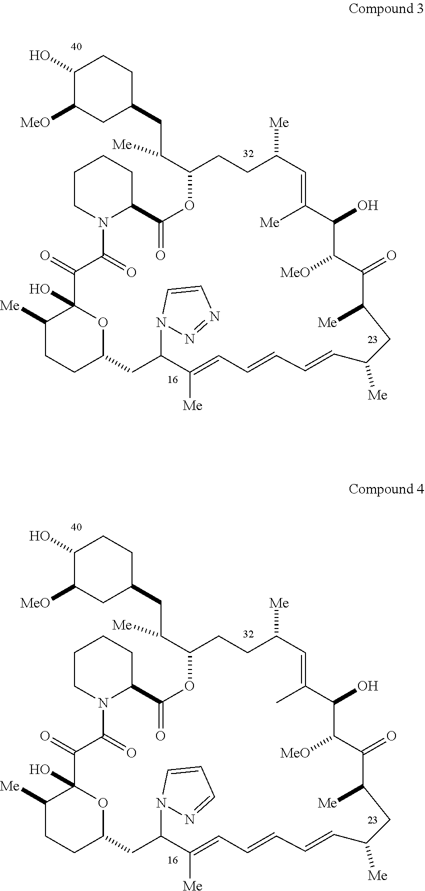 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(n3ccnn3)C[C@@H]3CC[C@@H](C)[C@@](O)(O3)C(=O)C(=O)N3CCCC[C@H]3C(=O)O2)CC[C@H]1O ; CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(n3cccn3)C[C@@H]3CC[C@@H](C)[C@@](O)(O3)C(=O)C(=O)N3CCCC[C@H]3C(=O)O2)CC[C@H]1O]

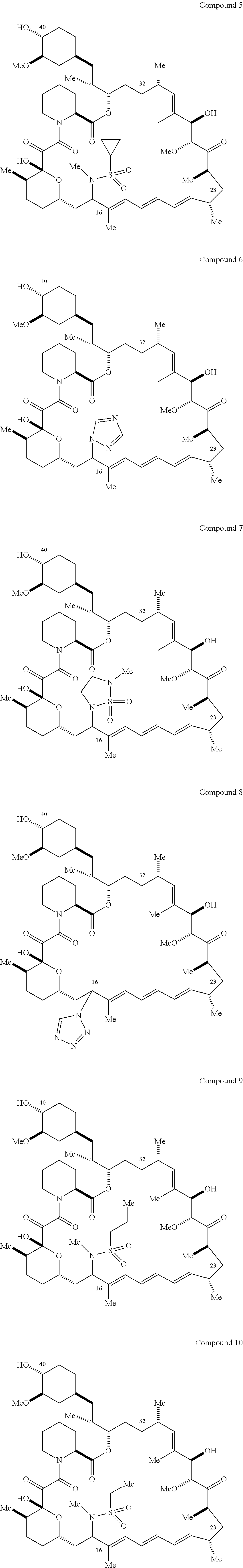

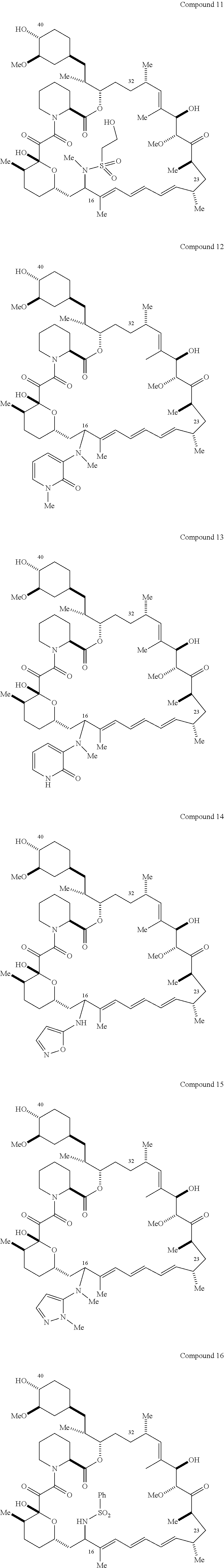

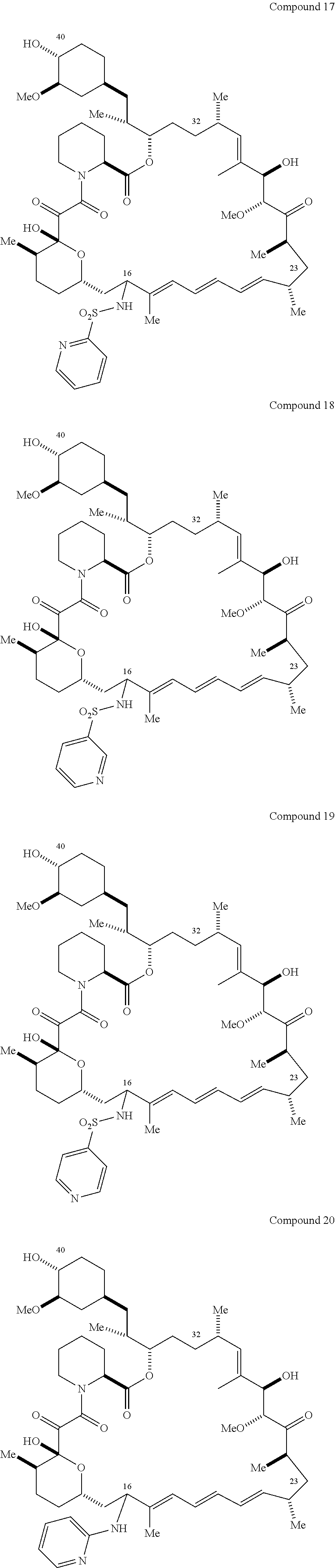

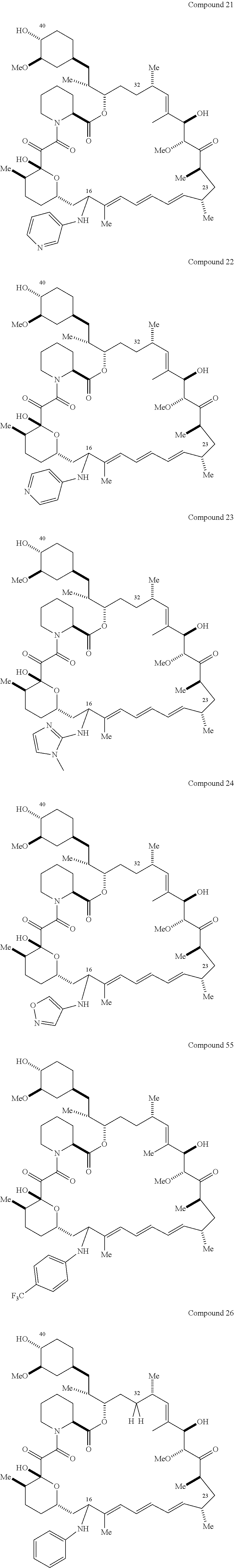

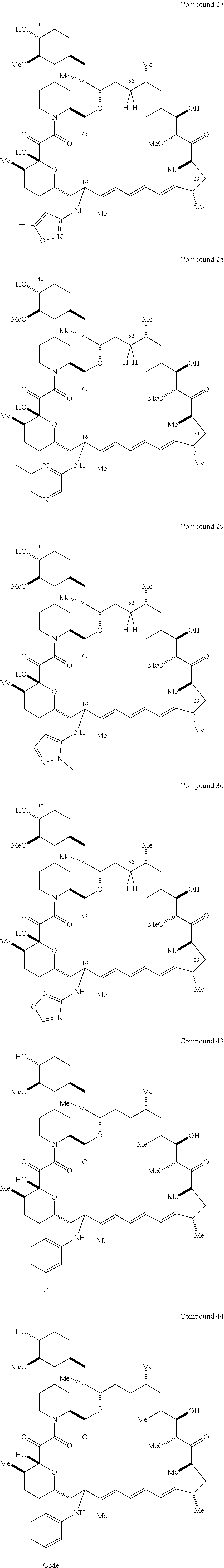

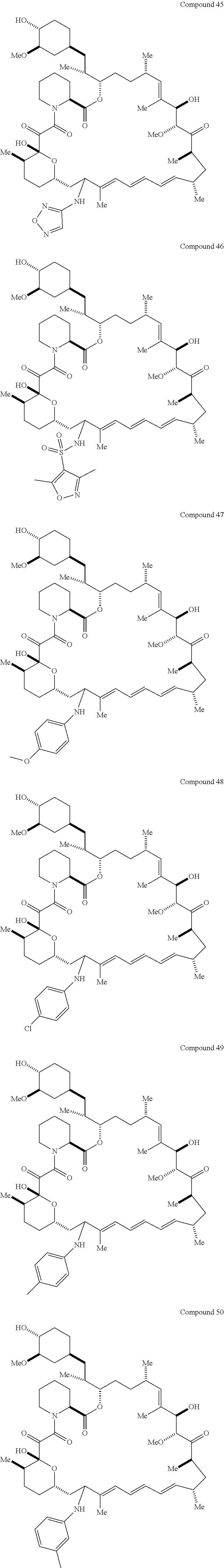

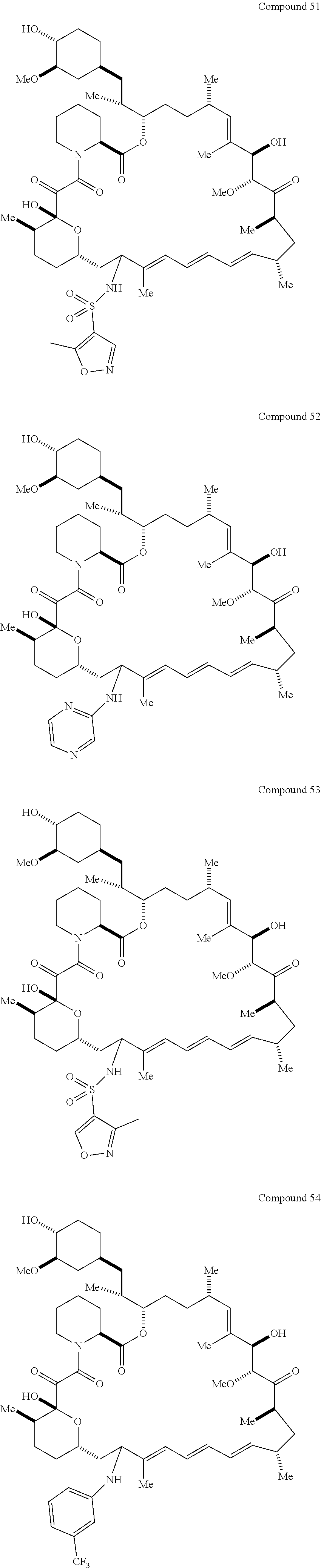

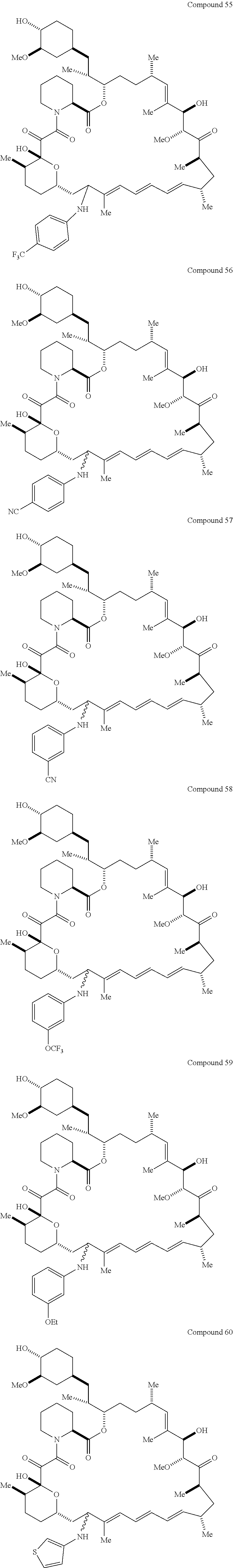

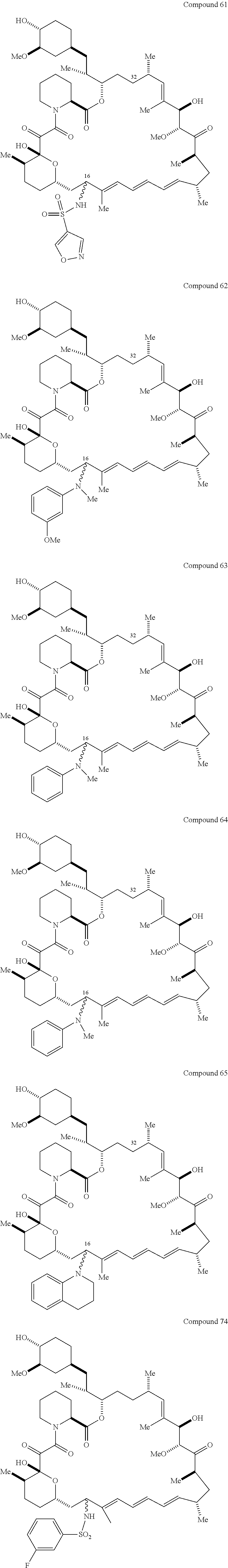

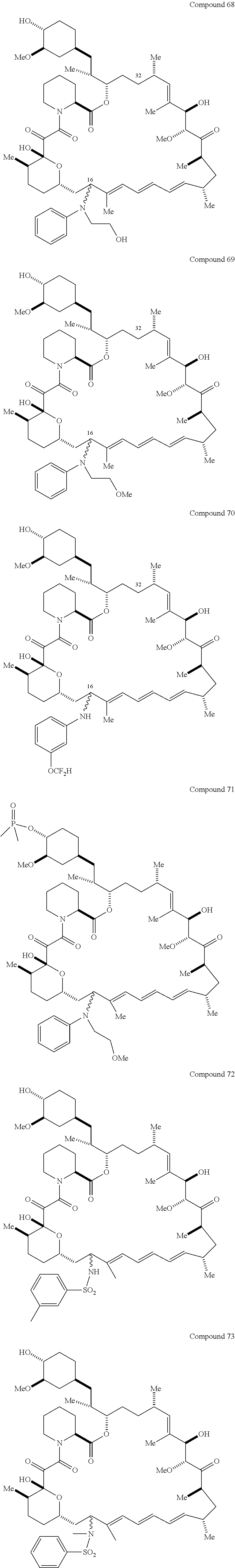

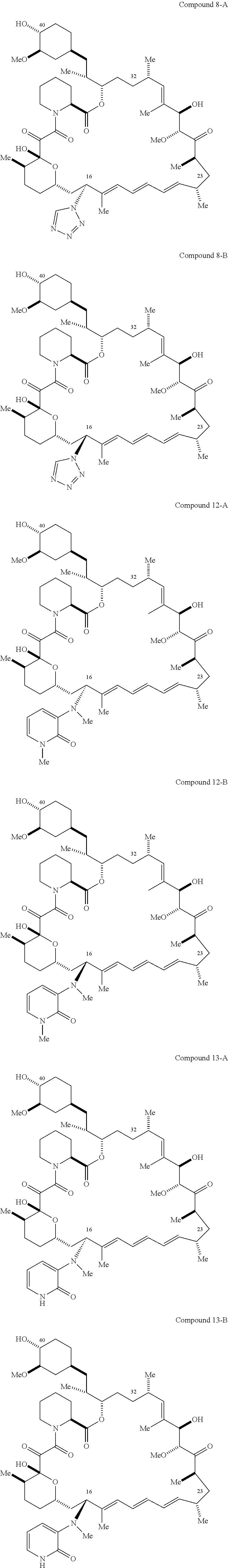

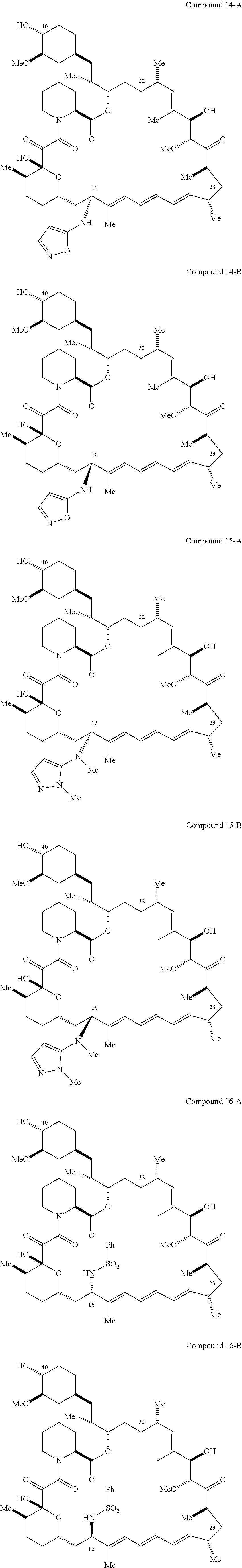

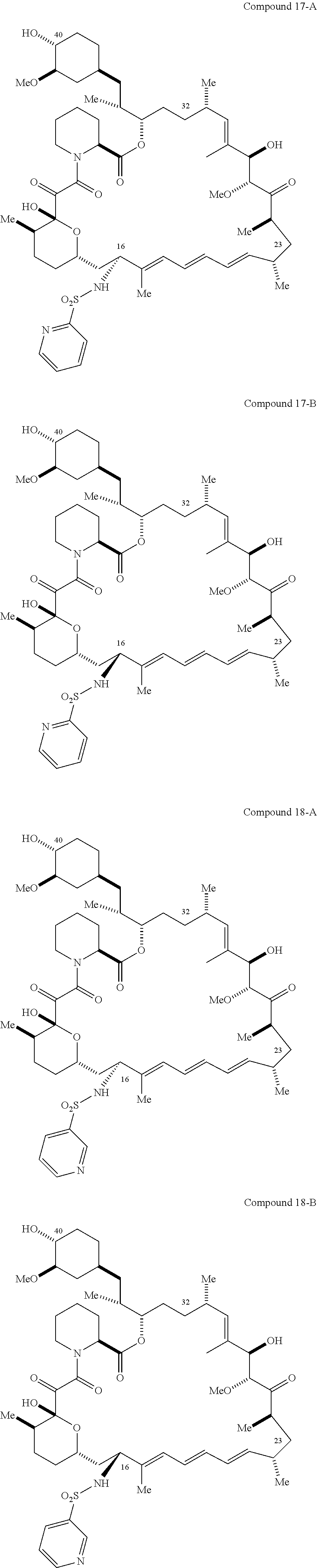

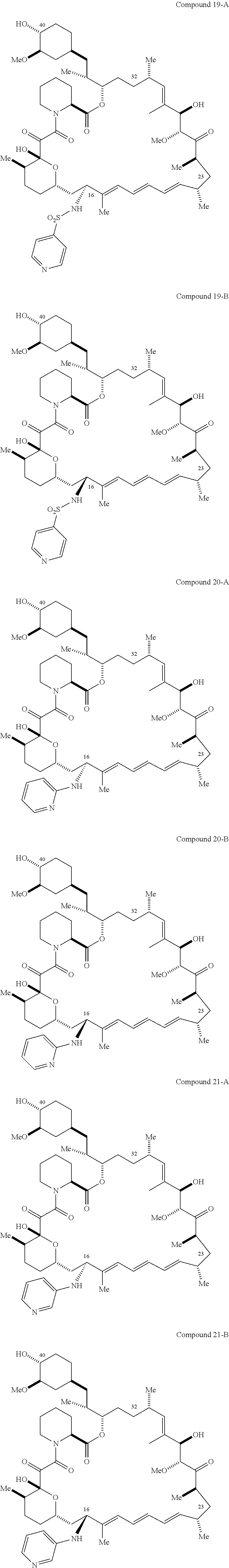

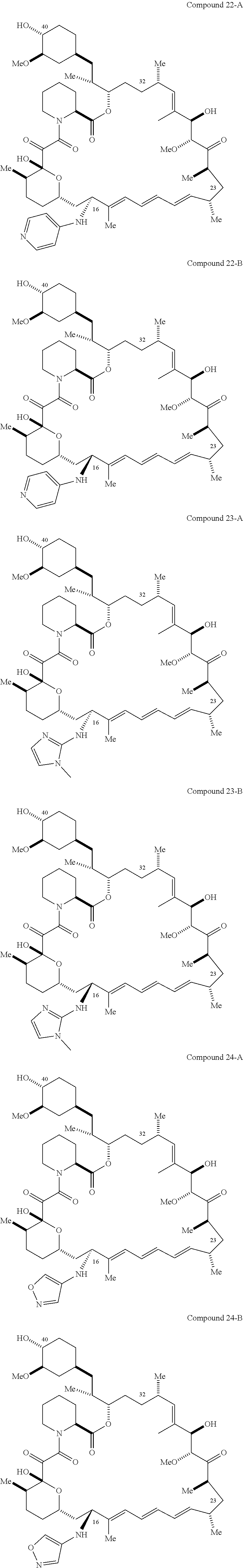

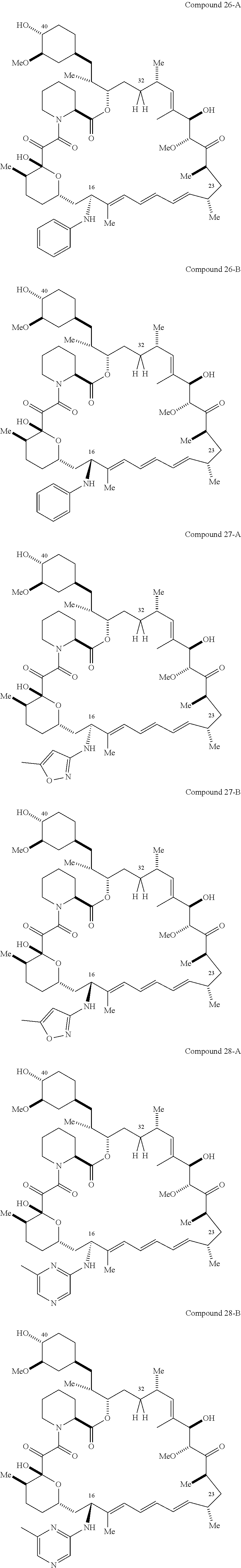

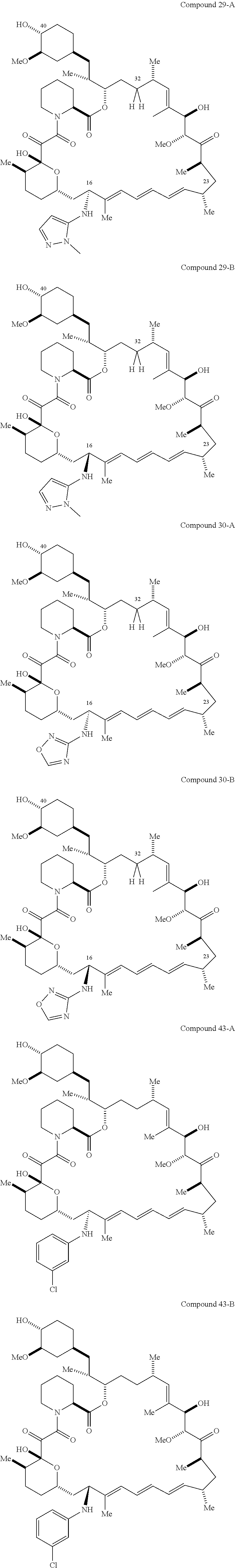

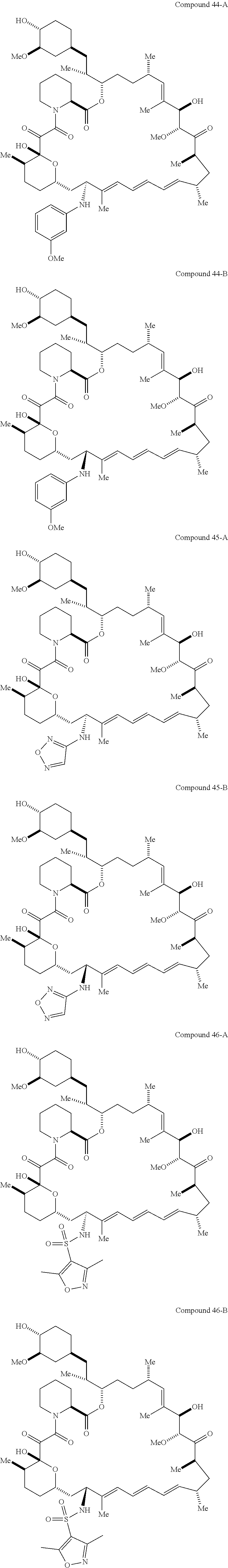

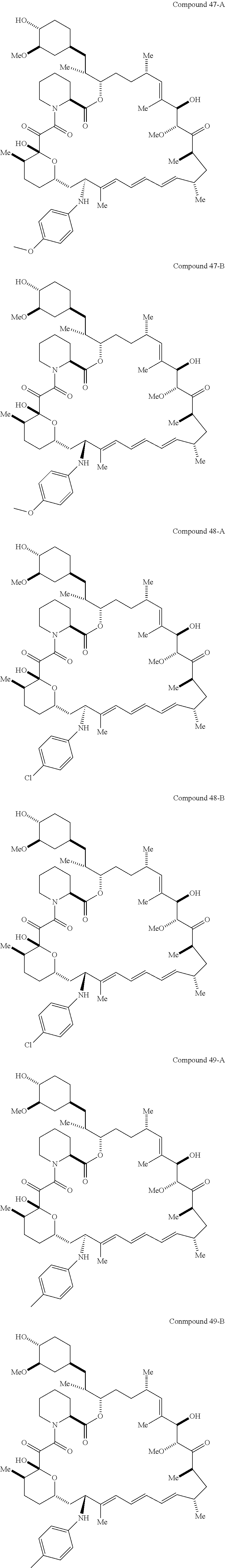

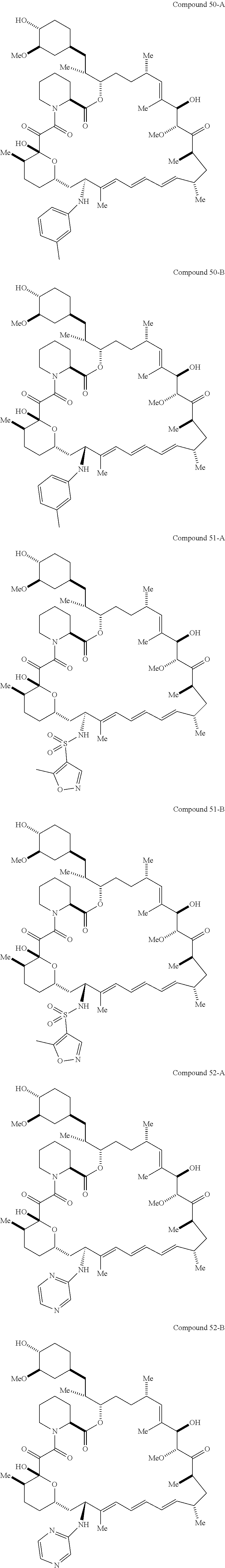

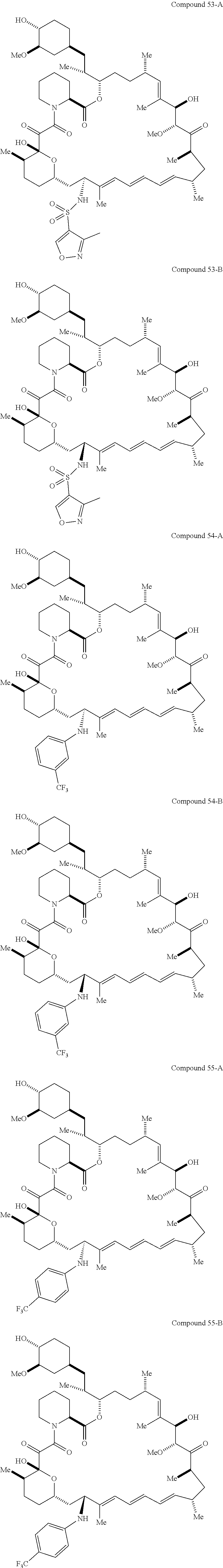

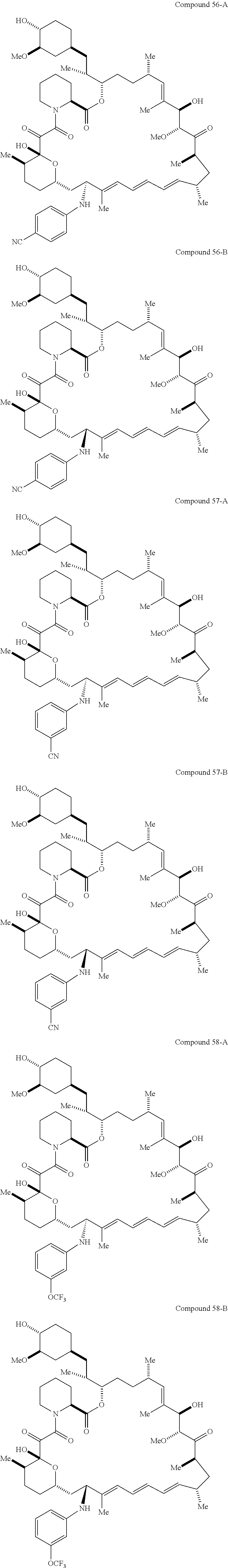

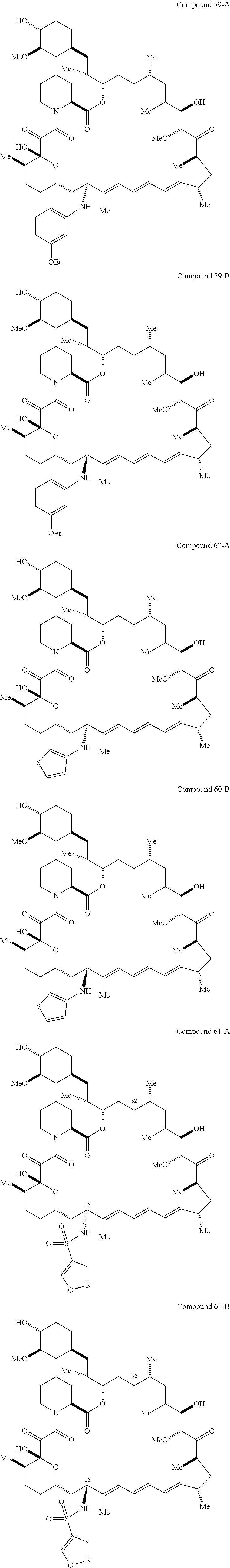

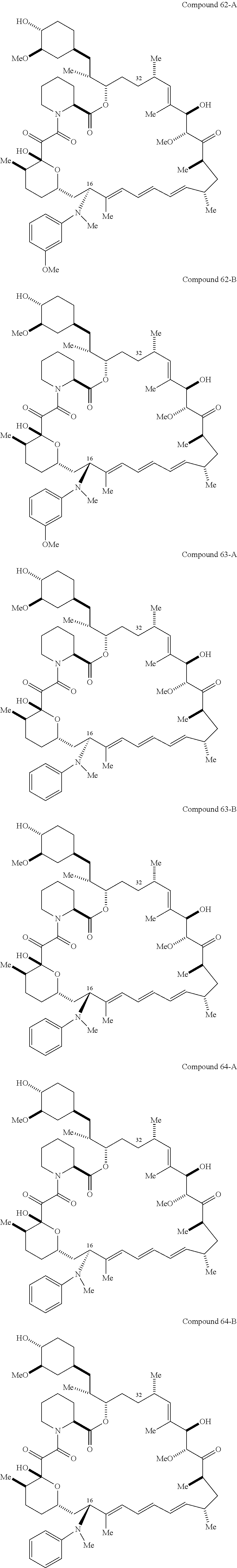

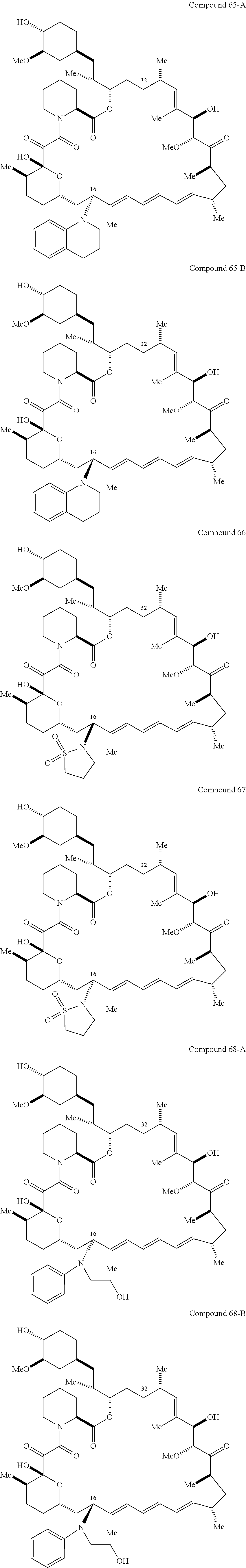

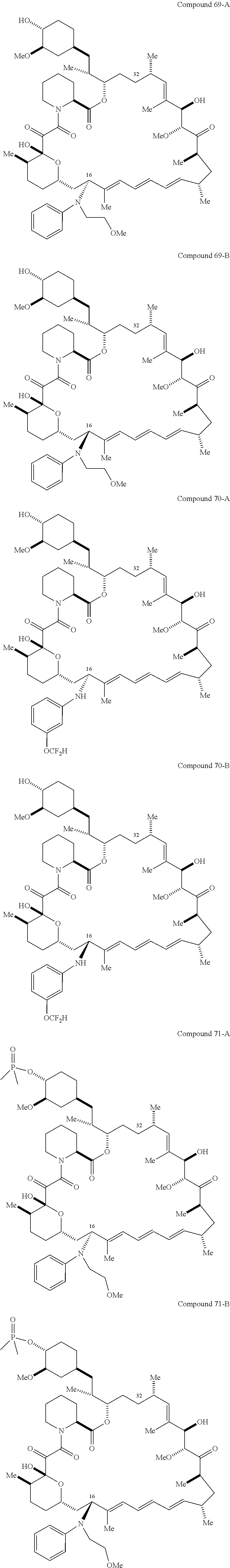

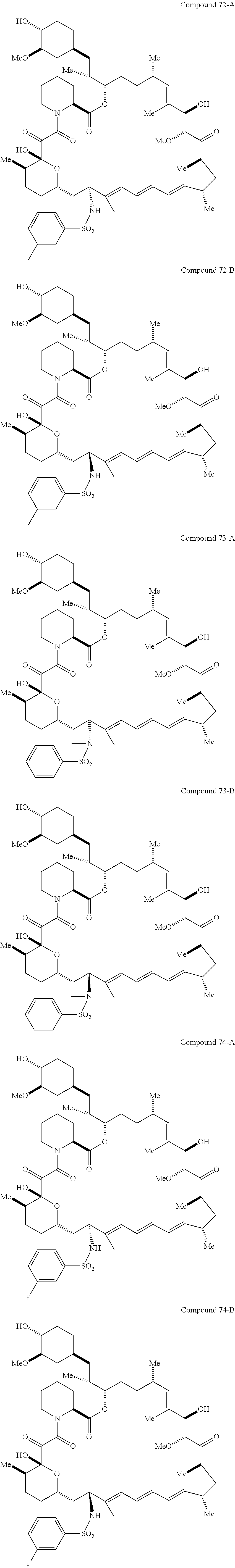

### Pharmaceutical Compositions and Methods of Treatment

Provided herein are methods of treating and preventing diseases, conditions, or disorders comprising administering a therapeutically or prophylactically effective amount or one or more of the compounds disclosed herein, for example, one or more of the compounds of a formula provided herein. Diseases, disorders, and/or conditions include, but are not limited to, those that are mediated by the mTOR pathway.

In some embodiments of the methods described herein, multiple doses of a compound described herein (or a pharmaceutical composition comprising a combination of a compound described herein and any of the additional therapeutic agents mentioned herein) may be administered to a subject over a defined time course. The methods according to this embodiment of the disclosure comprise sequentially administering to a subject multiple doses of a compound described herein. As used herein, “sequentially administering” means that each dose of the compound is administered to the subject at a different point in time, for example, on different days separated by a predetermined interval (e.g., hours, days, weeks, or months). This disclosure includes methods which comprise sequentially administering to the patient a single initial dose of a compound described herein, followed by one or more secondary doses of the compound, and optionally followed by one or more tertiary doses of the compound.

The terms “initial dose,” “secondary doses,” and “tertiary doses,” refer to the temporal sequence of administration of the compounds described herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses can all include the same amount the compound described herein, but generally can differ from one another in terms of frequency of administration. In certain embodiments, the amount of the compound included in the initial, secondary and/or tertiary doses varies from one another (e.g., adjusted up or down as appropriate) during the course of treatment. In certain embodiments, two or more (e.g., two, three, four, or five) doses are administered at the beginning of the treatment regimen as “loading doses” followed by subsequent doses that are administered on a less frequent basis (e.g., “maintenance doses”).

In certain exemplary embodiments of this disclosure, each secondary and/or tertiary dose is administered one to twenty-six (e.g., 1, 1½, 2, 2½, 3, 3½, 4, 4½, 5, 5½, 6, 6½, 7, 7½, 8, 8½, 9, 9½, 10, 104/2, 11, 11½, 12, 124/2, 13, 134/2, 14, 14½, 15, 15½, 16, 16½, 17, 17½, 18, 18½, 19, 19½, 20, 20½, 21, 21½, 22, 224/2, 23, 23½, 24, 24½, 25, 25½, 26, 26½, or more) weeks after the immediately preceding dose. The phrase “the immediately preceding dose,” as used herein, means, in a sequence of multiple administrations, the dose the compound which is administered to a patient prior to the administration of the very next dose in the sequence with no intervening doses.

The methods according to this embodiment of the disclosure may comprise administering to a patient any number of secondary and/or tertiary doses of the compound. For example, in certain embodiments, only a single secondary dose is administered to the patient. In other embodiments, two or more (e.g., two, three, four, five, six, seven, eight, or more) secondary doses are administered to the patient. Likewise, in certain embodiments, only a single tertiary dose is administered to the patient. In other embodiments, two or more (e.g., two, three, four, five, six, seven, eight, or more) tertiary doses are administered to the patient. The administration regimen may be carried out indefinitely over the lifetime of a particular subject, or until such treatment is no longer therapeutically needed or advantageous.

In embodiments involving multiple secondary doses, each secondary dose may be administered at the same frequency as the other secondary doses. For example, each secondary dose may be administered to the patient one to two weeks or one to two months after the immediately preceding dose. Similarly, in embodiments involving multiple tertiary doses, each tertiary dose may be administered at the same frequency as the other tertiary doses. For example, each tertiary dose may be administered to the patient two to twelve weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

This disclosure includes administration regimens in which two to six loading doses are administered to a patient at a first frequency (e.g., once a week, once every two weeks, once every three weeks, once a month, once every two months, etc.), followed by administration of two or more maintenance doses to the patient on a less frequent basis. For example, according to this embodiment of the disclosure, if the loading doses are administered at a frequency of once a month, then the maintenance doses may be administered to the patient once every six weeks, once every two months, once every three months, etc.

This disclosure includes pharmaceutical compositions of the compounds described herein, for example, compounds of Formula (I), (P-1), (P-2), (Ia)-(Ic), or (P-Ia)-(P-Ic) or Compounds 3-74, or a salt, and a pharmaceutically acceptable carrier, diluent, and/or excipient. Examples of suitable carriers, diluents and excipients include, but are not limited to, buffers for maintenance of proper composition pH (e.g., citrate buffers, succinate buffers, acetate buffers, phosphate buffers, lactate buffers, oxalate buffers, and the like), carrier proteins (e.g., human serum albumin), saline, polyols (e.g., trehalose, sucrose, xylitol, sorbitol, and the like), surfactants (e.g., polysorbate 20, polysorbate 80, polyoxolate, and the like), antimicrobials, and antioxidants.

This disclosure also includes combination therapy comprising 1) a compound of Formula (I), (P-1), (P-2), (Ia)-(Ic), or (P-Ia)-(P-Ic) or a pharmaceutical composition thereof and 2) an anti-CD40 antibody. In some embodiments, the compound of Formula (I), (P-1), (P-2), (Ia)-(Ic), or (P-Ia)-(P-Ic) or pharmaceutical composition thereof and the anti-CD40 antibody are administered in separate dosage forms. In some embodiments, the compound of Formula (I), (P-1), (P-2), (Ia)-(Ic), or (P-Ia)-(P-Ic) or pharmaceutical thereof and the anti-CD40 antibody are administered in a combined dosage form.

In some embodiments, set forth herein is a method of treating a disorder or a disease in a subject in need thereof comprising administering a compound of Formula (I), (P-1), (P-2), (Ia)-(Ic), or (P-Ia)-(P-Ic), or a pharmaceutically salt thereof.

In some embodiments, set forth herein is a method of treating a disorder or a disease in a subject in need thereof comprising administering combination therapy comprising 1) a compound of Formula (I), (P-1), (P-2), (Ia)-(Ic), or (P-Ia)-(P-Ic) or a pharmaceutical composition thereof and 2) an anti-CD40 antibody.

In some embodiments, set forth herein is a method of treating a disorder or a disease mediated by the mTOR pathway in a subject in need thereof comprising administering a compound of Formula (I), (P-1), (P-2), (Ia)-(Ic), or (P-Ia)-(P-Ic), or a pharmaceutically salt thereof. In one embodiment, the target tissue, organ, or cells associated with the pathology of the disease or disorder has FKBP12 levels sufficient to inhibit mTORC1.

In one embodiment, the disease or disorder is an age-related disorder or disease selected from sarcopenia; skin atrophy; cherry angiomas; seborrheic keratoses; brain atrophy; atherosclerosis; arteriosclerosis; pulmonary emphysema; osteoporosis; osteoarthritis; high blood pressure; erectile dysfunction; cataracts; macular degeneration; glaucoma; stroke; cerebrovascular disease (strokes); chronic kidney disease; diabetes-associated kidney disease; impaired hepatic function; liver fibrosis; autoimmune hepatitis; endometrial hyperplasia; metabolic dysfunction; renovascular disease; hearing loss; mobility disability; cognitive decline; tendon stiffness; heart dysfunction, such as cardiac hypertrophy and/or systolic and/or diastolic dysfunction and/or hypertension; heart dysfunction that results in a decline in ejection fraction; immune senescence; Parkinson's disease; Alzheimer's disease or a syndrome thereof; cancer; immune-senescence leading to cancer due to a decrease in immune-surveillance; infections due to an decline in immune-function; chronic obstructive pulmonary disease (COPD); obesity; loss of taste; loss of olfaction; arthritis; and, type II diabetes.

In one embodiment, the disease or disorder is cancer. In one embodiment, the cancer is selected from renal cancer, renal cell carcinoma, colorectal cancer, uterine sarcoma, endometrial uterine cancer, endometrial cancer, breast cancer, ovarian cancer, cervical cancer, gastric cancer, fibro-sarcoma, pancreatic cancer, liver cancer, melanoma, leukemia, multiple myeloma, nasopharyngeal cancer, prostate cancer, lung cancer, glioblastoma, bladder cancer, mesothelioma, head cancer, rhabdomyosarcoma, sarcoma, lymphoma, and neck cancer.

In one embodiment, the disease or disorder is Graft-versus-Host Disease (GvHD) or a syndrome thereof.

In one embodiment, the disease or disorder is facial angiofibroma associated with tuberous sclerosis complex.

In one embodiment, the disease or disorder is advanced unresectable or metastatic malignant perivascular epithelioid cell tumors.

In some embodiments, also set forth herein is a method of inducing immune tolerance and/or preventing transplant organ rejection in a subject in need thereof comprising administering a compound of Formula (I), (P-1), (Ia)-(Ic), or (P-Ia)-(P-Ic), or a pharmaceutically salt thereof.

The disclosure provides a compound of Formula (I), (P-1), (P-2), (Ia)-(Ic), or (P-Ia)-(P-Ic), or a pharmaceutically acceptable salt thereof for treatment of diseases and disorders described herein, e.g., age-related disorders, or diseases and disorders currently approved for treatment using rapalogs, such as RAD001.

In one aspect, the disclosure provides a method for treating a disorder or a disease mediated by the mTOR pathway in a subject in need thereof, the method comprising administering to the subject a therapeutically effective amount of a compound of Formula (I), (P-1), (P-2), (Ia)-(Ic), or (P-Ia)-(P-Ic), or a pharmaceutically acceptable salt thereof.

In an embodiment, efficacy of treatment is determined empirically, e.g., as compared to rapamycin or RAD001.

In another aspect, the disclosure provides a method of treating a disease or disorder in a subject having, or previously determined to have, FKBP12 levels sufficient to inhibit mTORC1, the method comprising administering to the subject in need thereof a therapeutically effective amount of a compound of Formula (I), (P-1), (P-2), (Ia)-(Ic), or (P-Ia)-(P-Ic), or a pharmaceutically acceptable salt thereof, or a pharmaceutical composition described herein, or a pharmaceutical combination described herein.

In an embodiment, the disease or disorder is selected from sarcopenia, skin atrophy, cherry angiomas, seborrheic keratoses, brain atrophy, atherosclerosis, arteriosclerosis, pulmonary emphysema, osteoporosis, osteoarthritis, high blood pressure, erectile dysfunction, cataracts, macular degeneration, glaucoma, stroke, cerebrovascular disease (strokes), chronic kidney disease, diabetes-associated kidney disease, impaired hepatic function, liver fibrosis, autoimmune hepatitis, endometrial hyperplasia, metabolic dysfunction, renovascular disease, hearing loss, mobility disability, cognitive decline, tendon stiffness, heart dysfunction such as cardiac hypertrophy and/or systolic and/or diastolic dysfunction and/or hypertension, heart dysfunction which results in a decline in ejection fraction, immune senescence, Parkinson's disease, Alzheimer's disease, cancer, immune-senescence leading to cancer due to a decrease in immune-surveillance, infections due to an decline in immune-function, chronic obstructive pulmonary disease (COPD), obesity, loss of taste, loss of olfaction, arthritis, and type II diabetes including complications stemming from diabetes, such as kidney failure, blindness and neuropathy.

In an embodiment, the disorder is liver fibrosis.

In another aspect, the disclosure provides a method of treating a disease or disorder in a subject in need thereof, the method comprising administering to the subject a therapeutically effective amount of a compound of Formula (I), (P-1), (P-2), (Ia)-(Ic), or (P-Ia)-(P-Ic), or a pharmaceutically acceptable salt thereof, a pharmaceutical composition comprising a compound of Formula (I), (P-1), (P-2), (Ia)-(Ic), or (P-Ia)-(P-Ic), or a pharmaceutically acceptable salt thereof, or a pharmaceutical combination comprising a compound of Formula (I), (P-1), (P-2), (Ia)-(Ic), or (P-Ia)-(P-Ic), or a pharmaceutically acceptable salt thereof, wherein the disorder or disease is selected from:
Acute or chronic organ or tissue transplant rejection;
Transplant vasculopathies;
Smooth muscle cell proliferation and migration leading to vessel intimal thickening, blood vessel obstruction, obstructive coronary atherosclerosis, restenosis;
Autoimmune diseases and inflammatory conditions;
Asthma;
Multi-drug resistance (MDR);
Fungal infections;
Inflammation;
Infection;
Age-related diseases;
Neurodegenerative diseases;
Proliferative disorders, in particular cancer;
Seizures and seizure related disorders; and.
Mitochondrial myopathy and mitochondrial stress.

In an embodiment, the disorder is a disorder that includes the process of fibrosis and/or inflammation.

In an embodiment, the disorder is selected from liver and kidney disorders.

In an embodiment, the liver disorder is selected from: liver fibrosis, which occurs in end-stage liver disease; liver cirrhosis; liver failure due to toxicity; non-alcohol-associated hepatic steatosis or NASH; and alcohol-associated steatosis.

In an embodiment, the kidney disorder is kidney fibrosis.

In an embodiment, the kidney fibrosis occurs as a result of acute kidney injury.

In an embodiment, the kidney disorder is chronic kidney disorder.

In an embodiment, the kidney disorder is diabetic nephropathy.

In another aspect, the disclosure provides a method of treating an age-related disorder or disease in a subject in need thereof, the method comprising administering to the subject a therapeutically effective amount of a compound of Formula (I), (P-1), (P-2), (Ia)-(Ic), or (P-Ia)-(P-Ic), or a pharmaceutically acceptable salt thereof, a pharmaceutical composition comprising a compound of Formula (I), (P-1), (P-2), (Ia)-(Ic), or (P-Ia)-(P-Ic), or a pharmaceutically acceptable salt thereof, or a pharmaceutical combination comprising a compound of Formula (I), (P-1), (P-2), (Ia)-(Ic), or (P-Ia)-(P-Ic), or a pharmaceutically acceptable salt thereof, wherein the disorder or disease is selected from: sarcopenia, skin atrophy, cherry angiomas, seborrheic keratoses, brain atrophy, atherosclerosis, arteriosclerosis, pulmonary emphysema, osteoporosis, osteoarthritis, high blood pressure, erectile dysfunction, cataracts, macular degeneration, glaucoma, stroke, cerebrovascular disease (strokes), chronic kidney disease, diabetes-associated kidney disease, impaired hepatic function, liver fibrosis, autoimmune hepatitis, endometrial hyperplasia, metabolic dysfunction, renovascular disease, hearing loss, mobility disability, cognitive decline, tendon stiffness, heart dysfunction such as cardiac hypertrophy and/or systolic and/or diastolic dysfunction and/or hypertension, heart dysfunction which results in a decline in ejection fraction, immune senescence, Parkinson's disease, Alzheimer's disease, cancer, immune-senescence leading to cancer due to a decrease in immune-surveillance, infections due to an decline in immune-function, chronic obstructive pulmonary disease (COPD), obesity, loss of taste, loss of olfaction, arthritis, and type II diabetes including complications stemming from diabetes, such as kidney failure, blindness and neuropathy.

In another aspect, the disclosure provides a method of treating cancer in a subject, the method comprising administering to the subject a therapeutically effective amount of a compound of Formula (I), (P-1), (P-2), (Ia)-(Ic), or (P-Ia)-(P-Ic), or a pharmaceutically acceptable salt thereof, a pharmaceutical composition comprising a compound of Formula (I), (P-1), (P-2), (Ia)-(Ic), or (P-Ia)-(P-Ic), or a pharmaceutically acceptable salt thereof, or a pharmaceutical combination comprising a compound of Formula (I), (P-1), (P-2), (Ia)-(Ic), or (P-Ia)-(P-Ic), or a pharmaceutically acceptable salt thereof.

In an embodiment, the method further comprises a PD-1/PDL-1 inhibitor.

In an embodiment, the cancer is selected from renal cancer, renal cell carcinoma, colorectal cancer, uterine sarcoma, endometrial uterine cancer, endometrial cancer, breast cancer, ovarian cancer, cervical cancer, gastric cancer, fibro-sarcoma, pancreatic cancer, liver cancer, melanoma, leukemia, multiple myeloma, nasopharyngeal cancer, prostate cancer, lung cancer, glioblastoma, bladder cancer, mesothelioma, head cancer, rhabdomyosarcoma, sarcoma, lymphoma, and neck cancer.

In another aspect, the disclosure provides a compound of Formula (I), (P-1), (P-2), (Ia)-(Ic), or (P-Ia)-(P-Ic), or a pharmaceutically acceptable salt thereof, a pharmaceutical composition comprising a compound of Formula (I), (P-1), (P-2), (Ia)-(Ic), or (P-Ia)-(P-Ic), or a pharmaceutically acceptable salt thereof, or a pharmaceutical combination comprising a compound of Formula (I), (P-1), (P-2), (Ia)-(Ic), or (P-Ia)-(P-Ic), or a pharmaceutically acceptable salt thereof, for use as a medicament.

In another aspect, the disclosure provides a compound of Formula (I), (P-1), (P-2), (Ia)-(Ic), or (P-Ia)-(P-Ic), or a pharmaceutically acceptable salt thereof, a pharmaceutical composition comprising a compound of Formula (I), (P-1), (P-2), (Ia)-(Ic), or (P-Ia)-(P-Ic), or a pharmaceutically acceptable salt thereof, or a pharmaceutical combination comprising a compound of Formula (I), (P-1), (P-2), (Ia)-(Ic), or (P-Ia)-(P-Ic), or a pharmaceutically acceptable salt thereof, for use in the prevention or treatment of a disorder or disease mediated by the mTOR pathway.

In another aspect, the disclosure provides a compound of Formula (I), (P-1), (P-2), (Ia)-(Ic), or (P-Ia)-(P-Ic), or a pharmaceutically acceptable salt thereof, a pharmaceutical composition comprising a compound of Formula (I), (P-1), (P-2), (Ia)-(Ic), or (P-Ia)-(P-Ic), or a pharmaceutically acceptable salt thereof, or a pharmaceutical combination comprising a compound of Formula (I), (P-1), (P-2), (Ia)-(Ic), or (P-Ia)-(P-Ic), or a pharmaceutically acceptable salt thereof, for use in the prevention or treatment of a disorder or disease selected from:
Acute or chronic organ or tissue transplant rejection;
Transplant vasculopathies;
Smooth muscle cell proliferation and migration leading to vessel intimal thickening, blood vessel obstruction, obstructive coronary atherosclerosis, restenosis;
Autoimmune diseases and inflammatory conditions;
Asthma;
Multi-drug resistance (MDR);
Fungal infections;
Inflammation;
Infection;
Age-related diseases;
Neurodegenerative diseases;
Proliferative disorders, in particular cancer;
Seizures and seizure related disorders; and
Mitochondrial myopathy and mitochondrial stress.

In another aspect, the disclosure provides a compound of Formula (I), (P-1), (P-2), (Ia)-(Ic), or (P-Ia)-(P-Ic), or a pharmaceutically acceptable salt thereof, a pharmaceutical composition comprising a compound of Formula (I), (P-1), (P-2), (Ia)-(Ic), or (P-Ia)-(P-Ic), or a pharmaceutically acceptable salt thereof, or a pharmaceutical combination comprising a compound of Formula (I), (P-1), (P-2), (Ia)-(Ic), or (P-Ia)-(P-Ic), or a pharmaceutically acceptable salt thereof, for use in the prevention or treatment of a disorder or disease that includes the process of fibrosis and/or inflammation.

In an embodiment, the disorder is selected from liver and kidney disorders.

In an embodiment, the liver disorder is selected from: liver fibrosis, which occurs in end-stage liver disease; liver cirrhosis; liver failure due to toxicity; non-alcohol-associated hepatic steatosis or NASH; and alcohol-associated steatosis.

In an embodiment, the kidney disorder is kidney fibrosis, which occurs as a result of acute kidney injury.

In an embodiment, the kidney disorder is chronic kidney disorder.

In an embodiment, the kidney disorder is diabetic nephropathy.

In another aspect, the disclosure provides a compound of Formula (I), (P-1), (P-2), (Ia)-(Ic), or (P-Ia)-(P-Ic), or a pharmaceutically acceptable salt thereof, a pharmaceutical composition comprising a compound of Formula (I), (P-1), (P-2), (Ia)-(Ic), or (P-Ia)-(P-Ic), or a pharmaceutically acceptable salt thereof, or a pharmaceutical combination comprising a compound of Formula (I), (P-1), (P-2), (Ia)-(Ic), or (P-Ia)-(P-Ic), or a pharmaceutically acceptable salt thereof, for use in the treatment of an age-related disorder or disease selected from sarcopenia, skin atrophy, cherry angiomas, seborrheic keratoses, brain atrophy (also referred to as dementia), atherosclerosis, arteriosclerosis, pulmonary emphysema, osteoporosis, osteoarthritis, high blood pressure, erectile dysfunction, cataracts, macular degeneration, glaucoma, stroke, cerebrovascular disease (strokes), chronic kidney disease, diabetes-associated kidney disease, impaired hepatic function, liver fibrosis, autoimmune hepatitis, endometrial hyperplasia, metabolic dysfunction, renovascular disease, hearing loss, mobility disability (e.g., frailty), cognitive decline, tendon stiffness, heart dysfunction such as cardiac hypertrophy and/or systolic and/or diastolic dysfunction and/or hypertension, heart dysfunction which results in a decline in ejection fraction, immune senescence, Parkinson's disease, Alzheimer's disease, cancer, immune-senescence leading to cancer due to a decrease in immune-surveillance, infections due to an decline in immune-function, chronic obstructive pulmonary disease (COPD), obesity, loss of taste, loss of olfaction, arthritis, and type II diabetes (including complications stemming from diabetes, such as kidney failure, blindness and neuropathy).

In another aspect, the disclosure provides a compound of Formula (I), (P-1), (P-2), (Ia)-(Ic), or (P-Ia)-(P-Ic), or a pharmaceutically acceptable salt thereof, a pharmaceutical composition comprising a compound of Formula (I), (P-1), (P-2), (Ia)-(Ic), or (P-Ia)-(P-Ic), or a pharmaceutically acceptable salt thereof, or a pharmaceutical combination comprising a compound of Formula (I), (P-1), (P-2), (Ia)-(Ic), or (P-Ia)-(P-Ic), or a pharmaceutically acceptable salt thereof, for use in the treatment of cancer.

In another aspect, the disclosure provides a compound of Formula (I), (P-1), (P-2), (Ia)-(Ic), or (P-Ia)-(P-Ic), or a pharmaceutically acceptable salt thereof, a pharmaceutical composition comprising a compound of Formula (I), (P-1), (P-2), (Ia)-(Ic), or (P-Ia)-(P-Ic), or a pharmaceutically acceptable salt thereof, or a pharmaceutical combination comprising a compound of Formula (I), (P-1), (P-2), (Ia)-(Ic), or (P-Ia)-(P-Ic), or a pharmaceutically acceptable salt thereof, for use in the treatment of renal cancer, renal cell carcinoma, colorectal cancer, uterine sarcoma, endometrial uterine cancer, endometrial cancer, breast cancer, ovarian cancer, cervical cancer, gastric cancer, fibro-sarcoma, pancreatic cancer, liver cancer, melanoma, leukemia, multiple myeloma, nasopharyngeal cancer, prostate cancer, lung cancer, glioblastoma, bladder cancer, mesothelioma, head cancer, rhabdomyosarcoma, sarcoma, lymphoma, or neck cancer.

In another aspect, the disclosure provides a use of a compound of Formula (I), (P-1), (P-2), (Ia)-(Ic), or (P-Ia)-(P-Ic), or a pharmaceutically acceptable salt thereof for the manufacture of a medicament.

In another aspect, the disclosure provides a use of a compound of Formula (I), (P-1), (P-2), (Ia)-(Ic), or (P-Ia)-(P-Ic), or a pharmaceutically acceptable salt thereof for the manufacture of a medicament for the treatment of a disorder or disease mediated by the mTOR pathway.

In another aspect, the disclosure provides a use of a compound of Formula (I), (P-1), (P-2), (Ia)-(Ic), or (P-Ia)-(P-Ic), or a pharmaceutically acceptable salt thereof for the manufacture of a medicament for the treatment of a disorder or disease selected from:
Acute or chronic organ or tissue transplant rejection;
Transplant vasculopathies;
Smooth muscle cell proliferation and migration leading to vessel intimal thickening, blood vessel obstruction, obstructive coronary atherosclerosis, restenosis;
Autoimmune diseases and inflammatory conditions;
Asthma;
Multi-drug resistance (MDR);
Fungal infections;
Inflammation;
Infection;
Age-related diseases;
Neurodegenerative diseases;
Proliferative disorders, e.g., cancer;
Seizures and seizure related disorders; and
Mitochondrial myopathy and mitochondrial stress.

In another aspect, the disclosure provides a use of a compound of Formula (I), (P-1), (P-2), (Ia)-(Ic), or (P-Ia)-(P-Ic), or a pharmaceutically acceptable salt thereof for the manufacture of a medicament for the treatment of a disorder or disease that includes the process of fibrosis or inflammation.

In an embodiment, the disorder is selected from liver and kidney disorders.

In an embodiment, the liver disorder is selected from: liver fibrosis, which occurs in end-stage liver disease; liver cirrhosis; liver failure due to toxicity; non-alcohol-associated hepatic steatosis or NASH; and alcohol-associated steatosis.

In an embodiment, the kidney disorder is kidney fibrosis, which occurs as a result of acute kidney injury.

In an embodiment, the kidney disorder is chronic kidney disorder.

In an embodiment, the kidney disorder is diabetic nephropathy.

In another aspect, the disclosure provides a use of a compound of Formula (I), (P-1), (P-2), (Ia)-(Ic), or (P-Ia)-(P-Ic), or a pharmaceutically acceptable salt thereof for the manufacture of a medicament for the manufacture of a medicament for the prevention or treatment of an age-related disorder or disease selected from sarcopenia, skin atrophy, cherry angiomas, seborrheic keratoses, brain atrophy (also referred to as dementia), atherosclerosis, arteriosclerosis, pulmonary emphysema, osteoporosis, osteoarthritis, high blood pressure, erectile dysfunction, cataracts, macular degeneration, glaucoma, stroke, cerebrovascular disease (strokes), chronic kidney disease, diabetes-associated kidney disease, impaired hepatic function, liver fibrosis, autoimmune hepatitis, endometrial hyperplasia, metabolic dysfunction, renovascular disease, hearing loss, mobility disability, cognitive decline, tendon stiffness, heart dysfunction such as cardiac hypertrophy and/or systolic and/or diastolic dysfunction and/or hypertension, heart dysfunction which results in a decline in ejection fraction, immune senescence, Parkinson's disease, Alzheimer's disease, cancer, immune-senescence leading to cancer due to a decrease in immune-surveillance, infections due to an decline in immune-function, chronic obstructive pulmonary disease (COPD), obesity, loss of taste, loss of olfaction, arthritis, and type II diabetes (including complications stemming from diabetes, such as kidney failure, blindness and neuropathy).

In another aspect, the disclosure provides a use of a compound of Formula (I), (P-1), (P-2), (Ia)-(Ic), or (P-Ia)-(P-Ic), or a pharmaceutically acceptable salt thereof for the manufacture of a medicament for the manufacture of a medicament for the prevention or treatment of cancer.

In another aspect, the disclosure provides a use of a compound of Formula (I), (P-1), (P-2), (Ia)-(Ic), or (P-la)-(P-Ic), or a pharmaceutically acceptable salt thereof for the manufacture of a medicament for the manufacture of a medicament for the treatment of renal cancer, renal cell carcinoma, colorectal cancer, uterine sarcoma, endometrial uterine cancer, endometrial cancer, breast cancer, ovarian cancer, cervical cancer, gastric cancer, fibro-sarcoma, pancreatic cancer, liver cancer, melanoma, leukemia, multiple myeloma, nasopharyngeal cancer, prostate cancer, lung cancer, glioblastoma, bladder cancer, mesothelioma, head cancer, rhabdomyosarcoma, sarcoma, lymphoma, or neck cancer.

The details of one or more embodiments of the disclosure are set forth herein. Other features, objects, and advantages of the disclosure will be apparent from the Examples, the Figures/Drawings, and the Claims.

### Examples

Certain embodiments of this disclosure are illustrated by the following non-limiting examples. As used herein, the symbols and conventions used in these processes, schemes, and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the Examples, and throughout the specification.

| Abbreviation | Term or Phrase |
|---|---|
| AcOH | Acetic acid |
| ACN | Acetonitrile |
| ADC | Antibody-drug conjugate |
| Aq. | Aqueous |
| DCM | Dichloromethane |
| DMF | N,N-dimethylformamide |
| DMSO | Dimethylsulfoxide |
| Et₂O | Diethylether. |
| EtOAc | Ethyl acetate |
| g | Gram |
| HC | Heavy chain of immunoglobulin |
| HPLC | High performance liquid chromatography |
| hr, h, or hrs | Hours |
| IC50 | The concentration that provides 50% inhibition of target |
| | activities/signaling |
| IC30 | The concentration that provides 30% inhibition of target |
| | activities/signaling |
| LC | Light chain of immunoglobulin |
| mg | milligrams |
| min | minutes |
| mL | milliliters |
| μL | microliters |
| mM | millimolar |
| μM | micromolar |
| MW | Molecular weight |
| nM | nanomolar |
| NMR | Nuclear magnetic resonance |
| PABC | Para-aminobenzyloxy(carbonyl) |
| PEG | Polyethyleneglycol |
| pTSA | p-Toluenesulfonic acid |
| ppm | Parts per million (chemical shift, δ) |
| RP | Reversed phase |
| rt | room temperature |
| Sat. | Saturated |
| TEA | Triethylamine |
| TESOTf | Triethylsilyl trifluoromethanesulfonate |
| TMS | tetramethylsilane |
| TFA | Trifluoroacetic acid |
| TG | Transglutaminase |
| THF | Tetrahydrofuran |

### Example 1. Synthesis of 32-Deoxorapamycin

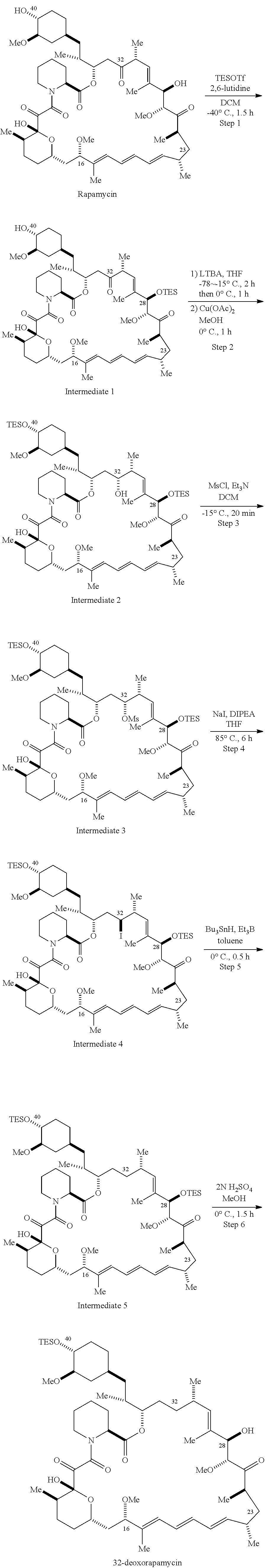

Step 1: To a solution of rapamycin (20 g, 21.88 mmol) in DCM (280 mL) was added 2,6-lutidine (15.29 mL, 131.26 mmol) and TESOTf (14.84 mL, 65.63 mmol) at −40° C. and stirred for 1.5 h. The resulting mixture was diluted with Et₂O and quenched with sat. aq. NaHCO₃. The organic phase was separated and washed with sat. aq. NaHCO₃, sat. aq. CuSO₄, and sat. aq. NH₄Cl. The combined aq. phase was re-extracted with Et₂O. The combined organic phase was washed with sat. aq. CuSO₄ and then dried with MgSO₄, filtered, and concentrated in vacuo. The residue was dissolved in DCM and subjected to ISCO silica column and eluted with gradient 0 to 25% EtOAc/hexane to obtain 95% of intermediate product 1 ((3S,6R,7E,9R,10R,12R,14S, 15E, 17E, 19E,21S,23S,26R,27R,34aS)-27-hydroxy-10,21-dimethoxy-3-((R)-1-((1S,3R,4R)-3-methoxy-4-((triethylsilyl)oxy)cyclohexyl) propan-2-yl)-6,8,12,14,20,26-hexamethyl-9-((triethylsilyl)oxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone) as a white solid. The ¹H-NMR data was matched with the reference (i.e. Tetrahedron Letters, 1994, 35 (41), 7557-7560).

Step 2: intermediate product 2 ((3S,5R,6R,7E,9R,10R,12R,14S,15E,17E,19E,21S,23S,26R,27R,34aS)-5,27-dihydroxy-10,21-dimethoxy-3-((R)-1-((1S,3R,4R)-3-methoxy-4-((triethylsilyl)oxy)cyclohexyl) propan-2-yl)-6,8,12,14,20,26-hexamethyl-9-((triethylsilyl)oxy)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone) was made following the procedure in patent WO 96/41807 and the ¹H-NMR data was matched.

Step 3: intermediate product 3 ((3S,5R,6R,7E,9R,10R,12R,14S, 15E, 17E,19E,21S,23S,26R,27R,34aS)-27-hydroxy-10,21-dimethoxy-3-((R)-1-((1S,3R,4R)-3-methoxy-4-((triethylsilyl)oxy)cyclohexyl) propan-2-yl)-6,8,12,14,20,26-hexamethyl-1,11,28,29-tetraoxo-9-((triethylsilyl)oxy)-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-5-yl methanesulfonate) was made following the procedure in patent WO 96/41807 and the ¹H-NMR data was matched.

Step 4 intermediate product 4 ((3S,5S,6R,7E,9R,10R,12R,14S,15E, 17E, 19E,21S,23S,26R,27R,34aS)-27-hydroxy-5-iodo-10,21-dimethoxy-3-((R)-1-((1S,3R,4R)-3-methoxy-4-((triethylsilyl)oxy)cyclohexyl) propan-2-yl)-6,8,12,14,20,26-hexamethyl-9-((triethylsilyl)oxy)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone) was made following the procedure in patent WO 96/41807 and the ¹H-NMR data was matched.

Step 5: intermediate product 5 ((3S,6R,7E,9R,10R,12R,14S, 15E, 17E, 19E,21S,23S,26R,27R,34aS)-27-hydroxy-10,21-dimethoxy-3-((R)-1-((1S,3R,4R)-3-methoxy-4-((triethylsilyl)oxy)cyclohexyl) propan-2-yl)-6,8,12,14,20,26-hexamethyl-9-((triethylsilyl)oxy)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone was made following the procedure in patent WO 96/41807 and the ¹H-NMR data was matched.

Step 6:32-deoxorapamycin was made following the procedure in patent WO 96/41807. The purification was performed by ISO silica column eluted with gradient 40 to 60% EtOAc/Hexane to afford 95% product as a white solid. The ¹H-NMR data and Mass was matched with the reference.

### Example 2. Synthesis of Representative 32-Deoxorapamycin Analogs

General Condition A1: To a solution of 32-deoxorapamycin (30.0 mg, 0.0333 mmol) and the appropriate amine nucleophile (5 eq.) in DCM (0.67 mL) was added ZnCl₂ (3 eq., 1.0 M solution in Et₂O) at 0° C. The reaction was warmed up to rt and stirred until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient 60 to 85% ACN/water (+0.05% AcOH as the modifier) to obtain the product.

General condition B1: To a solution of 32-deoxorapamycin (30.0 mg, 0.0333 mmol) and the appropriate amine nucleophile (20 eq.) in DCM (0.67 mL) was added pTSA·H₂O (5 eq.) at rt. The reaction was stirred at rt until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient 60 to 85% ACN/water (+0.05% AcOH as the modifier) to obtain the product.

### Compound 3:

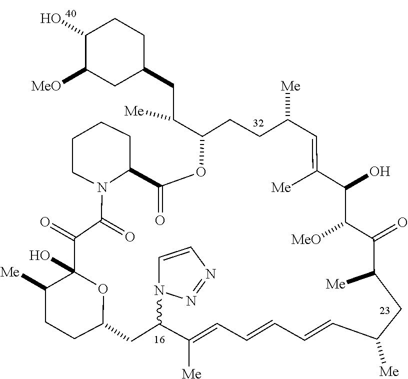 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(n3ccnn3)C[C@@H]3CC[C@@H](C)[C@@](O)(O3)C(=O)C(=O)N3CCCC[C@H]3C(=O)O2)CC[C@H]1O]

General condition A1 was applied. The procedure was modified with 20 eq. of triazole and 15 eq. of ZnCl₂. Additional 1.0 mL of ACN was added as the co-solvent. The reaction was stirred at 39° C. for 5 d to complete. Compound 3 was isolated in 6.1% as a white solid. Mass calculated 936.58, found 935.93 (M−H)⁻.

### Compound 4:

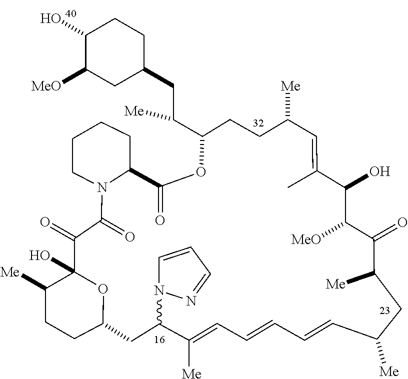 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(n3cccn3)C[C@@H]3CC[C@@H](C)[C@@](O)(O3)C(=O)C(=O)N3CCCC[C@H]3C(=O)O2)CC[C@H]1O]

General condition A1 was applied. The procedure was modified with 10 eq. of pyrazole and 10 eq. of ZnCl₂. The reaction was stirred at rt for 2 d to complete. Compound 4 was isolated in 13% as a white solid. Mass calculated 935.59, found 934.93 (M−H)⁻.

### Compound 5:

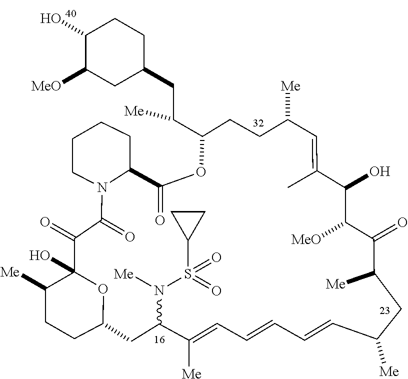 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(N(C)S(=O)(=O)C3CC3)C[C@@H]3CC[C@@H](C)[C@@](O)(O3)C(=O)C(=O)N3CCCC[C@H]3C(=O)O2)CC[C@H]1O]

General condition A1 was applied. The reaction was stirred at rt for 2 h to complete. Compound 5 was isolated in 32% as a white solid. Mass calculated 1002.59, found 1001.99 (M−H)⁻.

Compound 6:

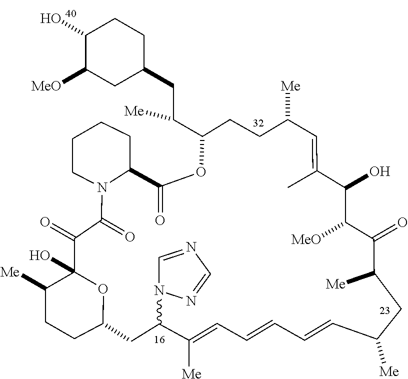 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(n3cncn3)C[C@@H]3CC[C@@H](C)[C@@](O)(O3)C(=O)C(=O)N3CCCC[C@H]3C(=O)O2)CC[C@H]1O]

General condition A1 was applied. The procedure was modified with 30 eq. of triazole and 10 eq. of ZnCl₂. Additional 2.0 mL of THF was added as the co-solvent. The reaction was stirred at 39° C. for 2 d to complete. Compound 6 was isolated in 10% as a white solid. Mass calculated 936.58, found 938.06 (M+H)⁺.

### Compound 8:

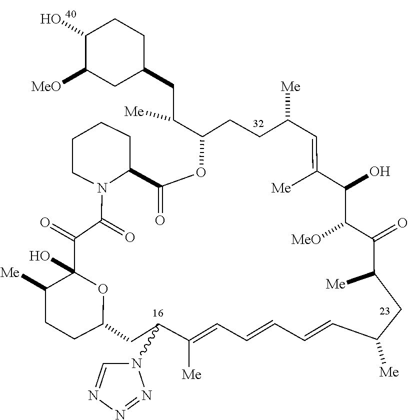 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(n3cnnn3)C[C@@H]3CC[C@@H](C)[C@@](O)(O3)C(=O)C(=O)N3CCCC[C@H]3C(=O)O2)CC[C@H]1O]

General condition A1 was applied. The procedure was modified with 20 eq. of tetrazole and 15 eq. of ZnCl₂. The reaction was stirred at rt for 1 h to complete. Compound 8 was isolated in 11% as a white solid. Mass calculated 937.58, found 939.05 (M+H)⁺.

Compound 9:

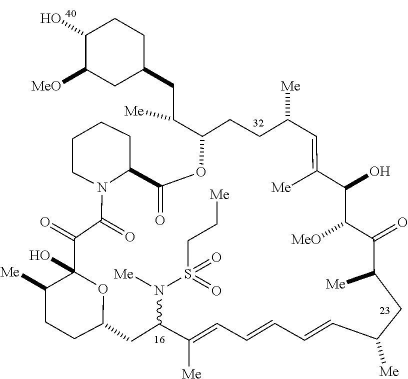 [SMILES: CCCS(=O)(=O)N(C)C1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC[C@H](C)/C=C(\C)[C@@H](O)[C@@H](OC)C(=O)[C@H](C)C[C@H](C)/C=C/C=C/C=C/1C]

General condition A1 was applied. The reaction was stirred at rt for 2 h to complete. Compound 9 was isolated in 25% as a white solid. Mass calculated 1004.60, found 1004.04 (M−H)⁻.

### Compound 10:

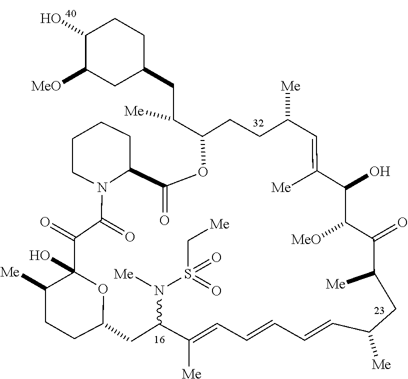 [SMILES: CCS(=O)(=O)N(C)C1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC[C@H](C)/C=C(\C)[C@@H](O)[C@@H](OC)C(=O)[C@H](C)C[C@H](C)/C=C/C=C/C=C/1C]

General condition A1 was applied. The reaction was stirred at rt for 2 h to complete. Compound 10 was isolated in 18% as a white solid. Mass calculated 990.59, found 989.68 (M−H)⁻.

### Compound 11:

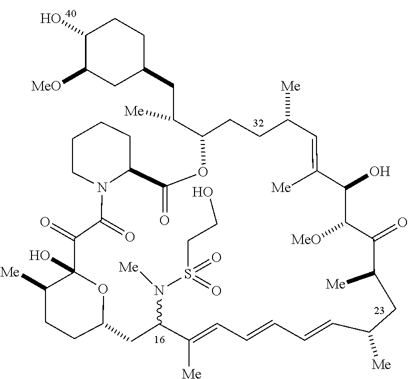 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(N(C)S(=O)(=O)CCO)C[C@@H]3CC[C@@H](C)[C@@](O)(O3)C(=O)C(=O)N3CCCC[C@H]3C(=O)O2)CC[C@H]1O]

General condition A1 was applied. Additional 0.6 mL ACN was added as the co-solvent. The reaction was stirred at rt for 6 h to complete. Compound 11 was isolated in 9.5% as a white solid. Mass calculated 1006.58, found 1006.06 (M−H)⁻.

### Compound 12:

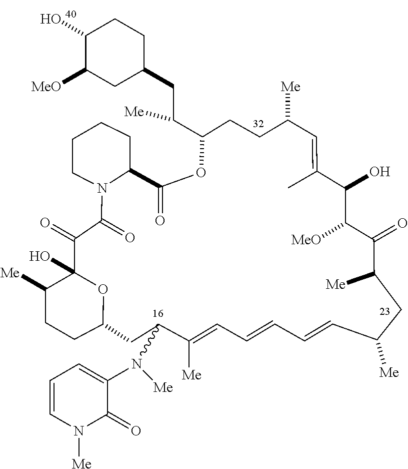 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(N(C)c3cccn(C)c3=O)C[C@@H]3CC[C@@H](C)[C@@](O)(O3)C(=O)C(=O)N3CCCC[C@H]3C(=O)O2)CC[C@H]1O]

General condition A1 was applied. Additional 0.6 mL ACN was added as the co-solvent. The reaction was stirred at rt for 2 h to complete. Compound 12 was isolated in 8.3% as a white solid. Mass calculated 1005.63, found 1006.73 (M+H)⁺.

### Compound 13:

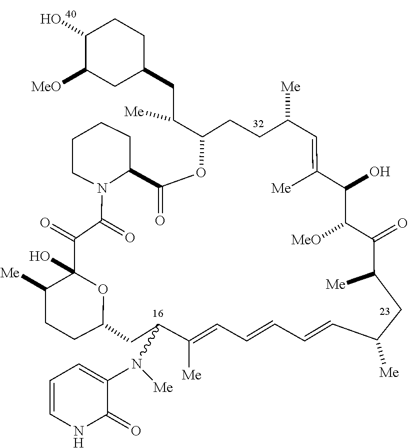 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(N(C)c3ccc[nH]c3=O)C[C@@H]3CC[C@@H](C)[C@@](O)(O3)C(=O)C(=O)N3CCCC[C@H]3C(=O)O2)CC[C@H]1O]

General condition A1 was applied. Additional 0.6 mL ACN was added as the co-solvent. The reaction was stirred at rt for 16 h to complete. Compound 14 was isolated in 22% as a white solid. Mass calculated 991.61, found 991.01 (M−H)⁻.

### Compound 14:

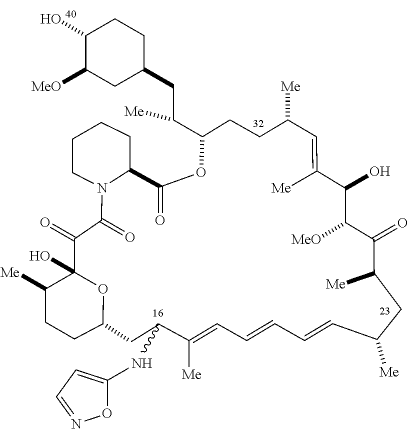 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(Nc3ccno3)C[C@@H]3CC[C@@H](C)[C@@](O)(O3)C(=O)C(=O)N3CCCC[C@H]3C(=O)O2)CC[C@H]1O]

General condition A1 was applied. The procedure was modified with 10 eq. of sulphonamide and 6 eq. of ZnCl₂. The reaction was stirred at rt for 5 d to complete. Compound 14 was isolated in 11% as a yellow solid. Mass calculated 951.58, found 950.89 (M−H)⁻.

### Compound 15:

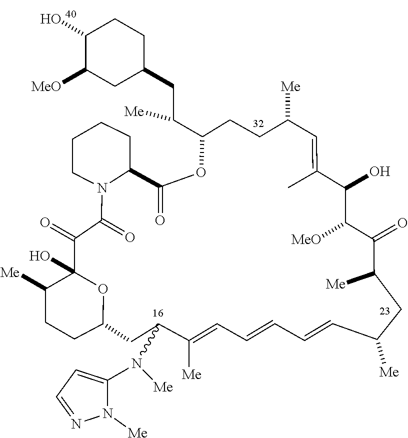 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(N(C)c3ccnn3C)C[C@@H]3CC[C@@H](C)[C@@](O)(O3)C(=O)C(=O)N3CCCC[C@H]3C(=O)O2)CC[C@H]1O]

General condition A1 was applied. The reaction was stirred at rt for 16 h to complete. Compound 15 was isolated in 5.7% as a white solid. Mass calculated 978.63, found 979.92 (M+H)⁺.

The following compounds were prepared via General Condition A2, B2, C2, D2, or E2.

General condition A2: To a solution of 32-deoxorapamycin (30.0 mg, 0.0333 mmol) and the appropriate amine nucleophile (10 eq.) in DCM (0.67 mL) was added ZnCl₂ (10 eq., 1.0 M solution in Et₂O) at rt. The reaction stirred until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient (30-60) to (80-90) % ACN/water (+0.05% TFA as the modifier) to obtain the product.

General condition B2: To a solution of 32-deoxorapamycin (30.0 mg, 0.0333 mmol) and the appropriate amine nucleophile (20 eq.) in DCM (0.67 mL) was added pTSA·H₂O (5 eq.) at rt. The reaction was stirred at rt until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient 60 to 85% ACN/water (+0.05% AcOH as the modifier) to obtain the product.

General condition C2: To a solution of 32-deoxorapamycin (30.0 mg, 0.0333 mmol) and the appropriate amine nucleophile (10 eq.) in THF (0.67 mL) was added BF₃·Et₂O (15 eq.) at rt. The reaction was stirred at rt until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient (30-60) to (80-90) % ACN/water (+0.05% AcOH as the modifier) to obtain the product.

General condition D2: To a solution of 32-deoxorapamycin (60.0 mg, 0.0667 mmol) and the appropriate amine nucleophile (10 eq.) in DCM/ACN (1:1)(2.7 mL) was added Zn(OTf)₂ (6 eq.) at rt. The reaction was stirred at rt until 32-deoxorapamycin was all consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient 50 to 95% ACN/water (+0.05% AcOH as the modifier) to obtain the product.

General condition E2: To a solution of 32-deoxorapamycin (60.0 mg, 0.0667 mmol) and the appropriate amine nucleophile (10 eq.) in DCM (3 mL) at −40° C. was added TFA (20 eq.) and the reaction was allowed to warm to rt. The reaction was stirred at rt until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient 30 to 80% ACN/water (+0.05% AcOH as the modifier) to obtain the product.

### Compound 16:

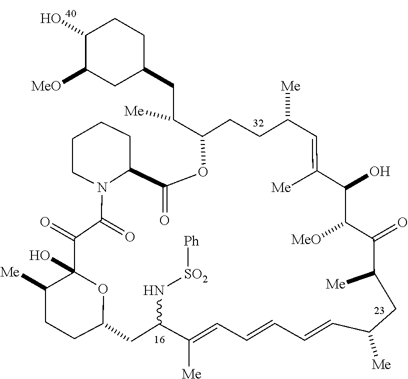 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(NS(=O)(=O)c3ccccc3)C[C@@H]3CC[C@@H](C)[C@@](O)(O3)C(=O)C(=O)N3CCCC[C@H]3C(=O)O2)CC[C@H]1O]

General condition A2 was applied. To a solution of 32-deoxorapamycin (30.0 mg, 0.0333 mmol) and the sulfonamide nucleophile (10 eq.) in DCM (0.67 mL) was added ZnCl₂ (10 eq., 1.0 M solution in Et₂O) at rt. The reaction stirred until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient (30-60) to (80-90) % ACN/water (+0.05% TFA as the modifier) to obtain the product. The reaction was stirred for 1 h to complete. Compound 16 was isolated and repurified by 150×30 Phenomenex Gemini reverse phase column eluted with 40 to 80% to give product as a white solid pure in 15.5% and mixture in 30.2% yield. Mass calculated 1023.56, found 1024.56 (M−H)⁻.

### Compound 17:

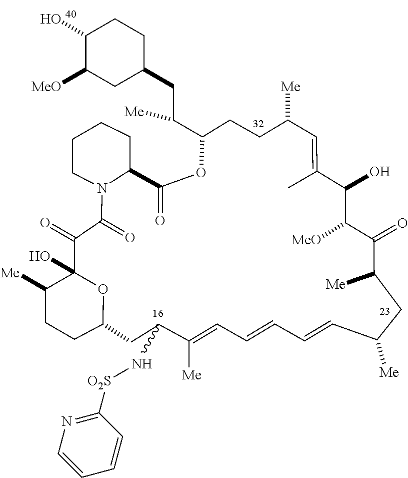 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(NS(=O)(=O)c3ccccn3)C[C@@H]3CC[C@@H](C)[C@@](O)(O3)C(=O)C(=O)N3CCCC[C@H]3C(=O)O2)CC[C@H]1O]

General condition A2 was applied. To a solution of 32-deoxorapamycin (30.0 mg, 0.0333 mmol) and the sulfonamide nucleophile (10 eq.) in DCM (0.67 mL) was added ZnCl₂ (10 eq., 1.0 M solution in Et₂O) at rt. The reaction stirred until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient (30-60) to (80-90) % ACN/water (+0.05% TFA as the modifier) to obtain the product. The reaction was stirred for 4 days to complete. Compound 17 was isolated and repurified by 150×30 Phenomenex Gemini reverse phase column eluted with 35 to 75% to give product as a white solid pure in 1.4% and mixture in 5% yield. Mass calculated 1026.57, found 1027.30 (M+H)⁺.

### Compound 18:

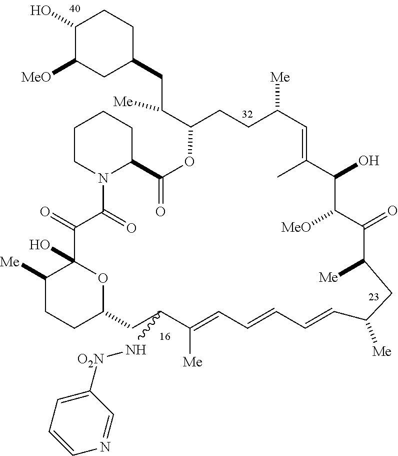

General condition C₂ was applied. To a solution of 32-deoxorapamycin (30.0 mg, 0.0333 mmol) and the sulfonamide nucleophile (10 eq.) in THF (0.67 mL) was added BF₃·Et₂O (15 eq.) at rt. The reaction was stirred at rt until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient (30-60) to (80-90) % ACN/water (+0.05% AcOH as the modifier) to obtain the product. The reaction was stirred for 5 hours to complete. Compound 18 was isolated and repurified by 150×30 Phenomenex Gemini reverse phase column eluted with 30 to 80% to give product as a light-yellow solid in 12.4% yield. Mass calculated 1024.56, found 1025.13 (M−H)⁻.

### Compound 19:

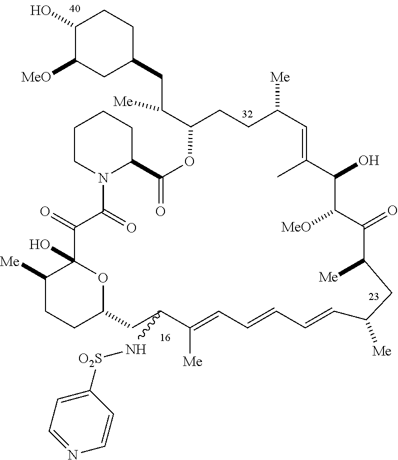

General condition C₂ was applied. To a solution of 32-deoxorapamycin (30.0 mg, 0.0333 mmol) and the sulfonamide nucleophile (10 eq.) in THF (0.67 mL) was added BF₃·Et₂O (15 eq.) at rt. The reaction was stirred at rt until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient (30-60) to (80-90) % ACN/water (+0.05% AcOH as the modifier) to obtain the product. The reaction was stirred for 5 hours to complete. Compound 19 was isolated and repurified by 150×30 Phenomenex Gemini reverse phase column eluted with 30 to 80% to give product as a light-yellow solid in 7% yield. Mass calculated 1024.56, found 1026.46 (M−H)⁻.

### Compound 20:

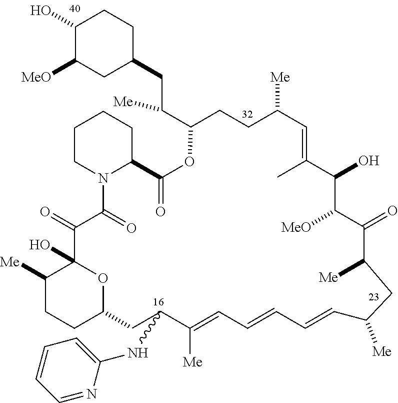

General condition A was applied. To the reaction was added ZnCl₂ (15 eq) and ACN (0.67 mL) and the reaction was stirred for 3 days to complete. Compound 20 was isolated and repurified by 150×30 Phenomenex Gemini reverse phase column eluted with 40 to 80% to give product as a yellow solid in 7.3% yield. Mass calculated 962.61, found 963.31 (M+H)⁺. Compound 21:

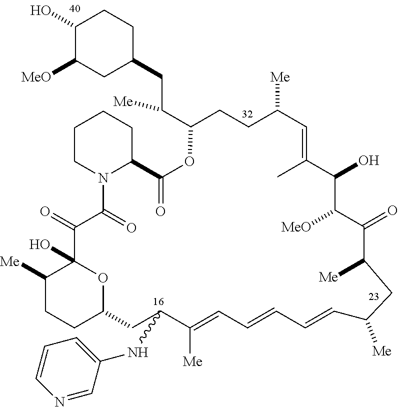

General condition C₂ was applied. To a solution of 32-deoxorapamycin (30.0 mg, 0.0333 mmol) and the amine nucleophile (10 eq.) in THF (0.67 mL) was added BF₃·Et₂O (15 eq.) at rt. The reaction was stirred at rt until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient (30-60) to (80-90) % ACN/water (+0.05% AcOH as the modifier) to obtain the product. The reaction was stirred for 3 hours to complete. Compound 21 was isolated and repurified by 150×30 Phenomenex Gemini reverse phase column eluted with 30 to 80% to give product as a white solid in 9% yield and mixture in 10.2% yield. Mass calculated 962.61, found 963.31 (M+H)⁺.

### Compound 22:

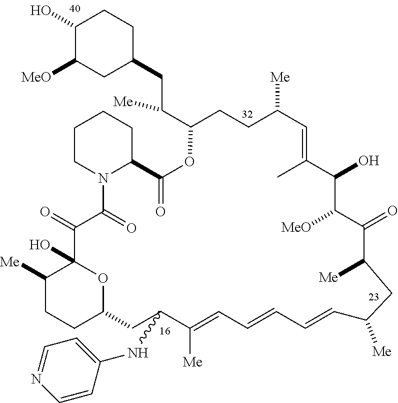

General condition C₂ was applied. To a solution of 32-deoxorapamycin (30.0 mg, 0.0333 mmol) and corresponding amine nucleophile (10 eq.) in THF (0.67 mL) was added BF₃·Et₂O (15 eq.) at rt. The reaction was stirred at rt until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient (30-60) to (80-90) % ACN/water (+0.05% AcOH as the modifier) to obtain the product. The reaction was stirred for 3 hours to complete. Compound 22 was isolated and repurified by 150×30 Phenomenex Gemini reverse phase column eluted with 30 to 80% to give product as a white solid in 1.54% yield and mixture in 2.89% yield. Mass calculated 962.61, found 963.28 (M+H)⁺.

### Compound 23:

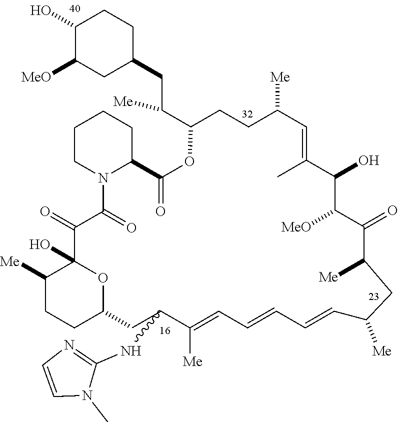 [0498] General condition E2 was applied. To a solution of 32-deoxorapamycin (60.0 mg, 0.0667 mmol) and the amine nucleophile (10 eq.) in DCM (3 mL) at −40° C. was added TFA (20 eq.) allowed to rt. The reaction was stirred at rt until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient 30 to 80% ACN/water (+0.05% AcOH as the modifier) to obtain the product. The reaction was stirred for 19 hours to complete. Compound 23 was isolated and repurified by 150×30 Phenomenex Gemini reverse phase column eluted with 30 to 80% to give product as a tan solid in 7.6% yield and mixture in 5.6% yield. Mass calculated 965.62, found 966.35 (M+H)⁺.

### Compound 24:

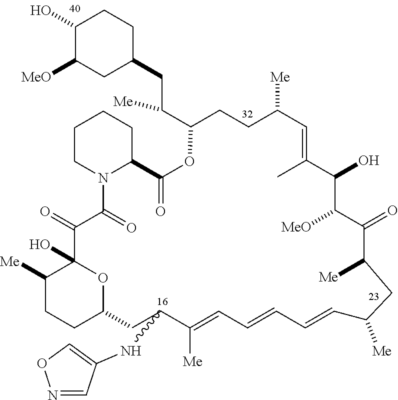

General condition E2 was applied. To a solution of 32-deoxorapamycin (60.0 mg, 0.0667 mmol) and the amine nucleophile (10 eq.) in DCM (3 mL) at −40° C. was added TFA (20 eq.) allowed to rt. The reaction was stirred at rt until 32-deoxorapamycin was consumed. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient 30 to 80% ACN/water (+0.05% AcOH as the modifier) to obtain the product. The reaction was stirred for 2 hours to complete. Compound 24 was isolated and repurified by 150×30 Phenomenex Gemini reverse phase column eluted with 30 to 80% to give product as a tan solid in 14.9% yield and mixture in 21.6% yield. Mass calculated 950.57, found 951.08 (M−H)⁻.

Compound 26 (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-(phenylamino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone;

### Aniline-32-Deoxorapamycin:

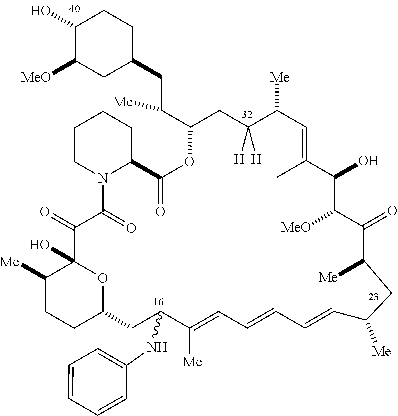

To a stirred solution of 32-deoxorapamycin (80 mg, 0.088 mmol) in DCM (2 mL) was added aniline (0.080 mL, 0.888 mmol) and TFA (0.136 mL, 1.776 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 7.3 mg (9%) of the white solid Compound 26. MS (ESI) calc'd for C₅₆H₈₄N₂O₁₁+H=960.61, found 960.08.

Compound 27 (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-((5-methylisoxazol-3-yl)amino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone; 16-(3-Amino-5-Methylisoxazole)-32-Deoxorapamycin:

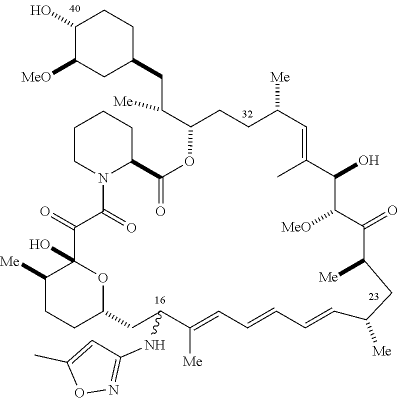

To a stirred solution of 32-deoxorapamycin (80 mg, 0.088 mmol) in DCM (2 mL) was added 3-amino-5-methylisoxazole (87 mg, 0.888 mmol) and TFA (0.136 mL, 1.779 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 5.8 mg (7%) of the white solid Compound 27. MS (ESI) calc'd for C₅₄H₈₃N₃O₁₂+H=965.61, found 965.10.

Compound 28 (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-((6-methylpyrazin-2-yl)amino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone; 16-(2-Amino-6-Methylpyrazine)-32-Deoxorapamycin:

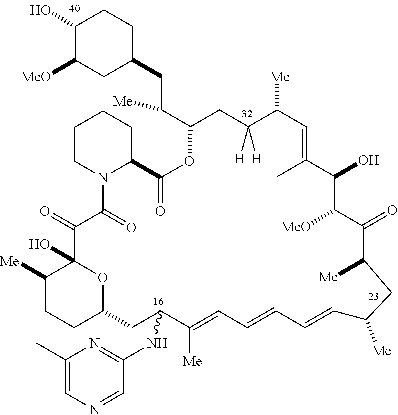

To a stirred solution of 32-deoxorapamycin (50 mg, 0.055 mmol) in DCM (2 mL) was added 2-amino-6-methylpyrazine (61 mg, 0.555 mmol) and TFA (0.085 mL, 1.112 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 7.2 mg (13%) of the white solid Compound 28. MS (ESI) calc'd for C₅₅H₈₄N₄O₁₁+H=976.61, found 976.31.

Compound 29, (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-((1-methyl-1H-pyrazol-5-yl)amino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone

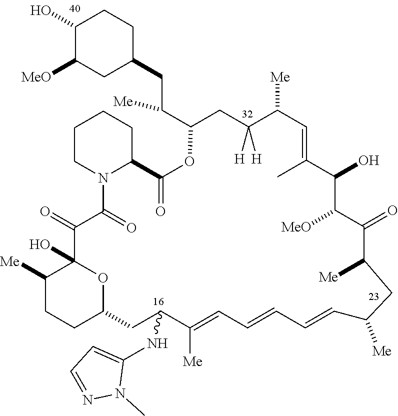

To a stirred solution of 32-deoxorapamycin (60 mg, 0.066 mmol) in DCM (2 mL) was added 1,2,5-oxadiazol-3-amine (66 mg, 0.666 mmol) and TFA (0.102 mL, 1.322 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 1.3 mg (2%) of the white solid. MS (ESI-) calc'd for C₅₂H₈₀N₄O₁₂—H=964.61, found 964.11.

Compound 30 (3S,6S,7E,9R,10R,12R,14S,15E,17E,19E,23S,26R,27R,34aS)-21-((1,2,4-oxadiazol-3-yl)amino)-9,27-dihydroxy-3-((R)-1-((1S,3R,4R)-4-hydroxy-3-methoxycyclohexyl) propan-2-yl)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone; (16-(1,2,4-Oxadiazol-3-Amine)-32-Deoxorapamcyin:

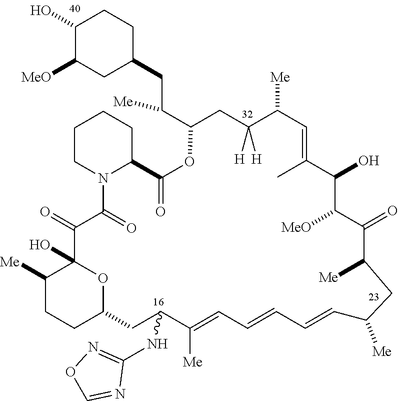

To a stirred solution of 32-deoxorapamycin (50 mg, 0.055 mmol) in DCM (2 mL) was added 1,2,4-oxadiazol-3-amine (55 mg, 0.555 mmol) and TFA (0.085 mL, 1.112 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 5.3 mg (10%) of the white solid Compound 30. MS (ESI) calc'd for C₅₂H₈₀N₄O₁₂+H=952.58, found 952.17.

### Additional Compounds are Synthesized as Described Below.

### Compound 31:

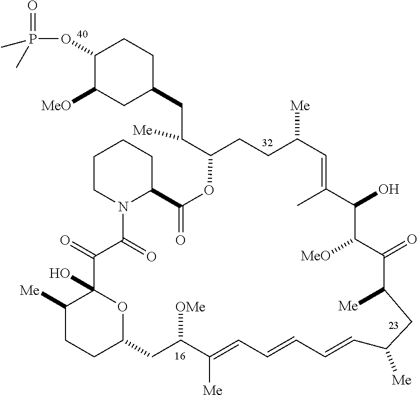

To a solution of 32-deoxorapamycin (20.0 mg, 0.0222 mmol) and 2,6-di-tert-butyl-4-methylpyridine (36.5 mg, 0.178 mmol) in DCM (0.22 mL) was added dimethylphosphinic chloride (12.5 mg, 0.111 mmol) at 0° C. and stirred for 2 h. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DCM and subjected to ISCO silica column and eluted with gradient 0 to 100% acetone/hexane to obtain 65% of Compound 31 as a white solid. Mass calculated 975.58, found 974.69 (M−H)⁻.

### Compound 32:

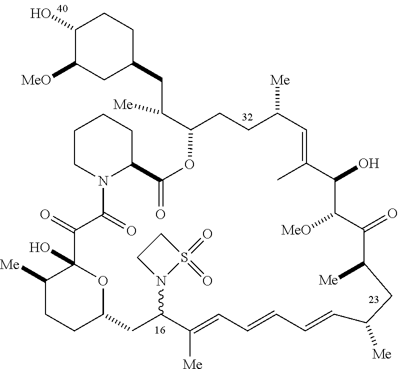

General condition A1 was applied. The reaction was stirred for 2 h to complete.

Compound 32 was isolated in 21% as a white solid. Mass calculated 974.55, found 973.25 (M−H)⁻.

### Compound 33:

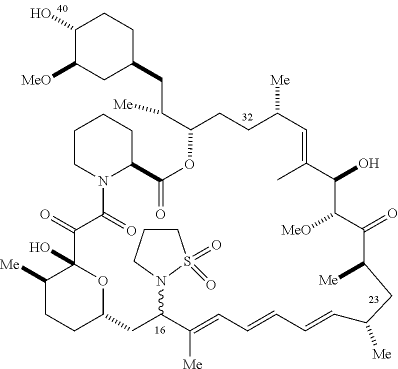

General condition B1 was applied. The reaction was stirred for 0.5 h to complete. Compound 33 was isolated in 53% as a white solid. Mass calculated 988.57, found 987.19 (M−H)⁻.

### Compound 34:

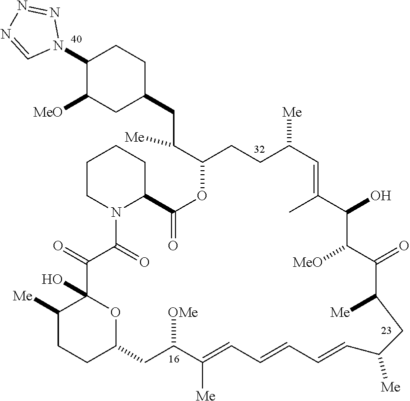

To a solution of 32-deoxorapamycin (20.0 mg, 0.0222 mmol) and 2,6-lutidine (6.4 μL, 0.0556 mmol) in DCM (0.28 mL) was added Tf₂O (6.0 μL, 0.0333 mmol) at −30° C. and stirred for 30 min. The reaction was warmed up to 0° C. and stirred for additional 30 min. Then, 1-H-tetrazole (5.4 mg, 0.0778 mmol) and DIPEA (0.019 mL, 0.111 mmol) were added into the solution and allowed to stir at rt for 16 hr. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DFM and subjected to ISCO C18 RP column and eluted with gradient 50 to 100% ACN/water (+0.5% AcOH as modifier) to obtain 69% of Compound 34 as a yellow solid. Mass calculated 951.59, found 951.26 (M−H)⁻.

### Compound 35:

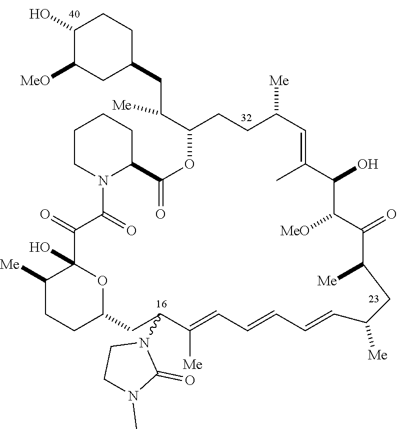

General condition B1 was applied. The reaction was stirred for 2 d to complete. Compound 35 was isolated in 39% as a white solid. Mass calculated 967.61, found 966.88 (M−H)⁻.

### Compound 36:

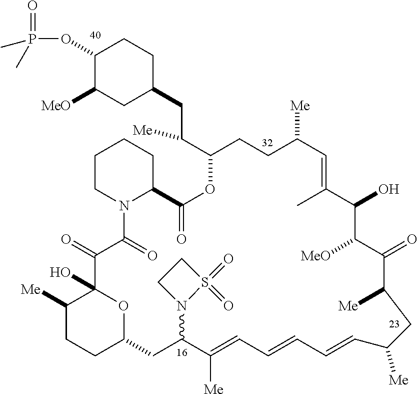 [0523] To a solution of 40-phosphinate-32-deoxorapamycin (19.0 mg, 0.0195 mmol) and ethane sultam (10.4 mg, 0.0974 mmol) in DCM (0.39 mL) was added ZnCl₂ (0.0584 mL, 0.0584 mmol, 1.0 M solution in Et₂O) at rt. The reaction was stirred at rt for 30 min. The resulting mixture was diluted with EtOAc and quenched with sat. aq. NaHCO₃. The organic phase was separated and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient 50 to 90% ACN/water (+0.5% AcOH as modifier) to obtain Compound 36 in 29% as a white solid. Mass calculated 1050.56, found 1049.67 (M−H)⁻.

### Compound 37:

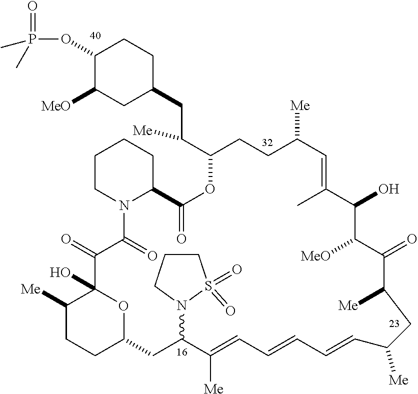

To a solution of 40-dimethylphosphinate-32-deoxorapamycin (Compound 31, 20.0 mg, 0.0205 mmol) and propyl sultam (24.8 mg, 0.205 mmol) in ACN (0.21 mL) was added pTSA·H₂O (0.4 mg, 0.0021 mmol) at rt. The reaction was stirred at rt for 2 h. The resulting mixture was diluted with EtOAc and quenched with sat. aq. NaHCO₃. The organic phase was separated, and the aq. phase was extracted with EtOAc three times. The combined organic phase was dried with Na₂SO₄, filtered, and concentrated in vacuo. The residue was dissolved in DMF and subjected to ISCO C18 RP column and eluted with gradient 50 to 90% ACN/water (+0.5% AcOH as modifier) to obtain Compound 37 in 49% as a white solid. Mass calculated 1064.58, found 1063.76 (M−H)⁻.

### Compound 38:

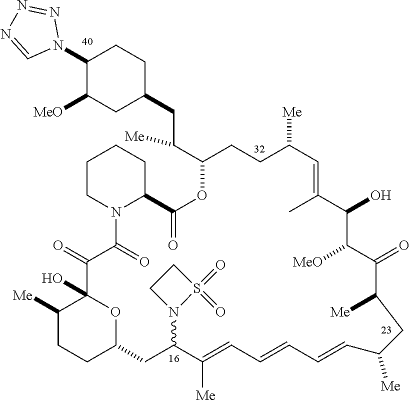

General condition A1 was applied. The reaction was stirred for 2 h to complete. Compound 38 was isolated in 20% as a white solid. Mass calculated 1026.57, found 1025.51 (M−H)⁻.

### Compound 39:

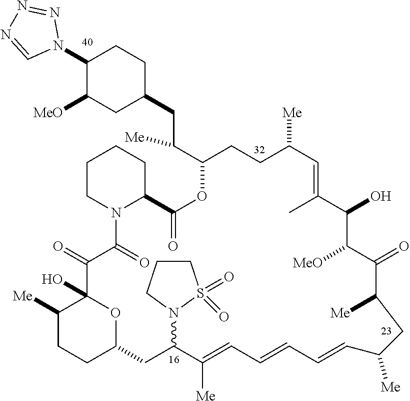

General condition B1 was applied. ACN was used as the solvent instead of DCM. The reaction was stirred for 1 h to complete. Compound 39 was isolated in 32% as a white solid. Mass calculated 1040.59, found 1063.85 (M+Na)+.

### Compound 40:

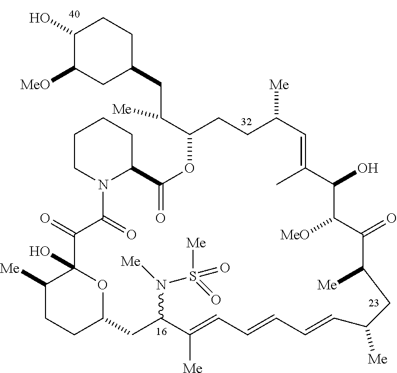

General condition A1 was applied. The reaction was stirred at rt for 1 h to complete. Compound 40 was isolated in 19% as a white solid. Mass calculated 976.57, found 975.70 (M−H)⁻.

### Compound 41:

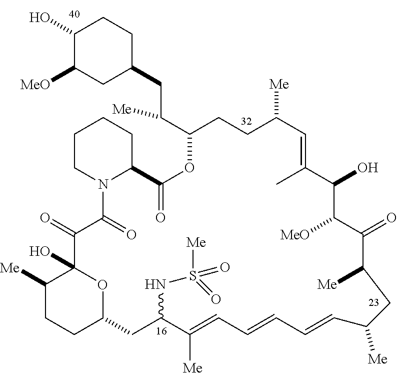

General condition A1 was applied. The procedure was modified with 10 eq. of sulfonamide and 8 eq. of ZnCl₂. Additional 2.0 mL of ACN was added as the co-solvent. The reaction was stirred at 39° C. for 2 d to complete. Compound 41 was isolated in 4.9% as a white solid. Mass calculated 962.55, found 962.26 (M−H)⁻.

### Compound 42:

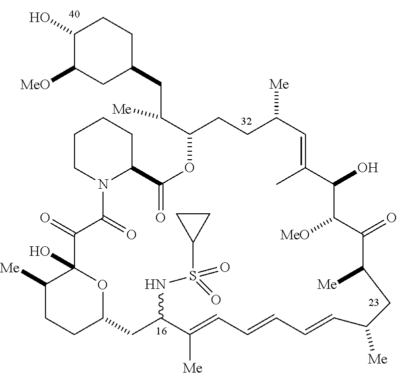

General condition A1 was applied. The procedure was modified with 20 eq. of sulphonamide and 15 eq. of ZnCl₂. THF was used as the solvent instead of DCM. The reaction was stirred at 39° C. for 5 d to complete. Compound 42 was isolated in 5.5% as a white solid. Mass calculated 988.57, found 987.94 (M−H)⁻.

Compound 43 (3S,6S,7E,9R,10R,12R,14S,15E,17E,19E,23S,26R,27R,34aS)-21-((3-chlorophenyl)amino)-9,27-dihydroxy-3-((R)-1-((1S,3R,4R)-4-hydroxy-3-methoxycyclohexyl) propan-2-yl)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone

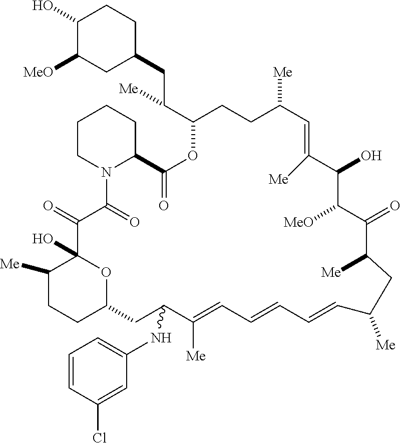

To a stirred solution of 32-deoxorapamycin (50 mg, 0.055 mmol) in DCM (2 mL) was added 3-chloroaniline (0.049 mL, 0.555 mmol) and TFA (0.085 mL, 1.112 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 8.1 mg (15%) of the white solid Compound 43. MS (ESI-) calc'd for C₅₆H₈₃ClN₂O₁₁—H=994.57, found 994.05.

Compound 44 (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-21-((3-methoxyphenyl)amino)-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone

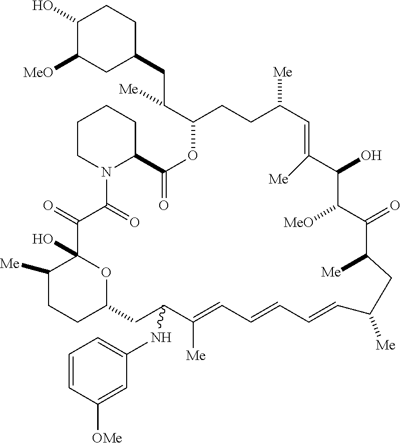

To a stirred solution of 32-deoxorapamycin (50 mg, 0.055 mmol) in DCM (2 mL) was added meta-anisidine (0.062 mL, 0.555 mmol) and TFA (0.085 mL, 1.112 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 3.0 mg (5%) of the white solid Compound 44. MS (ESI-) calc'd for C₅₇H₈₆N₂O₁₂—H=990.62, found 990.14.

Compound 45 (3S,6S,7E,9R,10R,12R,14S,15E,17E,19E,23S,26R,27R,34aS)-21-((1,2,5-oxadiazol-3-yl)amino)-9,27-dihydroxy-3-((R)-1-((1S,3R,4R)-4-hydroxy-3-methoxycyclohexyl) propan-2-yl)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone.

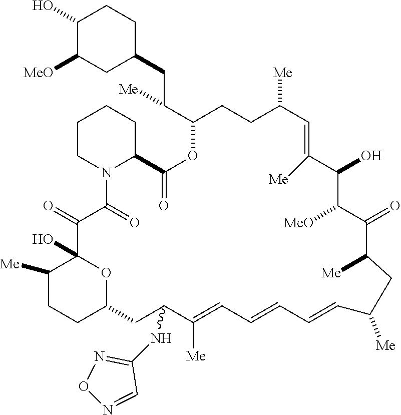

To a stirred solution of 32-deoxorapamycin (30 mg, 0.033 mmol) in DCM (2 mL) was added 1,2,5-oxadiazol-3-amine (30 mg, 0.333 mmol) and TFA (0.051 mL, 3.32 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 9.5 mg (30%) of the white solid Compound 45. MS (ESI-) calc'd for C₅₇H₈₆N₂O₁₂—H=952.58, found 952.03.

Compound 46. N-((3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-21-yl)-3,5-dimethylisoxazole-4-sulfonamide.

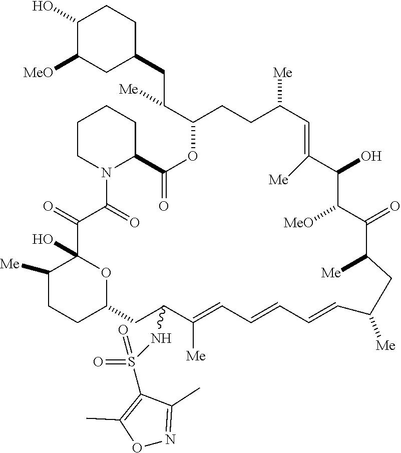

To a stirred solution of 32-deoxorapamycin (80 mg, 0.089 mmol) in DCM (2 mL) was added dimethyl-1,2-oxazole-4-sulfonamide (157 mg, 0.899 mmol) and ZnCl₂ (0.889 mL, 0.889 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 8.3 mg (9%) of the white solid Compound 46. MS (ESI-) calc'd for C₅₅H₈₅N₃O₁₄S—H=1043.58, found 1042.87.

Compound 47 (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-21-((4-methoxyphenyl)amino)-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone.

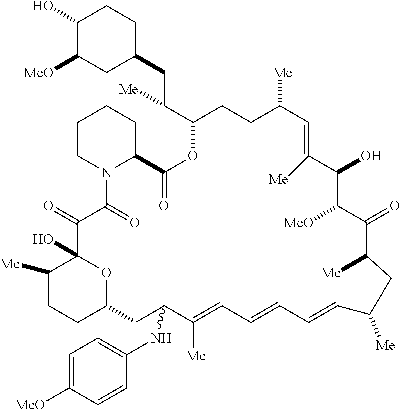

To a stirred solution of 32-deoxorapamycin (50 mg, 0.055 mmol) in DCM (2 mL) was added para-anisidine (0.064 mL, 0.556 mmol) and TFA (0.085 mL, 1.112 mmol) at −20° C. and stirred for 4 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 2.0 mg (4%) of the white solid. MS (ESI-) calc'd for C₅₇H₈₆N₂O₁₂—H=991.32, found 990.67.

Compound 48. (3S,6S,7E,9R,10R,12R,14S,15E,17E,19E,23S,26R,27R,34aS)-21-((4-chlorophenyl)amino)-9,27-dihydroxy-3-((R)-1-((1S,3R,4R)-4-hydroxy-3-methoxycyclohexyl) propan-2-yl)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone.

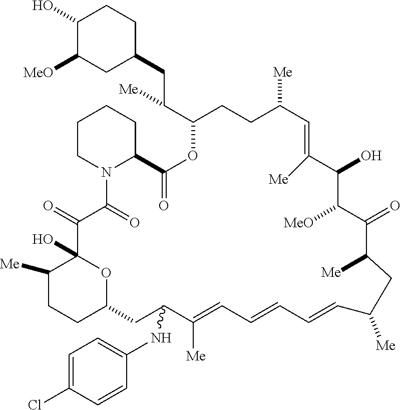

To a stirred solution of 32-deoxorapamycin (50 mg, 0.055 mmol) in DCM (2 mL) was added 4-chloroaniline (0.050 mL, 0.556 mmol) and TFA (0.085 mL, 1.112 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 5.2 mg (9%) of the white solid. MS (ESI-) calc'd for C₅₆H₈₃ClN₂O₁₁—H=994.57, found 993.86.

Compound 49 (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-(p-tolylamino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone.

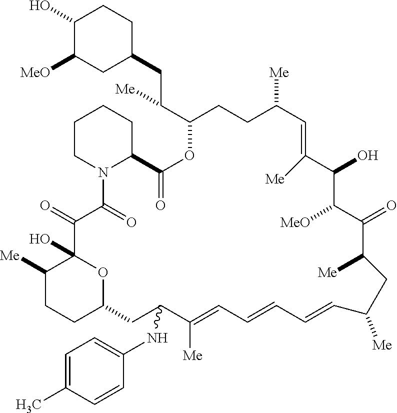

To a stirred solution of 32-deoxorapamycin (50 mg, 0.055 mmol) in DCM (2 mL) was added para-toluidine (0.060 mL, 0.556 mmol) and TFA (0.085 mL, 1.112 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 1.6 mg (3%) of the white solid. MS (ESI-) calc'd for C₅₇H₈₆N₂O₁₁—H=974.62, found 973.76.

Compound 50 (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-(m-tolylamino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone.

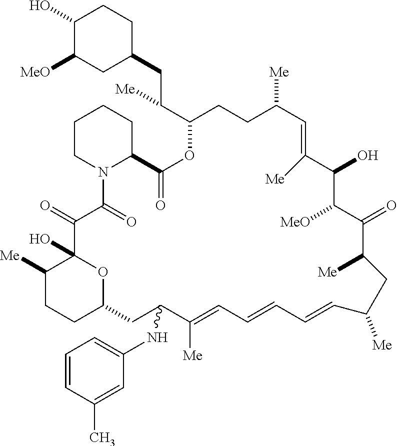

To a stirred solution of 32-deoxorapamycin (50 mg, 0.055 mmol) in DCM (2 mL) was added meta-toluidine (0.060 mL, 0.556 mmol) and TFA (0.085 mL, 1.112 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 2.5 mg (5%) of the white solid. MS (ESI-) calc'd for C₅₇H₈₆N₂O₁₁—H=974.62, found 973.95.

### Compound 51

N-((3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-21-yl)-5-methylisoxazole-4-sulfonamide.

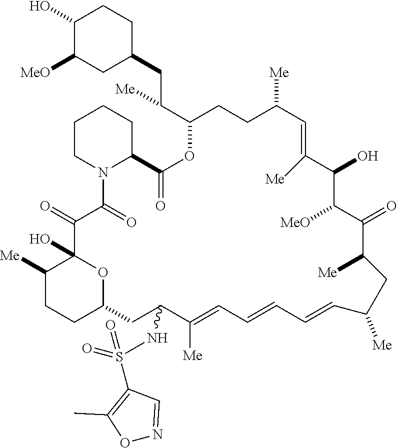

To a stirred solution of 32-deoxorapamycin (80 mg, 0.089 mmol) in DCM (2 mL) was added 5-methyl-1,2-oxazole-4-sulfonamide (144 mg, 0.889 mmol) and ZnCl₂ (0.889 mL, 0.889 mmol) at rt and stirred for 3 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 4.1 mg (9%) of the white solid. MS (ESI-) calc'd for C₅₄H₈₃N₃O₁₄S—H=1030.33, found 1029.16.

Compound 52 (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-(p-tolylamino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone.

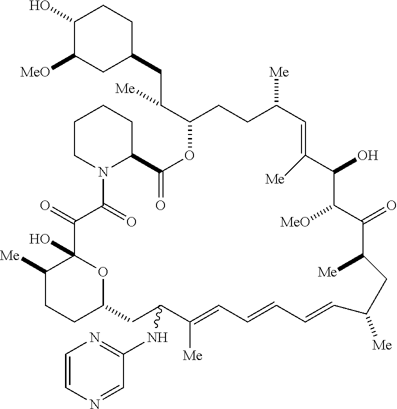

To a stirred solution of 32-deoxorapamycin (50 mg, 0.055 mmol) in DCM (2 mL) was added aminopyrazine (53 mg, 0.556 mmol) and TFA (0.085 mL, 1.112 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 4.2 mg (8%) of the white solid. MS (ESI-) calc'd for C₅₄H₈₂N₄O₁₁—H=962.60, found 961.91.

### Compound 53

N-((3S,6S,7E,9R,10R,12R,14S,15E, 17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-21-yl)-3-methylisoxazole-4-sulfonamide.

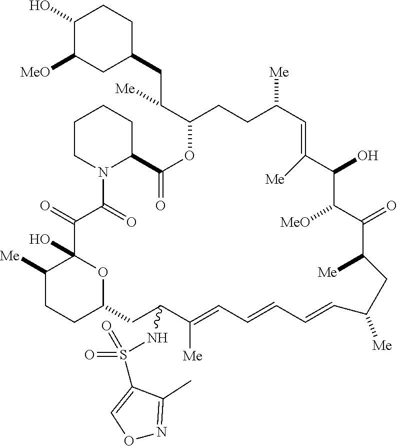

To a stirred solution of 32-deoxorapamycin (80 mg, 0.089 mmol) in DCM (2 mL) was added 3-methyl-1,2-oxazole-4-sulfonamide (144 mg, 0.889 mmol) and ZnCl₂ (0.889 mL, 0.889 mmol) at rt and stirred for 3 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 1.7 mg (2%) of the white solid. MS (ESI-) calc'd for C₅₄H₈₃N₃O₁₄S—H=1030.33, found 1029.27.

Compound 54 (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-((3-(trifluoromethyl)phenyl)amino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone.

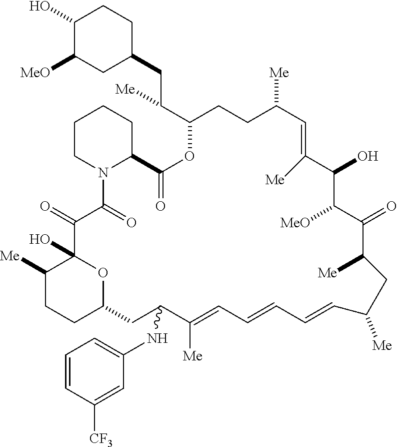

To a stirred solution of 32-deoxorapamycin (50 mg, 0.055 mmol) in DCM (2 mL) was added 3-trifluoromethylaniline (0.065 mL, 0.556 mmol) and TFA (0.085 mL, 1.112 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 6.5 mg (11%) of the white solid. MS (ESI-) calc'd for C₅₇H₈₃F₃N₂O₁₁—H=1028.57, found 1027.90.

Compound 55 (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-((4-(trifluoromethyl)phenyl)amino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone.

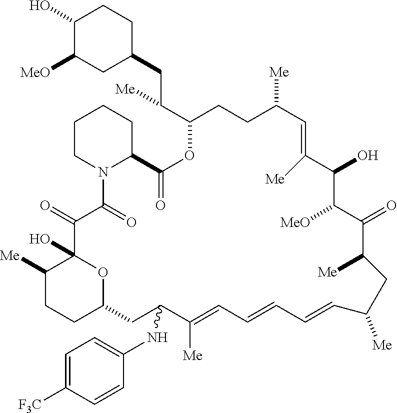

To a stirred solution of 32-deoxorapamycin (50 mg, 0.055 mmol) in DCM (2 mL) was added 4-trifluoromethylaniline (0.065 mL, 0.556 mmol) and TFA (0.085 mL, 1.112 mmol) at −20° C. and stirred for 2 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 2.3 mg (4%) of the white solid. MS (ESI-) calc'd for C₅₇H₈₃F₃N₂O₁₁—H=1028.57, found 1028.05.

Compound 56 (4-(((3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-21-yl)amino)benzonitrile)

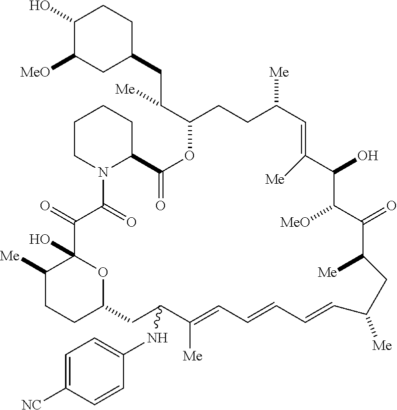

To a stirred solution of 32-deoxorapamycin (50 mg, 0.055 mmol) in DCM (8 mL) was added 4-aminobenzonitrile (0.065 mL, 0.556 mmol) and TFA (0.085 mL, 1.112 mmol) at −20° C. and the reaction was stirred for 3 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 2.4 mg (4%) of the white solid. MS (ESI-) calc'd for C₅₇H₈₃N₃O₁₁—H=985.60, found 985.08.

Compound 57 (3-(((3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-21-yl)amino)benzonitrile

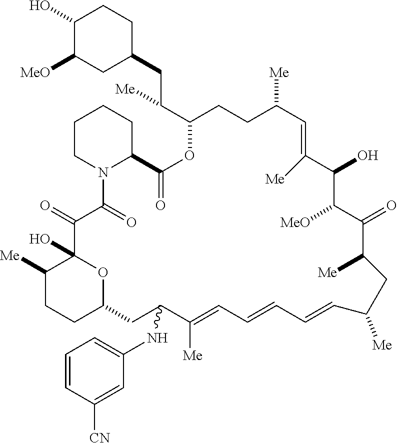

To a stirred solution of 32-deoxorapamycin (75 mg, 0.083 mmol) in DCM (10 mL) was added 3-aminobenzonitrile (0.065 mL, 0.556 mmol) and TFA (0.085 mL, 1.112 mmol) at −20° C. and stirred for 3 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 3.1 mg (6%) of the white solid. MS (ESI-) calc'd for C₅₇H₈₃N₃O₁₁—H=984.60, found 985.01.

Compound 58 ((3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-((3-(trifluoromethoxy)phenyl)amino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone)

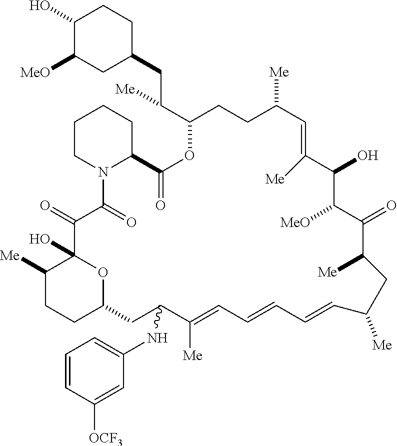

To a stirred solution of 32-deoxorapamycin (50 mg, 0.055 mmol) in DCM (10 mL) was added 3-trifluoromethoxyaniline (0.076 mL, 0.834 mmol) and TFA (0.128 mL, 1.667 mmol) at −20° C. and stirred for 3 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 10.9 mg (13%) of the white solid. MS (ESI-) calc'd for C₅₇H₈₃F₃N₂O₁₂—H=1043.59, found 1044.09.

Compound 59 ((3S,6S,7E,9R,10R,12R,14S,15E,17E,19E,23S,26R,27R,34aS)-21-((3-ethoxyphenyl)amino)-9,27-dihydroxy-3-((R)-1-((1S,3R,4R)-4-hydroxy-3-methoxycyclohexyl) propan-2-yl)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone)

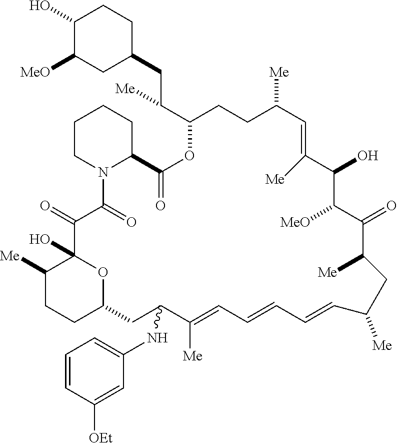

To a stirred solution of 32-deoxorapamycin (75 mg, 0.083 mmol) in DCM (10 mL) was added 3-ethoxyaniline (0.095 mL, 0.834 mmol) and TFA (0.128 mL, 1.667 mmol) at −20° C. and stirred for 3 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 6.5 mg (8%) of the white solid. MS (ESI-) calc'd for C₅₈H₈₈N₂O₁₂—H=1003.63, found 1003.97.

Compound 60 ((3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-(thiophen-3-ylamino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone)

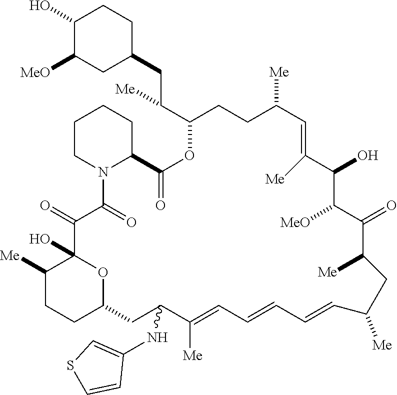

To a stirred solution of 32-deoxorapamycin (75 mg, 0.083 mmol) in DCM (10 mL) was added thiophene-3-amine hydrochloride (113 mg, 0.834 mmol) and TFA (0.128 mL, 1.667 mmol) at −20° C. and stirred for 3 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 2.0 mg (2%) of the white solid. MS (ESI-) calc'd for C₅₄H₈₂N₂O₁₁S—H=965.56, found 965.86.

Compound 61 (N-((3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-21-yl) isoxazole-4-sulfonamide)

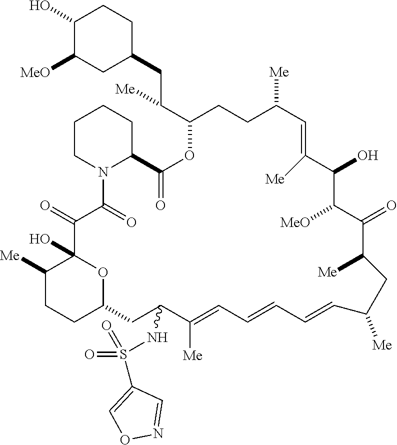

To a stirred solution of 32-deoxorapamycin (75 mg, 0.083 mmol) in DCM (10 mL) was added 1,2-oxazole-4-sulfonamide (124 mg, 0.834 mmol) and ZnCl₂ (0.834 mL, 0.834 mmol) at rt and stirred for 16 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 1.0 mg (1%) of the white solid. MS (ESI-) calc'd for C₅₃H₈₁N₃O₁₄S—H=1015.54, found 1015.14.

Compound 62 ((3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-21-((3-methoxyphenyl)(methyl)amino)-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone)

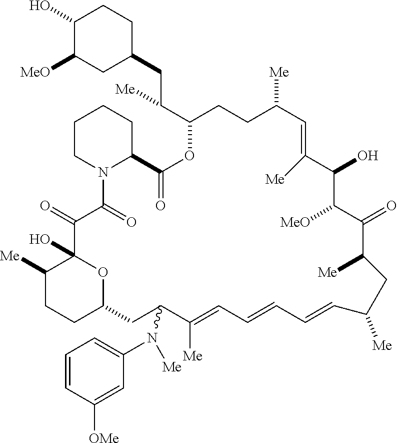

To a stirred solution of 32-deoxorapamycin (75 mg, 0.083 mmol) in DCM (10 mL) was added 3-methoxy-N-methylaniline (0.094 mL, 0.834 mmol) and TFA (0.128 mL, 1.667 mmol) at −20° C. and stirred for 6 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 5.8 mg (7%) of the white solid. MS (ESI-) calc'd for C₅₈H₈₈N₂O₁₂—H=1004.63, found 1004.39.

Compound 63 ((3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-(methyl(phenyl)amino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone)

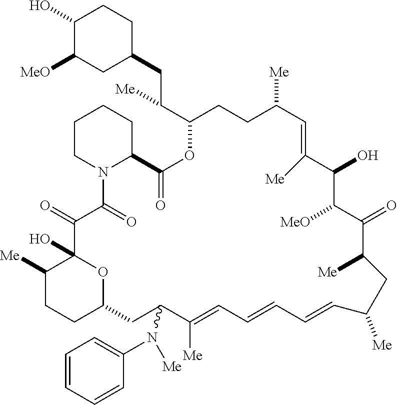

To a stirred solution of 32-deoxorapamycin (100 mg, 0.111 mmol) in DCM (10 mL) was added N-methylaniline (0.120 mL, 1.112 mmol) and TFA (0.170 mL, 2.223 mmol) at −20° C. and stirred for 6 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 2.6 mg (3%) of the white solid. MS (ESI-) calc'd for C₅₇H₈₆N₂O₁₁—H=974.62, found 974.07.

Compound 64 (3S,6S,7E,9R,10R,12R,14S,15E,17E,19E,21S,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-21-(methyl(phenyl)amino)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone:

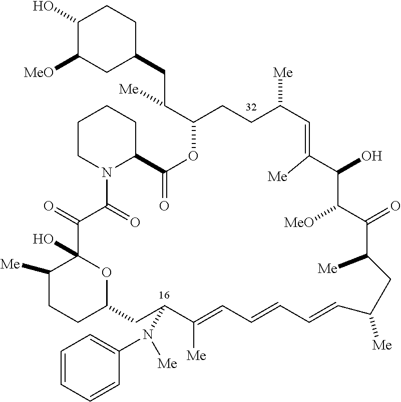

To a stirred solution of 32-deoxorapamycin (200 mg, 0.222 mmol) in DCM (10 mL) was added N-methylaniline (0.240 mL, 2.223 mmol) and TFA (0.340 mL, 4.445 mmol) at −20° C. and stirred for 16 h to rt. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 4.1 mg (2%) of the white solid. MS (ESI-) calc'd for C₅₇H₈₆N₂O₁₁—H=974.62, found 974.33.

Compound 65 (3S,6S,7E,9R,10R,12R,14S,15E,17E,19E,23S,26R,27R,34aS)-21-(3,4-dihydroquinolin-1 (2H)-yl)-9,27-dihydroxy-3-((R)-1-((1S,3R,4R)-4-hydroxy-3-methoxycyclohexyl) propan-2-yl)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone:

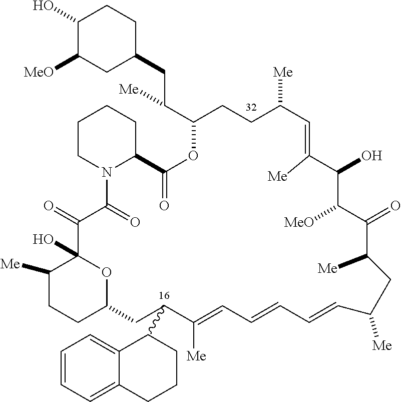

To a stirred solution of 32-deoxorapamycin (200 mg, 0.222 mmol) in DCM (10 mL) was added 1,2,3,4-tetrahydroquinoline (0.278 mL, 2.223 mmol) and TFA (0.340 mL, 4.445 mmol) at −20° C. and stirred for 16 h to rt. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 4.8 mg (2%) of the white solid. MS (ESI-) calc'd for C₅₇H₈₆N₂O₁₁—H=1000.64, found 1000.17.

Compound 66 (3S,6S,7E,9R,10R,12R,14S,15E,17E,19E,21R,23S,26R,27R,34S)-21-(1,1-dioxidoisothiazolidin-2-yl)-9,27-dihydroxy-3-((R)-1-((1S,3R,4R)-4-hydroxy-3-methoxycyclohexyl) propan-2-yl)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone:

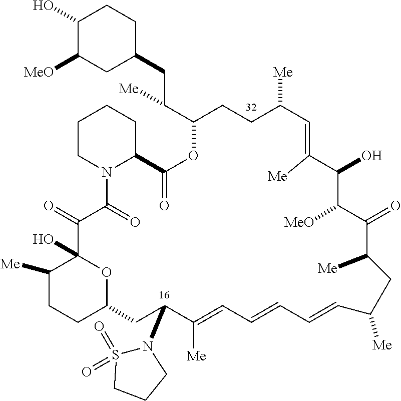

To a stirred solution of 32-deoxorapamycin (100 mg, 0.111 mmol) in DCM (10 mL) was added 1,3-propanesultam (0.136 mL, 1.112 mmol) and ZnCl₂ (1.112 mL, 1.112 mmol) at rt and stirred for 16 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 14.4 mg (13%) of the white solid. MS (ESI-) calc'd for C₅₃H₈₄N₂O₁₃S—H=988.57, found 988.16.

Compound 67: (3S,6S,7E,9R,10R,12R,14S,15E,17E,19E,21R,23S,26R,27R,34S)-21-(1,1-dioxidoisothiazolidin-2-yl)-9,27-dihydroxy-3-((R)-1-((1S,3R,4R)-4-hydroxy-3-methoxycyclohexyl) propan-2-yl)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone

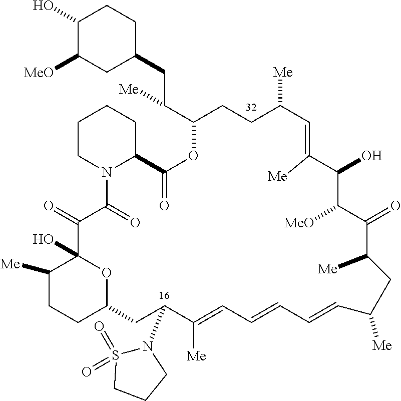

To a stirred solution of 32-deoxorapamycin (100 mg, 0.111 mmol) in DCM (10 mL) was added 1,3-propanesultam (0.136 mL, 1.112 mmol) and ZnCl₂ (1.112 mL, 1.112 mmol) at rt and stirred for 16 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 8.5 mg (8%) of the white solid. MS (ESI-) calc'd for C₅₃H₈₄N₂O₁₃S—H=988.57, found 988.01.

Compound 68: (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-21-((2-hydroxyethyl)(phenyl)amino)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone:

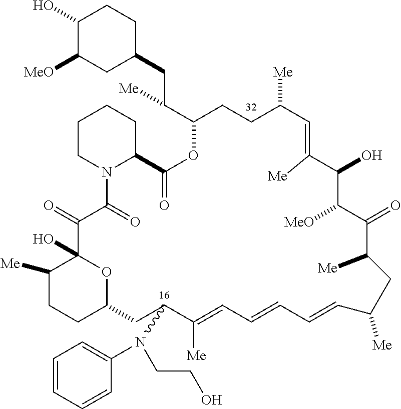

To a stirred solution of 32-deoxorapamycin (75 mg, 0.083 mmol) in DCM (10 mL) was added 2-(phenylamino) ethanol (0.122 mL, 0.834 mmol) and TFA (0.128 mL, 1.667 mmol) at −20° C. and stirred for 16 h to rt. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 15.5 mg (19%) of the white solid. MS (ESI-) calc'd for C₅₈H₈₈N₂O₁₂—H=1004.63, found 1004.08.

Compound 69: (3S,6S,7E,9R,10R,12R,14S,15E,17E,19E,23S,26R,27R,34aS)-9,27-dihydroxy-3-((R)-1-((1S,3R,4R)-4-hydroxy-3-methoxycyclohexyl) propan-2-yl)-10-methoxy-21-((2-methoxyethyl)(phenyl)amino)-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone:

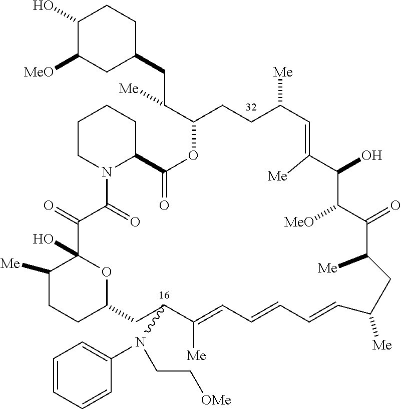

To a stirred solution of 32-deoxorapamycin (75 mg, 0.083 mmol) in DCM (10 mL) was added N-(2-methoxyethyl) aniline (0.125 mL, 0.834 mmol) and TFA (0.128 mL, 1.667 mmol) at −20° C. and stirred for 16 h to rt. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 2.7 mg (3%) of the white solid. MS (ESI-) calc'd for C₅₉H₉₀N₂O₁₂—H=1018.65, found 1018.21.

Compound 70: (3S,6S,7E,9R,10R,12R,14S,15E,17E,19E,23S,26R,27R,34aS)-21-((3-(difluoromethoxy)phenyl)amino)-9,27-dihydroxy-3-((R)-1-((1S,3R,4R)-4-hydroxy-3-methoxycyclohexyl) propan-2-yl)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone:

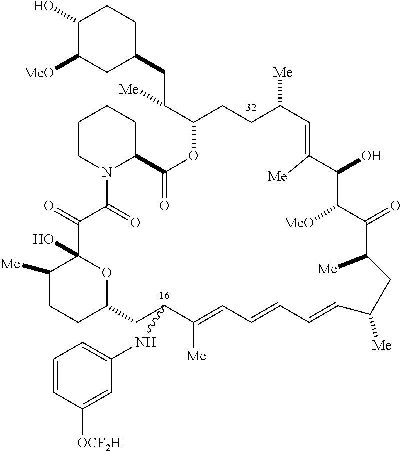

To a stirred solution of 32-deoxorapamycin (100 mg, 0.111 mmol) in DCM (10 mL) was added 3-difluoromethoxyaniline (0.139 mL, 1.112 mmol) and TFA (0.170 mL, 2.223 mmol) at −20° C. and stirred for 16 h at room temperature. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 7.7 mg (7%) of the white solid. MS (ESI-) calc'd for C₅₇H₈₄F₂N₂O₁₂—H=1026.60, found 1026.16.

### Compound 71

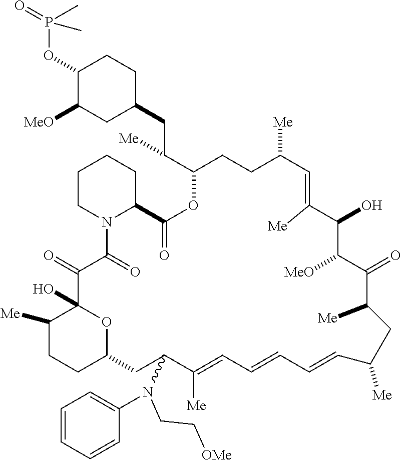

(1R,2R,4S)-4-((2R)-2-((3S,6S,7E,9R,10R,12R,14S,15E,17E,19E,23S,26R,27R,34aS)-9,27-dihydroxy-10-methoxy-21-((2-methoxyethyl)(phenyl)amino)-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl) propyl)-2-methoxycyclohexyl dimethylphosphinate.

Compound 71 is prepared in the same fashion as compound 69 using Compound 31 as the starting material.

### Compound 72

N-((3S,6S,7E,9R,10R,12R,14S,15E, 17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-21-yl)-3-methylbenzenesulfonamide.

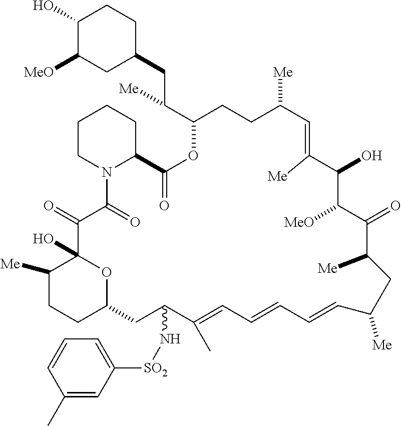

To a stirred solution of 32-deoxorapamycin (50 mg, 0.055 mmol) in DCM (6 mL) was added 3-methylbenzene-1-sulfonamide (95 mg, 0.555 mmol) and ZnCl₂ (0.56 mL, 0.555 mmol) at rt and stirred for 16 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 4.2 mg (7%) of the yellow solid RGN-3768. MS (ESI-) calc'd for C₅₇H86N2O13S—H=1038.59, found 1038.62.

### Compound 73

N-((3S,6S,7E,9R,10R,12R,14S,15E, 17E,19E,23S,26R,27R,34aS)-9,27-dihydroxy-3-((R)-1-((1S,3R,4R)-4-hydroxy-3-methoxycyclohexyl) propan-2-yl)-10-methoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-21-yl)-N-methylbenzenesulfonamide.

To a stirred solution of 32-deoxorapamycin (50 mg, 0.055 mmol) in DCM (6 mL) was added N-methylbenzenesulfonamide (0.080 mL, 0.555 mmol) and ZnCl₂ (0.56 mL, 0.555 mmol) at rt and stirred for 16 h. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H₂O. The pure product fractions were lyophilized to give 26.9 mg (52%) of the yellow solid RGN-3769. MS (ESI-) calc'd for C₅₇H86N2O13S—H=1038.59, found 1038.24.

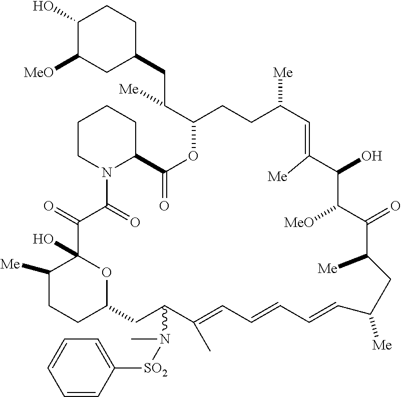

Compound 74. (3S,6S,7E,9R,10R,12R,14S,15E,17E,19E,23S,26R,27R,34aS)-21-((3-fluorophenyl)amino)-9,27-dihydroxy-3-((R)-1-((1S,3R,4R)-4-hydroxy-3-methoxycyclohexyl) propan-2-yl)-10-methoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone.

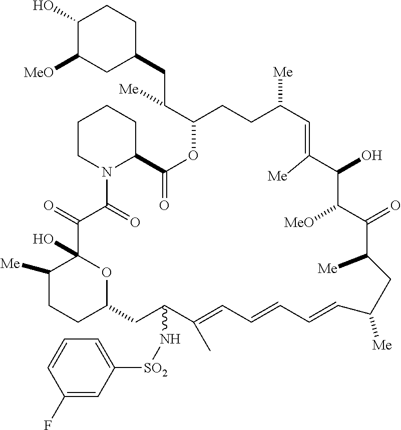

To a stirred solution of 32-deoxorapamycin (300 mg, 0.333 mmol) in DCM (10 mL) was added 3-fluoroaniline (0.322 mL, 3.335 mmol) and TFA (0.510 mL, 6.670 mmol) at −20° C. and stirred for 16 h to rt. The resulting mixture was diluted with EtOAc and quenched with sat. aq. NaHCO₃. The aqueous layer was extracted with EtOAc and the combined organic layers were dried with Na₂SO₄, filtered, and concentrated under reduced pressure. The crude product was dissolved in DMF and subjected to an ISCO C18 column and eluted from 60-80% ACN/H2O. The pure product fractions were lyophilized to give 2.9 mg (1%) of the white solid 74. MS (ESI-) calc'd for C₅₆H83FN2O11-H=978.60, found 978.85.

### Example 3. Inhibition of Representative Compounds Against Wild-Type (WT) and FKBP12 Knock-Out 293T Cells

Example Generation of FKBP 12 knock-out cells. A CRISPR/Cas9 system was used to deliver ribonucleoprotein complexes containing guide RNA sequences that target FKBP12 (acCGGTGTAGTGCACCACGC, location 1369062; ccactactcacCGTCTCCTG, location 1369181) in HEK293T cells using Lipofectamine CRISPRMAX transfection reagent (Invitrogen CMAX00008). Cell clones were screened by immunoblotting with anti-FKBP-specific antibody (Novus, NB300-508). Single cells that were deficient for FKBP12 protein were selected and cloned.

Treatment of wild-type (WT) and FKBP12 knock-out 293T cells with RAD001 (everolimus) and test compounds. WT and FKBP12 knock-out 293T cells were cultured in Dulbecco's modified Eagle's medium (Gibco #11971-025) supplemented with 10% fetal bovine serum (Gibco #26140-079). Cells were plated at a density of 15,000 cells per well in poly-D-Lysine coated 96-well plates (Corning, #354461) and were incubated at 37° C., 5% CO₂ for 24 hours until they reached ˜80% confluence. Cells were treated with RAD001 or test compounds reconstituted in dimethyl sulfoxide (DMSO) using a 8-11-point dose range, from 0.001 pM to 10 μM. All treatments were performed in duplicates for 2 hours at 37° C. Cell culture media supplemented with blank dimethyl sulfoxide (DMSO) was used as a negative control for all compounds. Phosphorylated amounts of S6K1 (Thr389) were measured using an ELISA kit (Invitrogen 85-86052-11), following the manufacture's protocol.

S6K1 is an immediate downstream target of mTORC1 and therefore phosphorylation of S6K1 at Thr389 residue is used to measure mTORC1 activity (PMID: 16968213). Amounts of phosphorylated S6K1 (Thr389) were measured in WT and FKBP12 knock-out 293T cells treated with either RAD001 or Compound 44 (FIGS. 1A and 1B), and Compound 45 (FIGS. 1C and 1D).

In WT cell, both RAD001 and Compound 44 completely inhibit S6K1 phosphorylation in a dose dependent manner (FIG. 1A). In WT cells, RAD001 inhibited 50% (IC50) of S6K1 phosphorylation at 0. 66 nM, while Compound 44 inhibited 50% (IC50) of S6K1 phosphorylation at 11 nM. Thus, in WT cells, Compound 44 is ˜ 17 fold less potent compared with RAD001 (FIG. 1A).

In FKBP12 knock-out cells (i.e. cells that lack FKBP12), RAD001 achieved ˜30% inhibition of S6K1 phosphorylation at 100 nM, while Compound 44 achieved ˜30% inhibition of S6K1 phosphorylation at 10 μM. Thus, in FKBP12 KO cells Compound 44 is ˜ 100 fold less potent compared with RAD001 at the highest tested concentration of 10 μM (FIG. 1B). In FKBP12 knock-out cells, Compound 44 did not inhibit S6K1 phosphorylation at any tested concentration lower than 10 μM (FIG. 1B). These data demonstrate that Compound 44 is relatively more selective for FKBP12 compared to RAD001 and require FKBP12 to inhibit mTORC1 signaling, when used at 1 μM or lower concentration in this cell system.

In WT cell, both RAD001 and Compound 45 completely inhibit S6K1 phosphorylation in a dose dependent manner (FIG. 1C). In WT cells, RAD001 inhibited 50% (IC50) of S6K1 phosphorylation at 0. 47 nM, while Compound 45 inhibited 50% (IC50) of S6K1 phosphorylation at 8.4 nM. Thus, in WT cells, Compound 45 is ˜ 18 fold less potent compared with RAD001 (FIG. 1C).

In FKBP12 knock-out cells (i.e. cells that lack FKBP12), RAD001 achieved ˜35% inhibition of S6K phosphorylation at 100 nM concentration and at the highest tested concentration of 10 μM, RAD001 inhibited S6K1 phosphorylation by ˜80%. In FKBP12 knock-out cells, Compound 45 at 10 μM concentration inhibited S6K1 phosphorylation by ˜ 25% (FIG. 1D). Similar level of S6K1 inhibition was achieved by RAD001 at ˜100 nM. Thus in FKBP12 knock-out cells, when tested at 10 μM concentration, Compound 45 is ˜ 100 fold less potent compared with RAD001 (FIG. 1D).

These data demonstrate that Compound 45 is relatively more selective for FKBP12 compared with RAD001.

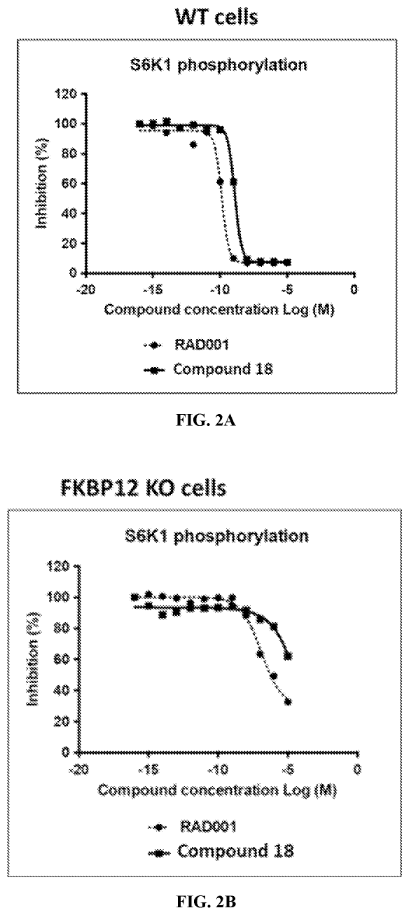

In WT HEK293T cells, which express FKBP12, both RAD001 and Compound 18 achieve complete inhibition of S6K1 (Thr389)phosphorylation (FIG. 2A). In WT cells, the potency of Compound 18 was ˜9 fold lower compared with that of RAD001. However, in the absence of FKBP12 (in FKBP12 knock out cells), RAD001 achieved ˜40% of S6K1 (Thr389) inhibition at 447 nM, while Compound 18 achieved similar levels of S6K1 (Thr389) inhibition at 9.5 μM (FIG. 2B). This indicates that in FKBP12 knock out cells, Compound 18 is ˜20 fold less potent compared with RAD001. These data demonstrate that Compound 18 is relatively more selective to FKBP12 compared with RAD001, however it still achieves about 40% inhibition of S6K1 in the absence of FKBP12 at the highest tested concentration.

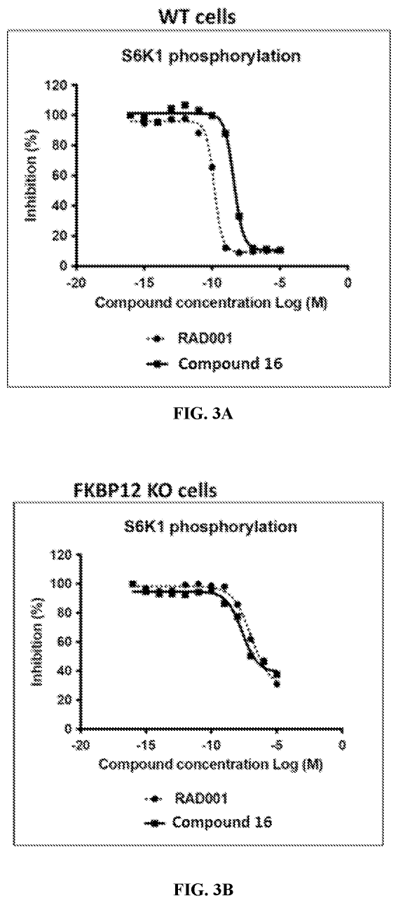

In WT HEK293T cells, which express FKBP12, both RAD001 and Compound 16 achieved complete inhibition of S6K1 (Thr389)phosphorylation (FIG. 3A). In WT cells, the potency of Compound 16 was ˜28 fold lower compared with that of RAD001. However, in the absence of FKBP12 (in FKBP12 knock out cells), the potency of Compound 16 was comparable to that of RAD001 (FIG. 3B). At the highest concentration tested (10 μM), both rapalogs achieved ˜ 70% inhibition of S6K1 (Thr389)phosphorylation. These data suggest that Compound 16 may have lower affinity to FKBP12 compared with RAD001, however it maintains affinity to other FKBPs which are expressed in HEK293T cells.

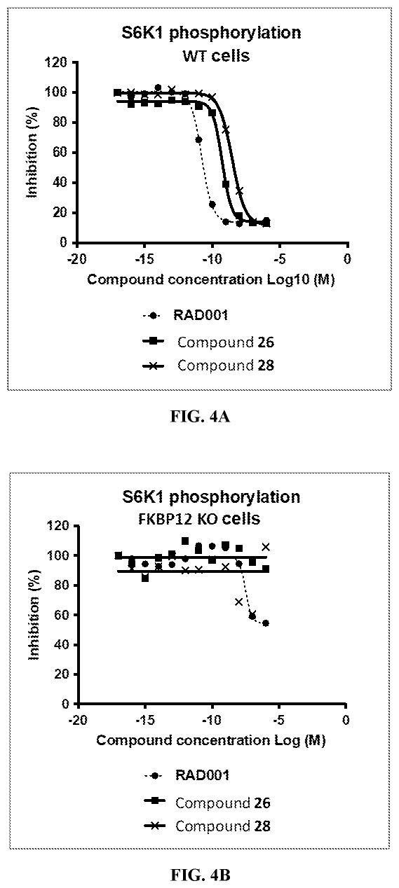

In WT HEK293T cells, which express FKBP12, RAD001, Compounds 26 and Compound 28 achieved complete inhibition of S6K1 (Thr389)phosphorylation (FIG. 4A). Cells were treated for 2 hours with the following compounds: RAD001 (everolimus; dotted line with circles, IC50 0.017 nM) and Compounds 26 (solid line with squares, IC50 0.55 nM) and Compound 28 (solid line with crosses, IC50 3.0 nM). In FKBP12 knock-out 293T cells, while RAD001 (everolimus; dotted line with circles, IC50 31.3 nM) achieves about 50% of S6K1 inhibition, Compounds 26 (solid line with squares, IC50 not measurable/detectable) docs not inhibit S6K1 at any tested concentrations, which indicates that Compound 26 is more selective to FKBP12 compared with RAD001. In FKBP12 KO cells, Compound 28 (solid line with crosses, IC50 not measurable/detectable) behaved in a similar fashion to RAD001, which indicates that selectivity of compound 28 to FKBP12 is not greater compared with RAD001.

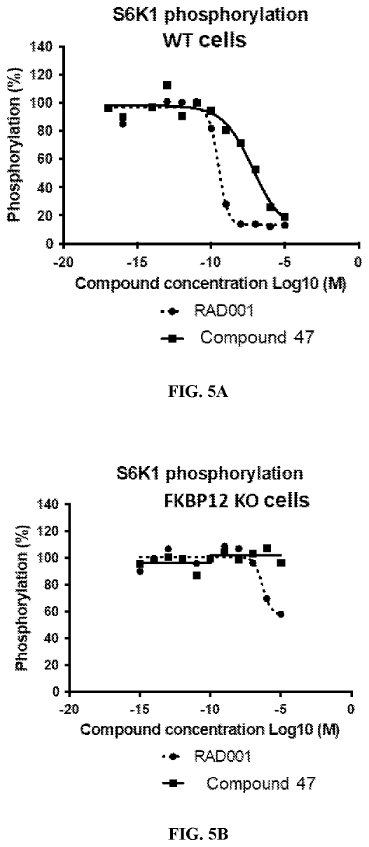

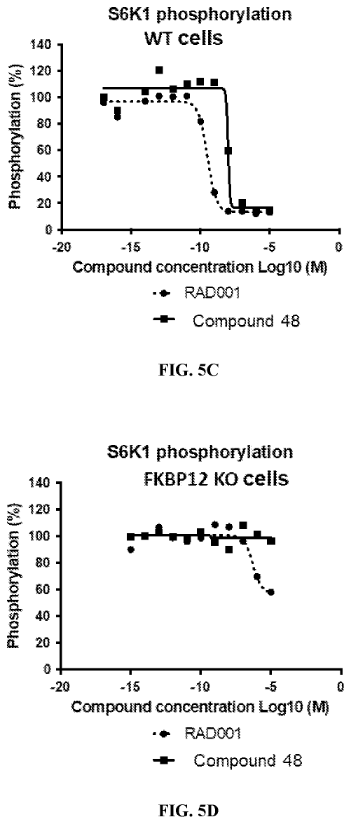

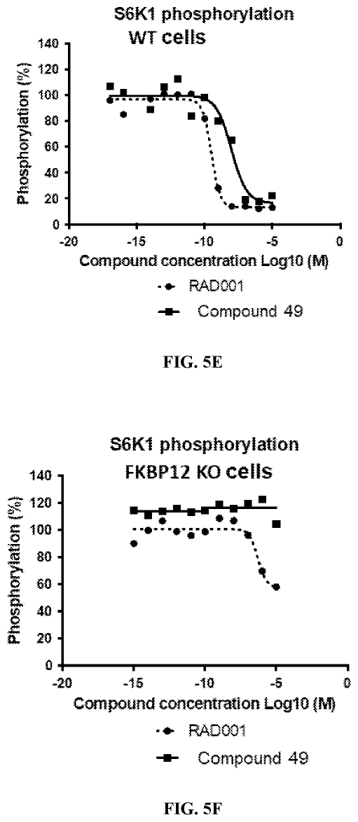

In wild-type HEK293T cells, which express FKBP12, RAD001 (IC50-0.319 nM) as well as Compounds 47 (IC50=69 nM), 48 (IC50-9.8 nM) and 49 (IC50=9.5 nM) inhibited mTORC1 signaling in a dose dependent manner and achieved near complete inhibition of S6K1 (Thr389)phosphorylation with increasing concentrations (FIGS. 5A, 5C, and 5E). Potency of the new compounds was lower relative to RAD001 by ×219 fold (47), ×30 fold (48) and ×29 fold (49) respectively, as indicated by their respective IC50 values.

In the absence of FKBP12, in FKBP12 knock out cells, RAD001 inhibited S6K1 (Thr389)phosphorylation, achieving 42% inhibition at 10 UM concentration. New compounds 47, 48 and 49 tested side-by-side with RAD001, did not inhibit S6K1 (Thr389)phosphorylation at any tested concentrations (FIGS. 5B, 5D, and 5F). These results indicate that unlike RAD001, activity of Compounds 47, 48 and 49 is dependent on FKBP12 and in the absence of FKBP12, these compounds do not inhibit mTORC1 signaling, i.e. Compounds 47, 48 and 49 are relatively more selective to FKBP12 compared with RAD001.

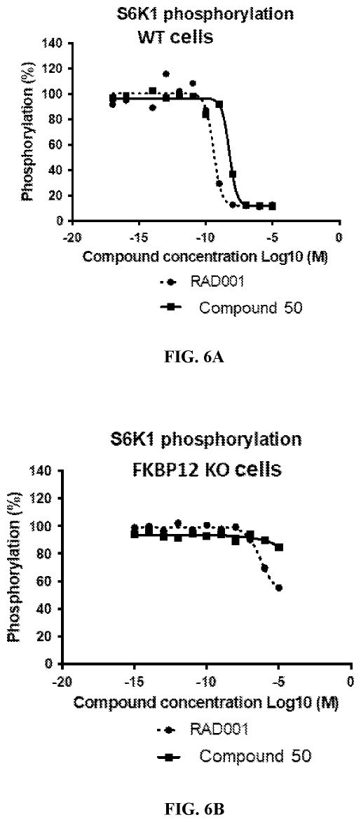

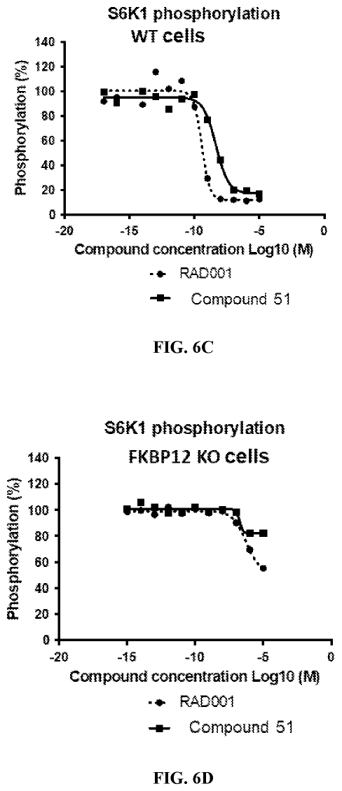

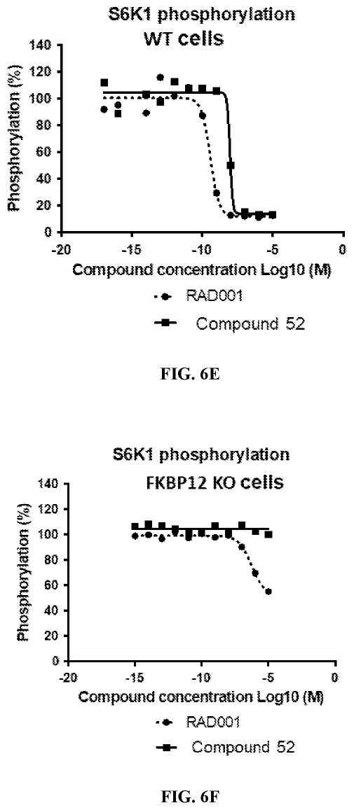

In wild-type HEK293T cells, which express FKBP12, RAD001 (IC50-0.365 nM) as well as Compounds 50 (IC50=5.72 nM), 51 (IC50-4.53 nM) and 52 (IC50=9.15 nM) inhibited mTORC1 signaling in a dose dependent manner and achieved near complete inhibition of S6K1 (Thr389)phosphorylation with increasing concentrations (FIGS. 6A, 6C, and 6E). Potency of these new compounds was lower relative to RAD001 by ×15 fold (50), ×12 fold (51) and ×25 fold (52) respectively, as indicated by their respective IC50 values.

In the absence of FKBP12, in FKBP12 knock out cells, RAD001 inhibited S6K1 (Thr389)phosphorylation in a dose dependent manner, achieving 45% inhibition at 10 μM. New compounds 50 and 52 tested side-by-side with RAD001, either slightly inhibited S6K1 (Thr389)phosphorylation only at the highest tested concentration (12% inhibition in case of 50), or did not inhibit S6K1 (Thr389)phosphorylation at any tested concentrations (seen for 52)(FIG. 6B, 6F). These results indicate that unlike RAD001, activity of Compounds 50 and 52 is dependent on FKBP12 and in the absence of FKBP12, these compounds do not inhibit mTORC1 signaling, i.e. Compounds 50 and 52 are selective to FKBP12.

In FKBP12 knock out cells, Compound 51 retained some potency and inhibited S6K1 (Thr389)phosphorylation by 18% at 1 μM concentration, with no further inhibition at 10 μM (FIG. 6D). In the same assay, RAD001 achieved 20% inhibition of S6K1 (Thr389)phosphorylation at 115 nM concentration. These results indicate that Compound 51 is not completely selective to FKBP12 and inhibitory effect of Compound 51 on the mTORC1 signaling may be mediated by FKBPs other than FKBP12.

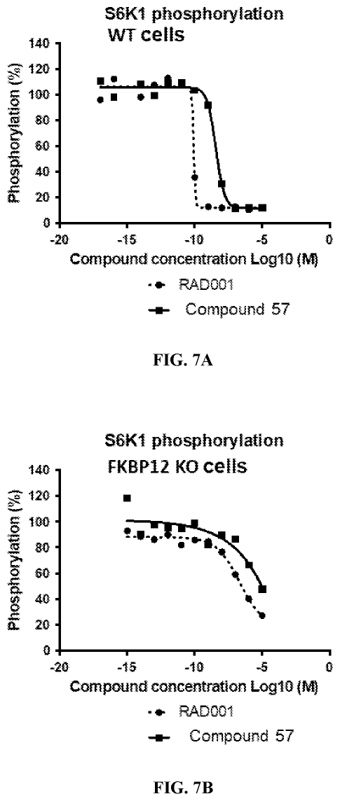

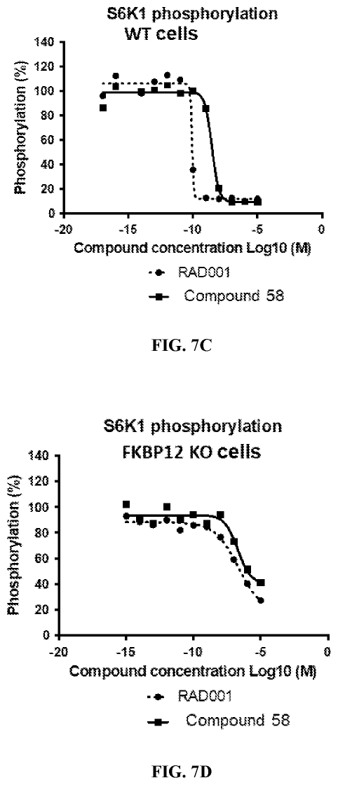

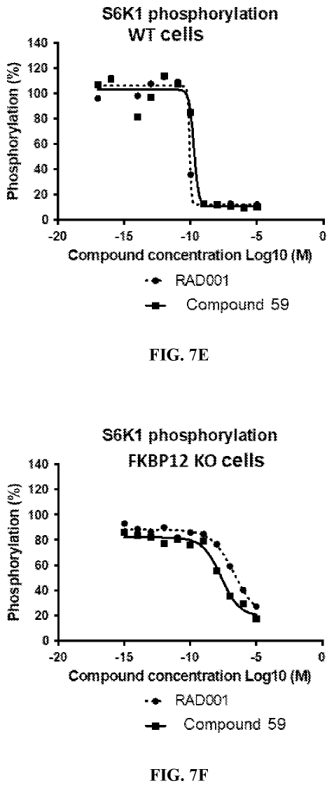

In wild-type HEK293T cells, which express FKBP12, RAD001 (IC50-0.084 nM) and Compounds 57 (IC50=3.68 nM), 58 (IC50-2.94 nM) and 59 (IC50=0.17 nM) inhibited the mTORC1 signaling in a dose dependent manner and achieved near complete inhibition of S6K1 (Thr389)phosphorylation at the highest tested concentrations (FIGS. 7A, 7C, and 7E). In this assay, potencies of the new compounds were lower relative to RAD001 by ×44 fold (57), ×35 fold (58) and ×2 fold (59) respectively, as indicated by their respective IC50 values.

In the absence of FKBP12, in FKBP12 knock out cells, RAD001 and all other tested compounds (57, 58 and 59), inhibited the mTORC1 signaling in a dose dependent manner (FIGS. 7B, 7D, and 7F). These result indicate, that similar to RAD001, compounds (57, 58 and 59) retain potency in the absence of FKBP12, meaning that their inhibitory effect on mTORC1 signaling may be mediated by FKBPs other than FKBP12, i.e. compounds 57, 58 and 59 are relatively not FKBP12 selective.

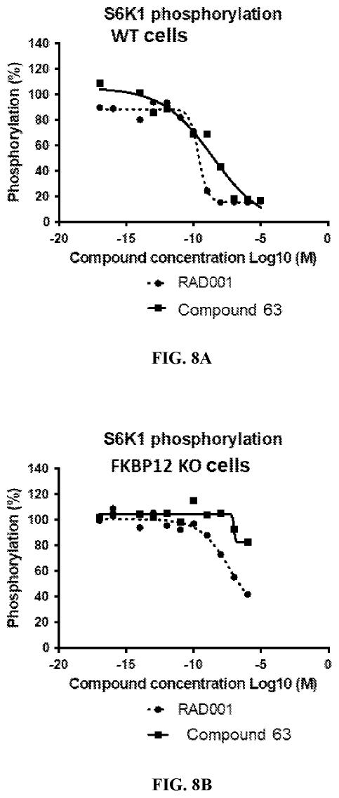

In wild-type HEK293T cells, which express FKBP12, both RAD001 (IC50=0.235 nM) and Compound 63 (IC50-2.43 nM) inhibited the mTORC1 signaling in a dose dependent manner and achieve near complete inhibition of S6K1 (Thr389)phosphorylation with increasing concentrations (FIG. 8A). In this assay, potency of 63 was ×10 fold lower compared with RAD001, as indicated by the respective IC50 values.

In FKBP12 knock out cells, Compound 63 retained some potency and inhibited S6K1 (Thr389)phosphorylation by 17% at the maximum tested concentration, 10 μM (FIG. 8B). In the same assay, RAD001 achieved 20% inhibition of S6K1 (Thr389)phosphorylation at 1.3 nM concentration and further 58% inhibition at 10 μM. These results indicate that even though Compound 63 is not exclusively selective to FKBP12, it is still more selective than RAD001. In wild-type cells that express FKBP12, 63 is ×10 fold less potent compared with RAD001, however in FKBP12 knock-out cells, 63 does not achieve more than 17% of S6K1 (Thr389) inhibition, while RAD001 inhibits S6K1 (Thr389) in a dose dependent manner by as much as 58%.

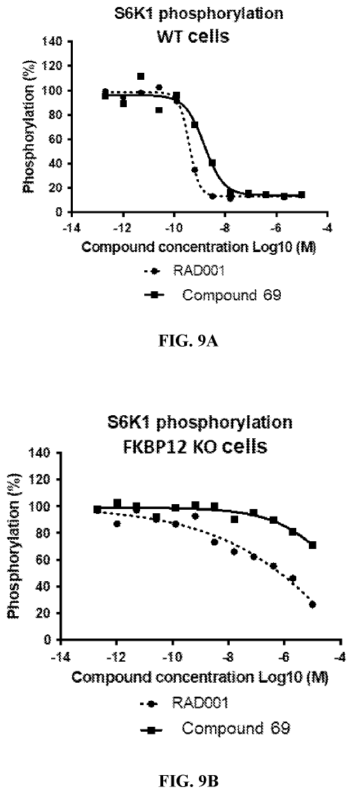

In wild-type HEK293T cells, which express FKBP12, both RAD001 (IC50=0.389 nM) and Compound 69 (IC50=1.53 nM) inhibited mTORC1 signaling and achieved near complete inhibition of the S6K1 (Thr389)phosphorylation with increasing concentrations (FIG. 9A), with potency of 69 being ×4 fold lower compared to RAD001, as indicated by the respective IC50 values.

In the absence of FKBP12, in FKBP12 knock out cells, increasing concentrations of RAD001 achieved dose dependent inhibition of S6K1 (Thr389)phosphorylation of up to 73.5% at the highest tested concentration (10 μM). In the absence of FKBP12, Compound 69 retained very low potency and achieved about 30% inhibition of S6K1 (Thr389)phosphorylation at the highest tested concentration (10 μM). In the same FKBP12 knock out cell assay, RAD001 achieved 30% inhibition of S6K1 (Thr389)phosphorylation at ˜3.0 nM concentration. These results indicate that even though Compound 69 is not exclusively selective to FKBP12, it is still more selective than RAD001. In wild-type cells that express FKBP12, Compound 69 is only 4 fold less potent compared with RAD001. However, in FKBP12 knock-out cells, Compound 69 inhibits 30% of phosphorylated S6K1 at about 3000 fold higher concentration compared with RAD001.

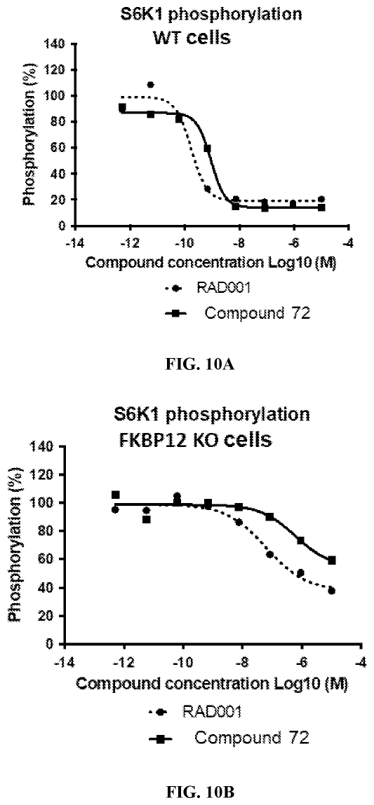

In wild-type HEK293T cells, which express FKBP12, both RAD001 (IC50=0.174 nM) and Compound 72 (IC50=0.914 nM) inhibited mTORC1 signaling and achieved near complete inhibition of the S6K1 (Thr389)phosphorylation with increasing concentrations (FIG. 10A). In this assay, potency of 72 was ×5.2 fold lower compared with RAD001, as indicated by the respective IC50 values.

In the absence of FKBP12, in FKBP12 knock out cells, increasing concentrations of RAD001 achieved dose dependent inhibition of S6K1 (Thr389)phosphorylation with up to 62% inhibition at the highest tested concentration (10 μM)(FIG. 10B). In the absence of FKBP12, Compound 72 also retained some of its potency and inhibited S6K1 (Thr389)phosphorylation in a dose dependent manner, with 40% inhibition at the highest tested concentration (10 μM)(FIG. 10B). These results indicate that although 72 is less potent than RAD001 in FKBP12 knockout cells, these two rapalogs behave in a similar way. I.e. they can induce mTORC1 inhibition when complexed with FKBPs, other than FKBP12.

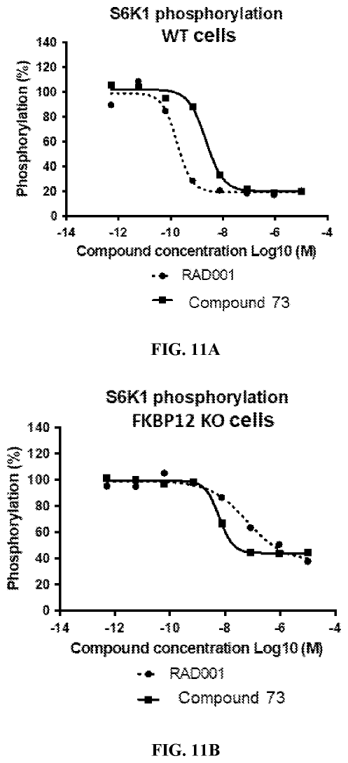

In wild-type HEK293T cells, which express FKBP12, both RAD001 (IC50=0.174 nM) and Compound 73 (IC50=2.22 nM) inhibited mTORC1 signaling and achieved near complete inhibition of the S6K1 (Thr389)phosphorylation with increasing concentrations (FIG. 11A). In this assay, potency of 73 was ×13 fold lower compared with RAD001, as indicated by the respective IC50 values.

In the absence of FKBP12, e.g. in FKBP12 knock out cells, increasing concentrations of RAD001 and 73 achieved dose dependent inhibition of S6K1 (Thr389)phosphorylation, with up to ˜ 60% inhibition at the highest tested concentration (10 μM)(FIG. 11B). Moreover, even though 73 was ×13 fold less potent compared with RAD001 in wild-type cells that express FKBP12, in the absence of FKBP12 (in FKBP12 knock out cells), 73 gained potency over RAD001. Specifically, in FKBP12 knock out cells, 73 inhibited S6K1 (Thr389)phosphorylation by 20% at 2.7 nM concentration, while RAD001 achieved 20% inhibition at 7.4 nM (FIG. 11B). However, at the highest tested concentration of 10 μM, both RAD001 and Compound 73 inhibited S6K1 phosphorylation by ˜60% (FIG. 11B).

These results indicate that 73 can induce mTORC1 inhibition when complexed with FKBPs, other than FKBP12, i.e. 73 is not FKBP12 selective. Moreover, higher potency (in the dose range less than 1 μM) of 73 vs RAD001 in FKBP12 knock out cells indicates that 73 may have gained affinity to FKBP(s) other than FKBP12, over RAD001.

### Example 4. Microsome Stability

### Materials and Methods

Buffers: PBK Buffer: 50 mM potassium phosphate buffer (PPB), pH 7.2, with 3.3 mM Magnesium Chloride.

### Compound Dilution:

TA Intermediate solution: Compound or control (3 μL) diluted from stock solution (10 mM) with 297 μL 90.0% methanol/water. (Conc: 100 μM; 1.0% DMSO, 89.1% MeOH).

Control cocktail intermediate solution: Each control (3 μL) was diluted with 291 μL 90.0% methanol/water.

Diluted TA intermediate stock solution and PC control stock were made by transferring 80 μL of 100 μM TA stock solution into new tubes and then adding 720 μL of 1.0% DMSO in 50 mM potassium phosphate buffer (conc. 10 μM; 1.0% DMSO, 8.91% MeOH).

### Preparation of Liver Mixture and NADPH Cofactor (Described in Table 1)

Preparation of Liver microsomes working solution (1.25×)(final Conc.: 0.5 mg/mL).

TABLE 1
Preparation of liver mixture and NADPH cofactor

| | | | | Vol. of 50 mM | | | |
|---|---|---|---|---|---|---|---|
| | | | Vol. of xLM | PBK Buffer with | Total vol | Working | Final |
| Species | Vendor | Description | (20 mg-protein/mL) | 3.3 mM MgCl₂ | (mL) | Conc. | Conc. |
| Mouse | Xenotech | 20 mg-protein/mL, | 0.100 mL | 3.100 mL | 3.200 mL | 0.625 mg/mL | 0.5 mg/mL |
| Liver | | n = 1400, | | | | | |
| Microsome | | Pooled Male | | | | | |
| Human | Xenotech | 20 mg-protein/mL, | 0.100 mL | 3.100 mL | 3.200 mL | 0.625 mg/mL | 0.5 mg/mL |
| Liver | | n = 200, | | | | | |
| Microsome | | Mixed Gender | | | | | |

| | | | | Vol. of | Working | |
|---|---|---|---|---|---|---|
| | | | NADPH | PBK | Solution | Final |
| NADPH | Product info | Source | (mg) | Buffer | Conc. | Conc. |
| NADPH | Cat | Sigma | 10.6 | 1.18 mL | 10 mM | 1 mM |
| | No. N1630 | | | | | |
| | Lot No. | | | | | |
| | SLCK6270 | | | | | |

### Stock Solution:

Acetonitrile:methanol (9:1, v/v)(including 200 ng/mL tolbutamide and 200 ng/ml labetalol hydrochloride as internal standards).

Working solution: Intermediate solution (80 μL) was diluted with 720 μL 1.0% DMSO in 50 mM potassium phosphate buffer (Conc.: 10 μM; 1.0% DMSO, 8.91% MeOH)

Procedure: Stock solution was added to each of the time point plates. Microsomes solution (360 μL/well), TA/PC (45 μL/well), and NADPH (45 μL/well) min. (Conc: 1.0 μM, 0.10% DMSO, 0.891% MeOH) were added to the incubation plates. To the T60 and NCF60 plate, TA (15 ul/well) and Microsomes (120 ul) were added. To the blank plate, microsome (50 ul/well) with stop solution (150 ul/well) were added. At the end of each time point, 50 ul/well was aliquoted from the incubation plate. The sampling plates were shaken for approximately 10 min. on a shaker and the samples were centrifuged at 4000 rpm for 15 min. Supernatant (120 μL) was transferred for LC/MS/MS.Compounds from Table 3 were incubated at 37° C. with liver microsomes (pooled from multiple donors) at 1 μM in the presence of a NADPH regenerating system at 0.5 mg/ml microsomal protein. Positive controls included testosterone (3A4 substrate), propafenone (2D6) and diclofenac (2C₉), which were incubated with microsomes in the presence of an NADPH regenerating system. At time points (0, 5, 10, 20, 30 and 60 minutes), samples were removed and immediately mixed with cold acetonitrile containing the internal standard (IS). Test compounds incubated with microsomes without the NADPH regenerating system for 60 min are also included. A single point for each test condition (n=1) was obtained, and samples were analyzed by LC/MS/MS. Disappearance of the test compound was assessed based on peak area ratios of analyte/IS (no standard curve). As shown in Table 1, a number of compounds showed good stability in human and mouse liver microsomes with a T_{1/2} of over 2 hrs and low microsome clearance.

TABLE 2
In Vitro Microsomal Metabolic Stability Assay of selected compounds in
CD-1 Mouse and Human Liver Microsomes.

| Comd | | T_{1/2} | CL | CLᵢₙₜ | Remaining | Remaining |
|---|---|---|---|---|---|---|
| #ID | Species | (min) | (μl/min/mg) | (mL/min/kg) | (T = 60 min) | (T = NCF60 min) |
| 26 | Mouse | 131 | 10.6 | 41.9 | 69.80% | 92.30% |
| | Human | 15.4 | 89.9 | 80.9 | 6.60% | 92.80% |
| 28 | Mouse | 83.5 | 16.6 | 65.7 | 59.40% | 106% |
| | Human | 26.3 | 52.8 | 47.5 | 19.40% | 84.00% |
| 44 | Mouse | 33.8 | 41 | 162 | 31% | 96% |
| | Human | 17.4 | 79.6 | 71.7 | 7.50% | 99% |
| 45 | Mouse | 84.6 | 16.4 | 64.8 | 64.30% | 81.40% |
| | Human | 7.13 | 194 | — | 0.00% | 71.30% |
| 48 | Mouse | >145 | <9.6 | — | 101% | 150% |
| | Human | 33 | 42.1 | 37.8 | 32% | 107% |
| 49 | Mouse | 130 | 10.7 | 42.3 | 67% | 139% |
| | Human | 20.9 | 66.2 | 59.6 | 15% | 102% |
| 50 | Mouse | >145 | <9.6 | — | 89% | 112% |
| | Human | 22.8 | 60.8 | 54.7 | 18% | 99% |
| 51 | Mouse | 21.5 | 64.3 | 255 | 15% | 85% |
| | Human | 6.86 | 202 | 182 | 0.20% | 89% |
| 58 | Mouse | 217 | 6.4 | 25.3 | 83% | 96% |
| | Human | 69.5 | 20.0 | 18.0 | 55% | 93% |
| 59 | Mouse | 135 | 10.3 | 40.8 | 76% | 112% |
| | Human | 56.2 | 24.7 | 22.2 | 47% | 102% |
| 60 | Mouse | 14.8 | 93.5 | 370 | 5.6% | 75% |
| | Human | 16.0 | 89.3 | 80.4 | 6.6% | 99% |
| 69 | Mouse | 492 | 2.81 | 11.1 | 113% | 99.6% |
| | Human | 49.0 | 28.3 | 25.5 | 63.3% | 115% |
| 32-Deoxo | Mouse | 39.5 | 35.1 | 139 | 36% | 110% |
| Rapamycin | Human | 3.4 | 405 | 364 | 0.00% | 96% |
| Rapamycin | Mouse | 32.1 | 43.1 | 171 | 27% | 102% |
| | Human | 10.7 | 130 | 117 | 1.40% | 116% |
| RAD001 | Mouse | 71.3 | 19.4 | 76.9 | 56% | 126% |
| | Human | 11.1 | 124 | 112 | 2.10% | 120% |

### Example 5. Pharmacokinetic Studies of Test Articles in Male CD-1 Mice Study Design

TABLE 3
Pharmacokinetic studies design*

| | | Treatment | | | | | |
|---|---|---|---|---|---|---|---|
| | No. of | | | Dose | | | |
| | Animals | Test** | Dose | Volume | Conc. | | |
| Group | (Gender) | Article | (mg/kg) | (mL/kg) | (mg/mL) | Vehicle | Route |
| 1 | 3 (Male) | 69 | 2 | 5 | 0.4 | 5% ethanol, 5% Tween- | IV |
| | | | | | | 80 and 5% PEG-400, | |
| | | | | | | 85% water | |
| 2 | 3 (Male) | 69 | 10 | 10 | 1.0 | 5% ethanol, 5% Tween- | PO |
| | | | | | | 80 and 5% PEG-400, | |
| | | | | | | 85% water | |

Feeding condition: □ Fed [symbol] Fasted
*For both groups (IV and PO) plasma samples are collected at times: 0.083, 0.25, 0.5, 1.0, 2.0, 4.0, 8.0, and 24 h post-dosing. The terminal collection (24 h) of plasma is carried out via cardiac puncture.
**For Compounds RAD001, 26 and 45, the studies were carried out in the same way as compound 69, with one difference: Compounds 26 and 45 were dosed per oral (PO) at 20 mg/kg Compound 69 and RAD001 were dosed PO at 10 mg/kg.

Animal experiments were carried out at Wuxi AppTec. Male 6-8 weeks old CDI mice were obtained from Hilltop Labs. Following arrival at Wuxi AppTec, mice were housed in animal rooms with environmental control (temperature: 20 to 26° C.; lighting: 12 hour light/dark cycle). Mice were fed with certified pellet diet (Certified Rodent Diet #5002 by LabDiet). Water was provided to the animals ad libitum. Mice were acclimated to the facility for at least 3 days prior to starting experiments.

### Dose Formulation

Appropriate amount of test article formulated in 5% ethanol, 5% Tween-80 and 5% PEG-400, 85% water. Formulation was prepared on the day of dosing and dosed within 2 hours following preparation of formulations. Dose accuracy was determined by LC-MS/MS.

### Dose Administration

Dose formulation(s) were administered following facility SOPs. Dose volumes were determined individually prior to dosing based on animals' body weights.

### Sample Collection and Plasma Isolation

About 40 μL of blood samples were collected via peripheral veins (e.g. saphenous veins) at each pre-defined time point. Blood samples were collected into tubes containing K2EDTA as anti-coagulant and kept on ice until centrifugation.

Blood samples were centrifuged at 4° C., 3000 g for 5 min within half an hour of collection. Plasma was transferred into polypropylene tubes or 96-well plates, promptly frozen on dry ice and stored at −70±10° C. until analysis by LC-MS/MS.

### Bioanalytical Method and Sample Analysis

Test compound concentration in mouse plasma was determined with LC-MS/MS method using a calibration curve with at least 6 non-zero calibration standards.

### Data Analysis

The pharmacokinetics (PK) of the test article was analyzed using Phoenix WinNonlin software (version 8.3) and non-compartmental analysis model. Derived PK parameters include, but not limited to, C₀, CLₚ, Vdss, Cₘₐₓ, Tₘₐₓ, T_{1/2}, AUC₍₀₋ₜ₎, AUC_{(0-inf)}, MRT₍₀₋ₜ₎, MRT_{(0-inf)} and % F (bioavailability).

Results Pharmacokinetic study results of selected compounds are shown in FIG. 12A (Everolimus, RAD001), 12B (Compound 26), 12C (Compound 45), and 12D (Compound 63), respectively.

FIG. 12A is a graph showing mouse plasma pharmacokinetic profile of RAD001 following IV (2 mg/kg) and PO (10 mg/kg) administration. Y axis, set to Log 10 scale, shows compound concentration (ng/ml) in plasma. X axis shows time points (hours) for plasma collection following compound administration.

FIG. 12B is a graph showing mouse plasma pharmacokinetic profile of 26 following IV (2 mg/kg) and PO (20 mg/kg) administration. Y axis, set to Log 10 scale shows compound concentration (ng/ml) in plasma. X axis shows time points (hours) for plasma collection following compound administration.

FIG. 12C is a graph showing mouse plasma pharmacokinetic profile of Compound 45 following IV (2 mg/kg) and PO (20 mg/kg) administration. Y axis, set to Log 10 scale shows compound concentration, ng/ml in plasma. X axis shows time points (hours) for plasma collection following compound administration.

FIG. 12D is a graph showing mouse plasma pharmacokinetic profile of 69 following IV (2 mg/kg) and PO (10 mg/kg) administration. Y axis, set to Log 10 scale, shows compound concentration, ng/ml in plasma. X axis shows time points (hours) for plasma collection following compound administration.

Compound 69 has an improved pharmacokinetic profile as compared with RAD001, such as higher oral bioavailability (30.7% for 69 vs 26% for RAD001), higher plasma Cmax following oral administration (12,933 ng/ml for 69 vs 7,224 ng/ml for RAD001) and slower plasma clearance rate (15.5 hours for 69 vs 5.85 hours for RAD001)(compare FIG. 12A with FIG. 12D), also see Table 4 below.

TABLE 4
Summary Table of Pharmacokinetic Studies of Selected Compounds in Mice. Note
that Compounds 26 and 45 were dosed per os (PO) at 20 mg/kg (FIG. 12B and 12C),
while Compound 69 and RAD001 were dosed PO at 10 mg/kg (FIG. 12A and 12D).

| | Everolimus/ | | Compound | | Compound | | Compound | |
|---|---|---|---|---|---|---|---|---|
| | RAD001 | | #26 | | #45 | | #69 | |
| IV T_{1/2}* | 5.85 | h | 5.25 | h | 5.63 | h | 15.5 | h |
| CI(mL/min/kg)** | 0.934 | | 2.31 | | 1.13 | | 0.176 | |
| Vdss (L/kg)*** | 0.433 | | 0.828 | | 0.475 | | 0.228 | |
| PO Cₘₐₓ**** | 7224 | ng/mL | 4118 | ng/mL | 9183 | ng/mL | 12933 | ng/mL |
| Compound concentration | 1959 | ng/mL | 1325 | ng/mL | 1517 | ng/mL | 10010 | ng/mL |
| at 8-hour, PO | | | | | | | | |
| administration | | | | | | | | |
| Compound concentration | 273 | ng/mL | 184 | ng/mL | 239 | ng/mL | 4670 | ng/mL |
| at 24-hour, PO | | | | | | | | |
| administration | | | | | | | | |

| | | | | |
|---|---|---|---|---|
| Oral Bioavailability | 26% | 28.5% | 17% | 30.7% |

*IV T_{1/2}—the amount of time required for compound plasma concentration do decline by 50% following IV administration.
**Cl(ml/min/kg)—compounds clearance following IV administration.
***Vdss (L/kg)—volume of distribution following IV administration.
****PO Cₘₐₓ—maximum (peak) plasma concentration of the compound achieved after PO administration.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

1. A compound of Formula I: 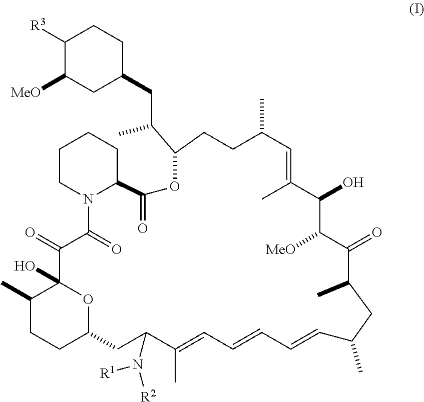
or a pharmaceutically acceptable salt thereof;
wherein R¹ is hydrogen, C₁-₆ alkyl, heterocyclyl, aryl, heteroaryl, —C₀₋₆alkylene-SO₂R⁴, or —C₀₋₆alkylene-SO₂R⁵; wherein the C₁₋₆ alkyl, heterocyclyl, aryl, heteroaryl are optionally substituted with one or two R^{1a} groups;
R² is heterocyclyl, aryl, heteroaryl, —C₀₋₆alkylene-SO₂R⁴, or —C₀₋₆alkylene-SO₂R⁵; wherein the heterocyclyl, aryl, heteroaryl are optionally substituted with one or two R^{2a} groups;
or R¹ and R² together with the attached nitrogen form a N-linked heteroaryl or N-linked heterocyclyl optionally substituted with one or two R^{1a} groups;
R³ is selected from the group consisting of —OR^{a}, 3-6 membered heterocyclyl, and 3-6 membered heteroaryl;
R^{a} is selected from the group consisting of H, —P(O)(R^{b})₂, —C(O)R^{c}, —C(O)OR^{c}, C₁₋₆ alkyl, and C₁₋₆ hydroxyalkyl;
each R^{b} is independently selected from the group consisting of H and C₁-₆ alkyl;
R^{c} is selected from the group consisting of H, C₁₋₆ alkyl, and C₁₋₆ hydroxyalkyl;
R⁴ is C₂₋₆alkyl, C₃₋₆cycloalkyl, C₁₋₆ hydroxyalkyl, heterocyclyl, aryl, or heteroaryl; wherein the heterocyclyl, aryl, and heteroaryl are optionally substituted with one or two R^{4a} groups; and
R⁵ is heterocyclyl, aryl, or heteroaryl; wherein the heterocyclyl, aryl, and heteroaryl are optionally substituted with one or two R^{5a} groups;
each of the one or two R^{1a}, R^{2a}, R^{4a}, and R^{5a} groups, when present, are independently selected from C₁₋₆alkyl, C₁₋₆ hydroxyalkyl, hydroxy, halo, C₁₋₆ haloalkyl, and C₁₋₆ alkoxy; or any two R^{1a}, R^{2a}, R^{4a}, and R^{5a} groups, when present on the same carbon, are taken together to form an oxo group;
wherein when R¹ is hydrogen, R² is not-C₀₋₆ alkylene-SO₂R⁴.

2. The compound of claim 1, wherein the compound is of Formula Ia: 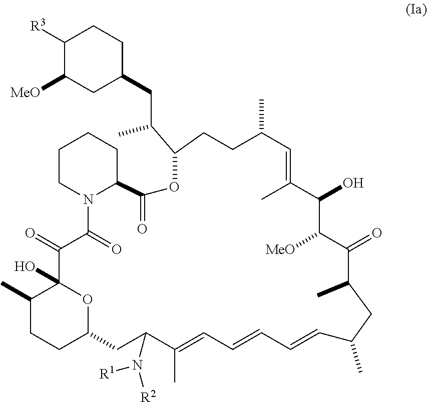
or a pharmaceutically acceptable salt thereof;
wherein R¹ is hydrogen or C₁-₆ alkyl;
R² is heterocyclyl, aryl, or heteroaryl optionally substituted with one or two R^{2a} groups;
R³ is selected from the group consisting of —OR^{a}, a 3-6 membered heterocyclyl, and 3-6 membered heteroaryl;
R^{a} is selected from the group consisting of H, —P(O)(R^{b})₂, —C(O)RE, —C(O)ORE, C₁₋₆ alkyl, and C₁₋₆ hydroxyalkyl;
each R^{b} is independently selected from the group consisting of H and C₁₋₆ alkyl;
R^{c} is selected from the group consisting of H, C₁-₆ alkyl, and C₁₋₆ hydroxyalkyl; and
each of the one or two R^{2a} groups, when present, are independently selected from C₁₋₆ alkyl, C₁₋₆ hydroxyalkyl, hydroxy, halo, C₁₋₆haloalkyl, and C₁₋₆ alkoxy; or any two R^{2a} groups, when present on the same carbon, are taken together to form an oxo group.

3. The compound of claim 1, wherein the compound is of Formula Ib: 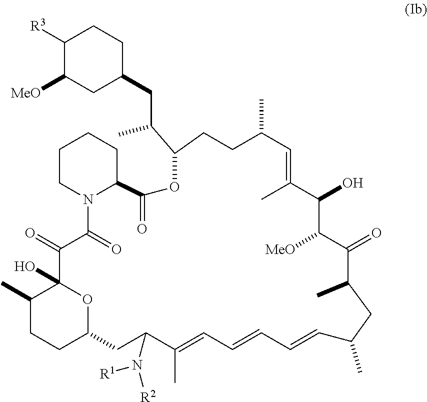
or a pharmaceutically acceptable salt thereof;
wherein R¹ and R² together with the attached nitrogen form a N-linked heteroaryl or N-linked heterocyclyl optionally substituted with one or two R^{1a} groups;
R³ is selected from the group consisting of —OR^{a}, a 3-6 membered heterocyclyl, and 3-6 membered heteroaryl;
R^{a} is selected from the group consisting of H, —P(O)(R^{b})₂, —C(O)RE, —C(O)ORE, C₁₋₆ alkyl, and C₁₋₆ hydroxyalkyl;
each R^{b} is independently selected from the group consisting of H and C₁-₆ alkyl;
R^{c} is selected from the group consisting of H, C₁-₆ alkyl, and C₁₋₆ hydroxyalkyl;
each of the one or two R^{1a} groups, when present, are independently selected from C₁₋₆ alkyl, C₁₋₆ hydroxyalkyl, hydroxy, halo, C₁₋₆haloalkyl, and C₁₋₆alkoxy; or two R^{1a} groups, when present on the same carbon, are taken together to form an oxo group.

4. The compound of claim 1, wherein the compound is of Formula Ic: 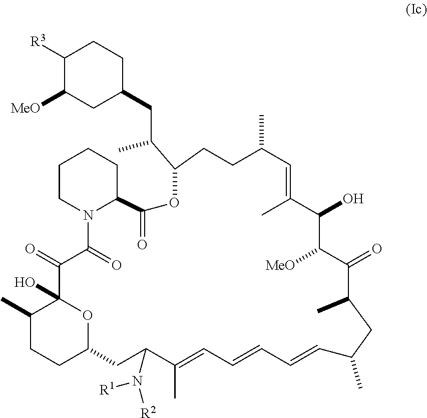
or a pharmaceutically acceptable salt thereof;
wherein R¹ is hydrogen or C₁-₆ alkyl;
R² is —C₀₋₆alkylene-SO₂R⁴ or —C₀₋₆alkylene-SO₂R⁵;
R³ is selected from the group consisting of —OR^{a}, a 3-6 membered heterocyclyl, and 3-6 membered heteroaryl;
R^{a} is selected from the group consisting of H, —P(O)(R^{b})₂, —C(O)R^{c}, —C(O)OR^{c}, C₁-₆ alkyl, and C₁₋₆ hydroxyalkyl;
each R^{b} is independently selected from the group consisting of H and C₁-₆ alkyl;
R^{c} is selected from the group consisting of H, C₁-₆ alkyl, and C₁₋₆ hydroxyalkyl;
R⁴ is C₂₋₆ alkyl, C₃₋₆cycloalkyl, C₁₋₆ hydroxyalkyl, heterocyclyl, aryl, or heteroaryl; wherein the heterocyclyl, aryl, and heteroaryl are optionally substituted with one or two R^{4a} groups;
R⁵ is heterocyclyl, aryl, or heteroaryl; wherein the heterocyclyl, aryl, and heteroaryl are optionally substituted with one or two R^{5a} groups;
each of the one or two R^{4a} and R^{5a} groups, when present, are independently selected from C₁₋₆ alkyl, C₁₋₆ hydroxyalkyl, hydroxy, halo, C₁₋₆haloalkyl, and C₁₋₆ alkoxy; or any two R^{4a} and R^{5a} groups, when present on the same carbon, are taken together to form an oxo group;
wherein when R¹ is hydrogen, R² is not-C₀₋₆ alkylene-SO₂R⁴.

5. (canceled)

6. The compound of claim 1, wherein R¹ is hydrogen or C₁-₆ alkyl and R² is aryl, heteroaryl, or a C₅₋₁₂heterocyclyl wherein the aryl, heteroaryl, and a C₅₋₁₂heterocyclyl are optionally substituted with one or two R^{2a} groups.

7. (canceled)

8. (canceled)

9. The compound of claim 6, wherein R² is selected from 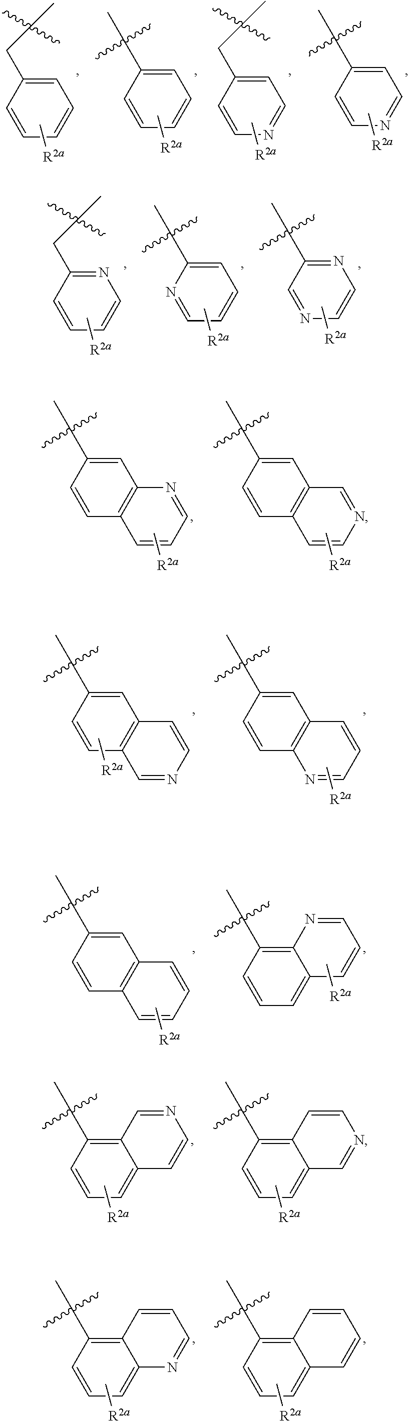 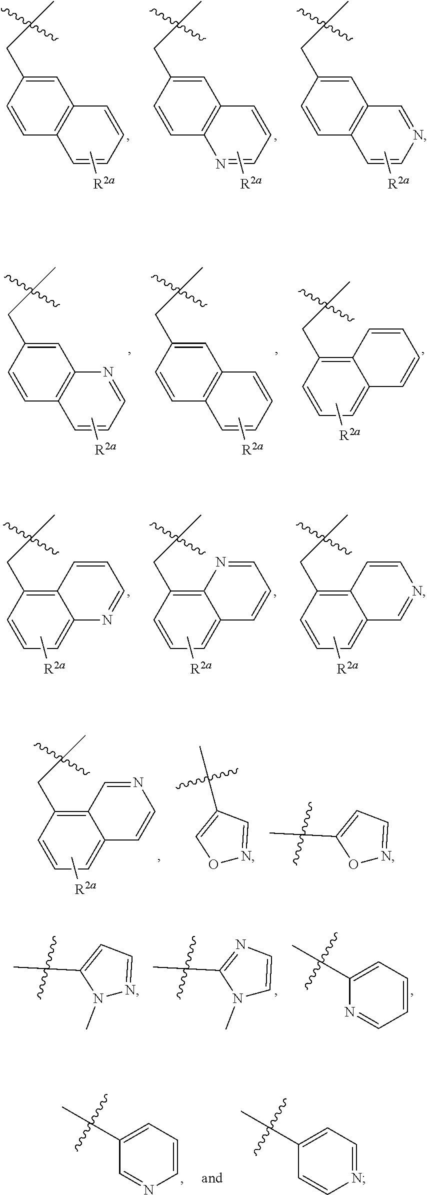
wherein [symbol] is the point of attachment to the rest of the compound.

10. The compound of claim 1, wherein R⁴ is heterocyclyl, aryl, or heteroaryl; wherein the heterocyclyl, aryl, heteroaryl are optionally substituted with one or two R^{4a} groups; or R⁵ is heterocyclyl, aryl, or heteroaryl; wherein the heterocyclyl, aryl, heteroaryl are optionally substituted with one or two R^{5a} groups.

11. The compound of claim 10, wherein R⁴ is 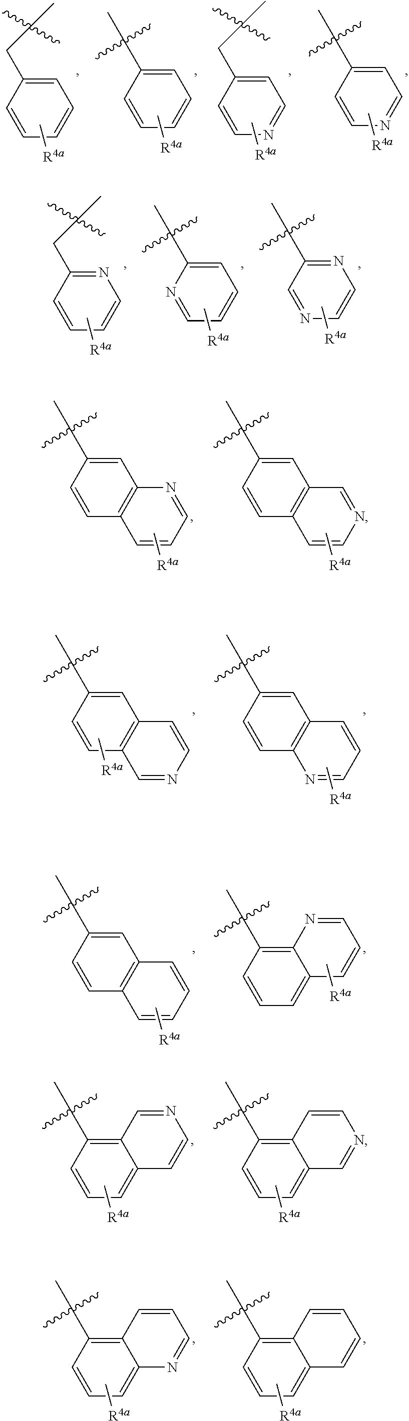 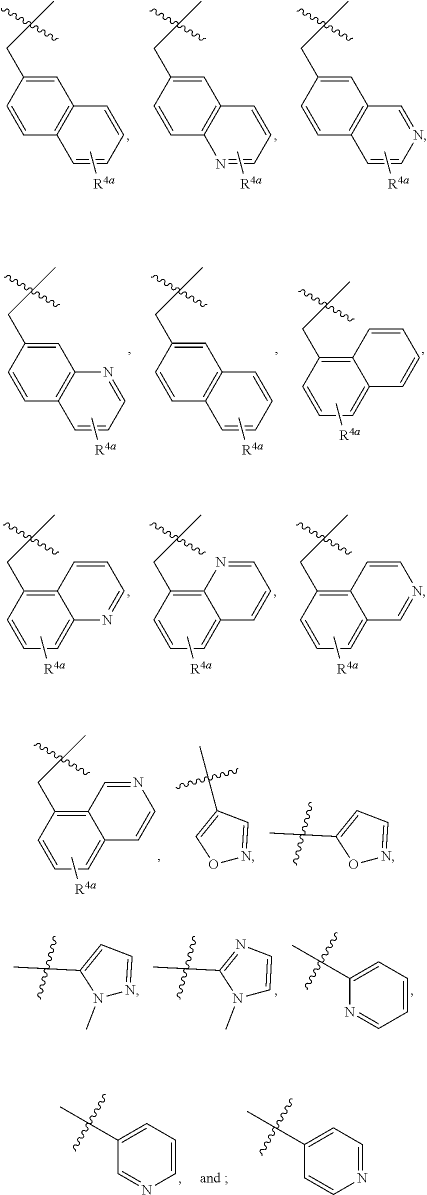
wherein [symbol] is the point of attachment to the rest of the compound.

12. The compound of claim 10, wherein R⁵ is 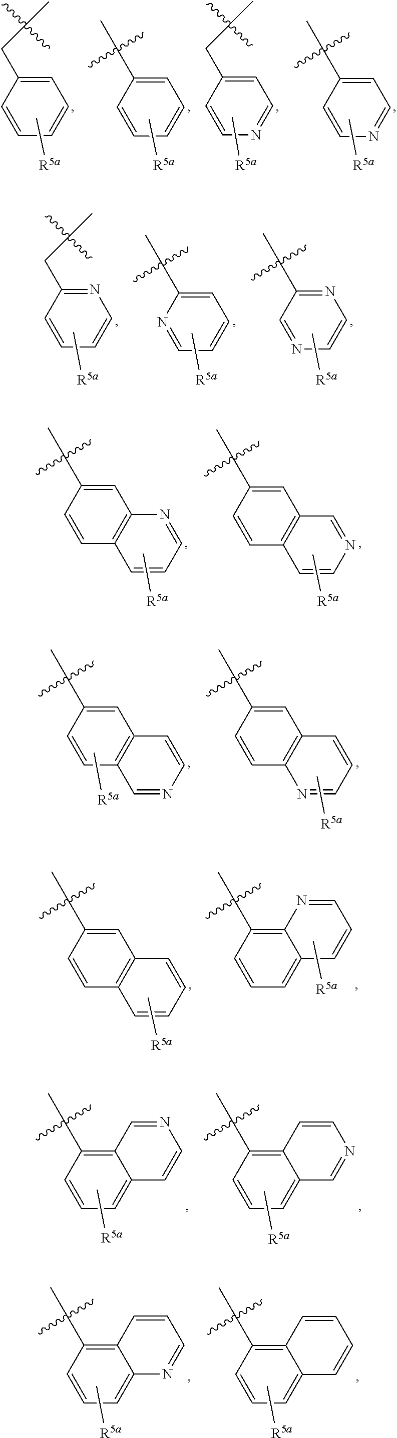 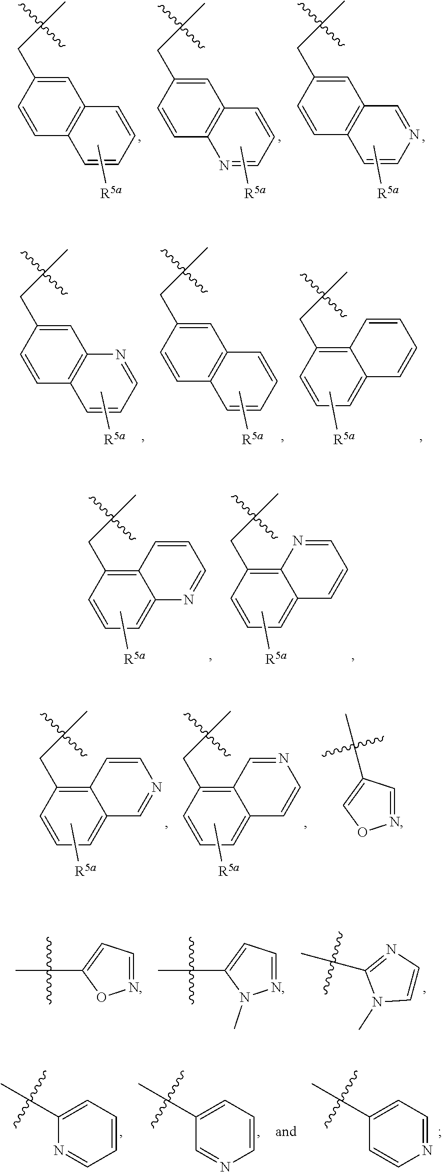
wherein [symbol] is the point of attachment to the rest of the compound.

13. The compound of claim 1, wherein R³ is —OR^{a} and R^{a} is C₁-₆ alkyl.

14. The compound of claim 1, wherein the compound is selected from 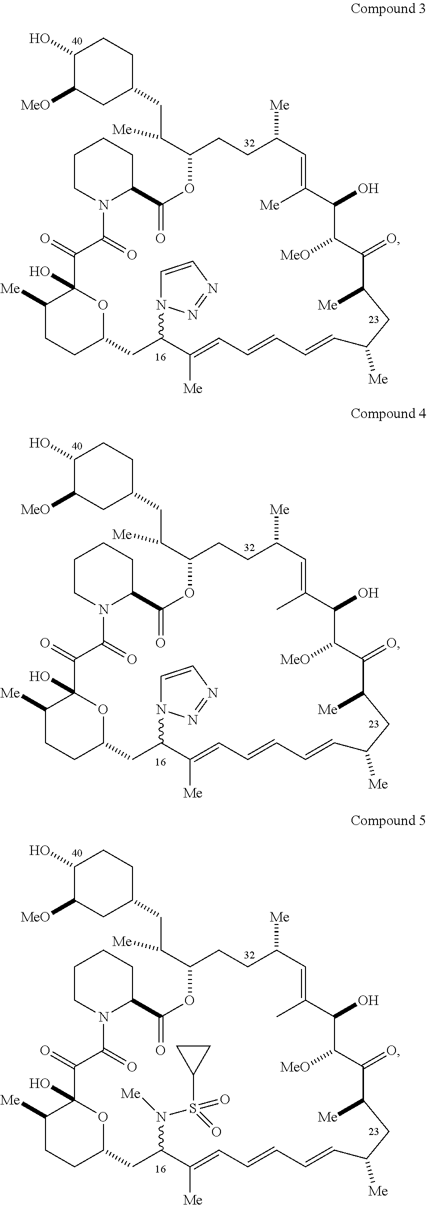 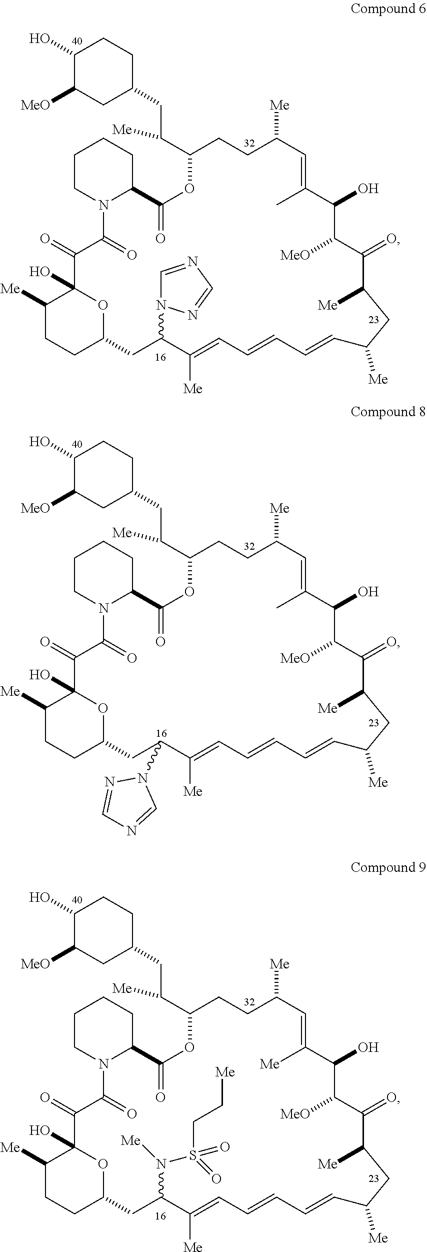 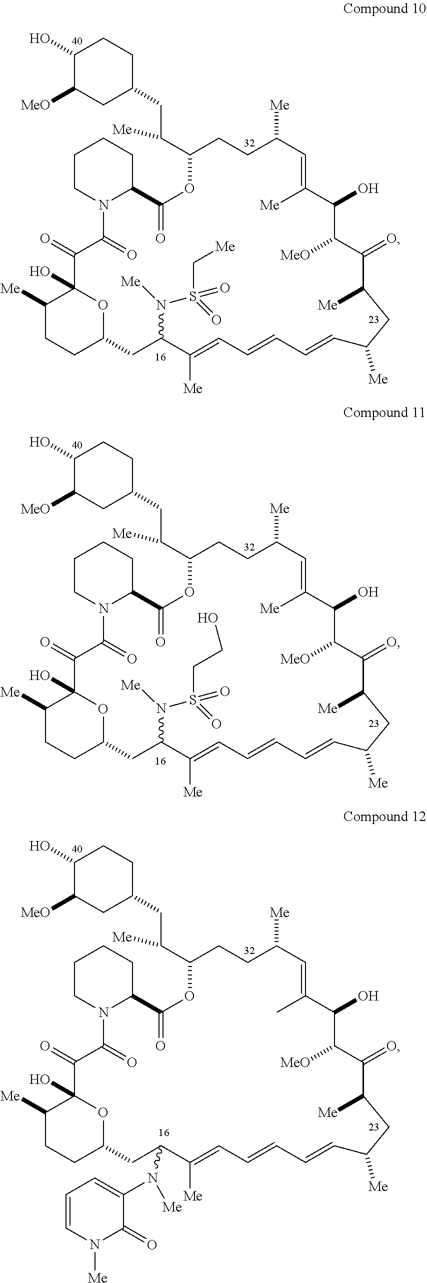 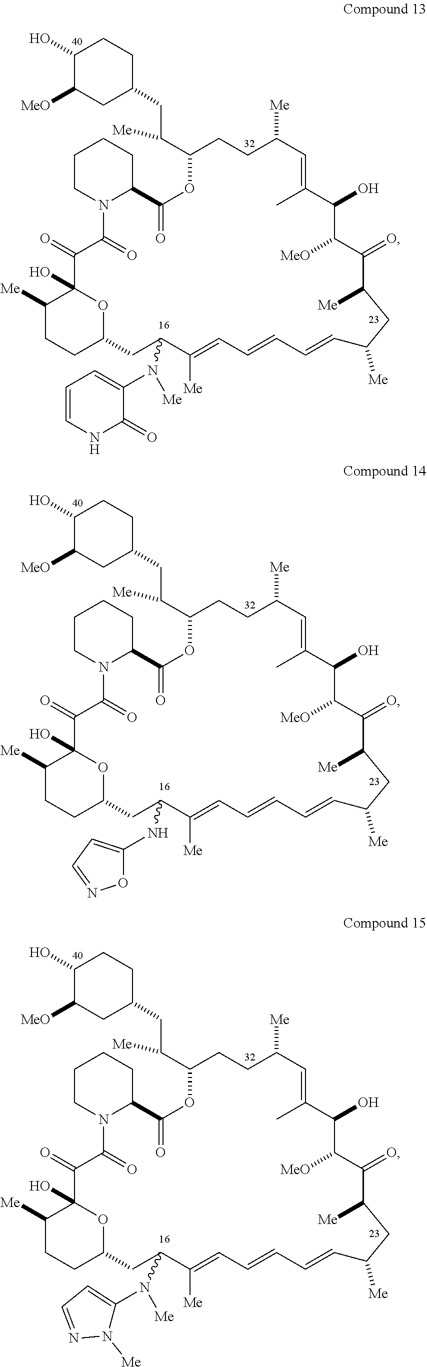 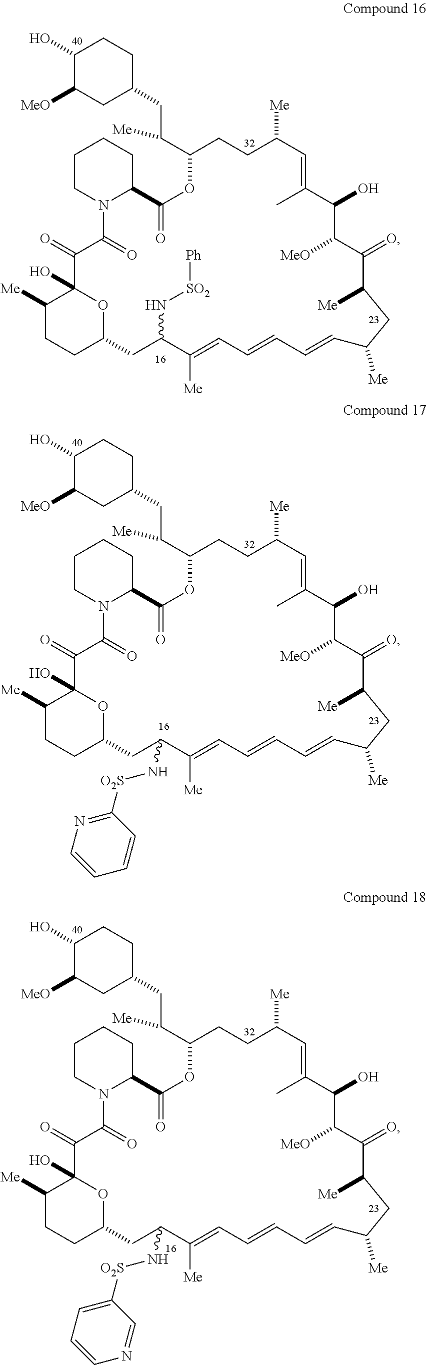 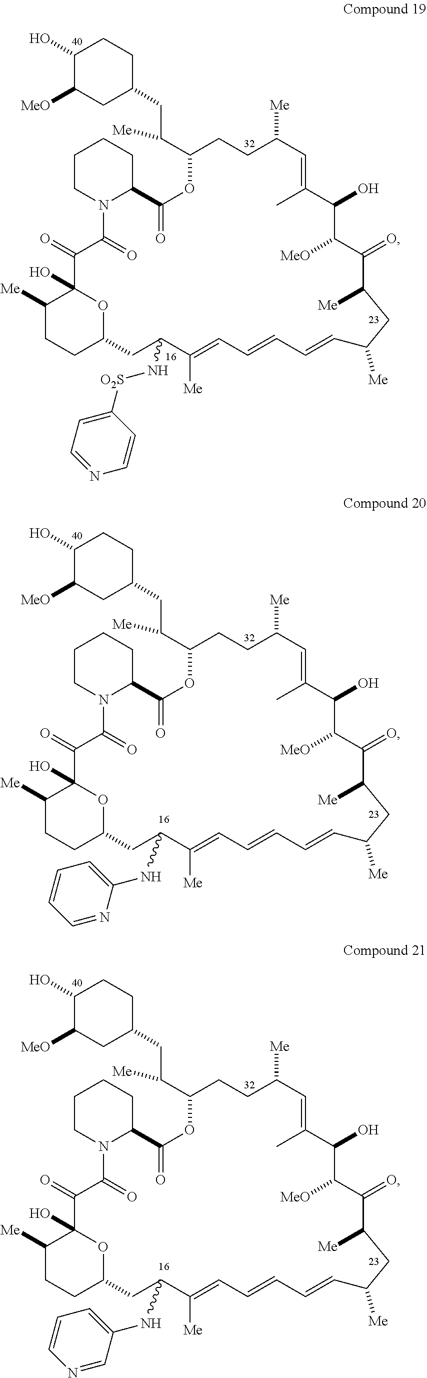 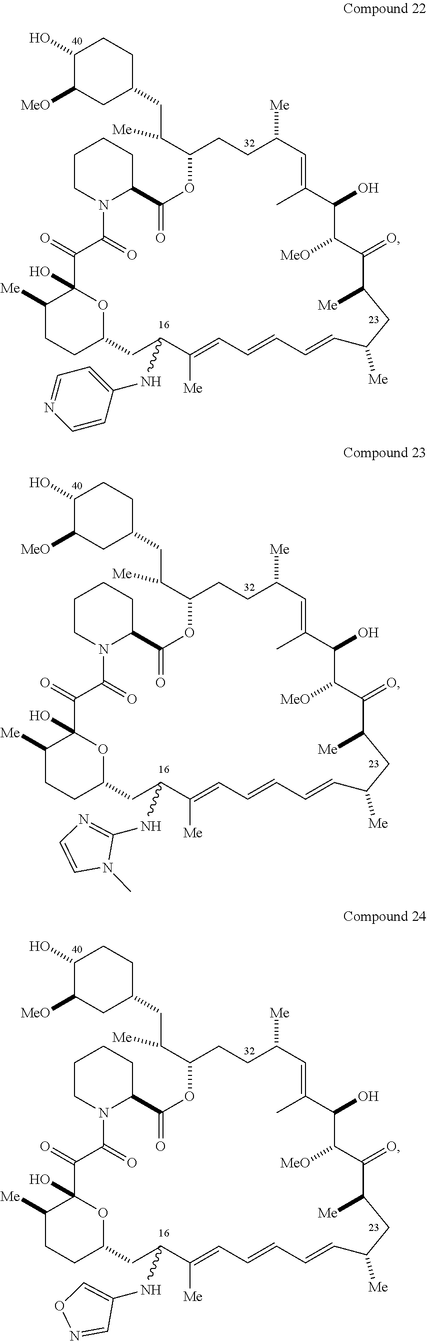 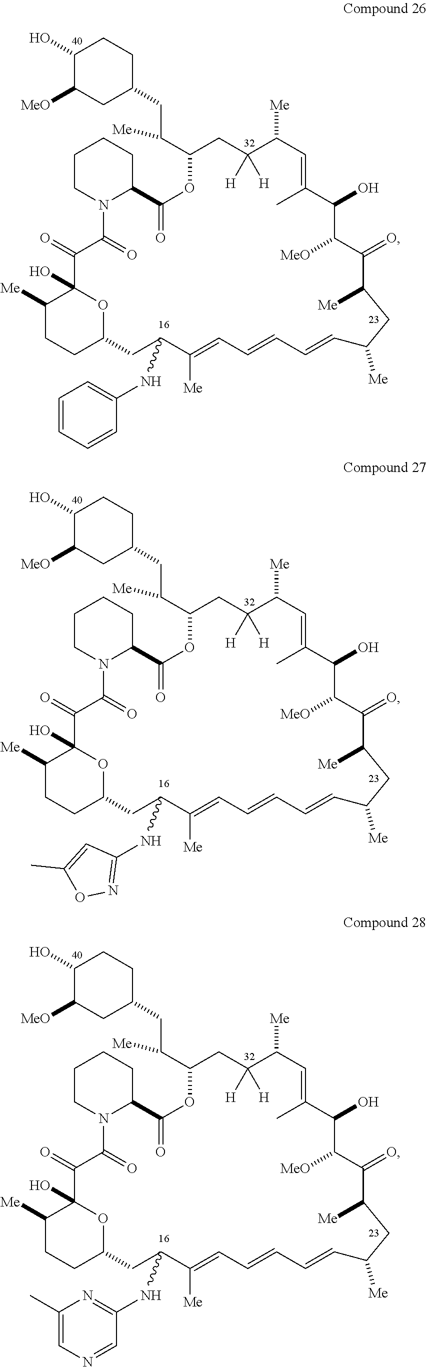 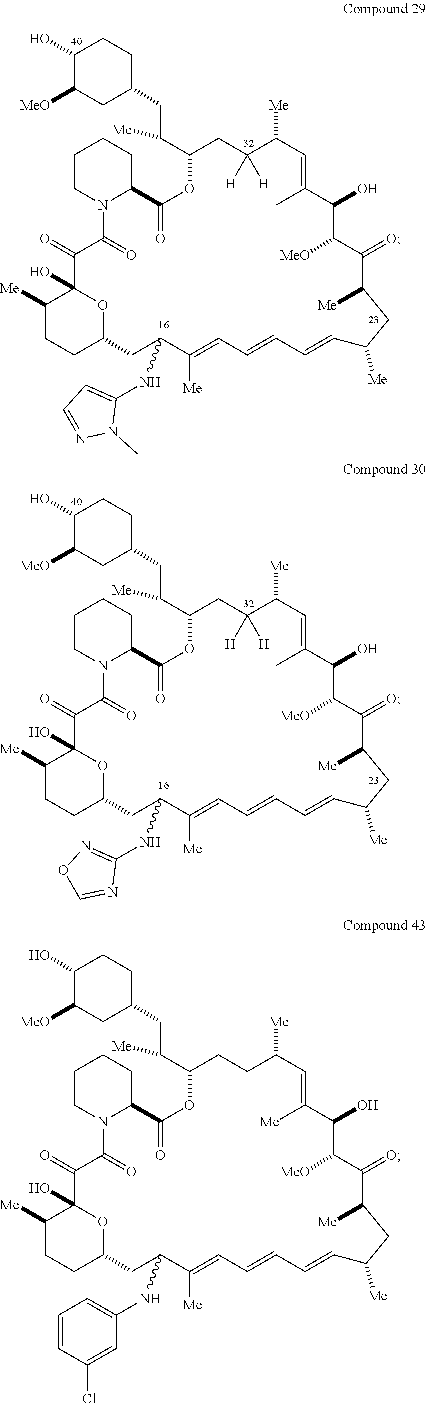 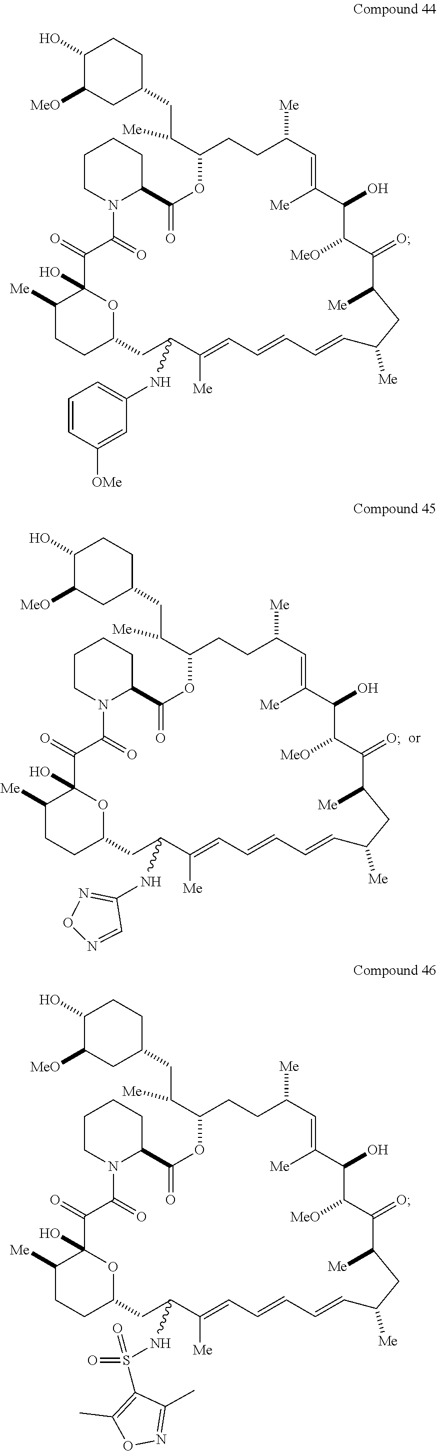 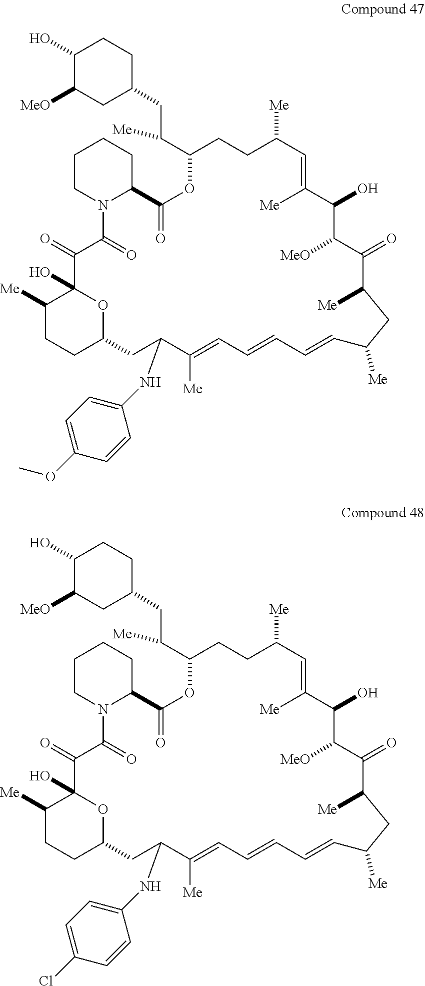 [SMILES: COc1ccc(NC2C[C@@H]3CC[C@@H](C)[C@@](O)(O3)C(=O)C(=O)N3CCCC[C@H]3C(=O)O[C@H]([C@H](C)C[C@H]3CC[C@@H](O)[C@H](OC)C3)CC[C@H](C)/C=C(\C)[C@@H](O)[C@@H](OC)C(=O)[C@H](C)C[C@H](C)/C=C/C=C/C=C/2C)cc1 ; CO[C@@H]1C[C@@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(Nc3ccc(Cl)cc3)C[C@@H]3CC[C@@H](C)[C@@](O)(O3)C(=O)C(=O)N3CCCC[C@H]3C(=O)O2)CC[C@H]1O] 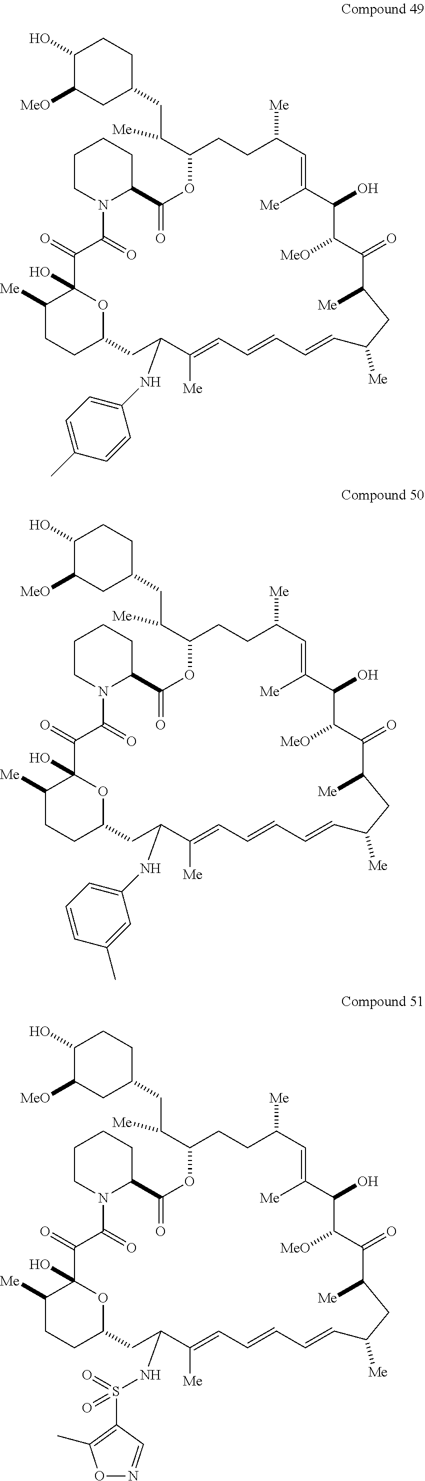 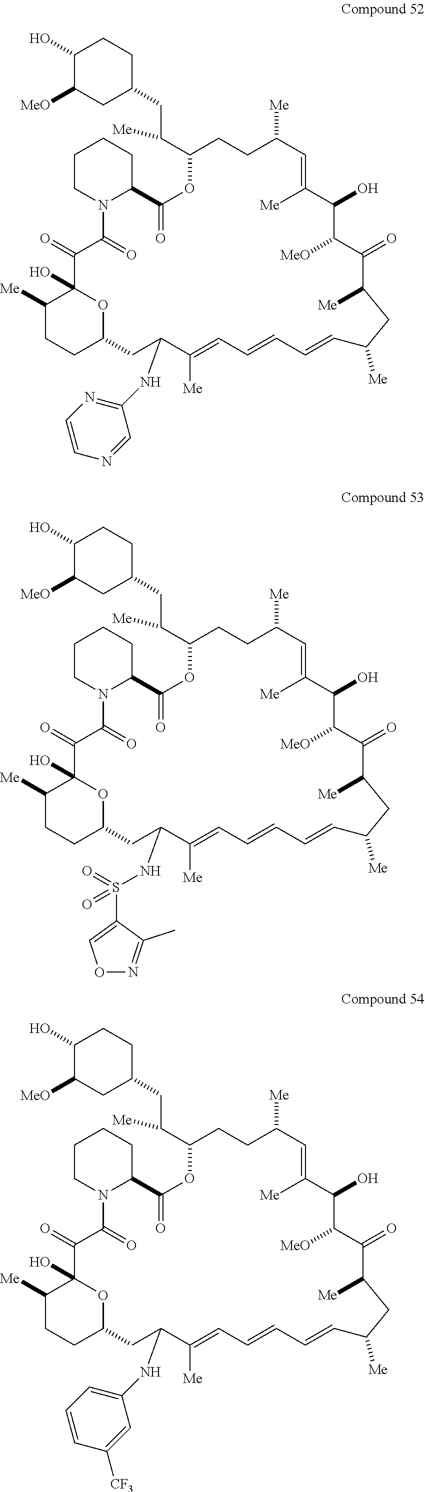 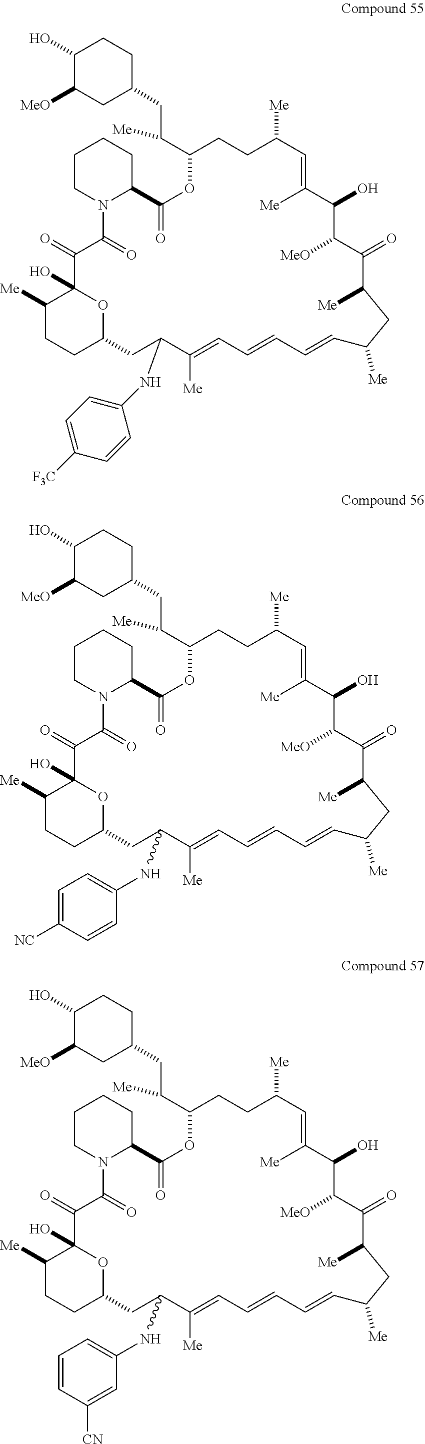 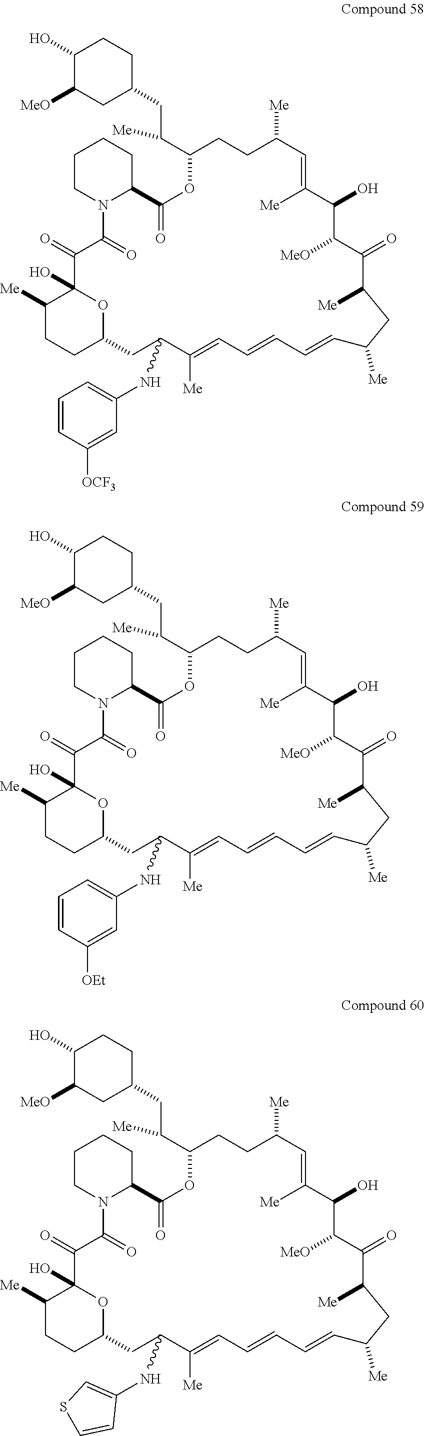 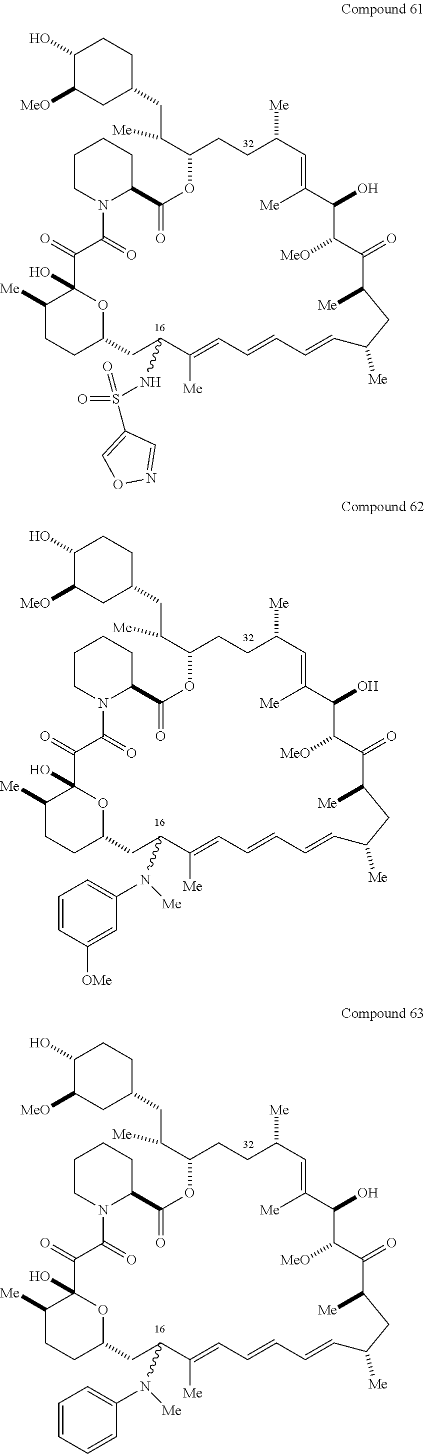 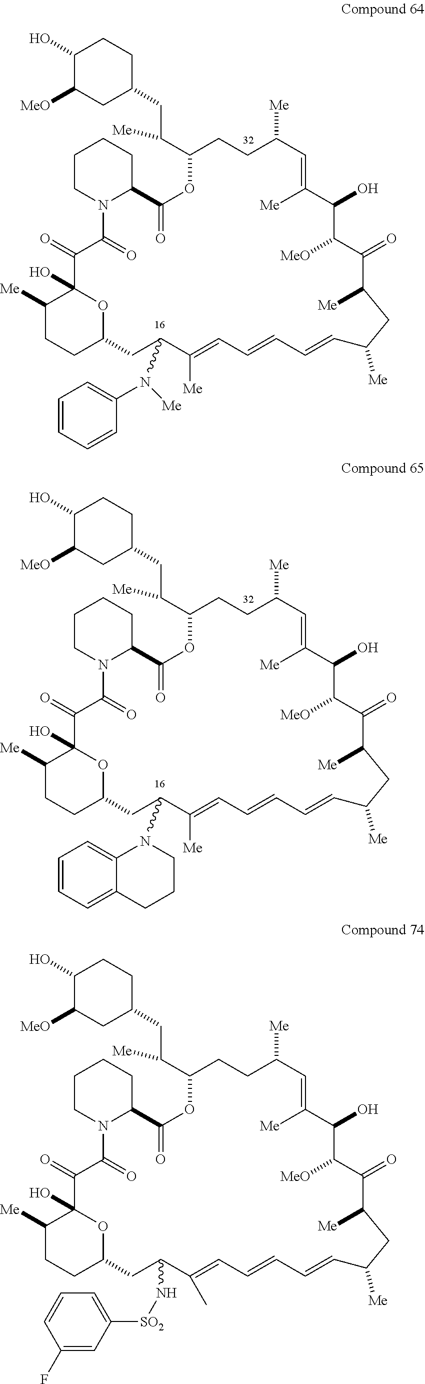 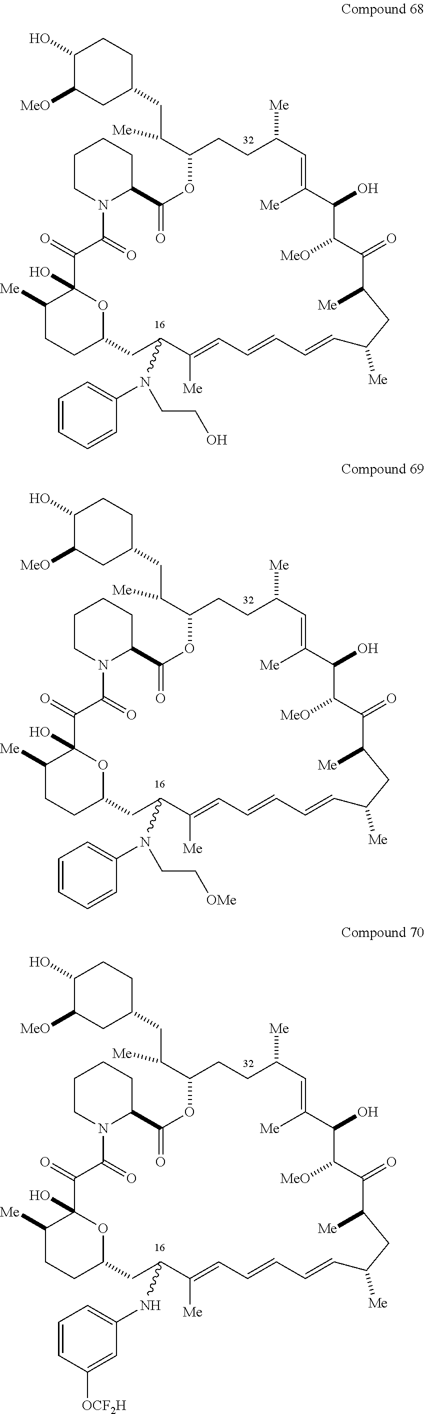 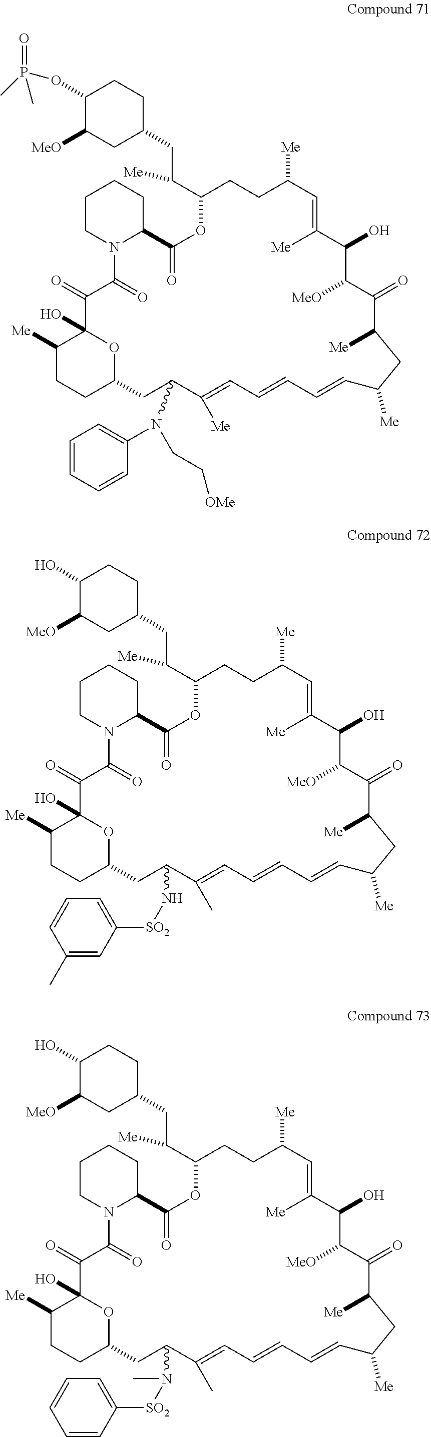 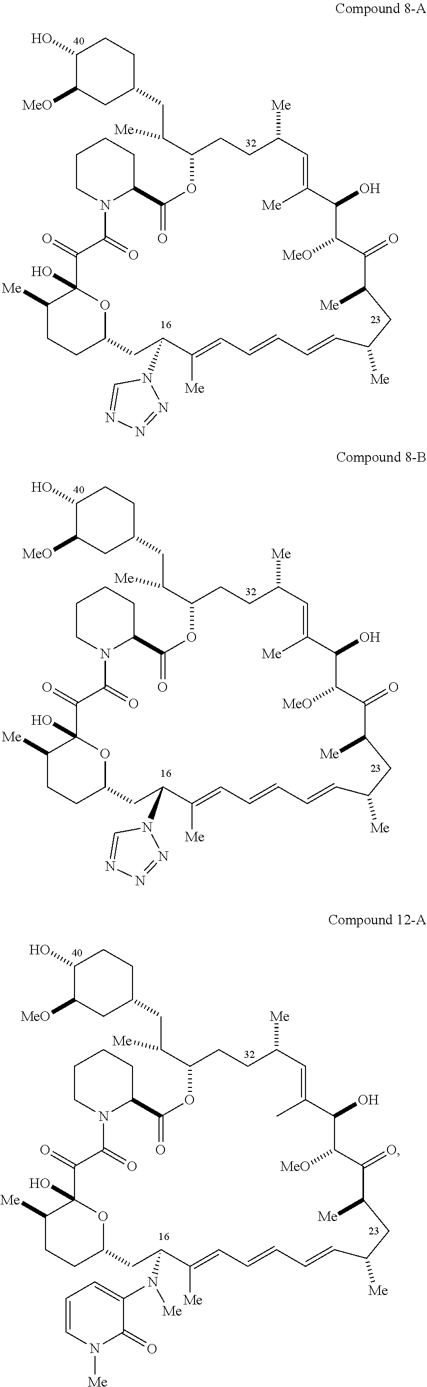 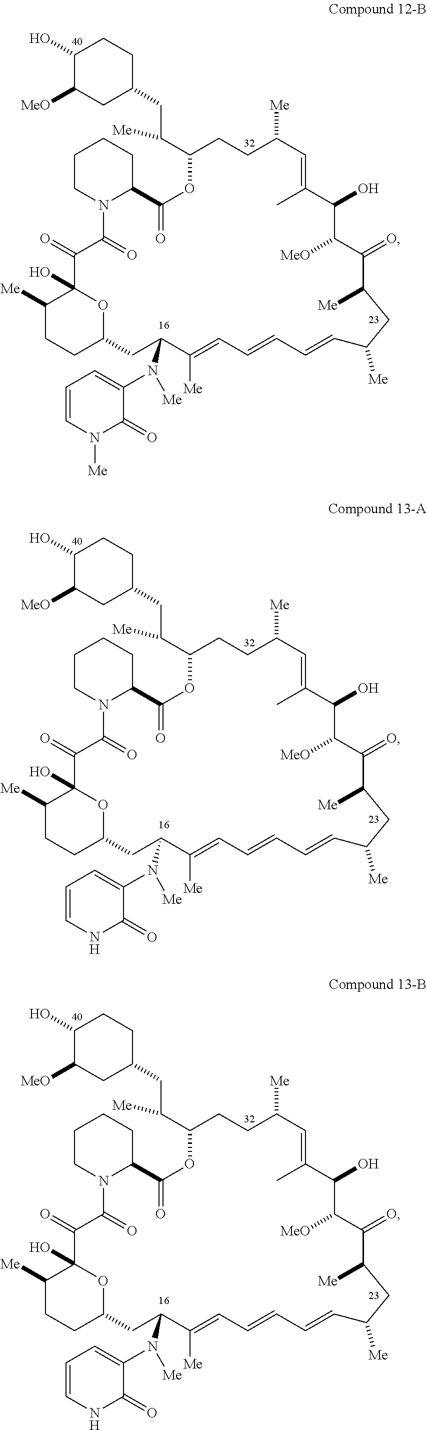 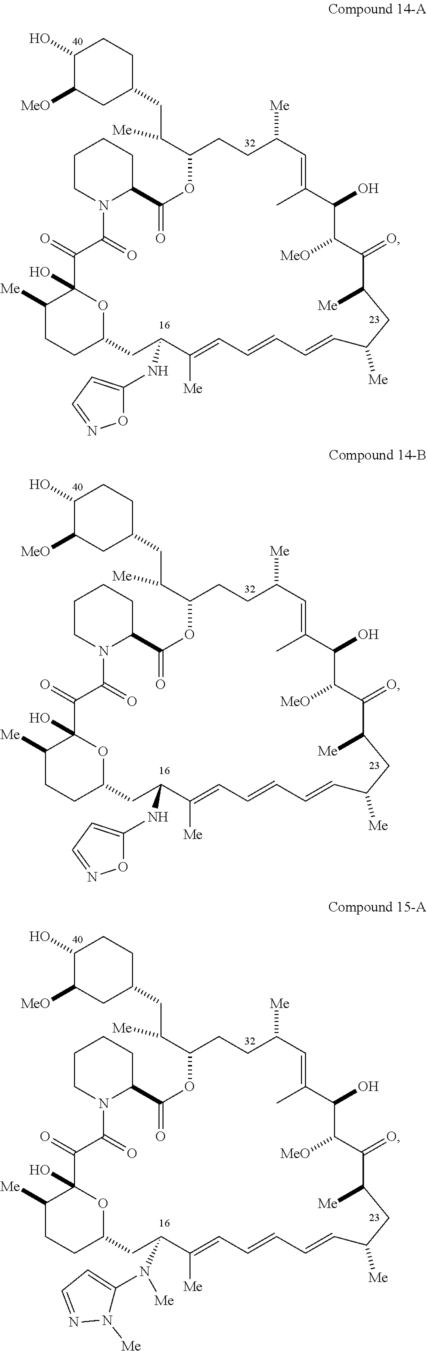 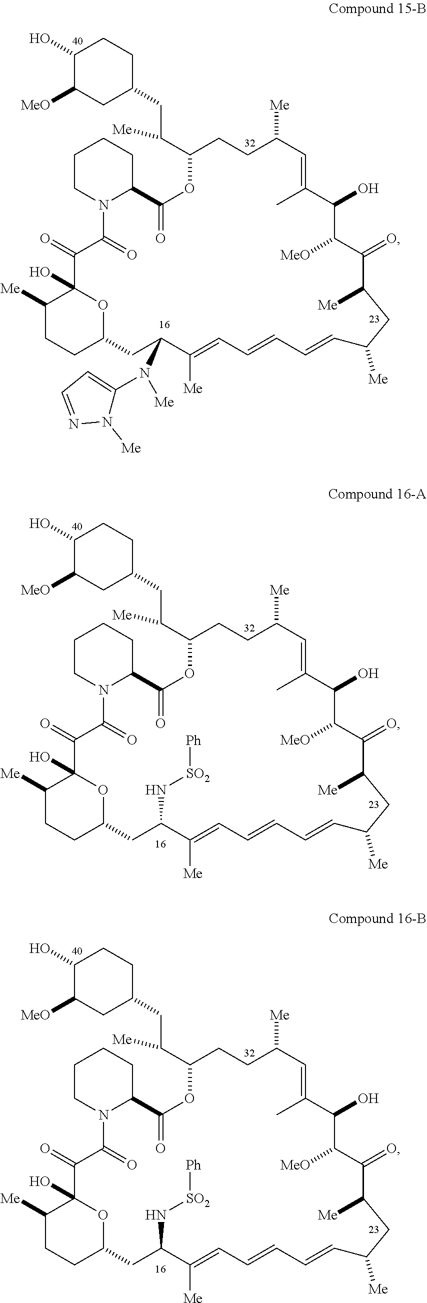 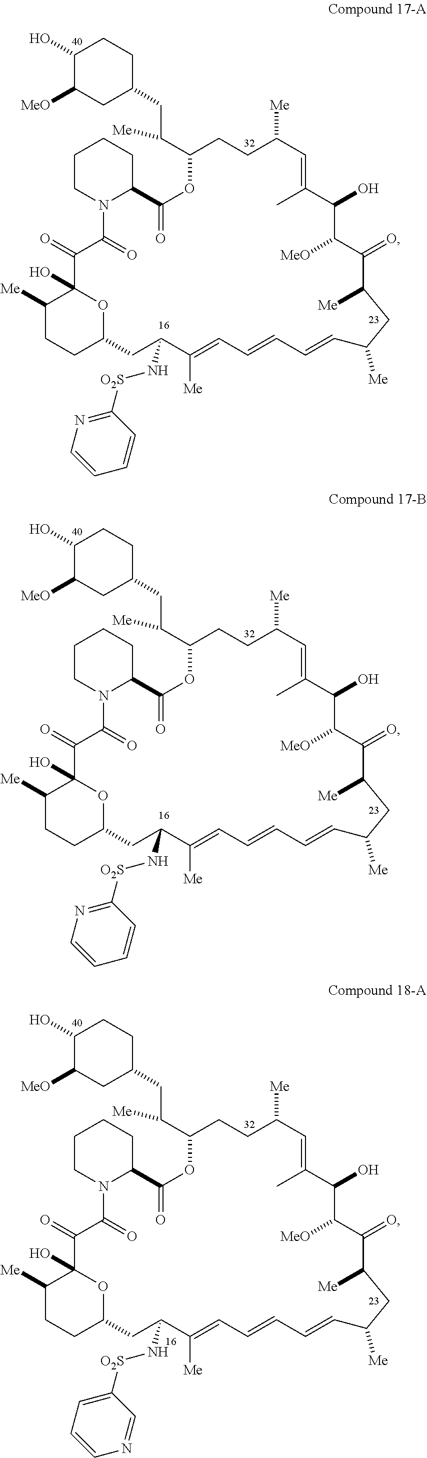 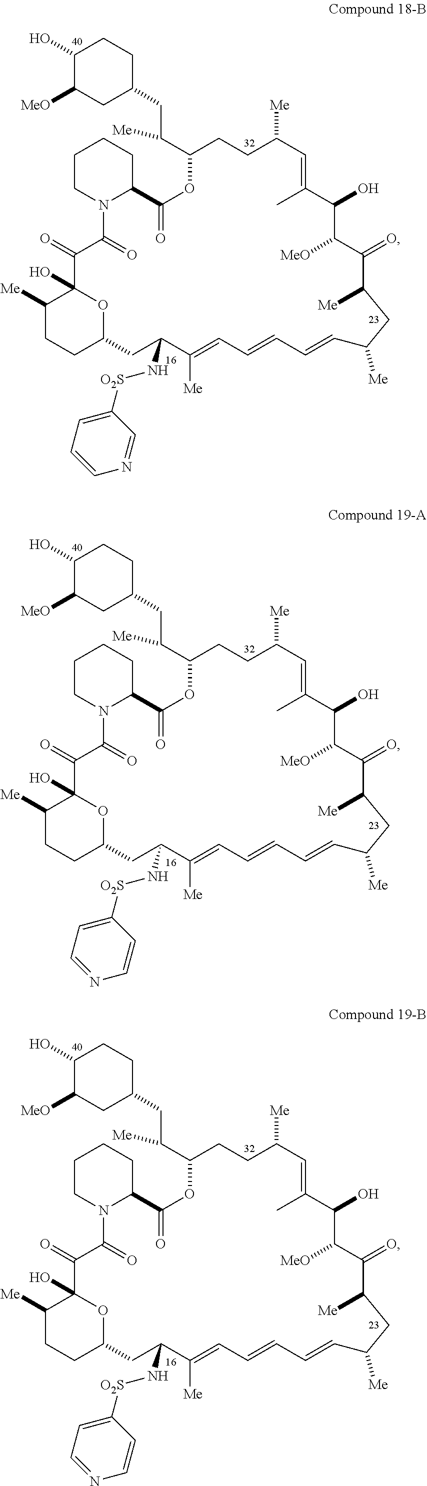 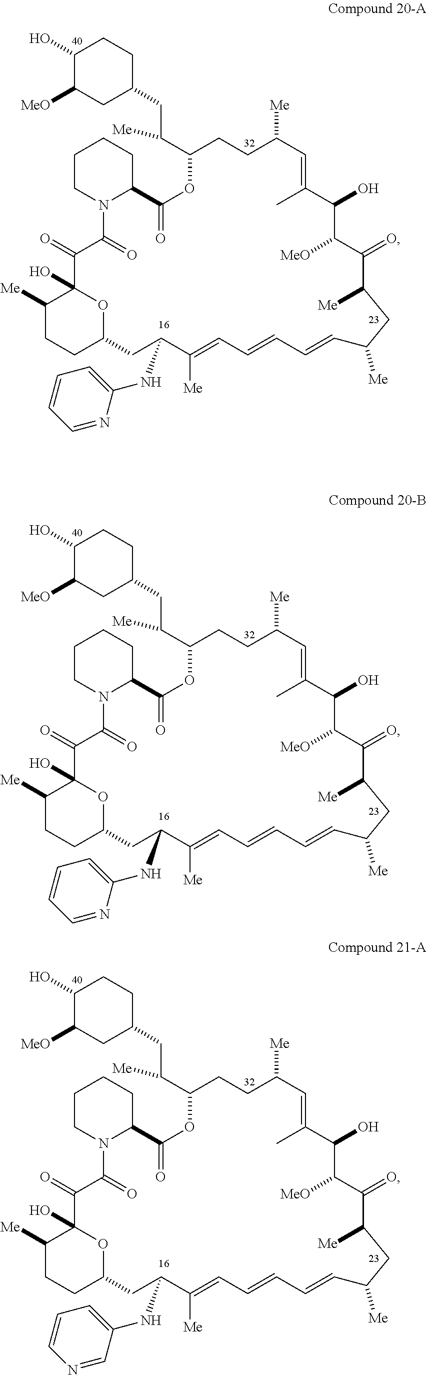 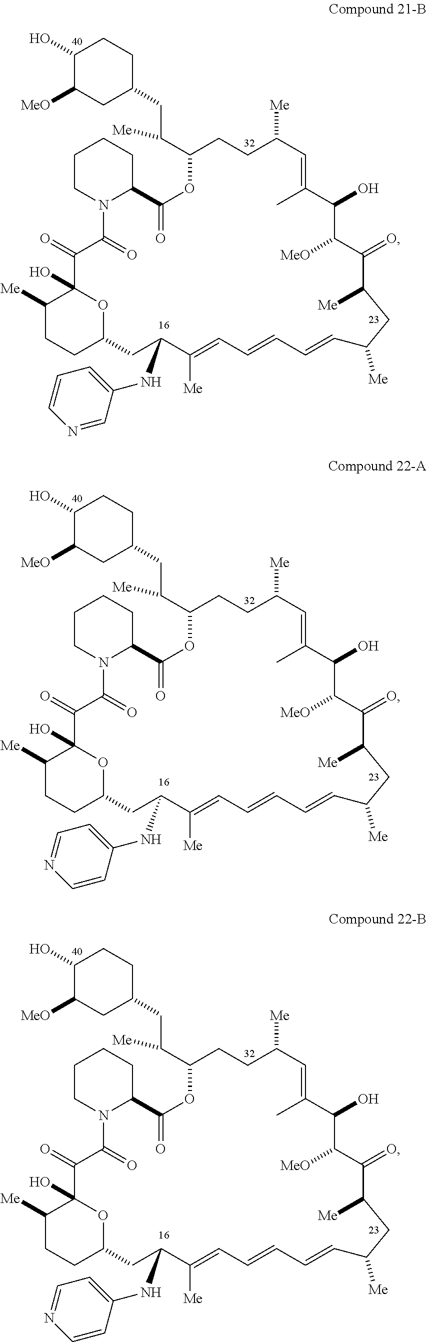 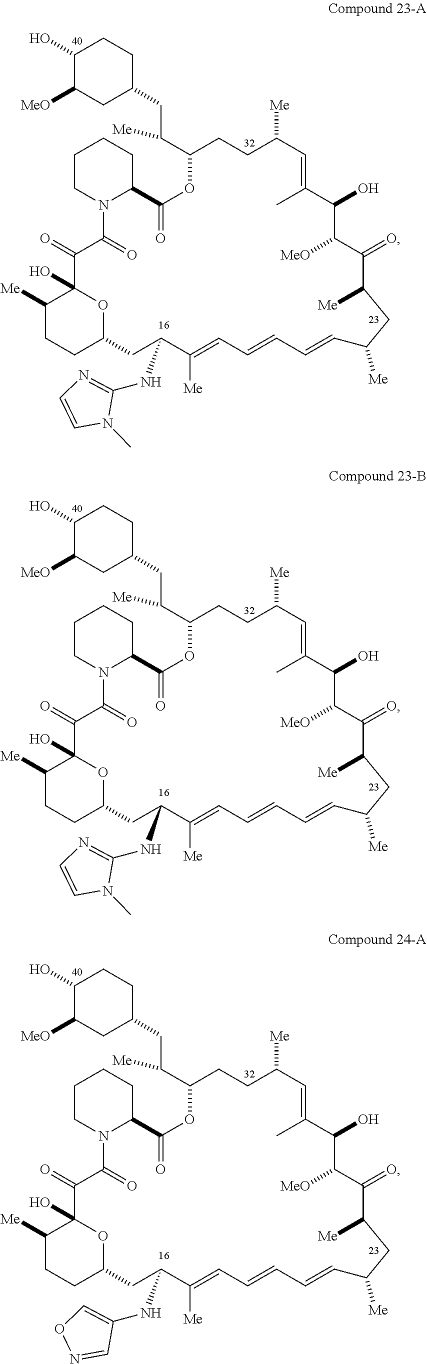 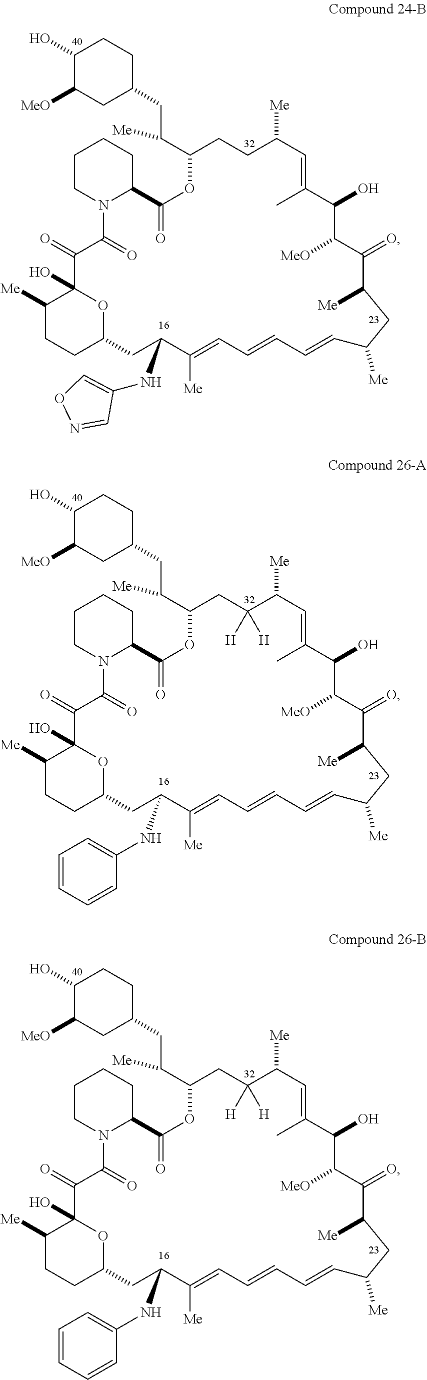 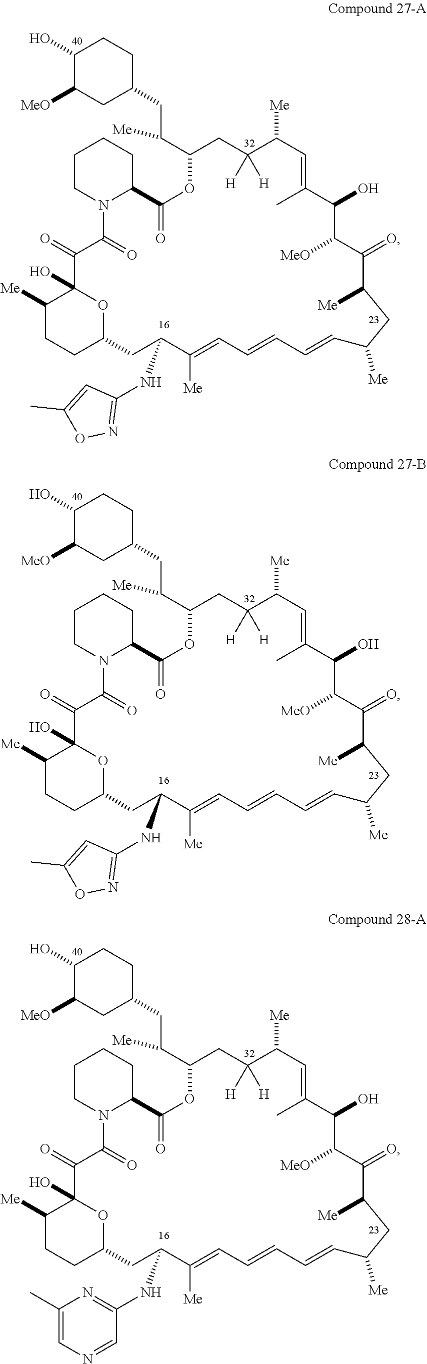 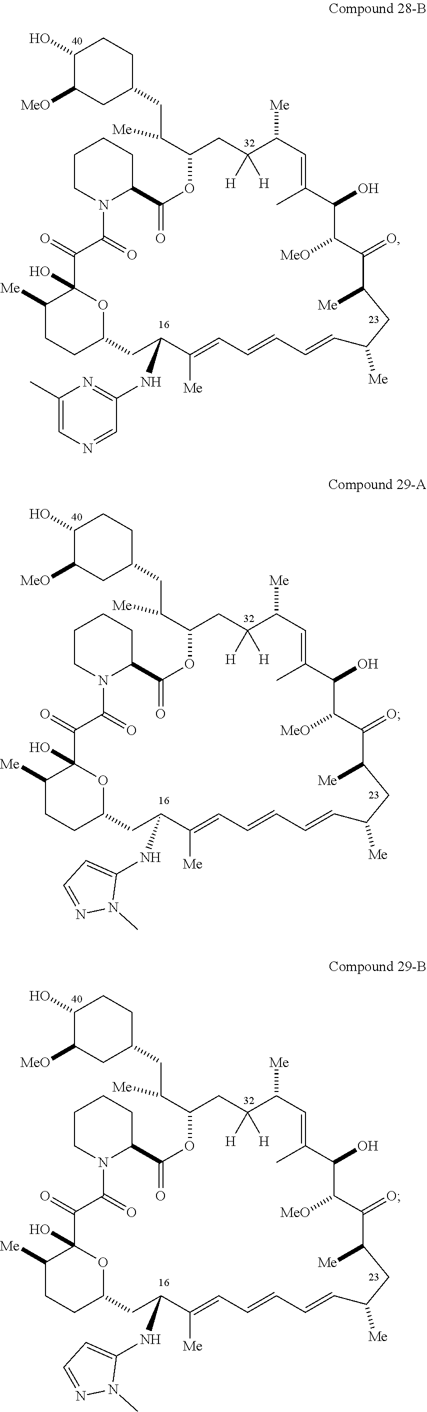 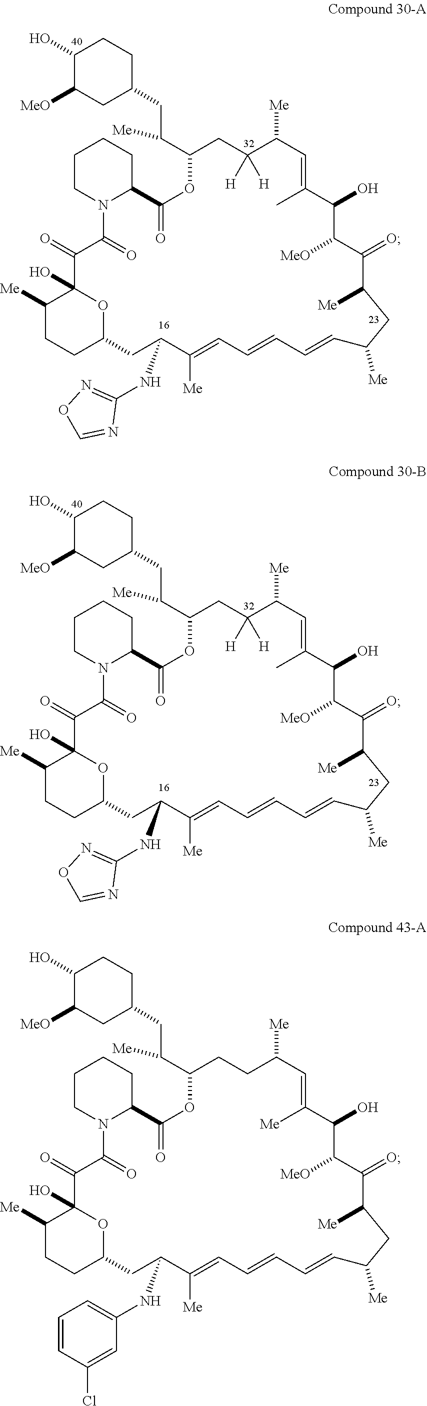 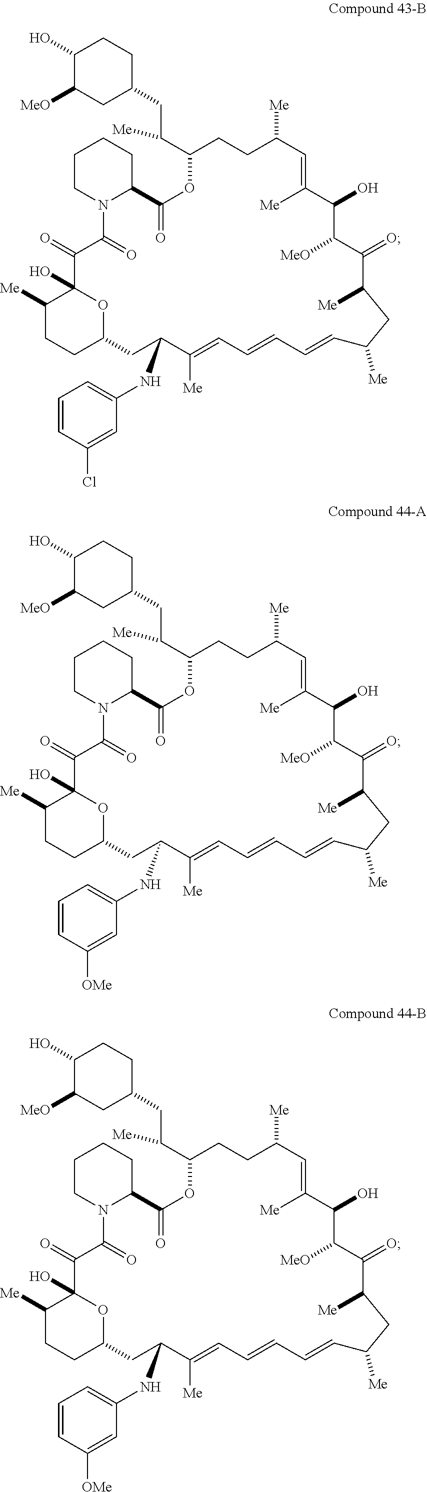 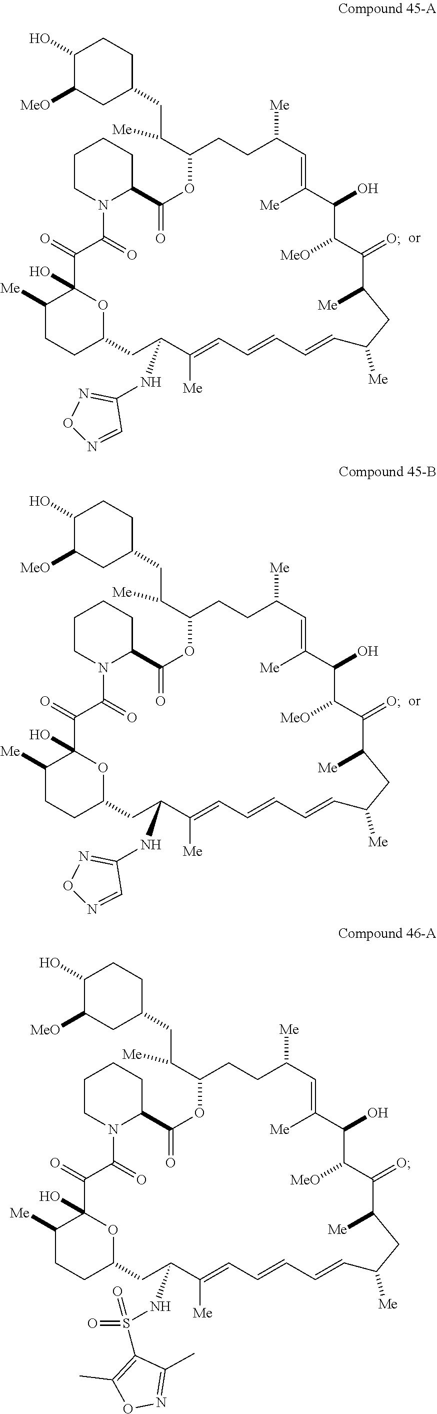 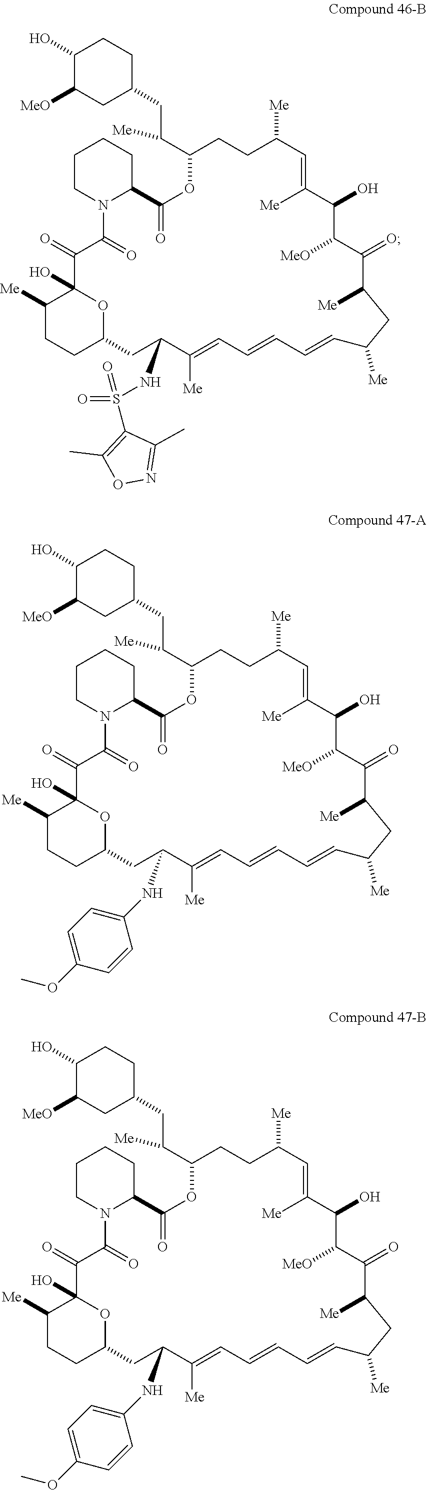 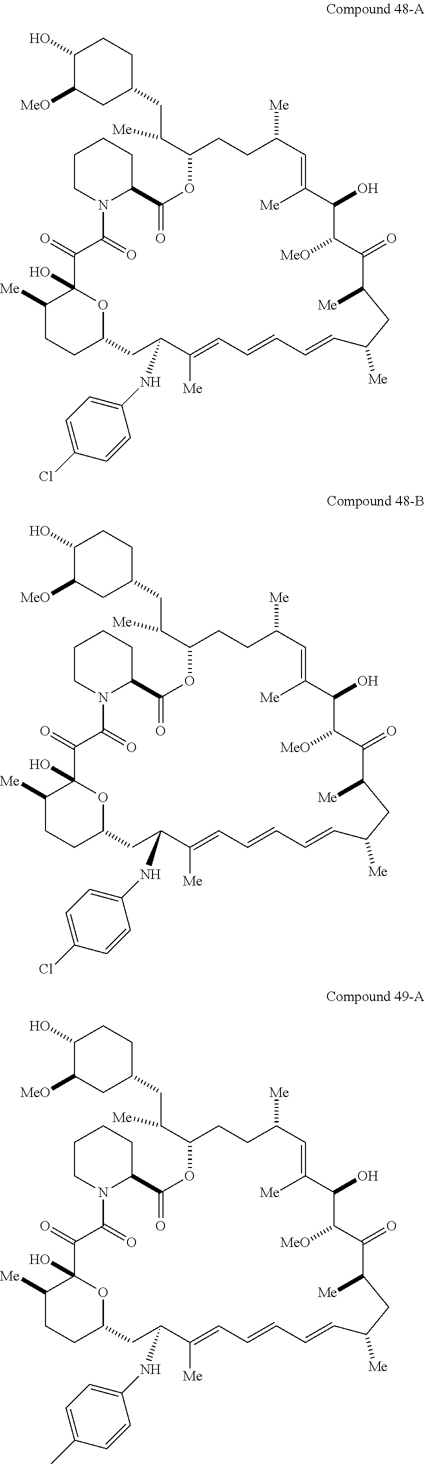 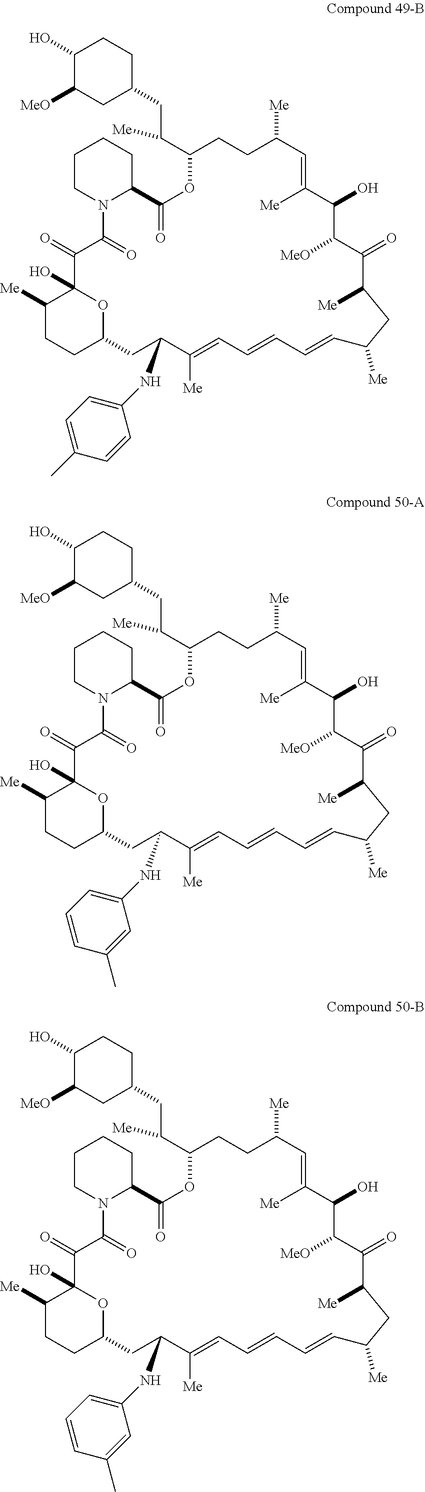 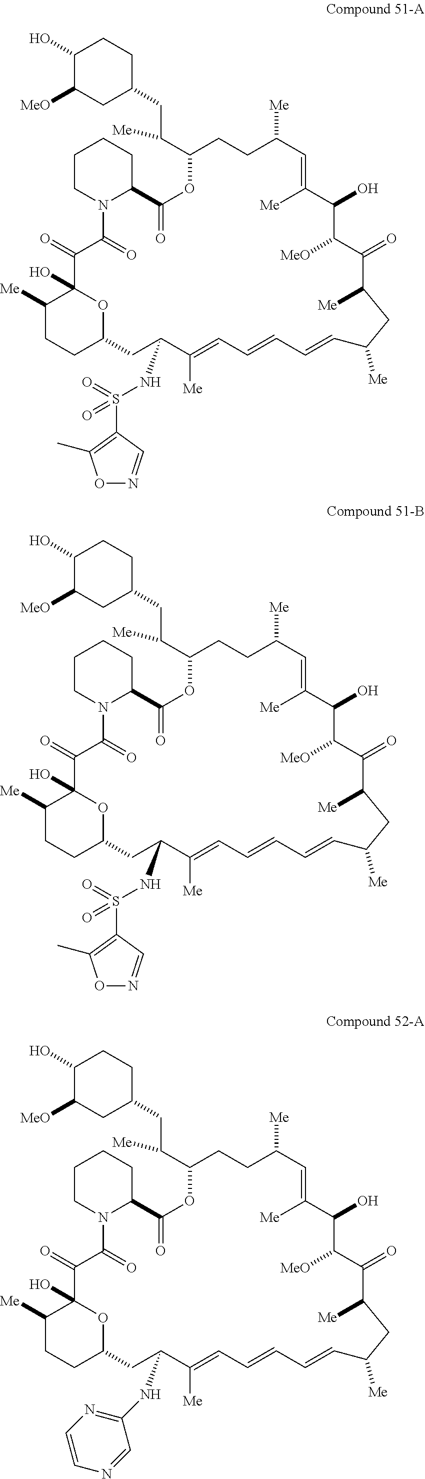 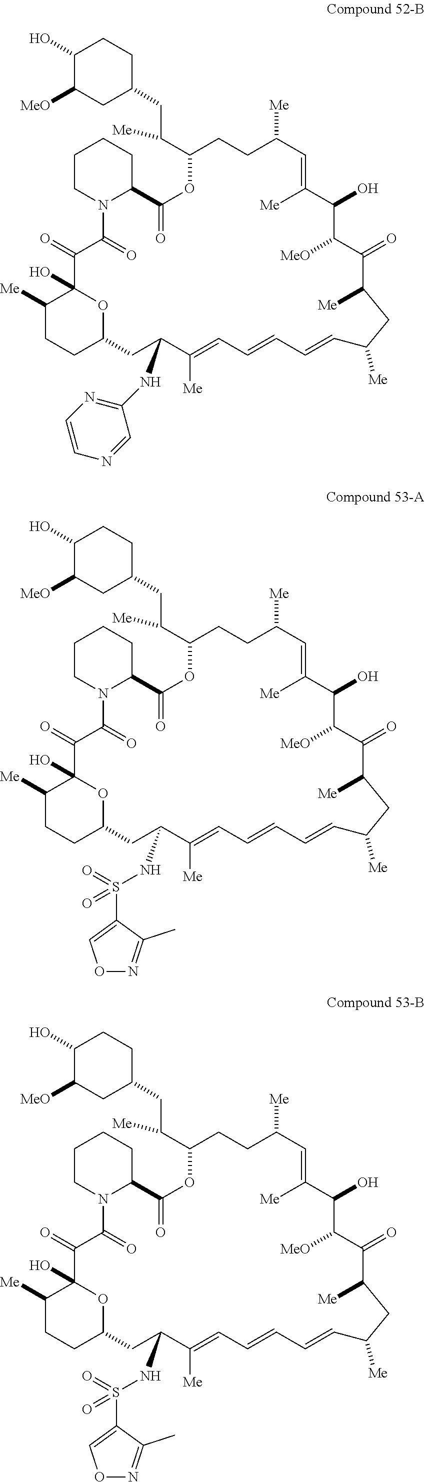 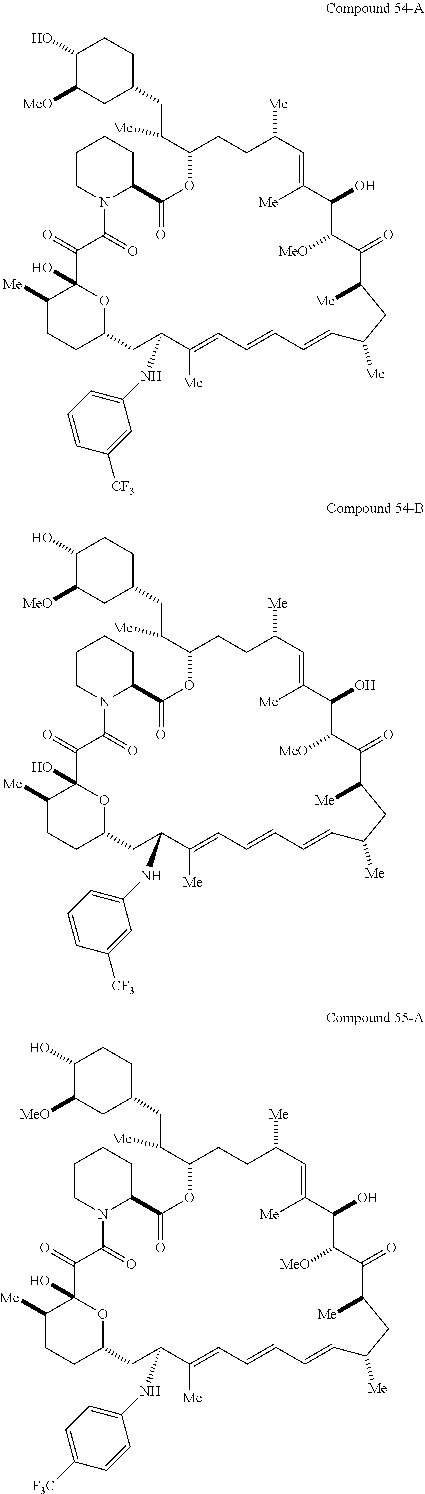 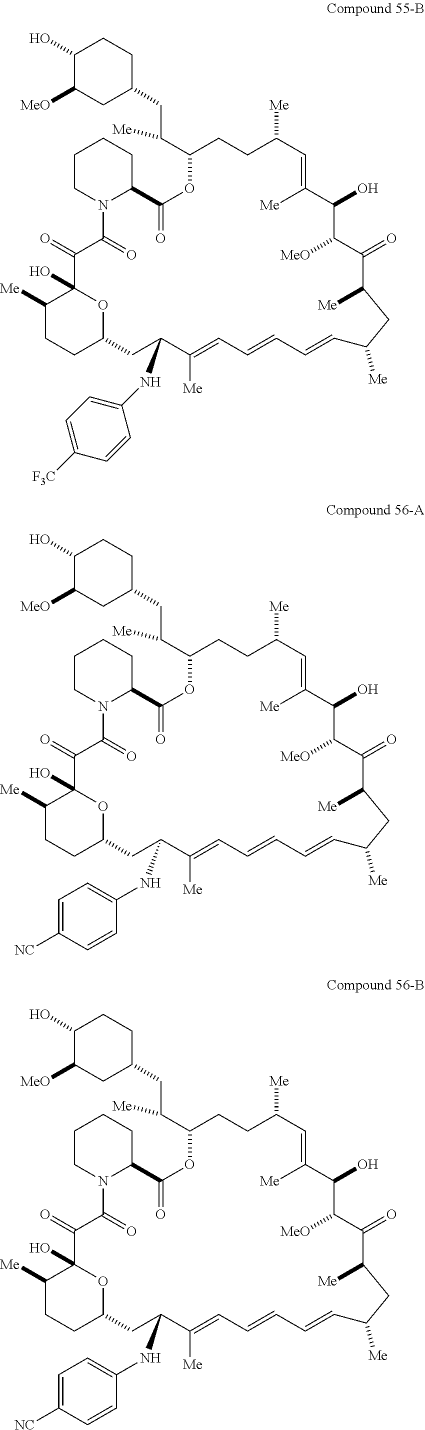 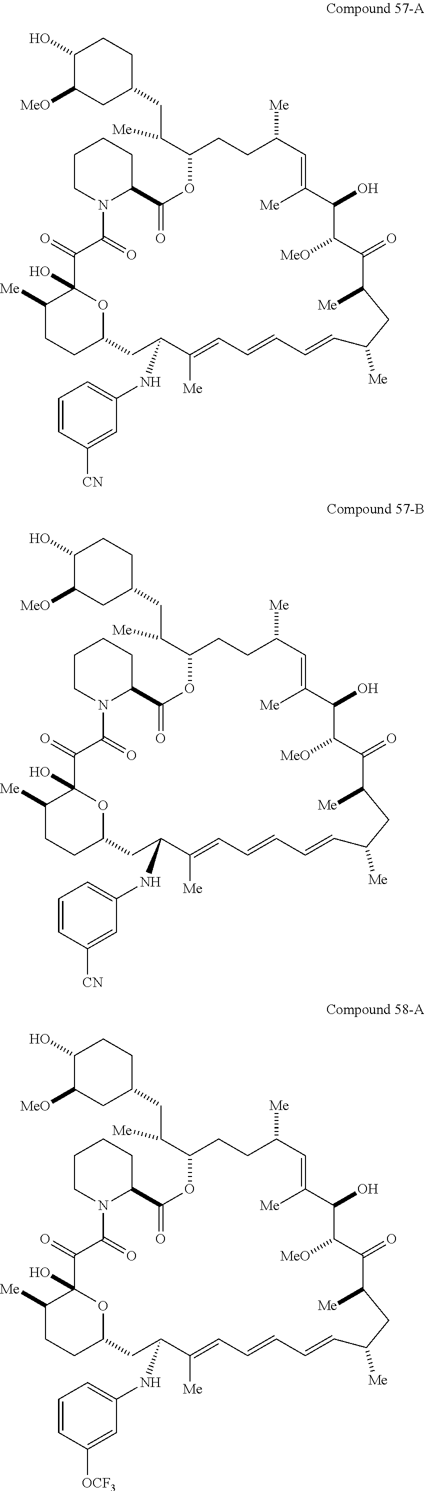 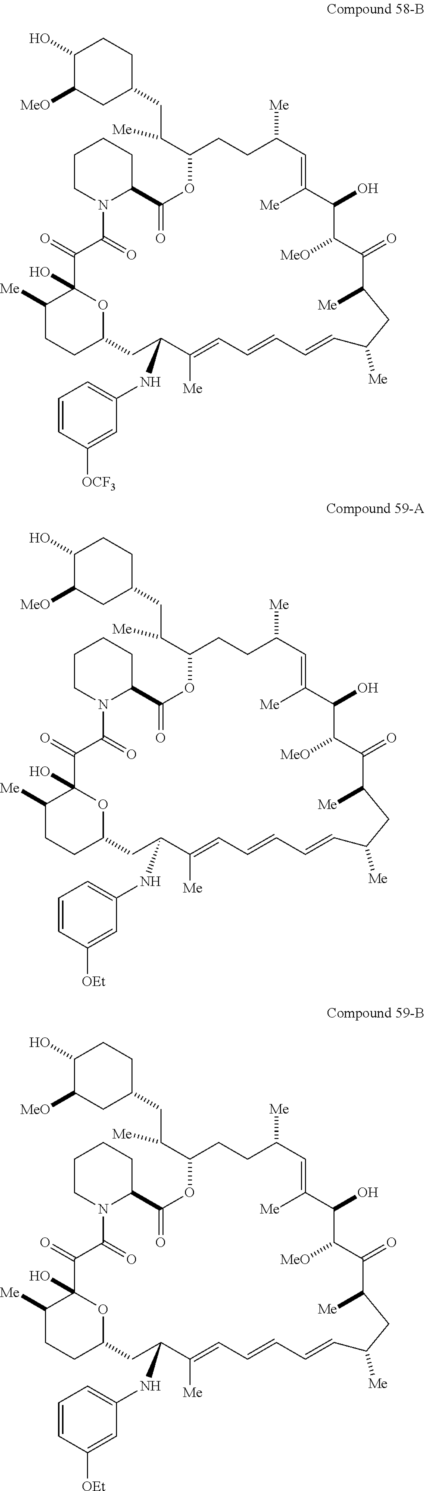 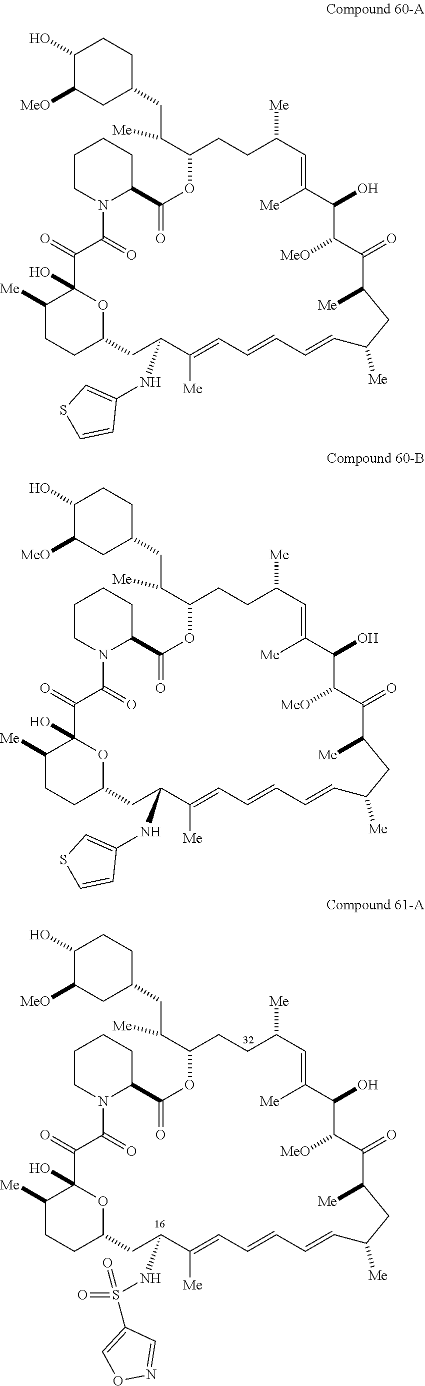 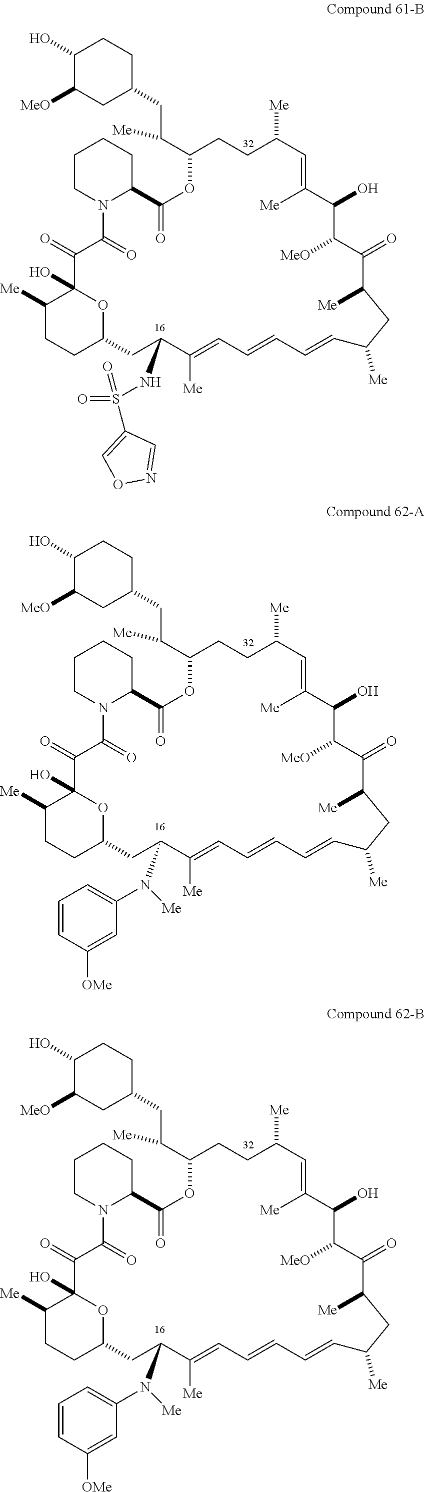 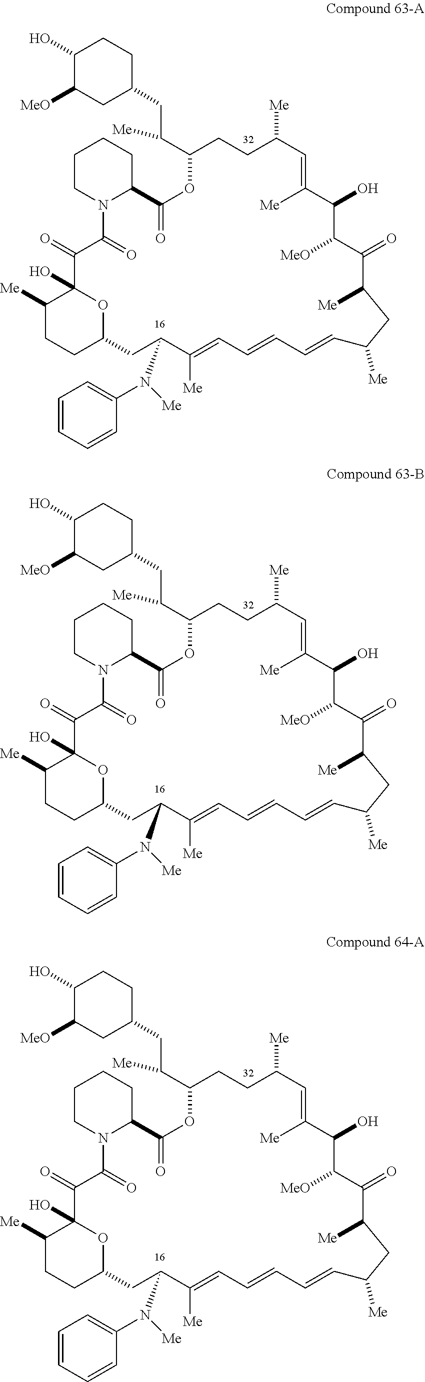 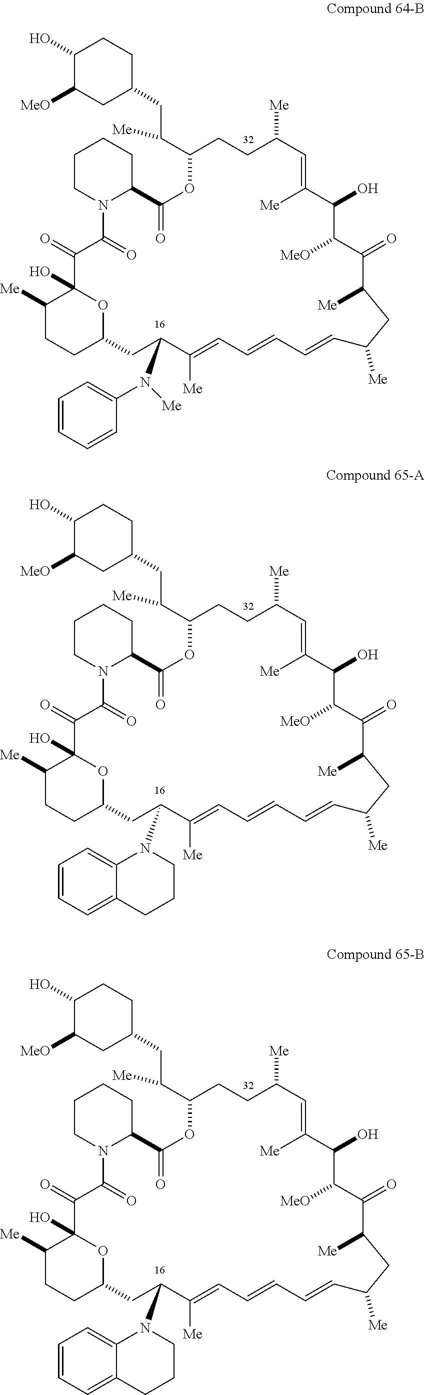 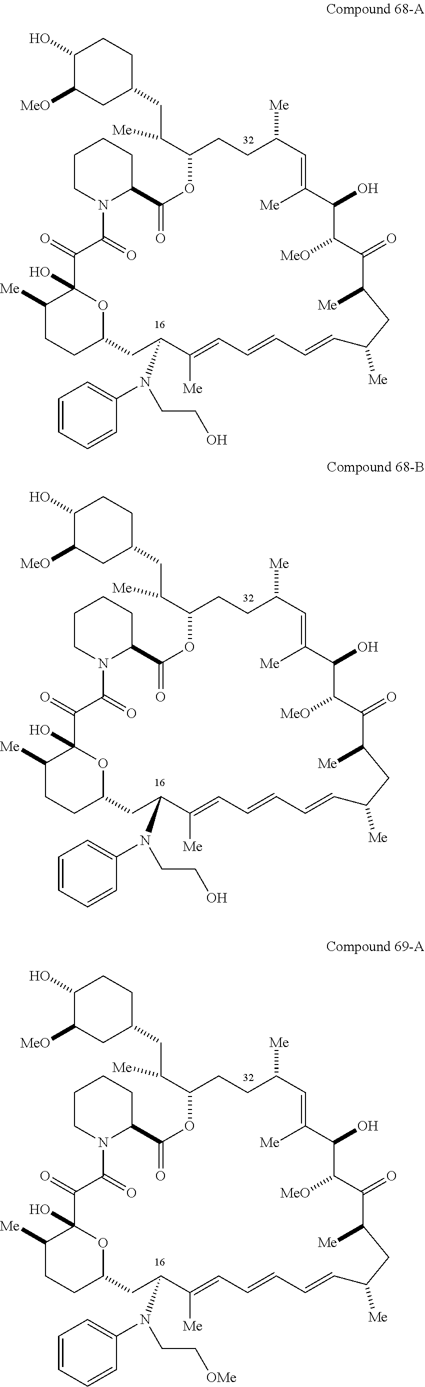 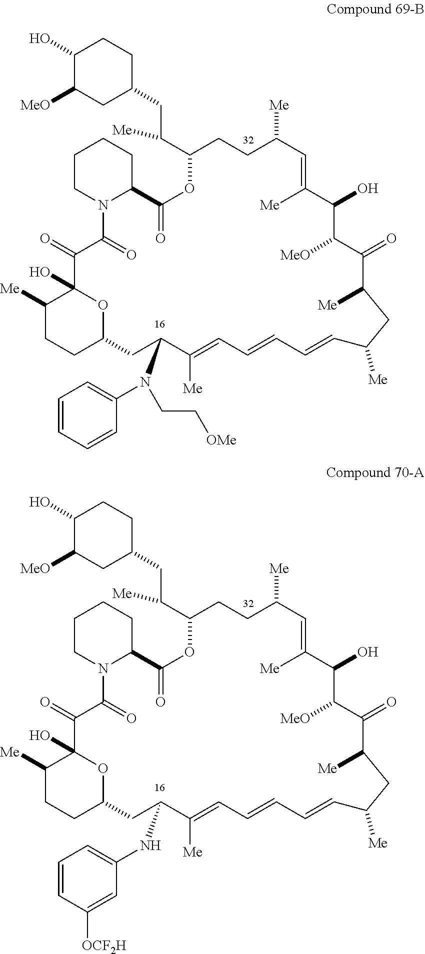 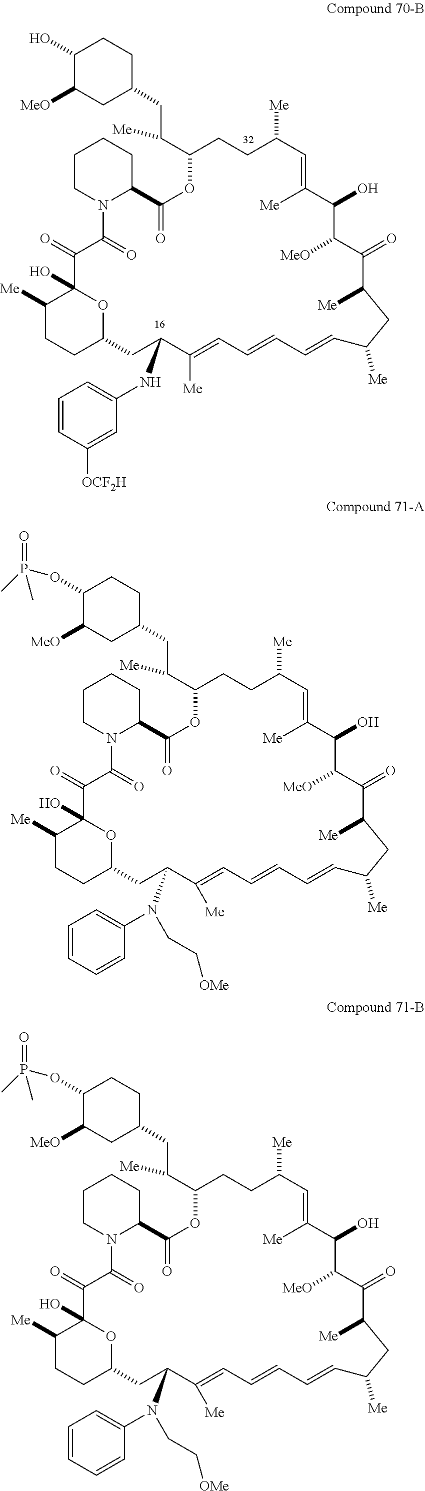 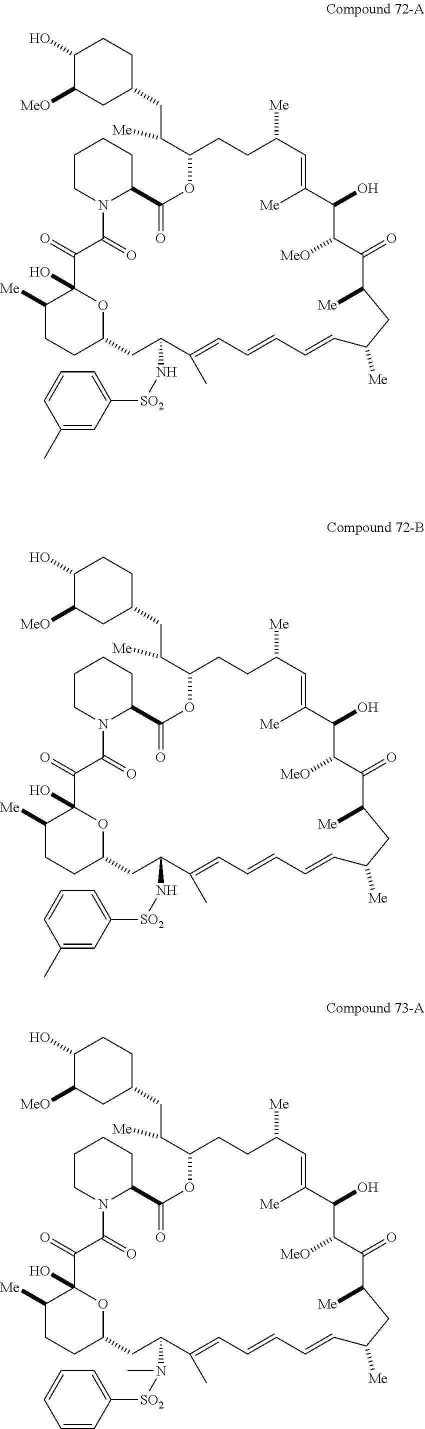 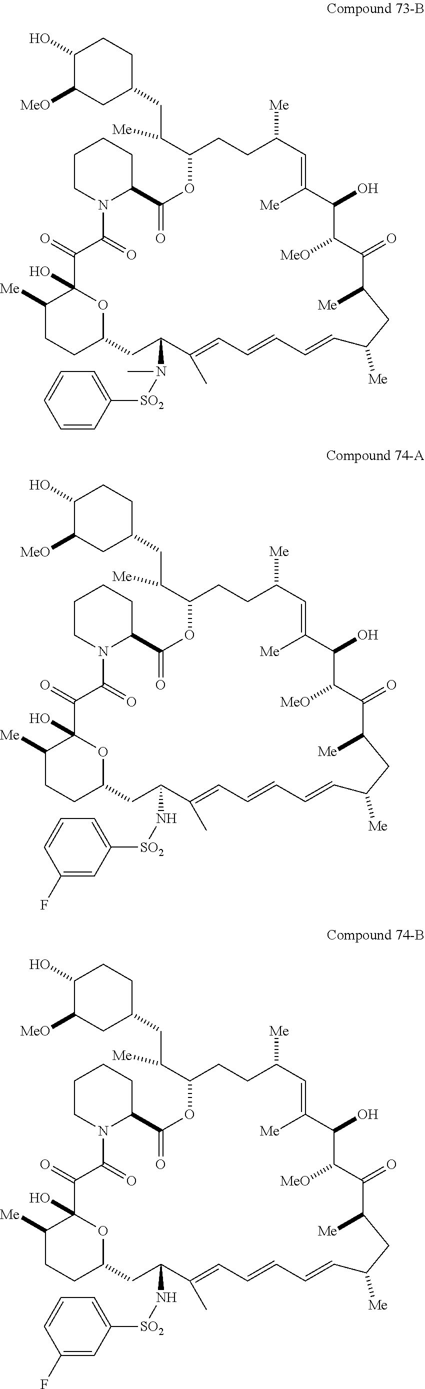
or a pharmaceutically acceptable salt thereof.

15. The compound of claim 14, wherein the compound is 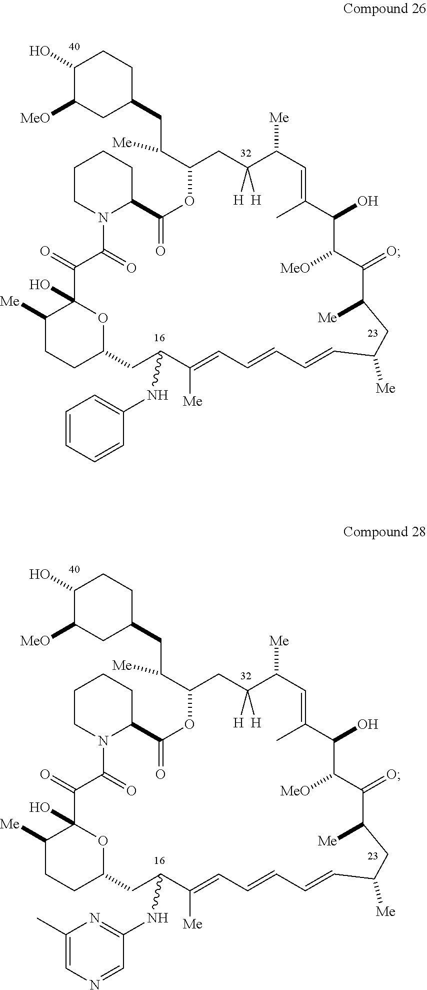 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(Nc3ccccc3)C[C@@H]3CC[C@@H](C)[C@@](O)(O3)C(=O)C(=O)N3CCCC[C@H]3C(=O)O2)CC[C@H]1O ; CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(Nc3cncc(C)n3)C[C@@H]3CC[C@@H](C)[C@@](O)(O3)C(=O)C(=O)N3CCCC[C@H]3C(=O)O2)CC[C@H]1O] 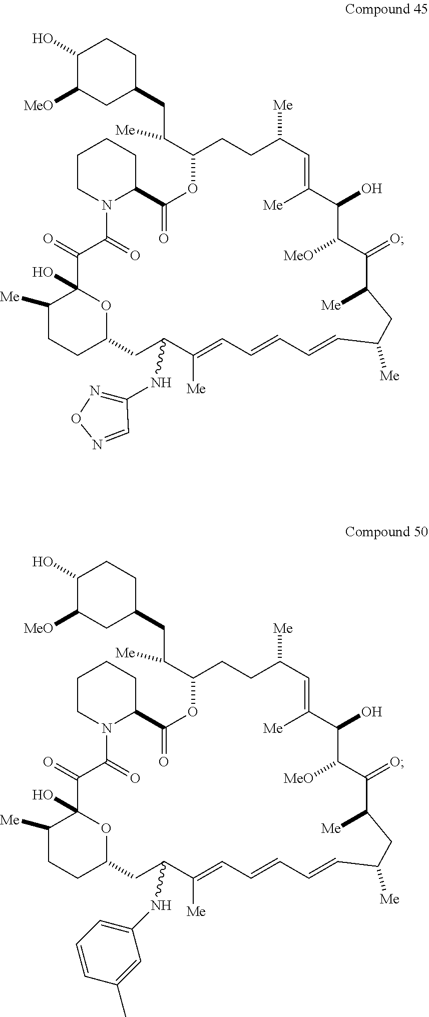 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(Nc3cnon3)C[C@@H]3CC[C@@H](C)[C@@](O)(O3)C(=O)C(=O)N3CCCC[C@H]3C(=O)O2)CC[C@H]1O ; CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(Nc3cccc(C)c3)C[C@@H]3CC[C@@H](C)[C@@](O)(O3)C(=O)C(=O)N3CCCC[C@H]3C(=O)O2)CC[C@H]1O] 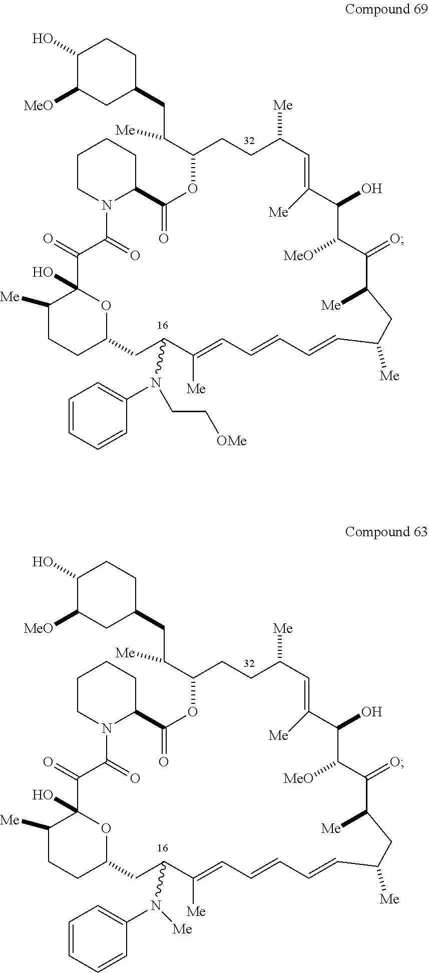 [SMILES: COCCN(c1ccccc1)C1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC[C@H](C)/C=C(\C)[C@@H](O)[C@@H](OC)C(=O)[C@H](C)C[C@H](C)/C=C/C=C/C=C/1C ; CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(N(C)c3ccccc3)C[C@@H]3CC[C@@H](C)[C@@](O)(O3)C(=O)C(=O)N3CCCC[C@H]3C(=O)O2)CC[C@H]1O] 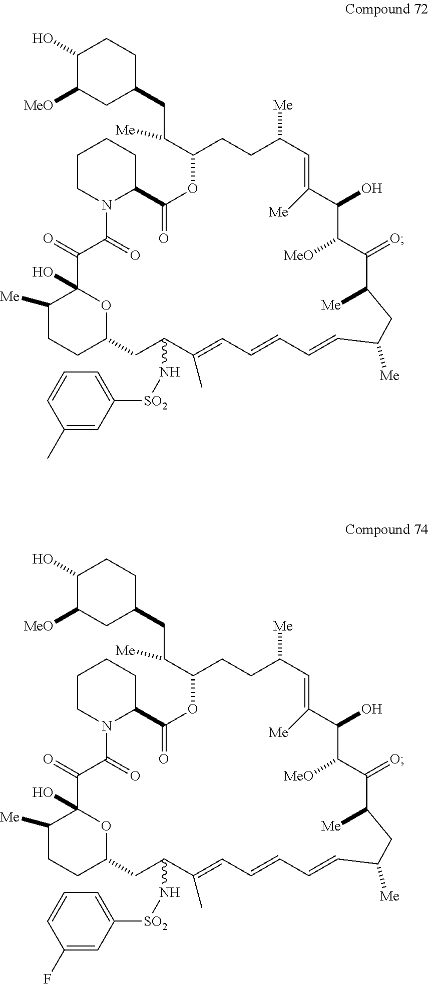 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(NS(=O)(=O)c3cccc(C)c3)C[C@@H]3CC[C@@H](C)[C@@](O)(O3)C(=O)C(=O)N3CCCC[C@H]3C(=O)O2)CC[C@H]1O ; CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(NS(=O)(=O)c3cccc(F)c3)C[C@@H]3CC[C@@H](C)[C@@](O)(O3)C(=O)C(=O)N3CCCC[C@H]3C(=O)O2)CC[C@H]1O]
or a pharmaceutically acceptable salt thereof.

16. A compound selected from the group consisting of: 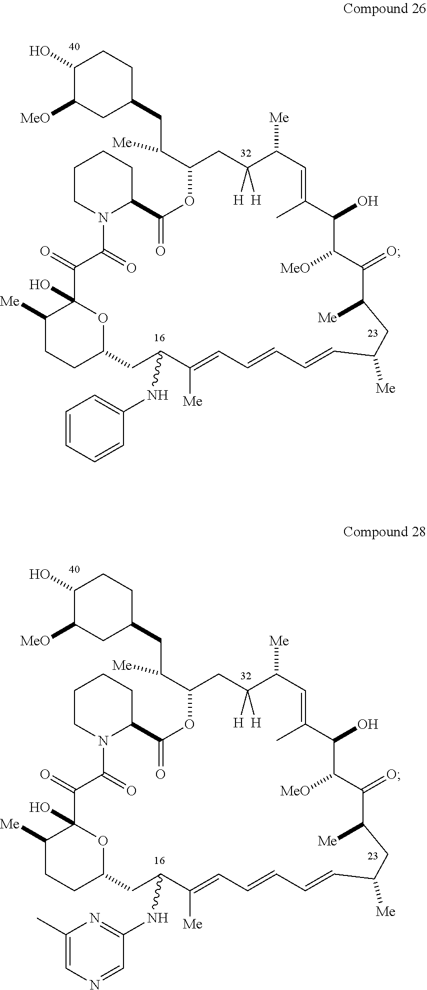 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(Nc3ccccc3)C[C@@H]3CC[C@@H](C)[C@@](O)(O3)C(=O)C(=O)N3CCCC[C@H]3C(=O)O2)CC[C@H]1O ; CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(Nc3cncc(C)n3)C[C@@H]3CC[C@@H](C)[C@@](O)(O3)C(=O)C(=O)N3CCCC[C@H]3C(=O)O2)CC[C@H]1O] 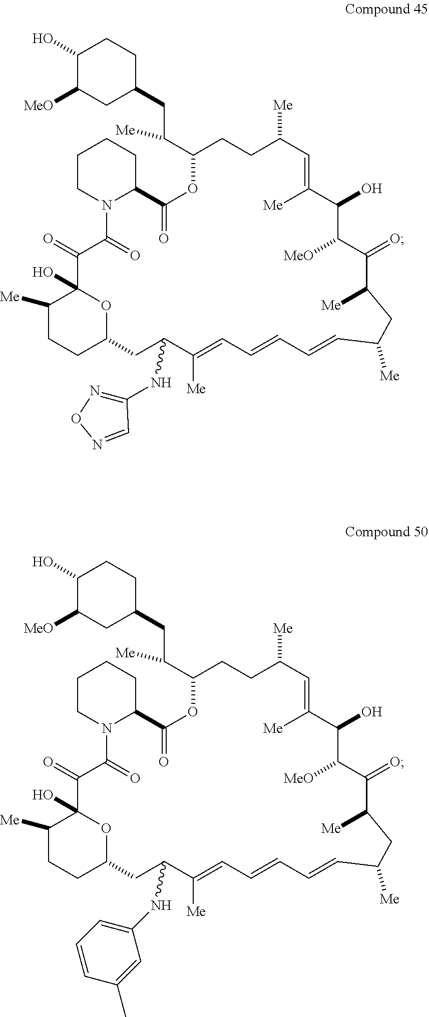 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(Nc3cnon3)C[C@@H]3CC[C@@H](C)[C@@](O)(O3)C(=O)C(=O)N3CCCC[C@H]3C(=O)O2)CC[C@H]1O ; CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(Nc3cccc(C)c3)C[C@@H]3CC[C@@H](C)[C@@](O)(O3)C(=O)C(=O)N3CCCC[C@H]3C(=O)O2)CC[C@H]1O] 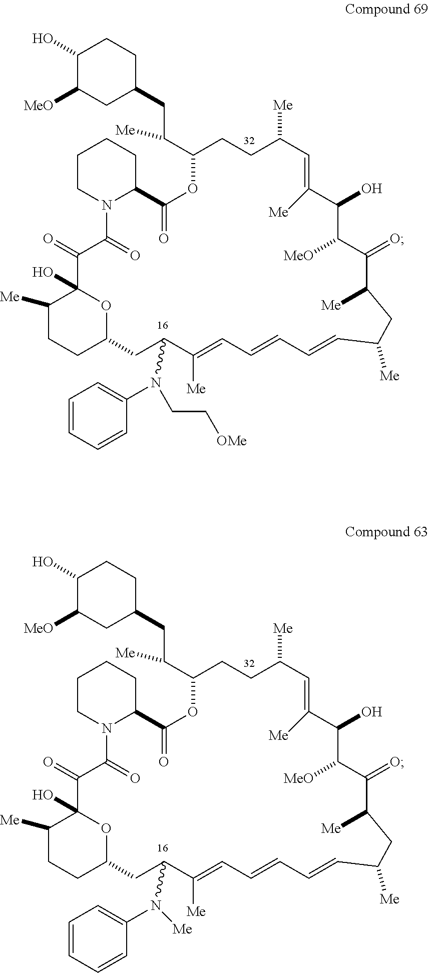 [SMILES: COCCN(c1ccccc1)C1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC[C@H](C)/C=C(\C)[C@@H](O)[C@@H](OC)C(=O)[C@H](C)C[C@H](C)/C=C/C=C/C=C/1C ; CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(N(C)c3ccccc3)C[C@@H]3CC[C@@H](C)[C@@](O)(O3)C(=O)C(=O)N3CCCC[C@H]3C(=O)O2)CC[C@H]1O] 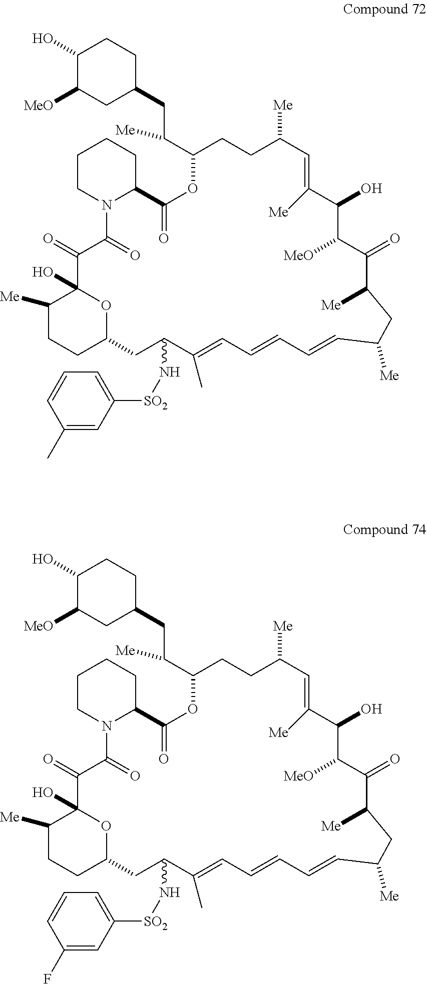 [SMILES: CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(NS(=O)(=O)c3cccc(C)c3)C[C@@H]3CC[C@@H](C)[C@@](O)(O3)C(=O)C(=O)N3CCCC[C@H]3C(=O)O2)CC[C@H]1O ; CO[C@@H]1C[C@H](C[C@@H](C)[C@@H]2CC[C@H](C)/C=C(\C)[C@@H](O)[C@@H](OC)C(=O)[C@H](C)C[C@H](C)/C=C/C=C/C=C(\C)C(NS(=O)(=O)c3cccc(F)c3)C[C@@H]3CC[C@@H](C)[C@@](O)(O3)C(=O)C(=O)N3CCCC[C@H]3C(=O)O2)CC[C@H]1O]
or a pharmaceutically acceptable salt thereof.

17. The compound of claim 1, wherein the compound binds selectively to FKBP12.

18. The compound of claim 17, wherein the compound inhibits S6K1 phosphorylation at least two-fold less efficiently than rapalog RAD001 in FKBP12 KO cells in comparison to the wild-type FKBP12 expressing cells.

19. The compound of claim 17, wherein the compound requires at least 10-fold higher concentration, in comparison to the rapalog RAD001, in FKBP12 KO cells in comparison to the wild-type FKBP12 expressing cells, to achieve 20% inhibition of S6K1 cell signaling.

20. The compound of claim 17, wherein the compound inhibits S6K1 phosphorylation at least ten-fold less efficiently than rapalog RAD001 in FKBP12 KO cells in comparison to the wild-type FKBP12 expressing cells.

21.-26. (canceled)

27. A pharmaceutical composition comprising a therapeutically effective amount of the compound of claim 1 or a pharmaceutically acceptable salt thereof in one or more pharmaceutically acceptable carriers.

28. A pharmaceutical combination comprising a therapeutically effective amount of the compound of claim 1 or a pharmaceutically acceptable salt thereof and one or more therapeutically active agents in one or more pharmaceutically acceptable carriers.

29. A method for treating a disorder or a disease mediated by the mTOR pathway in a subject in need thereof comprising administering to the subject a therapeutically effective amount of the compound of claim 1.

30. A method for treating a disease or disorder in a subject, wherein the target tissue, organ, or cells associated with the pathology of the disease or disorder has FKBP12 levels sufficient to inhibit mTORC1 comprising administering to the subject in need thereof a therapeutically effective amount of the compound of claim 1.

31. (canceled)

32. The method of claim 29, wherein the disease or disorder is selected from sarcopenia; skin atrophy; cherry angiomas; seborrheic keratoses; brain atrophy; atherosclerosis; arteriosclerosis; pulmonary emphysema; osteoporosis; osteoarthritis; high blood pressure; erectile dysfunction; cataracts; macular degeneration, glaucoma, stroke, cerebrovascular disease (strokes), chronic kidney disease, diabetes-associated kidney disease, impaired hepatic function, liver fibrosis, autoimmune hepatitis, endometrial hyperplasia, metabolic dysfunction, renovascular disease, hearing loss, mobility disability, cognitive decline, tendon stiffness, heart dysfunction such as cardiac hypertrophy and/or systolic and/or diastolic dysfunction and/or hypertension, heart dysfunction which results in a decline in ejection fraction, immune senescence, Parkinson's disease, Alzheimer's disease, cancer, immune-senescence leading to cancer due to a decrease in immune-surveillance, infections due to an decline in immune-function, chronic obstructive pulmonary disease (COPD), obesity, loss of taste, loss of olfaction, arthritis, epilepsy, cancer in which the tumor has elevated levels of mTORC1 signaling and/or sufficient levels of FKBP12 so as to allow inhibition of mTORC1, and type II diabetes.

33. A method of treating an age-related disorder or disease in a subject in need thereof comprising administering to the subject a therapeutically effective amount of the compound of claim 1, wherein the disorder or disease is selected from sarcopenia; skin atrophy; cherry angiomas; seborrheic keratoses; brain atrophy; atherosclerosis; arteriosclerosis; pulmonary emphysema; osteoporosis; osteoarthritis; high blood pressure; erectile dysfunction; cataracts; macular degeneration; glaucoma; stroke; cerebrovascular disease (strokes); chronic kidney disease; diabetes-associated kidney disease; impaired hepatic function; liver fibrosis; autoimmune hepatitis; endometrial hyperplasia; metabolic dysfunction; renovascular disease; hearing loss; mobility disability; cognitive decline; tendon stiffness; heart dysfunction, such as cardiac hypertrophy and/or systolic and/or diastolic dysfunction and/or hypertension; heart dysfunction that results in a decline in ejection fraction; immune senescence; Parkinson's disease; Alzheimer's disease; cancer; immune-senescence leading to cancer due to a decrease in immune-surveillance; infections due to an decline in immune-function; chronic obstructive pulmonary disease (COPD); obesity; loss of taste; loss of olfaction; arthritis; epilepsy, cancer in which the tumor has elevated levels of MTORC1 signaling and/or sufficient levels of FKBP12 so as to allow inhibition of mTORC1; and type II diabetes.

34. A method of treating cancer in a subject in need thereof comprising administering to the subject a therapeutically effective amount of the compound of claim 1.

35. The method of claim 34, wherein the cancer is selected from renal cancer, renal cell carcinoma, colorectal cancer, uterine sarcoma, endometrial uterine cancer, endometrial cancer, breast cancer, ovarian cancer, cervical cancer, gastric cancer, fibro-sarcoma, pancreatic cancer, liver cancer, melanoma, leukemia, multiple myeloma, nasopharyngeal cancer, prostate cancer, lung cancer, glioblastoma, bladder cancer, mesothelioma, head cancer, rhabdomyosarcoma, sarcoma, lymphoma, and neck cancer.

36.-53. (canceled)
